(19) United States
(12) Patent Application Publication
(10) Pub. No.: US 2016/0159789 A1
(43) Pub. Date: Jun. 9, 2016

(54) SUBSTITUTED PYRAZOLOPYRIDINES

(71) Applicant: BAYER PHARMA AKTIENGESELLSCHAFT, Berlin (DE)

(72) Inventors: Ulrich Klar, Berlin (DE); Lars Wortmann, Berlin (DE); Georg Kettschau, Berlin (DE); Keith Graham, Berlin (DE); Anja Richter, Berlin (DE); Philip Lienau, Berlin (DE); Florian Puehler, Wellesley, MA (US); Kirstin Petersen, Berlin (DE); Franziska Siegel, Berlin (DE); Detlev Sulzle, Berlin (DE)

(73) Assignee: BAYER PHARMA AKTIENGESELLSCHAFT, Berlin (DE)

(21) Appl. No.: 14/903,423

(22) PCT Filed: Jul. 4, 2014
(86) PCT No.: PCT/EP2014/064347

§ 371 Date: Jan. 7, 2016

(30) Foreign Application Priority Data
Jul. 8, 2013 (EP) 13175526.6
Nov. 28, 2013 (EP) 13194902.6
Nov. 29, 2013 (EP) 13195131.1

(51) Int. Cl. C07D 471/04; A61K 31/519; A61K 31/5377; C07D 519/00

(52) U.S. Cl. CPC C07D 471/04; C07D 519/00; A61K 31/519; A61K 31/5377

(57) ABSTRACT

The present invention relates to substituted pyrazolopyridine compounds of general formula I as described and defined herein, to methods of preparing said compounds, to intermediate compounds useful for preparing said compounds, to pharmaceutical compositions and combinations comprising said compounds and to the use of said compounds for manufacturing a pharmaceutical composition for the treatment or prophylaxis of a disease, in particular of a hyperproliferative and/or angiogenesis disorder, as a sole agent or in combination with other active ingredients.

The present invention relates to substituted pyrazolopyridine compounds of general formula I as described and defined herein, to methods of preparing said compounds, to intermediate compounds useful for preparing said compounds, to pharmaceutical compositions and combinations comprising said compounds and to the use of said compounds for manufacturing a pharmaceutical composition for the treatment or prophylaxis of a disease, in particular of a hyperproliferative and/or angiogenesis disorder, as a sole agent or in combination with other active ingredients.

### BACKGROUND OF THE INVENTION

The present invention relates to chemical compounds that inhibit MKNK1 kinase (also known as MAP Kinase interacting Kinase, Mnk1) and/or MKNK2 kinase (also known as MAP Kinase interacting Kinase, Mnk2).

Human MKNKs comprise a group of four proteins encoded by two genes (Gene symbols: MKNK1 and MKNK2) by alternative splicing. The b-forms lack a MAP kinase-binding domain situated at the C-terminus. The catalytic domains of the MKNK1 and MKNK2 are very similar and contain a unique DFD (Asp-Phe-Asp) motif in subdomain VII, which usually is DFG (Asp-Phe-Gly) in other protein kinases and suggested to alter ATP binding [Jauch et al., Structure 13, 1559-1568, 2005 and Jauch et al., EMBO J25, 4020-4032, 2006]. MKNK1a binds to and is activated by ERK and p38 MAP Kinases, but not by JNK1. MKNK2a binds to and is activated only by ERK. MKNK1b has low activity under all conditions and MKNK2b has a basal activity independent of ERK or p38 MAP Kinase. [Buxade M et al., Frontiers in Bioscience 5359-5374, May 1, 2008]

MKNKs have been shown to phosphorylate eukaryotic initiation factor 4E (eIF4E), heterogeneous nuclear RNA-binding protein A1 (hnRNP A1), polypyrimidine-tract binding protein-associated splicing factor (PSF), cytoplasmic phospholipase A2 (cPLA2) and Sprouty 2 (hSPRY2) [Buxade M et al., Frontiers in Bioscience 5359-5374, May 1, 2008].

eIF4E is an oncogene that is amplified in many cancers and is phosphorylated exclusively by MKNKs proteins as shown by KO-mouse studies [Konicek et al., Cell Cycle 7:16, 2466-2471, 2008; Ueda et al., Mol Cell Biol 24, 6539-6549, 2004]. eIF4E has a pivotal role in enabling the translation of cellular mRNAs. eIF4E binds the 7-methylguanosine cap at the 5′ end of cellular mRNAs and delivers them to the ribosome as part of the eIF4F complex, also containing eIF4G and eIF4A. Though all capped mRNAs require eIF4E for translation, a pool of mRNAs is exceptionally dependent on elevated eIF4E activity for translation. These so-called “weak mRNAs” are usually less efficiently translated due to their long and complex 5′UTR region and they encode proteins that play significant roles in all aspects of malignancy including VEGF, FGF-2, c-Myc, cyclin D1, survivin, BCL-2, MCL-1, MMP-9, heparanase, etc. Expression and function of eIF4E is elevated in multiple human cancers and directly related to disease progression [Konicek et al., Cell Cycle 7:16, 2466-2471, 2008].

MKNK1 and MKNK2 are the only kinases known to phosphorylate eIF4E at Ser209. Overall translation rates are not affected by eIF4E phosphorylation, but it has been suggested that eIF4E phosphorylation contributes to polysome formation (i.e. multiple ribosome on a single mRNA) that ultimately enables more efficient translation of “weak mRNAs” [Buxade M et al., Frontiers in Bioscience 5359-5374, May 1, 2008]. Alternatively, phosphorylation of eIF4E by MKNK proteins might facilitate eIF4E release from the 5′ cap so that the 48S complex can move along the “weak mRNA” in order to locate the start codon [Blagden S P and Willis A E, Nat Rev Clin Oncol. 8(5):280-91, 2011]. Accordingly, increased eIF4E phosphorylation predicts poor prognosis in non-small cell lung cancer patients [Yoshizawa et al., Clin Cancer Res. 16(1):240-8, 2010]. Further data point to a functional role of MKNK1 in carcinogenesis, as overexpression of constitutively active MKNK1, but not of kinase-dead MKNK1, in mouse embryo fibroblasts accelerates tumor formation [Chrestensen C. A. et al., Genes Cells 12, 1133-1140, 2007]. Moreover, increased phosphorylation and activity of MKNK proteins correlate with overexpression of HER2 in breast cancer [Chrestensen, C. A. et al., J. Biol. Chem. 282, 4243-4252, 2007]. Constitutively active, but not kinase-dead, MKNK1 also accelerated tumor growth in a model using Eμ-Myc transgenic hematopoietic stem cells to produce tumors in mice. Comparable results were achieved, when an eIF4E carrying a S209D mutation was analyzed. The S209D mutation mimicks a phosphorylation at the MKNK1 phosphorylation site. In contrast a non-phosphorylatable form of eIF4E attenuated tumor growth [Wendel H G, et al., Genes Dev. 21(24):3232-7, 2007]. A selective MKNK inhibitor that blocks eIF4E phosphorylation induces apoptosis and suppresses proliferation and soft agar growth of cancer cells in vitro. This inhibitor also suppresses outgrowth of experimental B16 melanoma pulmonary metastases and growth of subcutaneous HCT116 colon carcinoma xenograft tumors without affecting body weight [Konicek et al., Cancer Res. 71(5):1849-57, 2011]. Screening of a cohort of pancreatic ductal adenocarcinoma patients by immunohistochemistry showed that eIF4E phosphorylation correlated with disease grade, early onset of disease and worse prognosis. In addition it was suggested based on preclinical in vitro findings that the MNK/eIF4E pathway represents an escape route utilized by pancreatic ductal adenocarcinoma cells to withstand chemotherapeutic treatments (e.g Gemcitabine) [Adesso L, et al., Oncogene. 2012 Jul. 16]. Furthermore, it was observed that Rapamycin activated MKNK1 kinase activity in multiple myeloma cell lines and primary specimens by a MKNK-dependent mechanism. Pharmacological inhibition of MKNK activity or genetic silencing of MKNK1 prevented a rapalog-induced upregulation of c-myc IRES activity. Although Rapamycin, used alone, had little effect on myc protein expression, when combined with a MKNK inhibitor, myc protein expression was abrogated. These data provide a rationale for therapeutically targeting MKNK kinases for combined treatment with mTOR inhibitors [Shi Y et al., Oncogene. 2012 Feb. 27]. In summary, eIF4E phosphorylation through MKNK protein activity can promote cellular proliferation and survival and is critical for malignant transformation. Inhibition of MKNK activity may provide a tractable cancer therapeutic approach.

WO2006/136402(A1) and WO2007/059905(A2) (Develogen A G) disclose thienopyrimidin-4-amines and their use for the prophylaxis and/or treatment of diseases which can be influenced by the inhibition of the kinase activity of Mnk1 and/or Mnk2. The 4-amino-group is substituted by a substituted phenyl group. The WO publications do not disclose any biological data.

WO2010/023181(A1), WO2011/104334(A1), WO2011/104337(A1), WO2011/104338(A1) and WO2011/104340(A1) (Boehringer Ingelheim) relate to thienopyrimidin-4-amines for the prophylaxis and/or treatment of diseases which can be influenced by the inhibition of the kinase activity of Mnk1 and/or Mnk2.

WO2014/001973 (A1) discloses 4-(substituted-amino)-7H-pyrrolo[2,3-d]pyrimidines as LRRK2 inhibitors.

Specific substituted pyrazolopyridine compounds of general formula I of the present invention as defined herein, or a stereoisomer, a tautomer, an N-oxide, a hydrate, a solvate, or a salt thereof, or a mixture of same, as described and defined herein, and as hereinafter referred to as “compounds of the present invention”, or their pharmacological activity have not been disclosed so far.

It has now been found, and this constitutes the basis of the present invention, that said compounds of the present invention have surprising and advantageous properties.

In particular, said compounds of the present invention have surprisingly been found to effectively inhibit MKNK kinases and may therefore be used for the treatment or prophylaxis of diseases of uncontrolled cell growth, proliferation and/or survival, inappropriate cellular immune responses, or inappropriate cellular inflammatory responses or diseases which are accompanied with uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses is mediated by MKNK kinases, such as, for example, haematological tumours, solid tumours, and/or metastases thereof, e.g. leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof.

### SUMMARY OF THE INVENTION

The present invention covers compounds of general formula I:

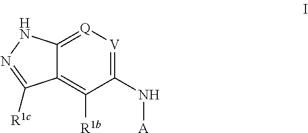

in which:
Q-V represents a group selected from: C(R^{1a})—N, N—C(R^{1a});
A represents a group selected from:

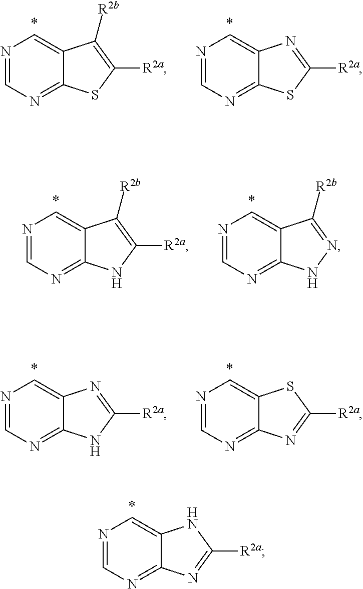

wherein * indicates the point of attachment of said groups to the rest of the molecule;
R^{1a} represents a hydrogen atom or a halogen atom or a group selected from: hydroxy-, cyano-, C₁-C₆-alkyl-, C₂-C₆-alkenyl-, C₂-C₆-alkynyl-, C₁-C₆-alkoxy-, halo-C₁-C₆-alkyl-, halo-C₁-C₆-alkoxy-, hydroxy-C₁-C₆-alkyl-, C₁-C₃-alkoxy-C₁-C₃-alkyl-, —N(R^{5b})R^{5c}, —SCF₃, —SF₅;
R^{1b} represents a hydrogen atom or a halogen atom or a group selected from: hydroxy-, cyano-, C₁-C₆-alkyl-, C₂-C₆-alkenyl-, C₂-C₆-alkynyl-, C₁-C₆-alkoxy-, halo-C₁-C₆-alkyl-, halo-C₁-C₆-alkoxy-, hydroxy-C₁-C₆-alkyl-, C₁-C₃-alkoxy-C₁-C₃-alkyl-, —N(R^{5b})R^{5c}, —SCF₃, —SF₅;
R^{1c} represents a hydrogen atom or a halogen atom or a group selected from: hydroxy-, cyano-, C₁-C₆-alkyl-, C₂-C₆-alkenyl-, C₂-C₆-alkynyl-, C₁-C₆-alkoxy-, halo-C₁-C₆-alkyl-, halo-C₁-C₆-alkoxy-, hydroxy-C₁-C₆-alkyl-, C₁-C₃-alkoxy-C₁-C₃-alkyl-, —N(R^{5b})R^{5c}, —SCF₃, —SF₅;
R^{2a} represents a hydrogen atom or a halogen atom or a group selected from:
   C₁-C₆-alkyl-, C₃-C₆-cycloalkyl-, 3- to 10-membered heterocycloalkyl-, 4- to 10-membered heterocycloalkenyl-, aryl-, heteroaryl-, cyano-, —(CH₂)_{q}—U—(CH₂)ₚ—R^{3a}; wherein said C₁-C₆-alkyl-, C₃-C₆-cycloalkyl-, 3- to 10-membered heterocycloalkyl-, 4- to 10-membered heterocycloalkenyl-, aryl- or heteroaryl-group is optionally substituted, identically or differently, with 1, 2, 3, 4 or 5 R⁴ groups;
R^{2b} represents a hydrogen atom or a halogen atom or a group selected from: C₁-C₆-alkyl-, C₃-C₆-cycloalkyl-, 3- to 10-membered heterocycloalkyl-, 4- to 10-membered heterocycloalkenyl-, aryl-, heteroaryl-, cyano-, —(CH₂)_{q}—U—(CH₂)ₚ—R^{3a}; wherein said C₁-C₆-alkyl-, C₃-C₆-cycloalkyl-, 3- to 10-membered heterocycloalkyl-, 4- to 10-membered heterocycloalkenyl-, aryl- or heteroaryl-group is optionally substituted, identically or differently, with 1, 2, 3, 4 or 5 R⁴ groups;
or
R^{2a} and R^{2b} together

represent —(CH₂)ᵣ-T-(CH₂)ₛ—;
T represents a group selected from: U, —C[R^{6a}][(C(R^{6b})(R^{6c}))ₜ—U—R^{3a}]—;
U represents a single bond or a bivalent group selected from: —O—, —S—, —S(═O)—, —S(═O)₂—, —S(═O)—N(R^{3b})—, —N(R^{3c})—S(═O)—, —S(═O)₂—N(R^{3b})—, —N(R^{3c})—S(═O)₂—, —C(═O)—, —N(R^{3b})—, —C(═O)—O—, —O—C(═O)—, —C(═S)—O—, —O—C(═S)—, —C(═O)—N(R^{3b})—, —N(R^{3c})—C(═O)—, —N(R^{3c})—C(═O)—N(R^{3b})—, —O—C(═O)—N(R^{3b})—, —N(R^{3c})—C(═O)—O—;
R^{3a} represents a hydrogen atom or a group selected from: C₁-C₆-alkyl-, C₃-C₆-cycloalkyl-, 3- to 10-membered heterocycloalkyl-, 4- to 10-membered heterocycloalkenyl-, aryl-, heteroaryl-; wherein said C₁-C₆-alkyl-, C₃-C₆-cycloalkyl-, 3- to 10-membered heterocycloalkyl-, 4- to 10-membered heterocycloalkenyl-, aryl- or heteroaryl-group is optionally substituted, identically or differently, with 1, 2, 3, 4 or 5 R⁴ groups;
R^{3b} represents a hydrogen atom or a group selected from: C₁-C₆-alkyl-, C₃-C₆-cycloalkyl-, 3- to 10-membered heterocycloalkyl-, 4- to 10-membered heterocycloalkenyl-, aryl-, heteroaryl-; wherein said C₁-C₆-alkyl-, C₃-C₆-cycloalkyl-, 3- to 10-membered heterocycloalkyl-, 4- to 10-membered heterocycloalkenyl-, aryl- or heteroaryl-group is optionally substituted, identically or differently, with 1, 2, 3, 4 or 5 R⁴ groups;
R^{3c} represents a hydrogen atom or a group selected from: C₁-C₆-alkyl-, C₃-C₆-cycloalkyl-, 3- to 10-membered heterocycloalkyl-, 4- to 10-membered heterocycloalkenyl-, aryl-, heteroaryl-, wherein said C₁-C₆-alkyl-, C₃-C₆-cycloalkyl-, 3- to 10-membered heterocycloalkyl-, 4- to 10-membered heterocycloalkenyl-, aryl- or heteroaryl-group is optionally substituted, identically or differently, with 1, 2, 3, 4 or 5 R⁴ groups;
or
N(R^{3b})R^{3a} together
   form a 3- to 10-membered heterocycloalkyl- or 4- to 10-membered heterocycloalkenyl-group, wherein said 3- to 10-membered heterocycloalkyl- or 4- to 10-membered heterocycloalkenyl-group is optionally substituted, identically or differently, with 1, 2, 3, 4 or 5 R⁴ groups;
R⁴ represents halo-, hydroxy-, oxo-(O═), cyano-, nitro-, C₁-C₆-alkyl-, C₂-C₆-alkenyl-, C₂-C₆-alkynyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R^{5c}—O—, —C(═O)—R^{5c}, —C(═O)—O—R^{5c}, —O—C(═O)—R^{5c}, —N(R^{5b})—C(═O)—R^{5c}, —N(R^{5c})—C(═O)—N(R^{5a})R^{5b}, —N(R^{5a})R^{5b}, —C(═O)—N(R^{5a})R^{5b}, R^{5c}—S—, R^{5c}—S(═O)—, R^{5c}—S(═O)₂—, —N(R^{5c})—S(═O)—R^{5b}, —S(═O)—N(R^{5a})R^{5b}, —N(R^{5c})—S(═O)₂—R^{5b}, —S(═O)₂—N(R^{5a})R^{5b}, —S(═O)═N(R^{5c})R^{5b}, —S(═O)═N(R^{5c})R^{5b} or —N═S(═O)(R^{5c})R^{5b};
R^{5a} represents a hydrogen atom, a C₁-C₆-alkyl-, C₃-C₆-cycloalkyl-, phenyl- or a 3- to 10-membered heterocycloalkyl-group; wherein said C₁-C₆-alkyl-group is optionally substituted once with phenyl-;
R^{5b} represents a hydrogen atom, a C₁-C₆-alkyl-, a C₃-C₆-cycloalkyl- or a 3- to 10-membered heterocycloalkyl-group;
R^{5C} represents a hydrogen atom, a C₁-C₆-alkyl-, a C₃-C₆-cycloalkyl- or a 3- to 10-membered heterocycloalkyl-group;
or
N(R^{5a})R^{5b}
   together form a 3- to 7-membered heterocycloalkyl-group;
R^{6a} represents a hydrogen atom or a group selected from: C₁-C₆-alkyl-, C₂-C₆-alkenyl-, C₂-C₆-alkynyl-; wherein said C₁-C₆-alkyl-, C₂-C₆-alkenyl- or C₂-C₆-alkynyl-group is optionally substituted, identically or differently, with 1, 2 or 3 R⁴ groups;
R^{6b} represents a hydrogen atom or a C₁-C₃-alkyl-group;
R^{6C} represents a hydrogen atom or a C₁-C₃-alkyl-group;
p represents an integer of 0, 1, 2 or 3;
q represents an integer of 0, 1, 2 or 3;
r represents an integer of 1, 2 or 3;
s represents an integer of 1, 2 or 3; and
t represents an integer of 0 or 1;
or a tautomer, an N-oxide, a hydrate, a solvate, or a salt thereof, or a mixture of same.

The present invention further relates to methods of preparing compounds of general formula I, to pharmaceutical compositions and combinations comprising said compounds, to the use of said compounds for manufacturing a pharmaceutical composition for the treatment or prophylaxis of a disease, as well as to intermediate compounds useful in the preparation of said compounds.

### DETAILED DESCRIPTION OF THE INVENTION

The terms as mentioned in the present text have preferably the following meanings:

The term “halogen atom”, “halo-” or “Hal-” is to be understood as meaning a fluorine, chlorine, bromine or iodine atom, preferably a fluorine, chlorine or bromine atom.

The term “C₁-C₁₀-alkyl” is to be understood as preferably meaning a linear or branched, saturated, monovalent hydrocarbon group having 1, 2, 3, 4, 5, 6, 7, 8, 9 or 10 carbon atoms, e.g. a methyl, ethyl, propyl, butyl, pentyl, hexyl, iso-propyl, iso-butyl, sec-butyl, tert-butyl, iso-pentyl, 2-methylbutyl, 1-methylbutyl, 1-ethylpropyl, 1,2-dimethylpropyl, neo-pentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl, or 1,2-dimethylbutyl group, or an isomer thereof. Particularly, said group has 1, 2, 3, 4, 5 or 6 carbon atoms (“C₁-C₆-alkyl”), more particularly, said group has 1, 2, 3 or 4 carbon atoms (“C₁-C₄-alkyl”), e.g. a methyl, ethyl, propyl, butyl, iso-propyl, iso-butyl, sec-butyl, tert-butyl group; even more particularly 1, 2 or 3 carbon atoms (“C₁-C₃-alkyl”), e.g. a methyl, ethyl, n-propyl- or iso-propyl group.

The term “C₁-C₁₀-alkylene” is to be understood as preferably meaning a linear or branched, saturated, bivalent hydrocarbon group having 1, 2, 3, 4, 5, 6, 7, 8, 9 or 10 carbon atoms, e.g. a methylene, ethylene, n-propylene, n-butylene, n-pentylene, 2-methylbutylene, n-hexylene, 3-methylpentylene group, or an isomer thereof. Particularly, said group is linear and has 2, 3, 4 or 5 carbon atoms (“C₂-C₅-alkylene”), e.g. an ethylene, n-propylene, n-butylene, n-pentylene group, more particularly 3 or 4 carbon atoms (“C₃-C₄-alkylene”), e.g. an n-propylene or n-butylene group.

The term “halo-C₁-C₆-alkyl” is to be understood as preferably meaning a linear or branched, saturated, monovalent hydrocarbon group in which the term “C₁-C₆-alkyl” is defined supra, and in which one or more hydrogen atoms is replaced by a halogen atom, in identically or differently, i.e. one halogen atom being independent from another. Particularly, said halogen atom is F. Said halo-C₁-C₆-alkyl group is, for example, —CF₃, —CHF₂, —CH₂F, —CF₂CF₃ or —CH₂CF₃.

The term “C₁-C₆-alkoxy” is to be understood as preferably meaning a linear or branched, saturated, monovalent, hydrocarbon group of formula —O—(C₁-C₆-alkyl), in which the term “C₁-C₆-alkyl” is defined supra, e.g. a methoxy, ethoxy, n-propoxy, iso-propoxy, n-butoxy, iso-butoxy, tert-butoxy, sec-butoxy, pentoxy, iso-pentoxy, or n-hexoxy group, or an isomer thereof.

The term “halo-C₁-C₆-alkoxy” is to be understood as preferably meaning a linear or branched, saturated, monovalent C₁-C₆-alkoxy group, as defined supra, in which one or more of the hydrogen atoms is replaced, in identically or differently, by a halogen atom. Particularly, said halogen atom is F. Said halo-C₁-C₆-alkoxy group is, for example, —OCF₃, —OCHF₂, —OCH₂F, —OCF₂CF₃ or —OCH₂CF₃.

The term “C₁-C₆-alkoxy-C₁-C₆-alkyl” is to be understood as preferably meaning a linear or branched, saturated, monovalent C₁-C₆-alkyl group, as defined supra, in which one or more of the hydrogen atoms is replaced, in identically or differently, by a C₁-C₆-alkoxy group, as defined supra, e.g. methoxyalkyl, ethoxyalkyl, propyloxyalkyl, iso-propoxyalkyl, butoxyalkyl, iso-butoxyalkyl, tert-butoxyalkyl, sec-butoxyalkyl, pentyloxyalkyl, iso-pentyloxyalkyl, hexyloxyalkyl group, or an isomer thereof.

The term “halo-C₁-C₆-alkoxy-C₁-C₆-alkyl” is to be understood as preferably meaning a linear or branched, saturated, monovalent C₁-C₆-alkoxy-C₁-C₆-alkyl group, as defined supra, in which one or more of the hydrogen atoms is replaced, in identically or differently, by a halogen atom. Particularly, said halogen atom is F. Said halo-C₁-C₆-alkoxy-C₁-C₆-alkyl group is, for example, —CH₂CH₂OCF₃, —CH₂CH₂OCHF₂, —CH₂CH₂OCH₂F, —CH₂CH₂OCF₂CF₃ or —CH₂CH₂OCH₂CF₃.

The term “C₂-C₁₀-alkenyl” is to be understood as preferably meaning a linear or branched, monovalent hydrocarbon group, which contains one or more double bonds, and which has 2, 3, 4, 5, 6, 7, 8, 9 or 10 carbon atoms, particularly 2, 3, 4, 5 or 6 carbon atoms (“C₂-C₆-alkenyl”), more particularly 2 or 3 carbon atoms (“C₂-C₃-alkenyl”), it being understood that in the case in which said alkenyl group contains more than one double bond, then said double bonds may be isolated from, or conjugated with, each other. Said alkenyl group is, for example, a vinyl, allyl, (E)-2-methylvinyl, (Z)-2-methylvinyl, homoallyl, (E)-but-2-enyl, (Z)-but-2-enyl, (E)-but-1-enyl, (Z)-but-1-enyl, pent-4-enyl, (E)-pent-3-enyl, (Z)-pent-3-enyl, (E)-pent-2-enyl, (Z)-pent-2-enyl, (E)-pent-1-enyl, (Z)-pent-1-enyl, hex-5-enyl, (E)-hex-4-enyl, (Z)-hex-4-enyl, (E)-hex-3-enyl, (Z)-hex-3-enyl, (E)-hex-2-enyl, (Z)-hex-2-enyl, (E)-hex-1-enyl, (Z)-hex-1-enyl, iso-propenyl, 2-methylprop-2-enyl, 1-methylprop-2-enyl, 2-methylprop-1-enyl, (E)-1-methylprop-1-enyl, (Z)-1-methylprop-1-enyl, 3-methylbut-3-enyl, 2-methylbut-3-enyl, 1-methylbut-3-enyl, 3-methylbut-2-enyl, (E)-2-methylbut-2-enyl, (Z)-2-methylbut-2-enyl, (E)-1-methylbut-2-enyl, (Z)-1-methylbut-2-enyl, (E)-3-methylbut-1-enyl, (Z)-3-methylbut-1-enyl, (E)-2-methylbut-1-enyl, (Z)-2-methylbut-1-enyl, (E)-1-methylbut-1-enyl, (Z)-1-methylbut-1-enyl, 1,1-dimethylprop-2-enyl, 1-ethylprop-1-enyl, 1-propylvinyl, 1-isopropylvinyl, 4-methylpent-4-enyl, 3-methylpent-4-enyl, 2-methylpent-4-enyl, 1-methylpent-4-enyl, 4-methylpent-3-enyl, (E)-3-methylpent-3-enyl, (Z)-3-methylpent-3-enyl, (E)-2-methylpent-3-enyl, (Z)-2-methylpent-3-enyl, (E)-1-methylpent-3-enyl, (Z)-1-methylpent-3-enyl, (E)-4-methylpent-2-enyl, (Z)-4-methylpent-2-enyl, (E)-3-methylpent-2-enyl, (Z)-3-methylpent-2-enyl, (E)-2-methylpent-2-enyl, (Z)-2-methylpent-2-enyl, (E)-1-methylpent-2-enyl, (Z)-1-methylpent-2-enyl, (E)-4-methylpent-1-enyl, (Z)-4-methylpent-1-enyl, (E)-3-methylpent-1-enyl, (Z)-3-methylpent-1-enyl, (E)-2-methylpent-1-enyl, (Z)-2-methylpent-1-enyl, (E)-1-methylpent-1-enyl, (Z)-1-methylpent-1-enyl, 3-ethylbut-3-enyl, 2-ethylbut-3-enyl, 1-ethylbut-3-enyl, (E)-3-ethylbut-2-enyl, (Z)-3-ethylbut-2-enyl, (E)-2-ethylbut-2-enyl, (Z)-2-ethylbut-2-enyl, (E)-1-ethylbut-2-enyl, (Z)-1-ethylbut-2-enyl, (E)-3-ethylbut-1-enyl, (Z)-3-ethylbut-1-enyl, 2-ethylbut-1-enyl, (E)-1-ethylbut-1-enyl, (Z)-1-ethylbut-1-enyl, 2-propylprop-2-enyl, 1-propylprop-2-enyl, 2-isopropylprop-2-enyl, 1-isopropylprop-2-enyl, (E)-2-propylprop-1-enyl, (Z)-2-propylprop-1-enyl, (E)-1-propylprop-1-enyl, (Z)-1-propylprop-1-enyl, (E)-2-isopropylprop-1-enyl, (Z)-2-isopropylprop-1-enyl, (E)-1-isopropylprop-1-enyl, (Z)-1-isopropylprop-1-enyl, (E)-3,3-dimethylprop-1-enyl, (Z)-3,3-dimethylprop-1-enyl, 1-(1,1-dimethylethyl)ethenyl, buta-1,3-dienyl, penta-1,4-dienyl, hexa-1,5-dienyl, or methylhexadienyl group. Particularly, said group is vinyl or allyl.

The term “C₂-C₁₀-alkynyl” is to be understood as preferably meaning a linear or branched, monovalent hydrocarbon group which contains one or more triple bonds, and which contains 2, 3, 4, 5, 6, 7, 8, 9 or 10 carbon atoms, particularly 2, 3, 4, 5 or 6 carbon atoms (“C₂-C₆-alkynyl”), more particularly 2 or 3 carbon atoms (“C₂-C₃-alkynyl”). Said C₂-C₁₀-alkynyl group is, for example, ethynyl, prop-1-ynyl, prop-2-ynyl, but-1-ynyl, but-2-ynyl, but-3-ynyl, pent-1-ynyl, pent-2-ynyl, pent-3-ynyl, pent-4-ynyl, hex-1-ynyl, hex-2-ynyl, hex-3-ynyl, hex-4-ynyl, hex-5-ynyl, 1-methylprop-2-ynyl, 2-methylbut-3-ynyl, 1-methylbut-3-ynyl, 1-methylbut-2-ynyl, 3-methylbut-1-ynyl, 1-ethylprop-2-ynyl, 3-methylpent-4-ynyl, 2-methylpent-4-ynyl, 1-methylpent-4-ynyl, 2-methylpent-3-ynyl, 1-methylpent-3-ynyl, 4-methylpent-2-ynyl, 1-methylpent-2-ynyl, 4-methylpent-1-ynyl, 3-methylpent-1-ynyl, 2-ethylbut-3-ynyl, 1-ethylbut-3-ynyl, 1-ethylbut-2-ynyl, 1-propylprop-2-ynyl, 1-isopropylprop-2-ynyl, 2,2-dimethylbut-3-ynyl, 1,1-dimethylbut-3-ynyl, 1,1-dimethylbut-2-ynyl, or 3,3-dimethylbut-1-ynyl group. Particularly, said alkynyl group is ethynyl, prop-1-ynyl, or prop-2-ynyl.

The term “C₃-C₁₀-cycloalkyl” is to be understood as meaning a saturated, monovalent, mono-, or bicyclic hydrocarbon ring which contains 3, 4, 5, 6, 7, 8, 9 or 10 carbon atoms (“C₃-C₁₀-cycloalkyl”). Said C₃-C₁₀-cycloalkyl group is for example, a monocyclic hydrocarbon ring, e.g. a cyclopropyl, cyclobutyl, cyclopentyl, cyclohexyl, cycloheptyl, cyclooctyl, cyclononyl or cyclodecyl, or a bicyclic hydrocarbon ring, e.g. a perhydropentalenylene or decalin ring. Particularly, said ring contains 3, 4, 5 or 6 carbon atoms (“C₃-C₆-cycloalkyl”).

The term “C₃-C₆-cycloalkyloxy” refers to a (C₃-C₆-cycloalkyl)-O— group in which “C₃-C₆-cycloalkyl” is as defined herein. Examples include, but are not limited to, cyclopropanoxy and cyclobutanoxy.

The term “C₄-C₁₀-cycloalkenyl” is to be understood as preferably meaning a non-aromatic, monovalent, mono- or bicyclic hydrocarbon ring which contains 4, 5, 6, 7, 8, 9 or 10 carbon atoms and one, two, three or four double bonds, in conjugation or not, as the size of said cycloalkenyl ring allows. Said C₄-C₁₀-cycloalkenyl group is for example, a monocyclic hydrocarbon ring, e.g. a cyclobutenyl, cyclopentenyl, or cyclohexenyl or a bicyclic hydrocarbon, e.g.:

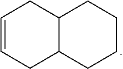

The term “C₅-C₈-cycloalkenyloxy” refers to a (C₅-C₈-cycloalkenyl)-O— group in which “C₅-C₈-cycloalkenyl” is as defined herein.

The term “3- to 10-membered heterocycloalkyl”, is to be understood as meaning a saturated, monovalent, mono- or bicyclic hydrocarbon ring which contains 2, 3, 4, 5, 6, 7, 8 or 9 carbon atoms, and one or more heteroatom-containing groups selected from —C(═O)—, —O—, —S—, —S(═O)—, —S(═O)₂—, —N(R^{a})—, in which R^{a} represents a hydrogen atom or a C₁-C₆-alkyl group; it being possible for said heterocycloalkyl group to be attached to the rest of the molecule via any one of the carbon atoms or, if present, the nitrogen atom. Heterospirocycloalkyl, heterobicycloalkyl and bridged heterocycloalkyl, as defined infra, are also included within the scope of this definition.

The term “heterospirocycloalkyl” is to be understood as meaning a saturated, monovalent bicyclic hydrocarbon radical in which the two rings share one common ring carbon atom, and wherein said bicyclic hydrocarbon radical contains 2, 3, 4, 5, 6, 7, 8 or 9 carbon atoms, and one or more heteroatom-containing groups selected from C(═O), O, S, S(═O), S(═O)₂, NR^{a}, in which R^{a} represents a hydrogen atom or a C₁-C₆-alkyl- or C₃-C₇-cycloalkyl-group; it being possible for said heterospirocycloalkyl-group to be attached to the rest of the molecule via any one of the carbon atoms or, if present, the nitrogen atom. Said heterospirocycloalkyl-group is, for example, azaspiro[2.3]hexyl-, azaspiro[3.3]heptyl-, oxaazaspiro[3.3]heptyl-, thiaazaspiro[3.3]heptyl-, oxaspiro[3.3]heptyl-, oxazaspiro[5.3]nonyl-, oxazaspiro[4.3]octyl-, oxazaspiro[5.5]undecyl-, diazaspiro[3.3]heptyl-, thiazaspiro[3.3]heptyl-, thiazaspiro[4.3]octyl-, or azaspiro[5.5]decyl-.

The term “heterobicycloalkyl” is to be understood as meaning a saturated, monovalent bicyclic hydrocarbon radical in which the two rings share two immediately adjacent ring atoms, and wherein said bicyclic hydrocarbon radical contains 2, 3, 4, 5, 6, 7, 8 or 9 carbon atoms, and one or more heteroatom-containing groups selected from C(═O), O, S, S(═O), S(═O)₂, NR^{a}, in which R^{a} represents a hydrogen atom or a C₁-C₆-alkyl- or C₃-C₇-cycloalkyl-group; it being possible for said heterobicycloalkyl-group to be attached to the rest of the molecule via any one of the carbon atoms or, if present, the nitrogen atom. Said heterobicycoalkyl-group is, for example, azabicyclo[3.3.0]octyl-, azabicyclo[4.3.0]nonyl-, diazabicyclo[4.3.0]nonyl-, oxazabicyclo[4.3.0]nonyl-, thiazabicyclo[4.3.0]nonyl-, or azabicyclo[4.4.0]decyl-.

The term “bridged heterocycloalkyl” is to be understood as meaning a saturated, monovalent bicyclic hydrocarbon radical in which the two rings share two common ring atoms which are not immediately adjacent, and wherein said bicyclic hydrocarbon radical contains 2, 3, 4, 5, 6, 7, 8 or 9 carbon atoms, and one or more heteroatom-containing groups selected from C(═O), O, S, S(═O), S(═O)₂, NR^{a}, in which R^{a} represents a hydrogen atom, or a C₁-C₆-alkyl- or C₃-C₇-cycloalkyl-group; it being possible for said bridged heterocycloalkyl-group to be attached to the rest of the molecule via any one of the carbon atoms or, if present, the nitrogen atom. Said bridged heterocycloalkyl-group is, for example, azabicyclo[2.2.1]heptyl-, oxazabicyclo[2.2.1]heptyl-, thiazabicyclo[2.2.1]heptyl-, diazabicyclo[2.2.1]heptyl-, azabicyclo[2.2.2]octyl-, diazabicyclo[2.2.2]octyl-, oxazabicyclo[2.2.2]octyl-, thiazabicyclo[2.2.2]octyl-, azabicyclo[3.2.1]octyl-, diazabicyclo[3.2.1]octyl-, oxazabicyclo[3.2.1]octyl-, thiazabicyclo[3.2.1]octyl-, azabicyclo[3.3.1]nonyl-, diazabicyclo[3.3.1]nonyl-, oxazabicyclo[3.3.1]nonyl-, thiazabicyclo[3.3.1]nonyl-, azabicyclo[4.2.1]nonyl-, diazabicyclo[4.2.1]nonyl-, oxazabicyclo[4.2.1]nonyl, thiazabicyclo[4.2.1]nonyl-, azabicyclo[3.3.2]decyl-, diazabicyclo[3.3.2]decyl-, oxazabicyclo[3.3.2]decyl-, thiazabicyclo[3.3.2]decyl-, or azabicyclo[4.2.2]decyl-.

Particularly, said 3- to 10-membered heterocycloalkyl can contain 2, 3, 4, or 5 carbon atoms, and one or more of the above-mentioned heteroatom-containing groups (a “3- to 6-membered heterocycloalkyl”), more particularly said 3- to 10-membered heterocycloalkyl can contain 4 or 5 carbon atoms, and one or more of the above-mentioned heteroatom-containing groups (a “5- to 6-membered heterocycloalkyl”).

Particularly, without being limited thereto, said 3- to 10-membered heterocycloalkyl can be a 4-membered ring, such as an azetidinyl, oxetanyl, or a 5-membered ring, such as tetrahydrofuranyl, dioxolinyl, pyrrolidinyl, imidazolidinyl, pyrazolidinyl or a 6-membered ring, such as tetrahydropyranyl, piperidinyl, morpholinyl, dithianyl, thiomorpholinyl, piperazinyl, or trithianyl, or a 7-membered ring, such as a diazepanyl ring, for example.

Said 3- to 10-membered heterocycloalkyl can be bicyclic, such as, without being limited thereto, a 5,5-membered ring, e.g. a hexahydrocyclopenta[c]pyrrol-2(1H)-yl ring, or a 5,6-membered bicyclic ring, e.g. a hexahydropyrrolo[1,2-a]pyrazin-2(1H)-yl ring.

The term “4- to 10-membered heterocycloalkenyl”, is to be understood as meaning an non-aromatic, unsaturated, monovalent, mono- or bicyclic hydrocarbon ring which contains 3, 4, 5, 6, 7, 8 or 9 carbon atoms, and one or more heteroatom-containing groups selected from —C(═O)—, —O—, —S—, —S(═O)—, —S(═O)₂—, —N(R^{a})—, in which R^{a} represents a hydrogen atom or a C₁-C₆-alkyl group; it being possible for said heterocycloalkenyl group to be attached to the rest of the molecule via any one of the carbon atoms or, if present, the nitrogen atom. Examples of said heterocycloalkenyl are e.g. 4H-pyranyl, 2H-pyranyl, 3H-diazirinyl, 2,5-dihydro-1H-pyrrolyl, [1,3]dioxolyl, 4H-[1,3,4]thiadiazinyl, 2,5-dihydrofuranyl, 2,3-dihydrofuranyl, 2,5-dihydrothiophenyl, 2,3-dihydrothiophenyl, 4,5-dihydrooxazolyl, or 4H-[1,4]thiazinyl group.

The term “aryl” is to be understood as preferably meaning a monovalent, aromatic or partially aromatic, mono-, bi- or tricyclic hydrocarbon ring having 6, 7, 8, 9, 10, 11, 12, 13 or 14 carbon atoms (a “C₆-C₁₄-aryl” group), particularly a ring having 6 carbon atoms (a “C₆-aryl” group), e.g. a phenyl group; or a biphenyl group, or a ring having 9 carbon atoms (a “C₉-aryl” group), e.g. an indanyl or indenyl group, or a ring having 10 carbon atoms (a “C₁₀-aryl” group), e.g. a tetralinyl, dihydronaphthyl, or naphthyl group, or a ring having 13 carbon atoms, (a “C₁₃-aryl” group), e.g. a fluorenyl group, or a ring having 14 carbon atoms, (a “C₁₄-aryl” group), e.g. an anthranyl group. Preferably, the aryl group is a phenyl group.

The term “heteroaryl” is understood as preferably meaning a monovalent, monocyclic, bicyclic or tricyclic aromatic ring system having 5, 6, 7, 8, 9, 10, 11, 12, 13 or 14 ring atoms (a “5- to 14-membered heteroaryl” group), particularly 5 or 6 or 9 or 10 atoms, and which contains at least one heteroatom which may be identical or different, said heteroatom being such as oxygen, nitrogen or sulfur, and in addition in each case can be benzocondensed. Particularly, heteroaryl is selected from thienyl, furanyl, pyrrolyl, oxazolyl, thiazolyl, imidazolyl, pyrazolyl, isoxazolyl, isothiazolyl, oxadiazolyl, triazolyl, thiadiazolyl, thia-4H-pyrazolyl etc., and benzo derivatives thereof, such as, for example, benzofuranyl, benzothienyl, benzoxazolyl, benzisoxazolyl, benzimidazolyl, benzotriazolyl, indazolyl, indolyl, isoindolyl, etc.; or pyridyl, pyridazinyl, pyrimidinyl, pyrazinyl, triazinyl, etc., and benzo derivatives thereof, such as, for example, quinolinyl, quinazolinyl, isoquinolinyl, etc.; or azocinyl, indolizinyl, purinyl, etc., and benzo derivatives thereof; or cinnolinyl, phthalazinyl, quinazolinyl, quinoxalinyl, naphthpyridinyl, pteridinyl, carbazolyl, acridinyl, phenazinyl, phenothiazinyl, phenoxazinyl, xanthenyl, or oxepinyl, etc.

In general, and unless otherwise mentioned, the heteroarylic or heteroarylenic radicals include all the possible isomeric forms thereof, e.g. the positional isomers thereof. Thus, for some illustrative non-restricting example, the term pyridinyl or pyridinylene includes pyridin-2-yl, pyridin-2-ylene, pyridin-3-yl, pyridin-3-ylene, pyridin-4-yl and pyridin-4-ylene; or the term thienyl or thienylene includes thien-2-yl, thien-2-ylene, thien-3-yl and thien-3-ylene.

The term “C₁-C₆”, as used throughout this text, e.g. in the context of the definition of “C₁-C₆-alkyl”, “C₁-C₆-haloalkyl”, “C₁-C₆-alkoxy”, or “C₁-C₆-haloalkoxy” is to be understood as meaning an alkyl group having a finite number of carbon atoms of 1 to 6, i.e. 1, 2, 3, 4, 5, or 6 carbon atoms. It is to be understood further that said term “C₁-C₆” is to be interpreted as any sub-range comprised therein, e.g. C₁-C₆, C₂-C₅, C₃-C₄, C₁-C₂, C₁-C₃, C₁-C₄, C₁-C₅; particularly C₁-C₂, C₁-C₃, C₁-C₄, C₁-C₅, C₁-C₆; more particularly C₁-C₄; in the case of “C₁-C₆-haloalkyl” or “C₁-C₆-haloalkoxy” even more particularly C₁-C₂.

Similarly, as used herein, the term “C₂-C₆”, as used throughout this text, e.g. in the context of the definitions of “C₂-C₆-alkenyl” and “C₂-C₆-alkynyl”, is to be understood as meaning an alkenyl group or an alkynyl group having a finite number of carbon atoms of 2 to 6, i.e. 2, 3, 4, 5, or 6 carbon atoms. It is to be understood further that said term “C₂-C₆” is to be interpreted as any sub-range comprised therein, e.g. C₂-C₆, C₃-C₅, C₃-C₄, C₂-C₃, C₂-C₄, C₂-C₅; particularly C₂-C₃.

Further, as used herein, the term “C₃-C₆”, as used throughout this text, e.g. in the context of the definition of “C₃-C₆-cycloalkyl”, is to be understood as meaning a cycloalkyl group having a finite number of carbon atoms of 3 to 6, i.e. 3, 4, 5 or 6 carbon atoms. It is to be understood further that said term “C₃-C₆” is to be interpreted as any sub-range comprised therein, e.g. C₃-C₆, C₄-C₅, C₃-C₅, C₃-C₄, C₄-C₆, C₅-C₆; particularly C₃-C₆.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds.

The term “optionally substituted” means optional substitution with the specified groups, radicals or moieties.

As used herein, the term “one or more”, e.g. in the definition of the substituents of the compounds of the general formulae of the present invention, is understood as meaning “one, two, three, four or five, particularly one, two, three or four, more particularly one, two or three, even more particularly one or two”.

As used herein, the term “leaving group” refers to an atom or a group of atoms that is displaced in a chemical reaction as stable species taking with it the bonding electrons. Preferably, a leaving group is selected from the group comprising: halo, in particular chloro, bromo or iodo, methanesulfonyloxy, p-toluenesulfonyloxy, trifluoromethanesulfonyloxy, nonafluorobutanesulfonyloxy, (4-bromo-benzene)sulfonyloxy, (4-nitro-benzene)sulfonyloxy, (2-nitro-benzene)-sulfonyloxy, (4-isopropyl-benzene)sulfonyloxy, (2,4,6-tri-isopropyl-benzene)-sulfonyloxy, (2,4,6-trimethyl-benzene)sulfonyloxy, (4-tertbutyl-benzene)sulfonyloxy, benzenesulfonyloxy, and (4-methoxy-benzene)sulfonyloxy.

As used herein, the term “protective group” is a protective group attached to a nitrogen in intermediates used for the preparation of compounds of the general formula I. Such groups are introduced e.g. by chemical modification of the respective amino group in order to obtain chemoselectivity in a subsequent chemical reaction. Protective groups for amino groups are descibed for example in T. W. Greene and P. G. M. Wuts in Protective Groups in Organic Synthesis, 3^{rd} edition, Wiley 1999; more specifically, said groups can be selected from substituted sulfonyl groups, such as mesyl-, tosyl- or phenylsulfonyl-, acyl groups such as benzoyl, acetyl or tetrahydropyranoyl-, or carbamate based groups, such as tert.-butoxycarbonyl (Boc), or can include silicon, as in e.g. 2-(trimethylsilyl)ethoxymethyl (SEM).

The invention includes all suitable isotopic variations of a compound of the invention. An isotopic variation of a compound of the invention is defined as one in which at least one atom is replaced by an atom having the same atomic number but an atomic mass different from the atomic mass usually or predominantly found in nature. Examples of isotopes that can be incorporated into a compound of the invention include isotopes of hydrogen, carbon, nitrogen, oxygen, phosphorus, sulphur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I, respectively. Certain isotopic variations of a compound of the invention, for example, those in which one or more radioactive isotopes such as ³H or ¹⁴O are incorporated, are useful in drug and/or substrate tissue distribution studies. Tritiated and carbon-14, i.e., ¹⁴C, isotopes are particularly preferred for their ease of preparation and detectability. Further, substitution with isotopes such as deuterium may afford certain therapeutic advantages resulting from greater metabolic stability, for example, increased in vivo half-life or reduced dosage requirements and hence may be preferred in some circumstances. Isotopic variations of a compound of the invention can generally be prepared by conventional procedures known by a person skilled in the art such as by the illustrative methods or by the preparations described in the examples hereafter using appropriate isotopic variations of suitable reagents.

Where the plural form of the word compounds, salts, polymorphs, hydrates, solvates and the like, is used herein, this is taken to mean also a single compound, salt, polymorph, isomer, hydrate, solvate or the like.

By “stable compound” or “stable structure” is meant a compound that is sufficiently robust to survive isolation to a useful degree of purity from a reaction mixture, and formulation into an efficacious therapeutic agent.

The compounds of this invention may contain one or more asymmetric centre, depending upon the location and nature of the various substituents desired. Asymmetric carbon atoms may be present in the (R) or (S) configuration, resulting in racemic mixtures in the case of a single asymmetric centre, and diastereomeric mixtures in the case of multiple asymmetric centres. In certain instances, asymmetry may also be present due to restricted rotation about a given bond, for example, the central bond adjoining two substituted aromatic rings of the specified compounds.

The compounds of the present invention may contain sulphur atoms which are asymmetric, such as an asymmetric sulphoxide or sulphoximine group, of structure:

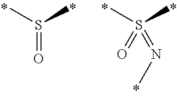

for example, in which * indicates atoms to which the rest of the molecule can be bound.

Substituents on a ring may also be present in either cis or trans form. It is intended that all such configurations (including enantiomers and diastereomers), are included within the scope of the present invention.

Preferred compounds are those which produce the more desirable biological activity. Separated, pure or partially purified isomers and stereoisomers or racemic or diastereomeric mixtures of the compounds of this invention are also included within the scope of the present invention. The purification and the separation of such materials can be accomplished by standard techniques known in the art.

Pure stereoisomers can be obtained by resolution of racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., chiral HPLC columns), with or without conventional derivatisation, optimally chosen to maximise the separation of the enantiomers. Suitable chiral HPLC columns are manufactured by Daicel, e.g., Chiracel OD and Chiracel OJ among many others, all routinely selectable. Enzymatic separations, with or without derivatisation, are also useful. The optically active compounds of this invention can likewise be obtained by chiral syntheses utilizing optically active starting materials.

In order to limit different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g. (R) or (S) isomers, or (E) or (Z) isomers, in any ratio. Isolation of a single stereoisomer, e.g. a single enantiomer or a single diastereomer, of a compound of the present invention may be achieved by any suitable state of the art method, such as chromatography, especially chiral chromatography, for example.

Further, the compounds of the present invention may exist as tautomers. For example, any compound of the present invention which contains a pyrazole moiety as a heteroaryl group for example can exist as a 1H tautomer, or a 2H tautomer, or even a mixture in any amount of the two tautomers, or a triazole moiety for example can exist as a 1H tautomer, a 2H tautomer, or a 4H tautomer, or even a mixture in any amount of said 1H, 2H and 4H tautomers, namely:

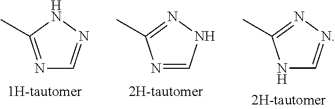

The present invention includes all possible tautomers of the compounds of the present invention as single tautomers, or as any mixture of said tautomers, in any ratio.

Further, the compounds of the present invention can exist as N-oxides, which are defined in that at least one nitrogen of the compounds of the present invention is oxidised. The present invention includes all such possible N-oxides.

The present invention also relates to useful forms of the compounds as disclosed herein, such as metabolites, hydrates, solvates, prodrugs, salts, in particular pharmaceutically acceptable salts, and co-precipitates.

The compounds of the present invention can exist as a hydrate, or as a solvate, wherein the compounds of the present invention contain polar solvents, in particular water, methanol or ethanol for example as structural element of the crystal lattice of the compounds. The amount of polar solvents, in particular water, may exist in a stoichiometric or non-stoichiometric ratio. In the case of stoichiometric solvates, e.g. a hydrate, hemi-, (semi-), mono-, sesqui-, di-, tri-, tetra-, penta-etc. solvates or hydrates, respectively, are possible. The present invention includes all such hydrates or solvates.

Further, the compounds of the present invention can exist in free form, e.g. as a free base, or as a free acid, or as a zwitterion, or can exist in the form of a salt. Said salt may be any salt, either an organic or inorganic addition salt, particularly any pharmaceutically acceptable organic or inorganic addition salt, customarily used in pharmacy.

The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, such as hydrochloric, hydrobromic, hydroiodic, sulfuric, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, persulfuric, 3-phenylpropionic, picric, pivalic, 2-hydroxyethanesulfonate, itaconic, sulfamic, trifluoromethanesulfonic, dodecylsulfuric, ethanesulfonic, benzenesulfonic, para-toluenesulfonic, methanesulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, hemisulfuric, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium or magnesium salt, an ammonium salt or a salt with an organic base which affords a physiologically acceptable cation, for example a salt with N-methyl-glucamine, dimethyl-glucamine, ethyl-glucamine, lysine, dicyclohexylamine, 1,6-hexadiamine, ethanolamine, glucosamine, sarcosine, serinol, tris-hydroxy-methyl-aminomethane, aminopropandiol, sovak-base, 1-amino-2,3,4-butantriol, or with a quarternary ammonium salt, such as tetramethylammonium, tetraethylammonium, tetra(n-propyl)ammonium, tetra (n-butyl)ammonium, or N-benzyl-N,N,N-trimethylammonium.

Those skilled in the art will further recognise that acid addition salts of the claimed compounds may be prepared by reaction of the compounds with the appropriate inorganic or organic acid via any of a number of known methods. Alternatively, alkali and alkaline earth metal salts of acidic compounds of the invention are prepared by reacting the compounds of the invention with the appropriate base via a variety of known methods.

The present invention includes all possible salts of the compounds of the present invention as single salts, or as any mixture of said salts, in any ratio.

As used herein, the term “in vivo hydrolysable ester” is understood as meaning an in vivo hydrolysable ester of a compound of the present invention containing a carboxy or hydroxy group, for example, a pharmaceutically acceptable ester which is hydrolysed in the human or animal body to produce the parent acid or alcohol. Suitable pharmaceutically acceptable esters for carboxy include for example alkyl, cycloalkyl and optionally substituted phenylalkyl, in particular benzyl esters, C₁-C₆ alkoxymethyl esters, e.g. methoxymethyl, C₁-C₆ alkanoyloxymethyl esters, e.g. pivaloyloxymethyl, phthalidyl esters, C₃-C₈ cycloalkoxy-carbonyloxy-C₁-C₆ alkyl esters, e.g. 1-cyclohexylcarbonyloxyethyl 1,3-dioxolen-2-onylmethyl esters, e.g. 5-methyl-1,3-dioxolen-2-onylmethyl; and C₁-C₆-alkoxycarbonyloxyethyl esters, e.g. 1-methoxycarbonyloxyethyl, and may be formed at any carboxy group in the compounds of this invention.

An in vivo hydrolysable ester of a compound of the present invention containing a hydroxy group includes inorganic esters such as phosphate esters and [alpha]-acyloxyalkyl ethers and related compounds which as a result of the in vivo hydrolysis of the ester breakdown to give the parent hydroxy group. Examples of [alpha]-acyloxyalkyl ethers include acetoxymethoxy and 2,2-dimethylpropionyloxymethoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. The present invention covers all such esters.

Furthermore, the present invention includes all possible crystalline forms, or polymorphs, of the compounds of the present invention, either as single polymorphs, or as a mixture of more than one polymorphs, in any ratio.

In accordance with a first aspect, the present invention covers compounds of general formula I:

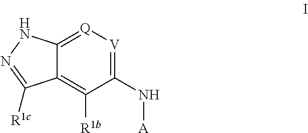

in which:
Q-V represents a group selected from: C(R^{1a})—N, N—C(R^{1a});
A represents a group selected from:

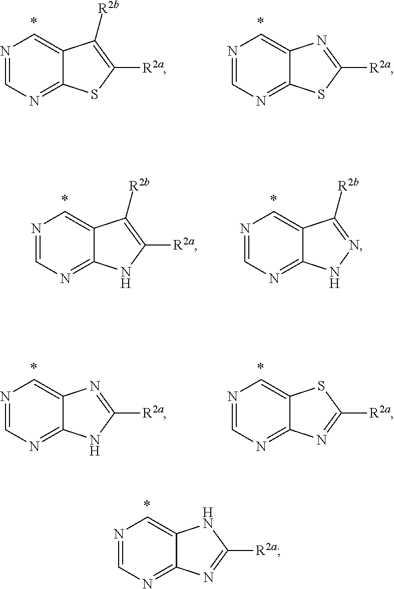

wherein * indicates the point of attachment of said groups to the rest of the molecule;
R^{1a} represents a hydrogen atom or a halogen atom or a group selected from: hydroxy-, cyano-, C₁-C₆-alkyl-, C₂-C₆-alkenyl-, C₂-C₆-alkynyl-, C₁-C₆-alkoxy-, halo-C₁-C₆-alkyl-, halo-C₁-C₆-alkoxy-, hydroxy-C₁-C₆-alkyl-, C₁-C₃-alkoxy-C₁-C₃-alkyl-, —N(R^{5b})R^{5c}, —SCF₃, —SF₅;
R^{1b} represents a hydrogen atom or a halogen atom or a group selected from: hydroxy-, cyano-, C₁-C₆-alkyl-, C₂-C₆-alkenyl-, C₂-C₆-alkynyl-, C₁-C₆-alkoxy-, halo-C₁-C₆-alkyl-, halo-C₁-C₆-alkoxy-, hydroxy-C₁-C₆-alkyl-, C₁-C₃-alkoxy-C₁-C₃-alkyl-, —N(R^{5b})R^{5c}, —SCF₃, —SF₅;
R^{1c} represents a hydrogen atom or a halogen atom or a group selected from: hydroxy-, cyano-, C₁-C₆-alkyl-, C₂-C₆-alkenyl-, C₂-C₆-alkynyl-, C₁-C₆-alkoxy-, halo-C₁-C₆-alkyl-, halo-C₁-C₆-alkoxy-, hydroxy-C₁-C₆-alkyl-, C₁-C₃-alkoxy-C₁-C₃-alkyl-, —N(R^{5b})R^{5c}, —SCF₃, —SF₅;
R^{2a} represents a hydrogen atom or a halogen atom or a group selected from: C₁-C₆-alkyl-, C₃-C₆-cycloalkyl-, 3- to 10-membered heterocycloalkyl-, 4- to 10-membered heterocycloalkenyl-, aryl-, heteroaryl-, cyano-, —(CH₂)_{q}—U—(CH₂)ₚ—R^{3a}; wherein said C₁-C₆-alkyl-, C₃-C₆-cycloalkyl-, 3- to 10-membered heterocycloalkyl-, 4- to 10-membered heterocycloalkenyl-, aryl- or heteroaryl-group is optionally substituted, identically or differently, with 1, 2, 3, 4 or 5 R⁴ groups;
R^{2b} represents a hydrogen atom or a halogen atom or a group selected from: C₁-C₆-alkyl-, C₃-C₆-cycloalkyl-, 3- to 10-membered heterocycloalkyl-, 4- to 10-membered heterocycloalkenyl-, aryl-, heteroaryl-, cyano-, —(CH₂)_{q}—U—(CH₂)ₚ—R^{3a}; wherein said C₁-C₆-alkyl-, C₃-C₆-cycloalkyl-, 3- to 10-membered heterocycloalkyl-, 4- to 10-membered heterocycloalkenyl-, aryl- or heteroaryl-group is optionally substituted, identically or differently, with 1, 2, 3, 4 or 5 R⁴ groups;
or
R^{2a} and R^{2b} together

represent —(CH₂)ᵣ-T-(CH₂)ₛ—
T represents a group selected from: U, —C[R^{6a}][(C(R^{6b})(R^{6c}))ₜ—U—R^{3a}]—;
U represents a single bond or a bivalent group selected from: —O—, —S—, —S(═O)—, —S(═O)₂—, —S(═O)—N(R^{3b})—, —N(R^{3c})—S(═O)—, —S(═O)₂—N(R^{3b})—, —N(R^{3c})—S(═O)₂—, —C(═O)—, —N(R^{3b})—, —C(═O)—O—, —O—C(═O)—, —C(═S)—O—, —O—C(═S)—, —C(═O)—N(R^{3b})—, —N(R^{3c})—C(═O)—, —N(R^{3c})—C(═O)—N(R^{3b})—, —O—C(═O)—N(R^{3b})—, —N(R^{3c})—C(═O)—O—;
R^{3a} represents a hydrogen atom or a group selected from: C₁-C₆-alkyl-, C₃-C₆-cycloalkyl-, 3- to 10-membered heterocycloalkyl-, 4- to 10-membered heterocycloalkenyl-, aryl-, heteroaryl-; wherein said C₁-C₆-alkyl-, C₃-C₆-cycloalkyl-, 3- to 10-membered heterocycloalkyl-, 4- to 10-membered heterocycloalkenyl-, aryl- or heteroaryl-group is optionally substituted, identically or differently, with 1, 2, 3, 4 or 5 R⁴ groups;
R^{3b} represents a hydrogen atom or a group selected from: C₁-C₆-alkyl-, C₃-C₆-cycloalkyl-, 3- to 10-membered heterocycloalkyl-, 4- to 10-membered heterocycloalkenyl-, aryl-, heteroaryl-; wherein said C₁-C₆-alkyl-, C₃-C₆-cycloalkyl-, 3- to 10-membered heterocycloalkyl-, 4- to 10-membered heterocycloalkenyl-, aryl- or heteroaryl-group is optionally substituted, identically or differently, with 1, 2, 3, 4 or 5 R⁴ groups;
R^{3c} represents a hydrogen atom or a group selected from: C₁-C₆-alkyl-, C₃-C₆-cycloalkyl-, 3- to 10-membered heterocycloalkyl-, 4- to 10-membered heterocycloalkenyl-, aryl-, heteroaryl-, wherein said C₁-C₆-alkyl-, C₃-C₆-cycloalkyl-, 3- to 10-membered heterocycloalkyl-, 4- to 10-membered heterocycloalkenyl-, aryl- or heteroaryl-group is optionally substituted, identically or differently, with 1, 2, 3, 4 or 5 R⁴ groups;
or
N(R^{3b})R^{3a} together
   form a 3- to 10-membered heterocycloalkyl- or 4- to 10-membered heterocycloalkenyl-group; wherein said 3- to 10-membered heterocycloalkyl- or 4- to 10-membered heterocycloalkenyl-group is optionally substituted, identically or differently, with 1, 2, 3, 4 or 5 R⁴ groups;
R⁴ represents halo-, hydroxy-, oxo-(O═), cyano-, nitro-, C₁-C₆-alkyl-, C₂-C₆-alkenyl-, C₂-C₆-alkynyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R^{5c}—O—, —C(═O)—R^{5c}, —C(═O)—O—R^{5c}, —O—C(═O)—R^{5c}, —N(R^{5b})—C(═O)—R^{5c}, —N(R^{5c})—C(═O)—N(R^{5a})R^{5b}, —N(R^{5a})R^{5b}, —C(═O)—N(R^{5a})R^{5b}, R^{5c}—S—, R^{5c}—S(═O)—, R^{5c}—S(═O)₂—, —N(R^{5c})—S(═O)—R^{5b}, —S(═O)—N(R^{5a})R^{5b}, —N(R^{5c})—S(═O)₂—R^{5b}, —S(═O)₂—N(R^{5a})R^{5b}, —S(═O)═N(R^{5c})R^{5b}, —S(═O)═N(R^{5c})R^{5b} or —N═S(═O)(R^{5c})R^{5b};
R^{5a} represents a hydrogen atom, a C₁-C₆-alkyl-, C₃-C₆-cycloalkyl-, phenyl- or a 3- to 10-membered heterocycloalkyl-group; wherein said C₁-C₆-alkyl-group is optionally substituted once with phenyl-;
R^{5b} represents a hydrogen atom, a C₁-C₆-alkyl-, a C₃-C₆-cycloalkyl- or a 3- to 10-membered heterocycloalkyl-group;
R^{5C} represents a hydrogen atom, a C₁-C₆-alkyl-, a C₃-C₆-cycloalkyl- or a 3- to 10-membered heterocycloalkyl-group;
or
N(R^{5a})R^{5b}

together form a 3- to 7-membered heterocycloalkyl-group;
R^{6a} represents a hydrogen atom or a group selected from: C₁-C₆-alkyl-, C₂-C₆-alkenyl-, C₂-C₆-alkynyl-; wherein said C₁-C₆-alkyl-, C₂-C₆-alkenyl- or C₂-C₆-alkynyl-group is optionally substituted, identically or differently, with 1, 2 or 3 R⁴ groups;
R^{6b} represents a hydrogen atom or a C₁-C₃-alkyl-group;
R^{6c} represents a hydrogen atom or a C₁-C₃-alkyl-group;
p represents an integer of 0, 1, 2 or 3;
q represents an integer of 0, 1, 2 or 3;
r represents an integer of 1, 2 or 3;
s represents an integer of 1, 2 or 3; and
t represents an integer of 0 or 1;
or a tautomer, an N-oxide, a hydrate, a solvate, or a salt thereof, or a mixture of same.

In a preferred embodiment, the invention relates to compounds of formula I, supra, wherein Q-V represents N—C(R^{1a}).

In another preferred embodiment, the invention relates to compounds of formula I, supra, wherein Q-V represents C(R^{1a})—N.

In another preferred embodiment, the invention relates to compounds of formula I, supra, wherein A represents a group selected from:

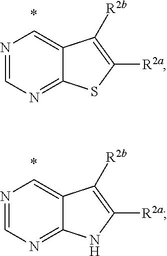

wherein * indicates the point of attachment of said groups to the rest of the molecule.

In another preferred embodiment, the invention relates to compounds of formula I, supra, wherein A represents:

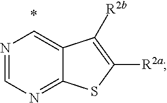

wherein * indicates the point of attachment of said group to the rest of the molecule.

In another preferred embodiment, the invention relates to compounds of formula I, supra, wherein A represents:

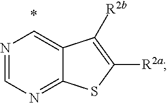

wherein * indicates the point of attachment of said group to the rest of the molecule, and wherein R^{2a} and R^{2b} together represent —(CH₂)ᵣ-T-(CH₂)ₛ—.

In another preferred embodiment, the invention relates to compounds of formula I, supra, wherein A represents

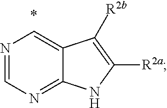

wherein * indicates the point of attachment of said group to the rest of the molecule.

In another preferred embodiment, the invention relates to compounds of formula I, supra, wherein R^{1a} represents a hydrogen atom or a halogen atom or a group selected from: cyano-, C₁-C₆-alkyl-, C₁-C₆-alkoxy-, halo-C₁-C₆-alkyl-, halo-C₁-C₆-alkoxy-, hydroxy-C₁-C₆-alkyl-, C₁-C₃-alkoxy-C₁-C₃-alkyl-.

In another preferred embodiment, the invention relates to compounds of formula I, supra, wherein R^{1a} represents a hydrogen atom or a group selected from: cyano-, C₁-C₆-alkyl-, C₁-C₆-alkoxy-, halo-C₁-C₆-alkyl-, halo-C₁-C₆-alkoxy-, hydroxy-C₁-C₆-alkyl-, C₁-C₃-alkoxy-C₁-C₃-alkyl-.

In another preferred embodiment, the invention relates to compounds of formula I, supra, wherein R^{1a} represents a hydrogen atom or a group selected from: C₁-C₃-alkyl-, C₁-C₃-alkoxy-, halo-C₁-C₃-alkyl-, halo-C₁-C₃-alkoxy-, hydroxy-C₁-C₃-alkyl-, C₁-C₃-alkoxy-C₁-C₃-alkyl-.

In another preferred embodiment, the invention relates to compounds of formula I, supra, wherein R^{1a} represents a hydrogen atom or a group selected from: C₁-C₃-alkyl-, C₁-C₃-alkoxy-, halo-C₁-C₃-alkyl-.

In another preferred embodiment, the invention relates to compounds of formula I, supra, wherein R^{1a} represents a hydrogen atom or a C₁-C₃-alkyl-group.

In another preferred embodiment, the invention relates to compounds of formula I, supra, wherein R^{1a} represents a hydrogen atom or a hydroxy-group or a C₁-C₃-alkoxy-group.

In another preferred embodiment, the invention relates to compounds of formula I, supra, wherein R^{1a} represents a hydrogen atom or a hydroxy- or methoxy-group.

In another preferred embodiment, the invention relates to compounds of formula I, supra, wherein R^{1a} represents a hydroxy-group.

In another preferred embodiment, the invention relates to compounds of formula I, supra, wherein R^{1a} represents a methoxy-group.

In another preferred embodiment, the invention relates to compounds of formula I, supra, wherein R^{1a} represents a hydrogen atom.

In another preferred embodiment, the invention relates to compounds of formula (I), supra, wherein Q-V represents N—C(R^{1a}) and R^{1a} represents a group selected from C₁-C₃-alkyl-, C₁-C₃-alkoxy-, halo-.

In another preferred embodiment, the invention relates to compounds of formula (I), supra, wherein Q-V represents N—C(R^{1a}) and R^{1a} represents a group selected from C₁-C₃-alkoxy-, halo-.

In another preferred embodiment, the invention relates to compounds of formula (I), supra, wherein Q-V represents N—C(R^{1a}) and R^{1a} represents a C₁-C₃-alkoxy-group, preferably a methoxy-group.

In another preferred embodiment, the invention relates to compounds of formula (I), supra, wherein Q-V represents C(R^{1a})—N and R^{1a} represents a hydrogen atom.

In another preferred embodiment, the invention relates to compounds of formula I, supra, wherein R^{1b} represents a hydrogen atom or a halogen atom or a group selected from: cyano-, C₁-C₆-alkyl-, C₁-C₆-alkoxy-, halo-C₁-C₆-alkyl-, halo-C₁-C₆-alkoxy-, hydroxy-C₁-C₆-alkyl-, C₁-C₃-alkoxy-C₁-C₃-alkyl-.

In another preferred embodiment, the invention relates to compounds of formula I, supra, wherein R^{1b} represents a hydrogen atom or a group selected from: cyano-, C₁-C₆-alkyl-, C₁-C₆-alkoxy-, halo-C₁-C₆-alkyl-, halo-C₁-C₆-alkoxy-, hydroxy-C₁-C₆-alkyl-, C₁-C₃-alkoxy-C₁-C₃-alkyl-.

In another preferred embodiment, the invention relates to compounds of formula I, supra, wherein R^{1b} represents a hydrogen atom or a group selected from: C₁-C₃-alkyl-, C₁-C₃-alkoxy-, halo-C₁-C₃-alkyl-, halo-C₁-C₃-alkoxy-, hydroxy-C₁-C₃-alkyl-, C₁-C₃-alkoxy-C₁-C₃-alkyl-.

In another preferred embodiment, the invention relates to compounds of formula I, supra, wherein R^{1b} represents a hydrogen atom or a group selected from: C₁-C₃-alkyl-, C₁-C₃-alkoxy-, halo-C₁-C₃-alkyl-.

In another preferred embodiment, the invention relates to compounds of formula I, supra, wherein R^{1b} represents a hydrogen atom or a C₁-C₃-alkyl-group.

In another preferred embodiment, the invention relates to compounds of formula I, supra, wherein R^{1b} represents a hydrogen atom.

In another preferred embodiment, the invention relates to compounds of formula I, supra, wherein R^{1c} represents a hydrogen atom or a halogen atom or a group selected from: cyano-, C₁-C₆-alkyl-, C₁-C₆-alkoxy-, halo-C₁-C₆-alkyl-, halo-C₁-C₆-alkoxy-, hydroxy-C₁-C₆-alkyl-, C₁-C₃-alkoxy-C₁-C₃-alkyl-.

In another preferred embodiment, the invention relates to compounds of formula I, supra, wherein R^{1c} represents a hydrogen atom or a group selected from: cyano-, C₁-C₆-alkyl-, C₁-C₆-alkoxy-, halo-C₁-C₆-alkyl-, halo-C₁-C₆-alkoxy-, hydroxy-C₁-C₆-alkyl-, C₁-C₃-alkoxy-C₁-C₃-alkyl-.

In another preferred embodiment, the invention relates to compounds of formula I, supra, wherein R^{1c} represents a hydrogen atom or a group selected from: C₁-C₃-alkyl-, C₁-C₃-alkoxy-, halo-C₁-C₃-alkyl-, halo-C₁-C₃-alkoxy-, hydroxy-C₁-C₃-alkyl-, C₁-C₃-alkoxy-C₁-C₃-alkyl-.

In another preferred embodiment, the invention relates to compounds of formula I, supra, wherein R^{1c} represents a hydrogen atom or a group selected from: C₁-C₃-alkyl-, C₁-C₃-alkoxy-, halo-C₁-C₃-alkyl-.

In another preferred embodiment, the invention relates to compounds of formula I, supra, wherein R^{1c} represents a hydrogen atom or a C₁-C₃-alkyl-group.

In another preferred embodiment, the invention relates to compounds of formula I, supra, wherein R^{1c} represents a hydrogen atom.

In another preferred embodiment, the invention relates to compounds of formula I, supra, wherein each of R^{1a}, R^{1b}, and R^{1c} represents a hydrogen atom.

In another preferred embodiment, the invention relates to compounds of formula I, supra, wherein each of R^{1b} and R^{1c} represents a hydrogen atom, and wherein R^{1a} represents a methoxy-group.

In another preferred embodiment, the invention relates to compounds of formula I, supra, wherein R^{2a} represents a hydrogen atom or a halogen atom or a group selected from: C₁-C₆-alkyl-, C₃-C₆-cycloalkyl-, 3- to 10-membered heterocycloalkyl-, halo-C₁-C₃-alkyl-, cyano-, —(CH₂)_{q}—U—(CH₂)ₚ—R^{3a}; wherein said C₁-C₆-alkyl-, C₃-C₆-cycloalkyl-, 3- to 10-membered heterocycloalkyl- or 4- to 10-membered heterocycloalkenyl-group is optionally substituted, identically or differently, with 1, 2 or 3 R⁴ groups, with the proviso that said halo-C₁-C₃-alkyl-group does not contain more than 5 halogen atoms.

In another preferred embodiment, the invention relates to compounds of formula I, supra, wherein R^{2a} represents a hydrogen atom or a halogen atom or a group selected from: C₁-C₆-alkyl-, cyano-, —(CH₂)_{q}—U—(CH₂)ₚ—R^{3a}; wherein said C₁-C₆-alkyl-group is optionally substituted, identically or differently, with 1, 2 or 3 R⁴ groups.

In another preferred embodiment, the invention relates to compounds of formula I, supra, wherein R^{2a} represents a hydrogen atom or a halogen atom or a group selected from: C₁-C₆-alkyl-, halo-C₁-C₃-alkyl-, cyano-, —(CH₂)_{q}—U—(CH₂)ₚ—R^{3a}; wherein said C₁-C₆-alkyl-group is optionally substituted with 1 R⁴ group,

with the proviso that said halo-C₁-C₃-alkyl-group does not contain more than 5 halogen atoms.

In another preferred embodiment, the invention relates to compounds of formula I, supra, wherein R^{2a} represents a hydrogen atom or a group selected from: C₁-C₆-alkyl-, —(CH₂)_{q}—U—(CH₂)ₚ—R^{3a}; wherein said C₁-C₆-alkyl-group is optionally substituted with 1 R⁴ group.

In another preferred embodiment, the invention relates to compounds of formula I, supra, wherein R^{2a} represents a hydrogen atom or a group selected from: C₁-C₆-alkyl-, —(CH₂)_{q}—U—(CH₂)ₚ—R^{3a}

In another preferred embodiment, the invention relates to compounds of formula I, supra, wherein R^{2a} represents a hydrogen atom or a C₁-C₆-alkyl-group.

In another preferred embodiment, the invention relates to compounds of formula I, supra, wherein R^{2a} represents a hydrogen atom or a C₁-C₃-alkyl-group.

In another preferred embodiment, the invention relates to compounds of formula I, supra, wherein R^{2a} represents a hydrogen atom.

In another preferred embodiment, the invention relates to compounds of formula I, supra, wherein R^{2a} represents a —(CH₂)_{q}—U—(CH₂)ₚ—R^{3a} group.

In another preferred embodiment, the invention relates to compounds of formula I, supra, wherein R^{2b} represents a hydrogen atom or a halogen atom or a group selected from: C₁-C₆-alkyl-, halo-C₁-C₃-alkyl-, cyano-, —(CH₂)_{q}—U—(CH₂)ₚ—R^{3a}; wherein said C₁-C₆-alkyl-group is optionally substituted, identically or differently, with 1, 2 or 3 R⁴ groups, with the proviso that said halo-C₁-C₃-alkyl-group does not contain more than 5 halogen atoms.

In another preferred embodiment, the invention relates to compounds of formula I, supra, wherein R^{2b} represents a hydrogen atom or a halogen atom or a group selected from: C₁-C₆-alkyl-, halo-C₁-C₃-alkyl-, —(CH₂)_{q}—U—(CH₂)ₚ—R^{3a}; wherein said C₁-C₆-alkyl-group is optionally substituted with 1 R⁴ group,

with the proviso that said halo-C₁-C₃-alkyl-group does not contain more than 5 halogen atoms.

In another preferred embodiment, the invention relates to compounds of formula I, supra, wherein R^{2b} represents a hydrogen atom or a halogen atom or a group selected from: C₁-C₃-alkyl-, halo-C₁-C₃-alkyl-; wherein said C₁-C₃-alkyl-group is optionally substituted with 1 R⁴ group,

with the proviso that said halo-C₁-C₃-alkyl-group does not contain more than 5 halogen atoms.

In another preferred embodiment, the invention relates to compounds of formula I, supra, wherein R^{2b} represents a —(CH₂)_{q}—U—(CH₂)ₚ—R^{3a} group.

In another preferred embodiment, the invention relates to compounds of formula I, supra, wherein R^{2b} represents a hydrogen atom or a C₁-C₃-alkyl-group.

In another preferred embodiment, the invention relates to compounds of formula I, supra, wherein R^{2b} represents a hydrogen atom.

In another preferred embodiment, the invention relates to compounds of formula I, supra, wherein one of R^{2a} and R^{2b} represents —(CH₂)_{q}—U—(CH₂)ₚ—R^{3a} and the other one represents a hydrogen atom or a C₁-C₃-alkyl-group.

In another preferred embodiment, the invention relates to compounds of formula I, supra, wherein R^{2a} and R^{2b} together represent —(CH₂)ᵣ-T-(CH₂)ₛ—.

In another preferred embodiment, the invention relates to compounds of formula I, supra, wherein R^{2a} and R^{2b} together represent —(CH₂)₂-T-CH₂—.

In another preferred embodiment, the invention relates to compounds of formula I, supra, wherein R^{2a} and R^{2b} together represent —CH₂-T-(CH₂)₂—.

In another preferred embodiment, the invention relates to compounds of formula I, supra, wherein T represents —C[R^{6a}][(C(R^{6b})(R^{6c}))ₜ—U—R^{3a}]—.

In another preferred embodiment, the invention relates to compounds of formula I, supra, wherein T represents —C[H][(CH₂)ₜ—U—R^{3a}]—.

In another preferred embodiment, the invention relates to compounds of formula I, supra, wherein T represents —C(R^{6a})(U—R^{3a})—.

In another preferred embodiment, the invention relates to compounds of formula I, supra, wherein T represents —C(H)(U—R^{3a})—.

In another preferred embodiment, the invention relates to compounds of formula I, supra, wherein U represents a single bond.

In another preferred embodiment, the invention relates to compounds of formula I, supra, wherein U represents a bivalent group selected from: —O—, —S—, —S(═O)—, —S(═O)₂—, —S(═O)—N(R^{3b})—, —N(R^{3c})—S(═O)—, —S(═O)₂—N(R^{3b})—, —N(R^{3c})—S(═O)₂—, —C(═O)—, —N(R^{3b}), —C(═O)—O—, —O—C(═O)—, —C(═S)—O—, —O—C(═S)—, —C(═O)—N(R^{3b})—, —N(R^{3c})—C(═O)—, —N(R^{3c})—C(═O)—N(R^{3b})—, —O—C(═O)—N(R^{3b})—, —N(R^{3c})—C(═O)—O—.

In another preferred embodiment, the invention relates to compounds of formula I, supra, wherein U represents a single bond or a bivalent group selected from: —O—, —C(═O)—, —N(R^{3b}), —C(═O)—O—, —O—C(═O)—, —C(═S)—O—, —C(═O)—N(R^{3b})—, —N(R^{3c})—C(═O)—, —N(R^{3c})—C(═O)—N(R^{3b})—, —O—C(═O)—N(R^{3b})—, —N(R^{3c})—C(═O)—O—.

In another preferred embodiment, the invention relates to compounds of formula I, supra, wherein U represents a bivalent group selected from: —C(═O)—, —N(R^{3b})—, —C(═O)—O—, —O—C(═O)—, —C(═O)—N(R^{3b})—, —N(R^{3c})—C(═O)—.

In another preferred embodiment, the invention relates to compounds of formula I, supra, wherein U represents a bivalent group selected from: —C(═O)—O—, —C(═O)—N(R^{3b})—.

In another preferred embodiment, the invention relates to compounds of formula I, supra, wherein U represents —C(═O)—O—.

In another preferred embodiment, the invention relates to compounds of formula I, supra, wherein U represents —C(═O)—N(R^{3b})—.

In another preferred embodiment, the invention relates to compounds of formula I, supra, wherein U represents —S(═O)₂—N(R^{3b})— or —N(R^{3c})—S(═O)₂—.

In another preferred embodiment, the invention relates to compounds of formula I, supra, wherein R^{3a} represents a hydrogen atom or a group selected from: C₁-C₆-alkyl-, C₃-C₆-cycloalkyl-, 3- to 10-membered heterocycloalkyl-; wherein said C₁-C₆-alkyl-, C₃-C₆-cycloalkyl- or 3- to 10-membered heterocycloalkyl-group is optionally substituted, identically or differently, with 1, 2 or 3 R⁴ groups.

In another preferred embodiment, the invention relates to compounds of formula I, supra, wherein R^{3a} represents a hydrogen atom or a C₁-C₆-alkyl-group; wherein said C₁-C₆-alkyl-group is optionally substituted, identically or differently, with 1, 2 or 3 R⁴ groups.

In another preferred embodiment, the invention relates to compounds of formula I, supra, wherein R^{3a} represents a hydrogen atom or a C₁-C₃-alkyl-group; wherein said C₁-C₃-alkyl-group is optionally substituted with 1 R⁴ group.

In another preferred embodiment, the invention relates to compounds of formula I, supra, wherein R^{3a} represents a C₃-C₆-cycloalkyl-group; wherein said C₃-C₆-cycloalkyl-group is optionally substituted, identically or differently, with 1, 2 or 3 R⁴ groups.

In another preferred embodiment, the invention relates to compounds of formula I, supra, wherein R^{3a} represents a 3- to 10-membered heterocycloalkyl-group; wherein said 3- to 10-membered heterocycloalkyl-group is optionally substituted, identically or differently, with 1, 2 or 3 R⁴ groups.

In another preferred embodiment, the invention relates to compounds of formula I, supra, wherein R^{3b} represents a hydrogen atom or a C₁-C₃-alkyl-group; wherein said C₁-C₃-alkyl-group is optionally substituted with 1 R⁴ group.

In another preferred embodiment, the invention relates to compounds of formula I, supra, wherein R^{3b} represents a hydrogen atom or a C₁-C₃-alkyl-group.

In another preferred embodiment, the invention relates to compounds of formula I, supra, wherein R^{3c} represents a hydrogen atom or a C₁-C₃-alkyl-group; wherein said C₁-C₃-alkyl-group is optionally substituted with 1 R⁴ group.

In another preferred embodiment, the invention relates to compounds of formula I, supra, wherein R^{3c} represents a hydrogen atom or a C₁-C₃-alkyl-group.

In another preferred embodiment, the invention relates to compounds of formula I, supra, wherein N(R^{3b})R^{3a} together form a 3- to 10-membered heterocycloalkyl- or 4- to 10-membered heterocycloalkenyl-group; wherein said 3- to 10-membered heterocycloalkyl- or 4- to 10-membered heterocycloalkenyl-group is optionally substituted, identically or differently, with 1, 2 or 3 R⁴ groups.

In another preferred embodiment, the invention relates to compounds of formula I, supra, wherein N(R^{3b})R^{3a} together form a 3- to 10-membered heterocycloalkyl-group, wherein said 3- to 10-membered heterocycloalkyl-group is optionally substituted, identically or differently, with 1, 2 or 3 R⁴ groups.

In another preferred embodiment, the invention relates to compounds of formula I, supra, wherein N(R^{3b})R^{3a} together form a 3- to 10-membered heterocycloalkyl-group, wherein said 3- to 10-membered heterocycloalkyl-group is optionally substituted with 1 R⁴ group.

In another preferred embodiment, the invention relates to compounds of formula I, supra, wherein R⁴ represents halo-, hydroxy-, cyano-, nitro-, C₁-C₃-alkyl-, halo-C₁-C₃-alkyl-, C₁-C₃-alkoxy-, halo-C₁-C₃-alkoxy-, hydroxy-C₁-C₃-alkyl-, C₁-C₃-alkoxy-C₁-C₃-alkyl-, halo-C₁-C₃-alkoxy-C₁-C₃-alkyl-, R^{5c}—O—, —C(═O)—R^{5c}, —C(═O)—O—R^{5c}, —O—C(═O)—R^{5c}, —N(R^{5b})—C(═O)—R^{5c}, —N(R^{5c})—C(═O)—N(R^{5a})R^{5b}, —N(R^{5a})R^{5b}, —C(═O)—N(R^{5a})R^{5b}, R^{5c}—S—, R^{5c}—S(═O)—, R^{5c}—S(═O)₂—, —N(R^{5c})—S(═O)—R^{5b}, —S(═O)—N(R^{5a})R^{5b}, —N(R⁵)—S(═O)₂—R^{5b} or —S(═O)₂—N(R^{5a})R^{5b}.

In another preferred embodiment, the invention relates to compounds of formula I, supra, wherein R⁴ represents halo-, hydroxy-, cyano-, C₁-C₃-alkyl-, halo-C₁-C₃-alkyl-, C₁-C₃-alkoxy-, halo-C₁-C₃-alkoxy-, hydroxy-C₁-C₃-alkyl-, C₁-C₃-alkoxy-C₁-C₃-alkyl-, R^{5c}—O—, —C(═O)—R^{5c}, —C(═O)—O—R^{5c}, —O—C(═O)—R^{5c}, —N(R^{5b})—C(═O)—R^{5c}, —N(R^{5c})—C(═O)—N(R^{5a})R^{5b}, —N(R^{5a})R^{5b}, —C(═O)—N(R^{5a})R^{5b} or R^{5c}—S(═O)₂—.

In another preferred embodiment, the invention relates to compounds of formula I, supra, wherein R⁴ represents halo-, hydroxy- or —N(R^{5a})R^{5b} In another preferred embodiment, the invention relates to compounds of formula I, supra, wherein R^{5a} represents a hydrogen atom or a C₁-C₆-alkyl- or benzyl-group.

In another preferred embodiment, the invention relates to compounds of formula I, supra, wherein R^{5a} represents a hydrogen atom or a C₁-C₆-alkyl-group.

In another preferred embodiment, the invention relates to compounds of formula I, supra, wherein R^{5a} represents a C₁-C₆-alkyl-group.

In another preferred embodiment, the invention relates to compounds of formula I, supra, wherein R^{5a} represents a hydrogen atom.

In another preferred embodiment, the invention relates to compounds of formula I, supra, wherein R^{5b} represents a hydrogen atom or a C₁-C₆-alkyl-group.

In another preferred embodiment, the invention relates to compounds of formula I, supra, wherein R^{5b} represents a C₁-C₆-alkyl-group.

In another preferred embodiment, the invention relates to compounds of formula I, supra, wherein R^{5b} represents a hydrogen atom.

In another preferred embodiment, the invention relates to compounds of formula I, supra, wherein R^{5c} represents a hydrogen atom or a C₁-C₆-alkyl-group.

In another preferred embodiment, the invention relates to compounds of formula I, supra, wherein R^{5c} represents a C₁-C₆-alkyl-group.

In another preferred embodiment, the invention relates to compounds of formula I, supra, wherein R^{5c} represents a hydrogen atom.

In another preferred embodiment, the invention relates to compounds of formula I, supra, wherein N(R^{5a})R^{5b} together form a 3- to 7-membered heterocycloalkyl-group.

In another preferred embodiment, the invention relates to compounds of formula I, supra, wherein R^{6a} represents a hydrogen atom or a C₁-C₆-alkyl-group; wherein said C₁-C₆-alkyl-group is optionally substituted, identically or differently, with 1, 2 or 3 R⁴ groups.

In another preferred embodiment, the invention relates to compounds of formula I, supra, wherein R^{6a} represents a C₂-C₆-alkenyl- or C₂-C₆-alkynyl-group; wherein said C₂-C₆-alkenyl- or C₂-C₆-alkynyl-group is optionally substituted, identically or differently, with 1, 2 or 3 R⁴ groups.

In another preferred embodiment, the invention relates to compounds of formula I, supra, wherein R^{6a} represents a hydrogen atom or a C₁-C₆-alkyl-, C₂-C₆-alkenyl- or C₂-C₆-alkynyl-group.

In another preferred embodiment, the invention relates to compounds of formula I, supra, wherein R^{6a} represents a hydrogen atom or a C₁-C₆-alkyl-group.

In another preferred embodiment, the invention relates to compounds of formula I, supra, wherein R^{6a} represents a C₂-C₆-alkenyl- or C₂-C₆-alkynyl-group.

In another preferred embodiment, the invention relates to compounds of formula I, supra, wherein R^{6a} represents a C₁-C₆-alkyl-group.

In another preferred embodiment, the invention relates to compounds of formula I, supra, wherein R^{6a} represents a C₁-C₃-alkyl-group.

In another preferred embodiment, the invention relates to compounds of formula I, supra, wherein R^{6a} represents a hydrogen atom.

In another preferred embodiment, the invention relates to compounds of formula I, supra, wherein R^{6a} represents a C₁-C₃-alkyl-group, and wherein t represents 0.

In another preferred embodiment, the invention relates to compounds of formula I, supra, wherein R^{6a} represents a hydrogen atom, and wherein t represents 0.

In another preferred embodiment, the invention relates to compounds of formula I, supra, wherein R^{6b} represents a hydrogen atom or a C₁-C₃-alkyl-group.

In another preferred embodiment, the invention relates to compounds of formula I, supra, wherein R^{6b} represents a C₁-C₃-alkyl-group.

In another preferred embodiment, the invention relates to compounds of formula I, supra, wherein R^{6b} represents a hydrogen atom.

In another preferred embodiment, the invention relates to compounds of formula I, supra, wherein R⁶ represents a hydrogen atom or a C₁-C₃-alkyl-group.

In another preferred embodiment, the invention relates to compounds of formula I, supra, wherein R^{6c} represents a C₁-C₃-alkyl-group.

In another preferred embodiment, the invention relates to compounds of formula I, supra, wherein R^{6c} represents a hydrogen atom.

In another preferred embodiment, the invention relates to compounds of formula I, supra, wherein p represents 0 or 1.

In another preferred embodiment, the invention relates to compounds of formula I, supra, wherein p represents 0.

In another preferred embodiment, the invention relates to compounds of formula I, supra, wherein q represents 0 or 1.

In another preferred embodiment, the invention relates to compounds of formula I, supra, wherein q represents 0.

In another preferred embodiment, the invention relates to compounds of formula I, supra, wherein r represents 1 or 2.

In another preferred embodiment, the invention relates to compounds of formula I, supra, wherein r represents 1.

In another preferred embodiment, the invention relates to compounds of formula I, supra, wherein r represents 2.

In another preferred embodiment, the invention relates to compounds of formula I, supra, wherein s represents 1 or 2.

In another preferred embodiment, the invention relates to compounds of formula I, supra, wherein s represents 1.

In another preferred embodiment, the invention relates to compounds of formula I, supra, wherein s represents 2.

In another preferred embodiment, the invention relates to compounds of formula I, supra, wherein s represents 1 and r represents 2.

In another preferred embodiment, the invention relates to compounds of formula I, supra, wherein s represents 2 and r represents 1.

In another preferred embodiment, the invention relates to compounds of formula I, supra, wherein t represents 0.

It is to be understood that the present invention relates also to any combination of the preferred embodiments described above.

Some examples of combinations are given hereinafter. However, the invention is not limited to these combinations.

In a preferred embodiment, the invention relates to compounds of formula I, supra, in which:
Q-V represents a group selected from: C(R^{1a})—N, N—C(R^{1a})
A represents a group selected from:

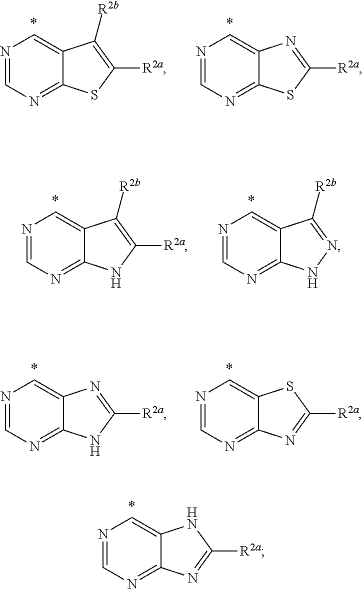

wherein * indicates the point of attachment of said groups to the rest of the molecule;
R^{1a} represents a hydrogen atom or a halogen atom or a group selected from: hydroxy-, cyano-, C₁-C₆-alkyl-, C₂-C₆-alkenyl-, C₂-C₆-alkynyl-, C₁-C₆-alkoxy-, halo-C₁-C₆-alkyl-, halo-C₁-C₆-alkoxy-, hydroxy-C₁-C₆-alkyl-, C₁-C₃-alkoxy-C₁-C₃-alkyl-, —N(R^{5b})R^{5c}, —SCF₃, —SF₅;
R^{1b} represents a hydrogen atom or a halogen atom or a group selected from: hydroxy-, cyano-, C₁-C₆-alkyl-, C₂-C₆-alkenyl-, C₂-C₆-alkynyl-, C₁-C₆-alkoxy-, halo-C₁-C₆-alkyl-, halo-C₁-C₆-alkoxy-, hydroxy-C₁-C₆-alkyl-, C₁-C₃-alkoxy-C₁-C₃-alkyl-, —N(R^{5b})R^{5c}, —SCF₃, —SF₅;
R^{1c} represents a hydrogen atom or a halogen atom or a group selected from: hydroxy-, cyano-, C₁-C₆-alkyl-, C₂-C₆-alkenyl-, C₂-C₆-alkynyl-, C₁-C₆-alkoxy-, halo-C₁-C₆-alkyl-, halo-C₁-C₆-alkoxy-, hydroxy-C₁-C₆-alkyl-, C₁-C₃-alkoxy-C₁-C₃-alkyl-, —N(R^{5b})R^{5c}, —SCF₃, —SF₅;
R^{2a} represents a hydrogen atom or a halogen atom or a group selected from: C₁-C₆-alkyl-, C₃-C₆-cycloalkyl-, 3- to 10-membered heterocycloalkyl-, 4- to 10-membered heterocycloalkenyl-, aryl-, heteroaryl-, cyano-, —(CH₂)_{q}—U—(CH₂)ₚ—R^{3a}; wherein said C₁-C₆-alkyl-, C₃-C₆-cycloalkyl-, 3- to 10-membered heterocycloalkyl-, 4- to 10-membered heterocycloalkenyl-, aryl- or heteroaryl-group is optionally substituted, identically or differently, with 1, 2, 3, 4 or 5 R⁴ groups;
R^{2b} represents a hydrogen atom or a halogen atom or a group selected from: C₁-C₆-alkyl-, C₃-C₆-cycloalkyl-, 3- to 10-membered heterocycloalkyl-, 4- to 10-membered heterocycloalkenyl-, aryl-, heteroaryl-, cyano-, —(CH₂)_{q}—U—(CH₂)ₚ—R^{3a}; wherein said C₁-C₆-alkyl-, C₃-C₆-cycloalkyl-, 3- to 10-membered heterocycloalkyl-, 4- to 10-membered heterocycloalkenyl-, aryl- or heteroaryl-group is optionally substituted, identically or differently, with 1, 2, 3, 4 or 5 R⁴ groups;
or
R^{2a} and R^{2b} together

represent —(CH₂)ᵣ-T-(CH₂)ₛ—
T represents a group selected from: U, —C[H][(CH₂)ₜ—U—R^{3a}]—;
U represents a single bond or a bivalent group selected from: —O—, —S—, —S(═O)—, —S(═O)₂—, —S(═O)—N(R^{3b})—, —N(R^{3c})—S(═O)—, —C(═O)—, —N(R^{3b})—, —C(═O)—O—, —O—C(═O)—, —C(═S)—O—, —O—C(═S)—, —C(═O)—N(R^{3b})—, —N(R^{3c})—C(═O)—, —N(R^{3c})—C(═O)N(R^{3b})—, —O—C(═O)—N(R^{3b})—, —N(R^{3c})—C(═O)—O—;
R^{3a} represents a hydrogen atom or a group selected from: C₁-C₆-alkyl-, C₃-C₆-cycloalkyl-, 3- to 10-membered heterocycloalkyl-, 4- to 10-membered heterocycloalkenyl-, aryl-, heteroaryl-; wherein said C₁-C₆-alkyl-, C₃-C₆-cycloalkyl-, 3- to 10-membered heterocycloalkyl-, 4- to 10-membered heterocycloalkenyl-, aryl- or heteroaryl-group is optionally substituted, identically or differently, with 1, 2, 3, 4 or 5 R⁴ groups;
R^{3b} represents a hydrogen atom or a group selected from: C₁-C₆-alkyl-, C₃-C₆-cycloalkyl-, 3- to 10-membered heterocycloalkyl-, 4- to 10-membered heterocycloalkenyl-, aryl-, heteroaryl-; wherein said C₁-C₆-alkyl-, C₃-C₆-cycloalkyl-, 3- to 10-membered heterocycloalkyl-, 4- to 10-membered heterocycloalkenyl-, aryl- or heteroaryl-group is optionally substituted, identically or differently, with 1, 2, 3, 4 or 5 R⁴ groups;
R^{3c} represents a hydrogen atom or a group selected from: C₁-C₆-alkyl-, C₃-C₆-cycloalkyl-, 3- to 10-membered heterocycloalkyl-, 4- to 10-membered heterocycloalkenyl-, aryl-, heteroaryl-; wherein said C₁-C₆-alkyl-, C₃-C₆-cycloalkyl-, 3- to 10-membered heterocycloalkyl-, 4- to 10-membered heterocycloalkenyl-, aryl- or heteroaryl-group is optionally substituted, identically or differently, with 1, 2, 3, 4 or 5 R⁴ groups;
or
N(R^{3b})R^{3a} together

form a 3- to 10-membered heterocycloalkyl- or 4- to 10-membered heterocycloalkenyl-group; wherein said 3- to 10-membered heterocycloalkyl- or 4- to 10-membered heterocycloalkenyl-group is optionally substituted, identically or differently, with 1, 2, 3, 4 or 5 R⁴ groups;
R⁴ represents halo-, hydroxy-, oxo-(O═), cyano-, nitro-, C₁-C₆-alkyl-, C₂-C₆-alkenyl-, C₂-C₆-alkynyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R^{5c}—O—, —C(═O)—R^{5c}, —C(═O)—O—R^{5c}, —O—C(═O)—R^{5c}, —N(R^{5b})—C(═O)—R^{5c}, —N(R^{5c})—C(═O)—N(R^{5a})R^{5b}, —N(R^{5a})R^{5b}, —C(═O)—N(R^{5a})R^{5b}, R^{5c}—S—, R^{5c}—S(═O)—, R^{5c}—S(═O)₂—, —N(R^{5c})—S(═O)—R^{5b}, —S(═O)—N(R^{5a})R^{5b}, —N(R^{5c})—S(═O)₂—R^{5b}, —S(═O)₂—N(R^{5a})R^{5b}, —S(═O)═N(R^{5c})R^{5b}, —S(═O)═N(R^{5c})R^{5b} or —N═S(═O)(R^{5c})R^{5b};
R^{5a} represents a hydrogen atom, a C₁-C₆-alkyl-, a C₃-C₆-cycloalkyl- or a 3- to 10-membered heterocycloalkyl-group;

R^{5b} represents a hydrogen atom, a C₁-C₆-alkyl-, a C₃-C₆-cycloalkyl- or a 3- to 10-membered heterocycloalkyl-group;
R^{5c} represents a hydrogen atom, a C₁-C₆-alkyl-, a C₃-C₆-cycloalkyl- or a 3- to 10-membered heterocycloalkyl-group;
or
N(R^{5a})R^{5b}

together form a 3- to 7-membered heterocycloalkyl-group;
p represents an integer of 0, 1, 2 or 3;
q represents an integer of 0, 1, 2 or 3;
r represents an integer of 1, 2 or 3;
s represents an integer of 1, 2 or 3; and
t represents an integer of 0 or 1;
   or a tautomer, an N-oxide, a hydrate, a solvate, or a salt thereof, or a mixture of same.

In another preferred embodiment, the invention relates to compounds of formula I, supra, in which:
Q-V represents a group selected from: C(R^{1a})—N, N—C(R^{1a})
A represents a group selected from:

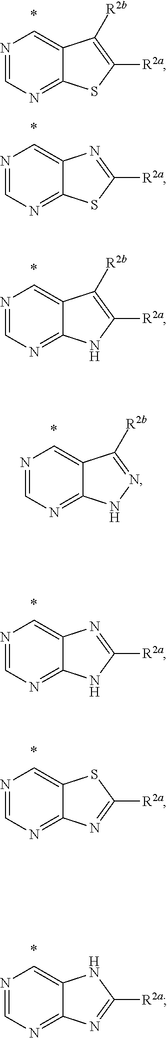

wherein * indicates the point of attachment of said groups to the rest of the molecule;
R^{1a} represents a hydrogen atom or a group selected from: cyano-, C₁-C₆-alkyl-, C₁-C₆-alkoxy-, halo-C₁-C₆-alkyl-, halo-C₁-C₆-alkoxy-, hydroxy-C₁-C₆-alkyl-, C₁-C₃-alkoxy-C₁-C₃-alkyl-;
R^{1b} represents a hydrogen atom or a group selected from: cyano-, C₁-C₆-alkyl-, C₁-C₆-alkoxy-, halo-C₁-C₆-alkyl-, halo-C₁-C₆-alkoxy-, hydroxy-C₁-C₆-alkyl-, C₁-C₃-alkoxy-C₁-C₃-alkyl-;
R^{1c} represents a hydrogen atom or a group selected from: cyano-, C₁-C₆-alkyl-, C₁-C₆-alkoxy-, halo-C₁-C₆-alkyl-, halo-C₁-C₆-alkoxy-, hydroxy-C₁-C₆-alkyl-, C₁-C₃-alkoxy-C₁-C₃-alkyl-;
R^{2a} represents a hydrogen atom or a halogen atom or a group selected from: C₁-C₆-alkyl-, C₃-C₆-cycloalkyl-, 3- to 10-membered heterocycloalkyl-, halo-C₁-C₃-alkyl-, cyano-, —(CH₂)_{q}—U—(CH₂)ₚ—R^{3a}; wherein said C₁-C₆-alkyl-, C₃-C₆-cycloalkyl-, 3- to 10-membered heterocycloalkyl- or 4- to 10-membered heterocycloalkenyl-group is optionally substituted, identically or differently, with 1, 2 or 3 R⁴ groups, with the proviso that said halo-C₁-C₃-alkyl-group does not contain more than 5 halogen atoms;
R^{2b} represents a hydrogen atom or a halogen atom or a group selected from: C₁-C₆-alkyl-, halo-C₁-C₃-alkyl-, cyano-, —(CH₂)_{q}—U—(CH₂)ₚ—R^{3a}; wherein said C₁-C₆-alkyl-group is optionally substituted, identically or differently, with 1, 2 or 3 R⁴ groups,
   with the proviso that said halo-C₁-C₃-alkyl-group does not contain more than 5 halogen atoms;
or
R^{2a} and R^{2b} together

represent —(CH₂)ᵣ-T-(CH₂)ₛ—
T represents —C[R^{6a}][(C(R^{6b})(R^{6c}))ₜ—U—R^{3a}]
U represents a single bond or a bivalent group selected from: —O—, —S—, —S(═O)—, —S(═O)₂—, —S(═O)—N(R^{3b})—, —N(R^{3c})—S(═O)—, —C(═O)—, —N(R^{3b})—, —C(═O)—O—, —O—C(═O)—, —C(═S)—O—, —O—C(═S)—, —C(═O)—N(R^{3b})—, —N(R^{3c})—C(═O)—, —N(R^{3c})—C(═O)—N(R^{3b})—, —O—C(═O)—N(R^{3b})—, —N(R^{3c})—C(═O)—O—;
R^{3a} represents a hydrogen atom or a group selected from: C₁-C₆-alkyl-, C₃-C₆-cycloalkyl-, 3- to 10-membered heterocycloalkyl-, 4- to 10-membered heterocycloalkenyl-, aryl-, heteroaryl-, halo-C₁-C₃-alkyl-; wherein said C₁-C₆-alkyl-, C₃-C₆-cycloalkyl-, 3- to 10-membered heterocycloalkyl-, aryl-, heteroaryl- or halo-C₁-C₃-alkyl-group is optionally substituted, identically or differently, with 1, 2, 3, 4 or 5 R⁴ groups, with the proviso that said halo-C₁-C₃-alkyl-group, together with the R⁴ groups optionally attached to it, does not contain more than 5 halogen atoms;
R^{3b} represents a hydrogen atom or a group selected from: C₁-C₆-alkyl-, C₃-C₆-cycloalkyl-, 3- to 10-membered heterocycloalkyl-, 4- to 10-membered heterocycloalkenyl-, aryl-, heteroaryl-, halo-C₁-C₃-alkyl-; wherein said C₁-C₆-alkyl-, C₃-C₆-cycloalkyl-, 3- to 10-membered heterocycloalkyl-, aryl-, heteroaryl- or halo-C₁-C₃-alkyl-group is optionally substituted, identically or differently, with 1, 2, 3, 4 or 5 R⁴ groups, with the proviso that said halo-C₁-C₃-alkyl-group, together with the R⁴ groups optionally attached to it, does not contain more than 5 halogen atoms;
R^{3c} represents a hydrogen atom or a group selected from: C₁-C₆-alkyl-, C₃-C₆-cycloalkyl-, 3- to 10-membered heterocycloalkyl-, 4- to 10-membered heterocycloalkenyl-, aryl-, heteroaryl-, halo-C₁-C₃-alkyl-; wherein said C₁-C₆-alkyl-, C₃-C₆-cycloalkyl-, 3- to 10-membered heterocycloalkyl-, aryl-, heteroaryl- or halo-C₁-C₃-alkyl-group is optionally substituted, identically or differently, with 1, 2, 3, 4 or 5 R⁴ groups, with the proviso that said halo-C₁-C₃-alkyl-group, together with the R⁴ groups optionally attached to it, does not contain more than 5 halogen atoms;
or
N(R^{3b})R^{3a}

together form a 3- to 10-membered heterocycloalkyl- or 4- to 10-membered heterocycloalkenyl-group; wherein said 3- to 10-membered heterocycloalkyl- or 4- to 10-membered heterocycloalkenyl-group is optionally substituted, identically or differently, with 1, 2, 3, 4 or 5 R⁴ groups;
R⁴ represents halo-, hydroxy-, oxo-(O═), cyano-, nitro-, C₁-C₆-alkyl-, C₂-C₆-alkenyl-, C₂-C₆-alkynyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R^{5c}—O—, —C(═O)—R^{5c}, —C(═O)—O—R^{5c}, —O—C(═O)—R^{5c}, —N(R^{5b})—C(═O)—R^{5c}, —N(R^{5c})—C(═O)—N(R^{5a})R^{5b}, —N(R^{5a})R^{5b}, —C(═O)—N(R^{5a})R^{5b}, R^{5c}—S—, R^{5c}—S(═O)—, R^{5c}—S(═O)₂—, —N(R^{5c})—S(═O)—R^{5b}, —S(═O)—N(R^{5a})R^{5b}, —N(R^{5c})—S(═O)₂—R^{5b}, —S(═O)₂—N(R^{5a})R^{5b}, —S(═O)═N(R^{5c})R^{5b}, —S(═O)═N(R^{5c})R^{5b} or —N═S(═O)(R^{5c})R^{5b};
R^{5a} represents a hydrogen atom, a C₁-C₆-alkyl-, a C₃-C₆-cycloalkyl- or a 3- to 10-membered heterocycloalkyl-group;
R^{5b} represents a hydrogen atom, a C₁-C₆-alkyl-, a C₃-C₆-cycloalkyl- or a 3- to 10-membered heterocycloalkyl-group;
R^{5C} represents a hydrogen atom, a C₁-C₆-alkyl-, a C₃-C₆-cycloalkyl- or a 3- to 10-membered heterocycloalkyl-group;
or
N(R^{5a})R^{5b} together form a 3- to 7-membered heterocycloalkyl-group;
R^{6a} represents a hydrogen atom or a group selected from: C₁-C₆-alkyl-, C₂-C₆-alkenyl-, C₂-C₆-alkynyl-; wherein said C₁-C₆-alkyl-, C₂-C₆-alkenyl- or C₂-C₆-alkynyl-group is optionally substituted, identically or differently, with 1, 2 or 3 R⁴ groups;
R^{6b} represents a hydrogen atom or a C₁-C₃-alkyl-group;
R^{6C} represents a hydrogen atom or a C₁-C₃-alkyl-group;
p represents an integer of 0, 1, 2 or 3;
q represents an integer of 0, 1, 2 or 3;
r represents an integer of 1, 2 or 3;
s represents an integer of 1, 2 or 3; and
t represents an integer of 0 or 1;
or a tautomer, an N-oxide, a hydrate, a solvate, or a salt thereof, or a mixture of same.

In another preferred embodiment, the invention relates to compounds of formula I, supra, in which:
Q-V represents a group selected from: C(R^{1a})—N, N—C(R^{1a})
A represents a group selected from:

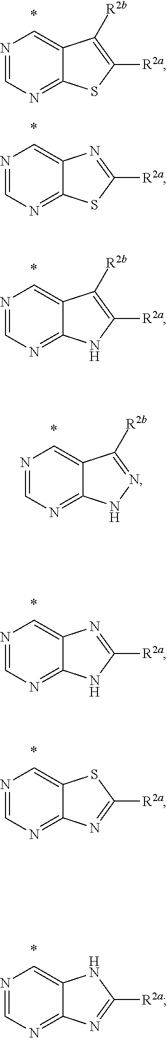

wherein * indicates the point of attachment of said group to the rest of the molecule;
R^{1a} represents a hydrogen atom or a halogen atom or a group selected from: hydroxy-, cyano-, C₁-C₆-alkyl-, C₂-C₆-alkenyl-, C₂-C₆-alkynyl-, C₁-C₆-alkoxy-, halo-C₁-C₆-alkoxy-, —N(R^{5b})R^{5c}, —SCF₃, —SF₅;
R^{1b} represents a hydrogen atom or a halogen atom or a group selected from: hydroxy-, cyano-, C₁-C₆-alkyl-, C₂-C₆-alkenyl-, C₂-C₆-alkynyl-, C₁-C₆-alkoxy-, halo-C₁-C₆-alkoxy-, —N(R^{5b})R^{5c}, —SCF₃, —SF₅;
R^{1b} represents a hydrogen atom or a halogen atom or a group selected from: hydroxy-, cyano-, C₁-C₆-alkyl-, C₂-C₆-alkenyl-, C₂-C₆-alkynyl-, C₁-C₆-alkoxy-, halo-C₁-C₆-alkoxy-, —N(R^{5b})R^{5c}, —SCF₃, —SF₅;
R^{2a} represents a hydrogen atom or a halogen atom or a group selected from: C₁-C₆-alkyl-, C₃-C₆-cycloalkyl-, 3- to 10-membered heterocycloalkyl-, 4- to 10-membered heterocycloalkenyl-, aryl-, heteroaryl-, halo-C₁-C₃-alkyl-, cyano-, —(CH₂)_{q}—U—(CH₂)ₚ—R^{3a}; wherein said C₁-C₆-alkyl-, C₃-C₆-cycloalkyl-, 3- to 10-membered heterocycloalkyl-, 4- to 10-membered heterocycloalkenyl-, aryl- or heteroaryl-group is optionally substituted, identically or differently, with 1, 2, 3, 4 or 5 R⁴ groups,
   with the proviso that said halo-C₁-C₃-alkyl-group does not contain more than 5 halogen atoms;
R^{2b} represents a hydrogen atom or a halogen atom or a group selected from: C₁-C₆-alkyl-, C₃-C₆-cycloalkyl-, 3- to 10-membered heterocycloalkyl-, 4- to 10-membered heterocycloalkenyl-, aryl-, heteroaryl-, halo-C₁-C₃-alkyl-, cyano-, —(CH₂)_{q}—U—(CH₂)ₚ—R^{3a}; wherein said C₁-C₆-alkyl-, C₃-C₆-cycloalkyl-, 3- to 10-membered heterocycloalkyl-, 4- to 10-membered heterocycloalkenyl-, aryl- or heteroaryl-group is optionally substituted, identically or differently, with 1, 2, 3, 4 or 5 R⁴ groups,
   with the proviso that said halo-C₁-C₃-alkyl-group does not contain more than 5 halogen atoms;
or
R^{2a} and R^{2b} together

represent —(CH₂)ᵣ-T-(CH₂)ₛ—
T represents a group selected from: U, —C[R^{6a}][(C(R^{6b})(R^{6c}))ₜ—U—R^{3a}];
U represents a single bond or a bivalent group selected from: —O—, —S—, —S(═O)—, —S(═O)₂—, —S(═O)—N(R^{3b})—, —N(R^{3c})—S(═O)—, —C(═O)—, —N(R^{3b})—, —C(═O)—O—, —O—C(═O)—, —C(═S)—O—, —O—C(═S)—, —C(═O)—N(R^{3b})—, —N(R^{3c})—C(═O)—, —N(R^{3c})—C(═O)N(R^{3b})—, —O—C(═O)—N(R^{3b})—, —N(R^{3c})—C(═O)—O—;
R^{3a} represents a hydrogen atom or a group selected from: C₁-C₆-alkyl-, C₃-C₆-cycloalkyl-, 3- to 10-membered heterocycloalkyl-; wherein said C₁-C₆-alkyl-, C₃-C₆-cycloalkyl- or 3- to 10-membered heterocycloalkyl-group is optionally substituted, identically or differently, with 1, 2 or 3 R⁴ groups;
R^{3b} represents a hydrogen atom or a C₁-C₃-alkyl-group; wherein said C₁-C₃-alkyl-group is optionally substituted with 1 R⁴ group;
R^{3c} represents a hydrogen atom or a C₁-C₃-alkyl-group;
or
N(R^{3b})R^{3a} together
   form a 3- to 10-membered heterocycloalkyl- or 4- to 10-membered heterocycloalkenyl-group; wherein said 3- to 10-membered heterocycloalkyl- or 4- to 10-membered heterocycloalkenyl-group is optionally substituted, identically or differently, with 1, 2 or 3 R⁴ groups;
R⁴ represents halo-, hydroxy-, oxo-(0=), cyano-, nitro-, C₁-C₃-alkyl-, halo-C₁-C₃-alkyl-, C₁-C₃-alkoxy-, halo-C₁-C₃-alkoxy-, hydroxy-C₁-C₃-alkyl-, C₁-C₃-alkoxy-C₁-C₃-alkyl-, halo-C₁-C₃-alkoxy-C₁-C₃-alkyl-, R^{5c}—O—, —C(═O)—R^{5c}, —C(═O)—O—R^{5c}—O—C(═O)—R^{5a}, —N(R^{5b})—C(═O)—R^{5c}, —N(R^{5c})—C(═O)—N(R^{5a})R^{5b}, —N(R^{5a})R^{5b}, —C(═O)—N(R^{5a})R^{5b}, R^{5c}—S—, R^{5c}—S(═O)—, R^{5c}—S(═O)₂—, —N(R^{5c})—S(═O)—R^{5b}, —S(═O)—N(R^{5a})R^{5b}, —N(R^{5c})—S(═O)₂—R^{5b} or —S(═O)₂—N(R^{5a})R^{5b};
R^{5a} represents a hydrogen atom or a C₁-C₃-alkyl-group;
R^{5b} represents a hydrogen atom or a C₁-C₃-alkyl-group;
R^{5C} represents a hydrogen atom or a C₁-C₃-alkyl-group;
R^{6a} represents a hydrogen atom or a group selected from: C₁-C₆-alkyl-, C₂-C₆-alkenyl-, C₂-C₆-alkynyl-; wherein said C₁-C₆-alkyl-, C₂-C₆-alkenyl- or C₂-C₆-alkynyl-group is optionally substituted, identically or differently, with 1, 2 or 3 R⁴ groups;
R^{6b} represents a hydrogen atom or a C₁-C₃-alkyl-group;
R^{6C} represents a hydrogen atom or a C₁-C₃-alkyl-group;
or
N(R^{5a})R^{5b}

together form a 3- to 7-membered heterocycloalkyl-group;
p represents an integer of 0, 1, 2 or 3;
q represents an integer of 0, 1, 2 or 3;
r represents an integer of 1, 2 or 3;
s represents an integer of 1, 2 or 3; and
t represents an integer of 0 or 1;
or a tautomer, an N-oxide, a hydrate, a solvate, or a salt thereof, or a mixture of same.

In another preferred embodiment, the invention relates to compounds of formula I, supra, in which:
Q-V represents a group selected from: C(R^{1a})—N, N—C(R^{1a})
A represents a group selected from:

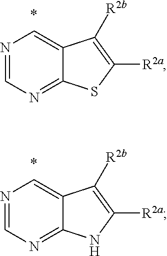

wherein * indicates the point of attachment of said groups to the rest of the molecule;
R^{1a} represents a hydrogen atom or a halogen atom or a group selected from: hydroxy-, C₁-C₃-alkyl-C₁-C₃-alkoxy-, halo-C₁-C₃-alkoxy-;
R^{1b} represents a hydrogen atom;
R^{1c} represents a hydrogen atom;
R^{2a} represents a hydrogen atom or a halogen atom or a group selected from: C₁-C₆-alkyl-, C₃-C₆-cycloalkyl-, 3- to 10-membered heterocycloalkyl-, 4- to 10-membered heterocycloalkenyl-, aryl-, heteroaryl-, halo-C₁-C₃-alkyl-, cyano-, —(CH₂)_{q}—U—(CH₂)ₚ—R^{3a}; wherein said C₁-C₆-alkyl-, C₃-C₆-cycloalkyl-, 3- to 10-membered heterocycloalkyl-, 4- to 10-membered heterocycloalkenyl-, aryl- or heteroaryl-group is optionally substituted, identically or differently, with 1, 2, 3, 4 or 5 R⁴ groups,
   with the proviso that said halo-C₁-C₃-alkyl-group does not contain more than 5 halogen atoms;
R^{2b} represents a hydrogen atom or a halogen atom or a group selected from: C₁-C₆-alkyl-, C₃-C₆-cycloalkyl-, 3- to 10-membered heterocycloalkyl-, 4- to 10-membered heterocycloalkenyl-, aryl-, heteroaryl-, halo-C₁-C₃-alkyl-, cyano-, —(CH₂)_{q}—U—(CH₂)ₚ—R^{3a}; wherein said C₁-C₆-alkyl-, C₃-C₆-cycloalkyl-, 3- to 10-membered heterocycloalkyl-, 4- to 10-membered heterocycloalkenyl-, aryl- or heteroaryl-group is optionally substituted, identically or differently, with 1, 2, 3, 4 or 5 R⁴ groups,
   with the proviso that said halo-C₁-C₃-alkyl-group does not contain more than 5 halogen atoms;
or
R^{2a} and R^{2b} together

represent —(CH₂)ᵣ-T-(CH₂)ₛ—
T represents a group selected from: U, —C[R^{6a}][(C(R^{6b})(R^{6c}))ₜ—U—R^{3a}];
U represents a single bond or a bivalent group selected from: —O—, —S—, —S(═O)—, —S(═O)₂—, —S(═O)—N(R^{3b})—, —N(R^{3c})—S(═O)—, —C(═O)—, —N(R^{3b})—, —C(═O)—O—, —O—C(═O)—, —C(═S)—O—, —O—C(═S)—, —C(═O)—N(R^{3b})—, —N(R^{3c})—C(═O)—, —N(R^{3c})—C(═O)N(R^{3b})—, —O—C(═O)—N(R^{3b})—, —N(R^{3c})—C(═O)—O—;
R^{3a} represents a hydrogen atom or a group selected from: C₁-C₆-alkyl-, C₃-C₆-cycloalkyl-, 3- to 10-membered heterocycloalkyl-; wherein said C₁-C₆-alkyl-, C₃-C₆-cycloalkyl- or 3- to 10-membered heterocycloalkyl-group is optionally substituted, identically or differently, with 1, 2 or 3 R⁴ groups;
R^{3b} represents a hydrogen atom or a C₁-C₃-alkyl-group; wherein said C₁-C₃-alkyl-group is optionally substituted with 1 R⁴ group;
R^{3c} represents a hydrogen atom or a C₁-C₃-alkyl-group;
or
N(R^{3b})R^{3a} together
   form a 3- to 10-membered heterocycloalkyl- or 4- to 10-membered heterocycloalkenyl-group; wherein said 3- to 10-membered heterocycloalkyl- or 4- to 10-membered heterocycloalkenyl-group is optionally substituted, identically or differently, with 1, 2 or 3 R⁴ groups;
R⁴ represents halo-, hydroxy-, oxo-(0=), cyano-, nitro-, C₁-C₃-alkyl-, halo-C₁-C₃-alkyl-, C₁-C₃-alkoxy-, halo-C₁-C₃-alkoxy-, hydroxy-C₁-C₃-alkyl-, C₁-C₃-alkoxy-C₁-C₃-alkyl-, halo-C₁-C₃-alkoxy-C₁-C₃-alkyl-, R^{5c}—O—, —C(═O)—R^{5c}—C(═O)—O—R^{5c}, —O—C(═O)—R^{5c}, —N(R^{5b})—C(═O)—R^{5c}, —N(R^{5c})—C(═O)—N(R^{5a})R^{5b}, —N(R^{5a})R^{5b}, —C(═O)—N(R^{5a})R^{5b}, R^{5c}—S—, R^{5c}—S(═O)—, R^{5c}—S(═O)₂—, —N(R^{5c})—S(═O)—R^{5b}, —S(═O)—N(R^{5a})R^{5b}, —N(R^{5c})—S(═O)₂—R^{5b} or —S(═O)₂—N(R^{5a})R^{5b};
R^{5a} represents a hydrogen atom or a C₁-C₃-alkyl-group;
R^{5b} represents a hydrogen atom or a C₁-C₃-alkyl-group;

R^{5c} represents a hydrogen atom or a C₁-C₃-alkyl-group;
or
N(R^{5a})R^{5b}

together form a 3- to 7-membered heterocycloalkyl-group;
R^{6a} represents a hydrogen atom or a C₁-C₆-alkyl-group; wherein said C₁-C₆-alkyl group is optionally substituted, identically or differently, with 1, 2 or 3 R⁴ groups;
R^{6b} represents a hydrogen atom or a C₁-C₃-alkyl-group;
R^{6c} represents a hydrogen atom or a C₁-C₃-alkyl-group;
p represents an integer of 0, 1, 2 or 3;
q represents an integer of 0, 1, 2 or 3;
r represents an integer of 1, 2 or 3;
s represents an integer of 1, 2 or 3; and
t represents an integer of 0 or 1;
or a tautomer, an N-oxide, a hydrate, a solvate, or a salt thereof, or a mixture of same.

In another preferred embodiment, the invention relates to compounds of formula I, supra, in which:
Q-V represents a group selected from: C(R^{1a})—N, N—C(R^{1a})
A represents a group selected from:

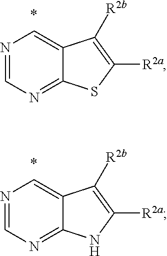

wherein * indicates the point of attachment of said groups to the rest of the molecule;
R^{1a} represents a hydrogen atom or a halogen atom or a group selected from: C₁-C₃-alkyl-C₁-C₃-alkoxy-, halo-C₁-C₃-alkoxy-;
R^{1b} represents a hydrogen atom;
R^{1c} represents a hydrogen atom;
R^{2a} represents a hydrogen atom or a halogen atom or a group selected from: C₁-C₆-alkyl-, C₃-C₆-cycloalkyl-, 3- to 10-membered heterocycloalkyl-, —(CH₂)_{q}—U—(CH₂)ₚ—R^{3a}; wherein said C₁-C₆-alkyl-, C₃-C₆-cycloalkyl- or 3- to 10-membered heterocycloalkyl-group is optionally substituted, identically or differently, with 1, 2, 3, 4 or 5 R⁴ groups;
R^{2b} represents a hydrogen atom or a halogen atom or a group selected from: C₁-C₆-alkyl-, C₃-C₆-cycloalkyl-, 3- to 10-membered heterocycloalkyl-, halo-C₁-C₃-alkyl-, —(CH₂)_{q}—U—(CH₂)ₚ—R^{3a}; wherein said C₁-C₆-alkyl-, C₃-C₆-cycloalkyl- or 3- to 10-membered heterocycloalkyl-group is optionally substituted, identically or differently, with 1, 2, 3, 4 or 5 R⁴ groups,
   with the proviso that said halo-C₁-C₃-alkyl-group does not contain more than 5 halogen atoms;
or
R^{2a} and R^{2b} together

represent —(CH₂)ᵣ-T-(CH₂)ₛ—
T represents a group selected from: U, —C[R^{6a}][(C(R^{6b})(R^{6c}))ₜ—U—R^{3a}]—;
U represents a single bond or a bivalent group selected from: —O—, —C(═O)—, —N(R^{3b})—, —C(═O)—O—, —O—C(═O)—, —C(═S)—O—, —C(═O)—N(R^{3b})—, —N(R^{3c})—C(═O)—, —N(R^{3c})—C(═O)—N(R^{3b})—, —O—C(═O)—N(R^{3b})—, —N(R^{3c})—C(═O)—O—;
R^{3a} represents a hydrogen atom or a group selected from: C₁-C₆-alkyl-, C₃-C₆-cycloalkyl-, 3- to 10-membered heterocycloalkyl-; wherein said C₁-C₆-alkyl-, C₃-C₆-cycloalkyl- or 3- to 10-membered heterocycloalkyl-group is optionally substituted, identically or differently, with 1, 2 or 3 R⁴ groups;
R^{3b} represents a hydrogen atom or a C₁-C₃-alkyl-group; wherein said C₁-C₃-alkyl-group is optionally substituted with 1 R⁴ group;
R^{3c} represents a hydrogen atom or a C₁-C₃-alkyl-group;
or
N(R^{3b})R^{3a} together
   form a 3- to 10-membered heterocycloalkyl- or 4- to 10-membered heterocycloalkenyl-group; wherein said 3- to 10-membered heterocycloalkyl- or 4- to 10-membered heterocycloalkenyl-group is optionally substituted, identically or differently, with 1, 2 or 3 R⁴ groups;
R⁴ represents halo-, hydroxy-, cyano-, nitro-, C₁-C₃-alkyl-, halo-C₁-C₃-alkyl-, C₁-C₃-alkoxy-, halo-C₁-C₃-alkoxy-, hydroxy-C₁-C₃-alkyl-, C₁-C₃-alkoxy-C₁-C₃-alkyl-, halo-C₁-C₃-alkoxy-C₁-C₃-alkyl-, R^{5c}—O—, —C(═O)—R^{5c}, —C(═O)—O—R^{5c}, —O—C(═O)—R^{5c}, —N(R^{5b})—C(═O)—R^{5c}, —N(R^{5c})—C(═O)—N(R^{5a})R^{5b}, —N(R^{5a})R^{5b}, —C(═O)—N(R^{5a})R^{5b}, R^{5c}—S(═O)₂—, —N(R^{5c})—S(═O)—R^{5b}, —S(═O)—N(R^{5a})R^{5b}, —N(R^{5c})—S(═O)₂R^{5b} or —S(═O)₂—N(R^{5a})R^{5b};
R^{5a} represents a hydrogen atom or a C₁-C₃-alkyl-group;
R^{5b} represents a hydrogen atom or a C₁-C₃-alkyl-group;
R^{5c} represents a hydrogen atom or a C₁-C₃-alkyl-group;
or
N(R^{5a})R^{5b}

together form a 3- to 7-membered heterocycloalkyl-group;
R^{6a} represents a hydrogen atom or a group selected from: C₁-C₆-alkyl-, C₂-C₆-alkenyl-, C₂-C₆-alkynyl-; wherein said C₁-C₆-alkyl-, C₂-C₆-alkenyl- or C₂-C₆-alkynyl-group is optionally substituted, identically or differently, with 1, 2 or 3 R⁴ groups;
R^{6b} represents a hydrogen atom or a C₁-C₃-alkyl-group;
R^{6c} represents a hydrogen atom or a C₁-C₃-alkyl-group;
p represents 0;
q represents 0;
r represents 1 or 2;
s represents 1 or 2; and
t represents 0;
or a tautomer, an N-oxide, a hydrate, a solvate, or a salt thereof, or a mixture of same.

In another preferred embodiment, the invention relates to compounds of formula I, supra, in which:
A represents a group selected from:

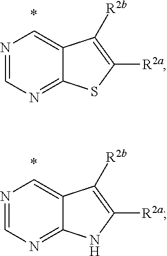

wherein * indicates the point of attachment of said groups to the rest of the molecule;
Q-V represents C(R^{1a})—N, and R^{1a} represents a hydrogen atom;
or
Q-V represents N—C(R^{1a}), and R^{1a} represents a hydrogen atom or a halogen atom or a group selected from: hydroxy-, C₁-C₃-alkyl-C₁-C₃-alkoxy-, halo-C₁-C₃-alkoxy-;
R^{1b} represents a hydrogen atom;
R^{1c} represents a hydrogen atom;
R^{2a} represents a hydrogen atom or a halogen atom or a group selected from: C₁-C₆-alkyl-, C₃-C₆-cycloalkyl-, 3- to 10-membered heterocycloalkyl-, 4- to 10-membered heterocycloalkenyl-, aryl-, heteroaryl-, halo-C₁-C₃-alkyl-, cyano-, —(CH₂)_{q}—U—(CH₂)ₚ—R^{3a}; wherein said C₁-C₆-alkyl-, C₃-C₆-cycloalkyl-, 3- to 10-membered heterocycloalkyl-, 4- to 10-membered heterocycloalkenyl-, aryl- or heteroaryl-group is optionally substituted, identically or differently, with 1, 2, 3, 4 or 5 R⁴ groups, with the proviso that said halo-C₁-C₃-alkyl-group does not contain more than 5 halogen atoms;
R^{2b} represents a hydrogen atom or a halogen atom or a group selected from: C₁-C₆-alkyl-, C₃-C₆-cycloalkyl-, 3- to 10-membered heterocycloalkyl-, 4- to 10-membered heterocycloalkenyl-, aryl-, heteroaryl-, halo-C₁-C₃-alkyl-, cyano-, —(CH₂)_{q}—U—(CH₂)ₚ—R^{3a}; wherein said C₁-C₆-alkyl-, C₃-C₆-cycloalkyl-, 3- to 10-membered heterocycloalkyl-, 4- to 10-membered heterocycloalkenyl-, aryl- or heteroaryl-group is optionally substituted, identically or differently, with 1, 2, 3, 4 or 5 R⁴ groups, with the proviso that said halo-C₁-C₃-alkyl-group does not contain more than 5 halogen atoms;
or
R^{2a} and R^{2b} together

represent —(CH₂)ᵣ-T-(CH₂)ₛ—
T represents —C[R^{6a}][(C(R^{6b})(R^{6c}))ₜ—U— R^{3a}];
U represents a single bond or a bivalent group selected from: —O—, —C(═O)—, —N(R^{3b})—, —C(═O)—O—, —O—C(═O)—, —C(═O)—N(R^{3b})—, —N(R^{3c})—C(═O)—, —N(R^{3c})—C(═O)—N(R^{3b})—, —O—C(═O)—N(R^{3b})—, —N(R^{3c})—C(═O)—O—;
R^{3a} represents a hydrogen atom or a group selected from: C₁-C₆-alkyl-, C₃-C₆-cycloalkyl-, 3- to 10-membered heterocycloalkyl-; wherein said C₁-C₆-alkyl-, C₃-C₆-cycloalkyl- or 3- to 10-membered heterocycloalkyl-group is optionally substituted, identically or differently, with 1, 2 or 3 R⁴ groups;
R^{3b} represents a hydrogen atom or a C₁-C₃-alkyl-group; wherein said C₁-C₃-alkyl-group is optionally substituted with 1 R⁴ group;
R^{3c} represents a hydrogen atom or a C₁-C₃-alkyl-group;
or
N(R^{3b})R^{3a} together
   form a 3- to 10-membered heterocycloalkyl- or 4- to 10-membered heterocycloalkenyl-group; wherein said 3- to 10-membered heterocycloalkyl- or 4- to 10-membered heterocycloalkenyl-group is optionally substituted, identically or differently, with 1, 2 or 3 R⁴ groups;
R⁴ represents halo-, hydroxy-, oxo-(O═), cyano-, nitro-, C₁-C₃-alkyl-, halo-C₁-C₃-alkyl-, C₁-C₃-alkoxy-, halo-C₁-C₃-alkoxy-, hydroxy-C₁-C₃-alkyl-, C₁-C₃-alkoxy-C₁-C₃-alkyl-, halo-C₁-C₃-alkoxy-C₁-C₃-alkyl-, R^{5c}—O—, —C(═O)—R^{5c}, —C(═O)—O—R^{5c}, —O—C(═O)—R^{5c}, —N(R^{5b})—C(═O)—R^{5c}, —N(R^{5c})—C(═O)—N(R^{5a})R^{5b}, —N(R^{5a})R^{5b}, —C(═O)—N(R^{5a})R^{5b}, R^{5c}—S—, R^{5c}—S(═O)—, R^{5c}—S(═O)₂—, —N(R^{5c})—S(═O)—R^{5b}, —S(═O)—N(R^{5a})R^{5b}, —N(R⁵)—S(═O)₂—R^{5b} or —S(═O)₂—N(R^{5a})R^{5b};
R^{5a} represents a hydrogen atom or a C₁-C₃-alkyl-group;
R^{5b} represents a hydrogen atom or a C₁-C₃-alkyl-group;
R^{5c} represents a hydrogen atom or a C₁-C₃-alkyl-group;
or
N(R^{5a})R^{5b}

together form a 3- to 7-membered heterocycloalkyl-group;
R^{6a} represents a hydrogen atom or a group selected from: C₁-C₆-alkyl-, C₂-C₆-alkenyl-, C₂-C₆-alkynyl-; wherein said C₁-C₆-alkyl-, C₂-C₆-alkenyl- or C₂-C₆-alkynyl-group is optionally substituted, identically or differently, with 1, 2 or 3 R⁴ groups;
R^{6b} represents a hydrogen atom or a C₁-C₃-alkyl-group;
R^{6C} represents a hydrogen atom or a C₁-C₃-alkyl-group;
p represents an integer of 0, 1, 2 or 3;
q represents an integer of 0, 1, 2 or 3;
r represents an integer of 1, 2 or 3;
s represents an integer of 1, 2 or 3; and
t represents an integer of 0 or 1;
or a tautomer, an N-oxide, a hydrate, a solvate, or a salt thereof, or a mixture of same.

In another preferred embodiment, the invention relates to compounds of formula I, supra, in which:
A represents a group selected from:

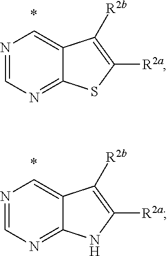

wherein * indicates the point of attachment of said groups to the rest of the molecule;
Q-V represents C(R^{1a})—N, and R^{1a} represents a hydrogen atom;
or
Q-V represents N—C(R^{1a}), and R^{1a} represents a hydrogen atom or a halogen atom or a group selected from: hydroxy-, C₁-C₃-alkyl-C₁-C₃-alkoxy-, halo-C₁-C₃-alkoxy-;
R^{1b} represents a hydrogen atom;
R^{1c} represents a hydrogen atom;
R^{2a} represents a hydrogen atom or a halogen atom or a group selected from: C₁-C₆-alkyl-, C₃-C₆-cycloalkyl-, 3- to 10-membered heterocycloalkyl-, 4- to 10-membered heterocycloalkenyl-, aryl-, heteroaryl-, halo-C₁-C₃-alkyl-, cyano-, —(CH₂)_{q}—U—(CH₂)ₚ—R^{3a}; wherein said C₁-C₆-alkyl-, C₃-C₆-cycloalkyl-, 3- to 10-membered heterocycloalkyl-, 4- to 10-membered heterocycloalkenyl-, aryl- or heteroaryl-group is optionally substituted, identically or differently, with 1, 2, 3, 4 or 5 R⁴ groups, with the proviso that said halo-C₁-C₃-alkyl-group does not contain more than 5 halogen atoms;
R^{2b} represents a hydrogen atom or a halogen atom or a group selected from: C₁-C₆-alkyl-, C₃-C₆-cycloalkyl-, 3- to 10-membered heterocycloalkyl-, 4- to 10-membered heterocycloalkenyl-, aryl-, heteroaryl-, halo-C₁-C₃-alkyl-, cyano-, —(CH₂)_{q}—U—(CH₂)ₚ—R^{3a}; wherein said C₁-C₆-alkyl-, C₃-C₆-cycloalkyl-, 3- to 10-membered heterocycloalkyl-, 4- to 10-membered heterocycloalkenyl-, aryl- or heteroaryl-group is optionally substituted, identically or differently, with 1, 2, 3, 4 or 5 R⁴ groups, with the proviso that said halo-C₁-C₃-alkyl-group does not contain more than 5 halogen atoms;
or
R^{2a} and R^{2b} together

represent —(CH₂)ᵣ-T-(CH₂)ₛ—
T represents —C[R^{6a}][(C(R^{6b})(R^{6c}))ₜ—U—R^{3a}];
U represents a single bond or a bivalent group selected from: —O—, —C(═O)—, —N(R^{3b})—, —C(═O)—O—, —O—C(═O)—, —C(═O)—N(R^{3b})—, —N(R^{3c})—C(═O)—, —N(R^{3c})—C(═O)—N(R^{3b})—, —O—C(═O)—N(R^{3b})—, —N(R^{3c})—C(═O)—O—;
R^{3a} represents a hydrogen atom or a group selected from: C₁-C₆-alkyl-, C₃-C₆-cycloalkyl-, 3- to 10-membered heterocycloalkyl-; wherein said C₁-C₆-alkyl-, C₃-C₆-cycloalkyl- or 3- to 10-membered heterocycloalkyl-group is optionally substituted, identically or differently, with 1, 2 or 3 R⁴ groups;
R^{3b} represents a hydrogen atom or a C₁-C₃-alkyl-group; wherein said C₁-C₃-alkyl-group is optionally substituted with 1 R⁴ group;
R^{3c} represents a hydrogen atom or a C₁-C₃-alkyl-group;
or
N(R^{3b})R^{3a} together
   form a 3- to 10-membered heterocycloalkyl-group; wherein said 3- to 10-membered heterocycloalkyl-group is optionally substituted, identically or differently, with 1, 2 or 3 R⁴ groups;
R⁴ represents halo-, hydroxy-, oxo-(O═), cyano-, nitro-, C₁-C₃-alkyl-, halo-C₁-C₃-alkyl-, C₁-C₃-alkoxy-, halo-C₁-C₃-alkoxy-, hydroxy-C₁-C₃-alkyl-, C₁-C₃-alkoxy-C₁-C₃-alkyl-, halo-C₁-C₃-alkoxy-C₁-C₃-alkyl-, R^{5c}—O—, —C(═O)—R^{5c}—C(═O)—O—R^{5c}, —O—C(═O)—R^{5c}, —N(R^{5b})—C(═O)—R^{5c}, —N(R^{5c})—C(═O)—N(R^{5a})R^{5b}, —N(R^{5a})R^{5b}, —C(═O)—N(R^{5a})R^{5b}, R^{5c}—S—, R^{5c}—S(═O)—, R^{5c}—S(═O)₂—, —N(R^{5c})—S(═O)—R^{5b}, —S(═O)—N(R^{5a})R^{5b}, —N(R^{5c})—S(═O)₂—R^{5b} or —S(═O)₂—N(R^{5a})R^{5b};
R^{5a} represents a hydrogen atom or a C₁-C₃-alkyl-group;
R^{5b} represents a hydrogen atom or a C₁-C₃-alkyl-group;
R^{5c} represents a hydrogen atom or a C₁-C₃-alkyl-group;
R^{6a} represents a hydrogen atom or a group selected from: C₁-C₆-alkyl-, C₂-C₆-alkenyl-, C₂-C₆-alkynyl-; wherein said C₁-C₆-alkyl-, C₂-C₆-alkenyl- or C₂-C₆-alkynyl-group is optionally substituted, identically or differently, with 1, 2 or 3 R⁴ groups;
R^{6b} represents a hydrogen atom or a C₁-C₃-alkyl-group;
R^{6c} represents a hydrogen atom or a C₁-C₃-alkyl-group;
p represents 0;
q represents 0;
r represents 1 or 2;
s represents 1 or 2; and
t represents 0;
or a tautomer, an N-oxide, a hydrate, a solvate, or a salt thereof, or a mixture of same.

In another preferred embodiment, the invention relates to compounds of formula I, supra, in which:
Q-V represents a group selected from: C(R^{1a})—N, N—C(R^{1a})
A represents a group selected from:

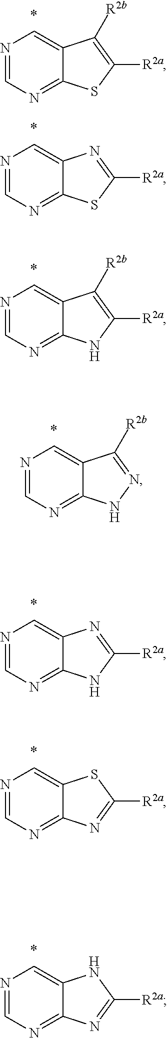

wherein * indicates the point of attachment of said groups to the rest of the molecule;
R^{1a} represents a hydrogen atom or a halogen atom or a group selected from: hydroxy-, cyano-, C₁-C₆-alkyl-, C₂-C₆-alkenyl-, C₂-C₆-alkynyl-, C₁-C₆-alkoxy-, halo-C₁-C₆-alkoxy-, —N(R^{5b})R^{5c}, —SCF₃, —SF₅;
R^{1b} represents a hydrogen atom;
R^{1c} represents a hydrogen atom;
one of R^{2a} and R^{2b} represents —(CH₂)_{q}—U—(CH₂)ₚ—R^{3a}, whereas the other one of R^{2a} and R^{2b} represents a hydrogen atom or a C₁-C₃-alkyl-group;
or
R^{2a} and R^{2b} together

represent —(CH₂)ᵣ-T-(CH₂)ₛ—
T represents a group selected from: U, —C[R^{6a}][(C(R^{6b})(R^{6c}))ₜ—U—R^{3a}]
U represents a single bond or a bivalent group selected from: —C(═O)—, —N(R^{3b})—, —C(═O)—O—, —O—C(═O)—, —C(═O)—N(R^{3b})—, —N(R^{3c})—C(═O)—;
R^{3a} represents a hydrogen atom or a group selected from: C₁-C₆-alkyl-, C₃-C₆-cycloalkyl-, 3- to 10-membered heterocycloalkyl-, 4- to 10-membered heterocycloalkenyl-, aryl-, heteroaryl-, halo-C₁-C₃-alkyl-; wherein said C₁-C₆-alkyl-, C₃-C₆-cycloalkyl-, 3- to 10-membered heterocycloalkyl-, aryl-, heteroaryl- or halo-C₁-C₃-alkyl-group is optionally substituted, identically or differently, with 1, 2, 3, 4 or 5 R⁴ groups, with the proviso that said halo-C₁-C₃-alkyl-group, together with the R⁴ groups optionally attached to it, does not contain more than 5 halogen atoms;
R^{3b} represents a hydrogen atom or a group selected from: C₁-C₆-alkyl-, C₃-C₆-cycloalkyl-, 3- to 10-membered heterocycloalkyl-, 4- to 10-membered heterocycloalkenyl-, aryl-, heteroaryl-, halo-C₁-C₃-alkyl-; wherein said C₁-C₆-alkyl-, C₃-C₆-cycloalkyl-, 3- to 10-membered heterocycloalkyl-, aryl-, heteroaryl- or halo-C₁-C₃-alkyl-group is optionally substituted, identically or differently, with 1, 2, 3, 4 or 5 R⁴ groups, with the proviso that said halo-C₁-C₃-alkyl-group, together with the R⁴ groups optionally attached to it, does not contain more than 5 halogen atoms;
R^{3c} represents a hydrogen atom or a group selected from: C₁-C₆-alkyl-, C₃-C₆-cycloalkyl-, 3- to 10-membered heterocycloalkyl-, 4- to 10-membered heterocycloalkenyl-, aryl-, heteroaryl-, halo-C₁-C₃-alkyl-; wherein said C₁-C₆-alkyl-, C₃-C₆-cycloalkyl-, 3- to 10-membered heterocycloalkyl-, aryl-, heteroaryl- or halo-C₁-C₃-alkyl-group is optionally substituted, identically or differently, with 1, 2, 3, 4 or 5 R⁴ groups, with the proviso that said halo-C₁-C₃-alkyl-group, together with the R⁴ groups optionally attached to it, does not contain more than 5 halogen atoms;
or
N(R^{3b})R^{3a} together
   form a 3- to 10-membered heterocycloalkyl- or 4- to 10-membered heterocycloalkenyl-group, wherein said 3- to 10-membered heterocycloalkyl- or 4- to 10-membered heterocycloalkenyl-group is optionally substituted, identically or differently, with 1, 2, 3, 4 or 5 R⁴ groups;
R⁴ represents halo-, hydroxy-, oxo-(O═), cyano-, nitro-, C₁-C₆-alkyl-, C₂-C₆-alkenyl-, C₂-C₆-alkynyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R^{5c}—O—, —C(═O)—R^{5c}, —C(═O)—O—R^{5c}, —O—C(═O)—R^{5c}, —N(R^{5b})—C(═O)—R^{5c}, —N(R^{5c})—C(═O)—N(R^{5a})R^{5b}, —N(R^{5a})R^{5b}, —C(═O)—N(R^{5a})R^{5b}, R^{5c}—S—, R^{5c}—S(═O)—, R^{5c}—S(═O)₂—, —N(R^{5c})—S(═O)—R^{5b}, —S(═O)—N(R^{5a})R^{5b}, —N(R^{5c})—S(═O)₂—R^{5b}, —S(═O)₂—N(R^{5a})R^{5b}, —S(═O)═N(R^{5c})R^{5b}, —S(═O)═N(R^{5c})R^{5b} or —N═S(═O)(R^{5c})R^{5b};
R^{5a} represents a hydrogen atom, a C₁-C₆-alkyl-, a C₃-C₆-cycloalkyl- or a 3- to 10-membered heterocycloalkyl-group;
R^{5b} represents a hydrogen atom, a C₁-C₆-alkyl-, a C₃-C₆-cycloalkyl- or a 3- to 10-membered heterocycloalkyl-group;
R^{5c} represents a hydrogen atom, a C₁-C₆-alkyl-, a C₃-C₆-cycloalkyl- or a 3- to 10-membered heterocycloalkyl-group;
or
N(R^{5a})R^{5b}

together form a 3- to 7-membered heterocycloalkyl-group;
R^{6a} represents a hydrogen atom or a group selected from: C₁-C₆-alkyl-, C₂-C₆-alkenyl-, C₂-C₆-alkynyl-; wherein said C₁-C₆-alkyl-, C₂-C₆-alkenyl- or C₂-C₆-alkynyl-group is optionally substituted, identically or differently, with 1, 2 or 3 R⁴ groups;
R^{6b} represents a hydrogen atom or a C₁-C₃-alkyl-group;
R^{6c} represents a hydrogen atom or a C₁-C₃-alkyl-group;
p represents 0 or 1;
q represents 0 or 1;
r represents 1 and s represents 2;
or
s represents 1 and r represents 2;
and
t represents an integer of 0 or 1;
or a tautomer, an N-oxide, a hydrate, a solvate, or a salt thereof, or a mixture of same.

In another preferred embodiment, the invention relates to compounds of formula I, supra, in which:
Q-V represents a group selected from: C(R^{1a})—N, N—C(R^{1a});
A represents a group selected from:

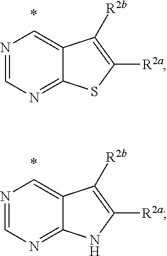

wherein * indicates the point of attachment of said groups to the rest of the molecule;
R^{1a} represents a hydrogen atom;
R^{1b} represents a hydrogen atom;
R^{1c} represents a hydrogen atom;
one of R^{2a} and R^{2b} represents —(CH₂)_{q}—U—(CH₂)ₚ—R^{3a}, whereas the other one of R^{2a} and R^{2b} represents a hydrogen atom or a C₁-C₃-alkyl-group;
or
R^{2a} and R^{2b} together

represent —(CH₂)ᵣ-T-(CH₂)ₛ—;
T represents a group selected from: U, —C[R^{6a}][(C(R^{6b})(R))ₜ—U—R^{3a}];
U represents a single bond or a bivalent group selected from: —C(═O)—, —N(R^{3b})—, —C(═O)—O—, —O—C(═O)—, —C(═O)—N(R^{3b})—, —N(R^{3c})—C(═O)—;
R^{3a} represents a hydrogen atom or a group selected from: C₁-C₆-alkyl-, C₃-C₆-cycloalkyl-, 3- to 10-membered heterocycloalkyl-, 4- to 10-membered heterocycloalkenyl-, aryl-, heteroaryl-, halo-C₁-C₃-alkyl-; wherein said C₁-C₆-alkyl-, C₃-C₆-cycloalkyl-, 3- to 10-membered heterocycloalkyl-, aryl-, heteroaryl- or halo-C₁-C₃-alkyl-group is optionally substituted, identically or differently, with 1, 2, 3, 4 or 5 R⁴ groups, with the proviso that said halo-C₁-C₃-alkyl-group, together with the R⁴ groups optionally attached to it, does not contain more than 5 halogen atoms;
R^{3b} represents a hydrogen atom or a group selected from: C₁-C₆-alkyl-, C₃-C₆-cycloalkyl-, 3- to 10-membered heterocycloalkyl-, 4- to 10-membered heterocycloalkenyl-, aryl-, heteroaryl-, halo-C₁-C₃-alkyl-; wherein said C₁-C₆-alkyl-, C₃-C₆-cycloalkyl-, 3- to 10-membered heterocycloalkyl-, aryl-, heteroaryl- or halo-C₁-C₃-alkyl-group is optionally substituted, identically or differently, with 1, 2, 3, 4 or 5 R⁴ groups, with the proviso that said halo-C₁-C₃-alkyl-group, together with the R⁴ groups optionally attached to it, does not contain more than 5 halogen atoms;
R^{3c} represents a hydrogen atom or a group selected from: C₁-C₆-alkyl-, C₃-C₆-cycloalkyl-, 3- to 10-membered heterocycloalkyl-, 4- to 10-membered heterocycloalkenyl-, aryl-, heteroaryl-, halo-C₁-C₃-alkyl-; wherein said C₁-C₆-alkyl-, C₃-C₆-cycloalkyl-, 3- to 10-membered heterocycloalkyl-, aryl-, heteroaryl- or halo-C₁-C₃-alkyl-group is optionally substituted, identically or differently, with 1, 2, 3, 4 or 5 R⁴ groups, with the proviso that said halo-C₁-C₃-alkyl-group, together with the R⁴ groups optionally attached to it, does not contain more than 5 halogen atoms;
or
N(R^{3b})R^{3a} together
   form a 3- to 10-membered heterocycloalkyl- or 4- to 10-membered heterocycloalkenyl-group; wherein said 3- to 10-membered heterocycloalkyl- or 4- to 10-membered heterocycloalkenyl-group is optionally substituted, identically or differently, with 1, 2, 3, 4 or 5 R⁴ groups;
R⁴ represents halo-, hydroxy-, oxo-(O═), cyano-, nitro-, C₁-C₆-alkyl-, C₂-C₆-alkenyl-, C₂-C₆-alkynyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R^{5c}—O, —C(═O)—R^{5c}, —C(═O)—O—R^{5c}, —O—C(═O)—R^{5c}, —N(R^{5b})—C(═O)—R^{5c}, —N(R^{5c})—C(═O)—N(R^{5a})R^{5b}, —N(R^{5a})R^{5b}, —C(═O)—N(R^{5a})R^{5b}, R^{5c}—S—, R^{5c}—S(═O)—, R^{5c}—S(═O)₂—, —N(R^{5c})—S(═O)—R^{5b}, —S(═O)—N(R^{5a})R^{5b}, —N(R^{5c})—S(═O)₂—R^{5b}, —S(═O)₂—N(R^{5a})R^{5b}, —S(═O)═N(R^{5c})R^{5b}, —S(═O)═N(R^{5c})R^{5b} or —N═S(═O)(R^{5c})R^{5b};
R^{5a} represents a hydrogen atom, a C₁-C₆-alkyl-, a C₃-C₆-cycloalkyl- or a 3- to 10-membered heterocycloalkyl-group;
R^{5b} represents a hydrogen atom, a C₁-C₆-alkyl-, a C₃-C₆-cycloalkyl- or a 3- to 10-membered heterocycloalkyl-group;
R^{5c} represents a hydrogen atom, a C₁-C₆-alkyl-, a C₃-C₆-cycloalkyl- or a 3- to 10-membered heterocycloalkyl-group;
or
N(R^{5a})R^{5b}

together form a 3- to 7-membered heterocycloalkyl-group;
R^{6a} represents a hydrogen atom or a C₁-C₆-alkyl-group; wherein said C₁-C₆-alkyl-group is optionally substituted, identically or differently, with 1, 2 or 3 R⁴ groups;
R^{6b} represents a hydrogen atom or a C₁-C₃-alkyl-group;
R^{6c} represents a hydrogen atom or a C₁-C₃-alkyl-group;
p represents 0 or 1;
q represents 0 or 1;
r represents 1 and s represents 2;
or
s represents 1 and r represents 2;
and
t represents an integer of 0 or 1;
or a tautomer, an N-oxide, a hydrate, a solvate, or a salt thereof, or a mixture of same.

It is to be understood that the present invention relates to any sub-combination within any embodiment or aspect of the present invention of compounds of general formula I, supra.

More particularly still, the present invention covers compounds of general formula I which are disclosed in the Examples section of this text, infra.

In accordance with another aspect, the present invention covers methods of preparing compounds of the present invention, said methods comprising the steps as described in the Experimental Section herein.

In a preferred embodiment, the present invention relates to a method of preparing compounds of general formula I, supra, in which method an intermediate compound of general formula IV:

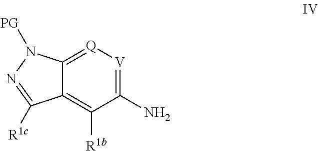

in which Q-V, R^{1b}, and R^{1c} are as defined for general formula I, supra, and PG represents a protective group or a hydrogen atom;
is allowed to react with an intermediate compound of general formula II:

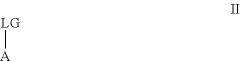

in which A is as defined for general formula I, supra, and LG represents a leaving group.

In another aspect, the present invention relates to intermediate compounds which are useful for the preparation of the compounds of general formula I, supra.

In particular, the present invention relates to intermediate compounds of general formula IV:

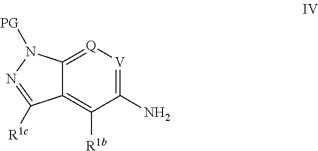

in which Q-V, R^{1b}, and R^{1c} are as defined for general formula I, supra, and PG represents a protective group or a hydrogen atom.

### Synthesis of Compounds of General Formula I of the Present Invention

Compounds of general formula I, I′, II, III and IV wherein R^{1b}, R^{1c}, Q-V, and A have the meaning as given for general formula I, supra, LG represents a leaving group and PG represents a protective group or a hydrogen atom, can be synthesized according to the procedures depicted in Scheme 1.

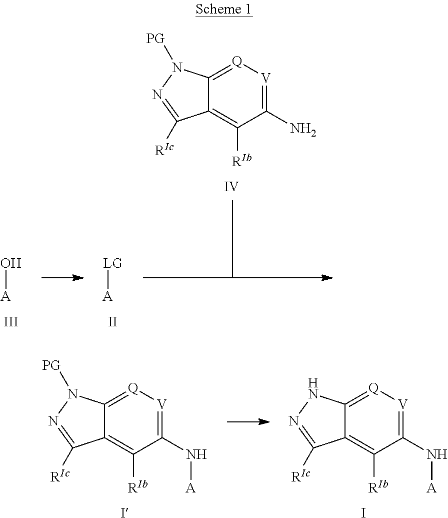

Scheme 1 exemplifies one route that allows variations and modifications in A, R^{1b}, and R^{1c} at different stages of the synthesis. However, also other routes may be used to synthesise the target compounds, in accordance with common general knowledge of a person skilled in the art of organic synthesis. The order of transformations exemplified in the Scheme is therefore not intended to be limiting. In addition, interconversion of any of the substituents as defined herein for A, R^{1b}, and R^{1c} can be achieved before and/or after the exemplified transformations.

These modifications can be such as the introduction of protective groups (PG), cleavage of protective groups, reduction or oxidation of functional groups, halogenation, metallation, substitution or other reactions known to a person skilled in the art.

These transformations include those which introduce a functionality which allows for further interconversion of substituents. Appropriate protective groups and their introduction and cleavage are well-known to a person skilled in the art (see for example T. W. Greene and P. G. M. Wuts in Protective Groups in Organic Synthesis, 3^{rd} edition, Wiley 1999). Specific examples are described in the subsequent paragraphs. Further, it is possible that two or more successive steps may be performed without work-up being performed between said steps, e.g. a “one-pot” reaction, as it is well-known to a person skilled in the art.

Compounds of formulae II, III, or IV are commercially available or can be synthesized according to procedures known to a person skilled in the art.

Compounds of formulae II in which LG represents a leaving group like, for example, a halogen atom as, for example, a chlorine or bromine atom may be commercially available or are obtained from compounds of formula III by reacting the alcohol with a halogenation agent like, for example, phosphorus trichloride or phosphorus tribromide with or without an additional inert solvent as, for example, toluene at temperatures ranging from room temperature to the boiling point of the solvent, for example.

Compounds of formula II in which LG represents a leaving group like, for example, an alkylsulfonate as, for example, methanesulfonate or trifluoromethanesulfonate or 1,1,2,2,3,3,4,4,4-nonafluorobutane-1-sulfonate or an arylsulfonate like, for example, benzenesulfonate or 4-methylbenzenesulfonate are obtained from compounds of formula III by reacting the alcohol with a suitable alkylsulfonyl halide as, for example, methanesulfonyl chloride or trifluoromethanesulfonyl chloride or 1,1,2,2,3,3,4,4,4-nonafluorobutane-1-sulfonyl fluoride or by reacting the alcohol with a suitable arylsulfonyl halide as, for example, benzenesulfonyl chloride or 4-methylbenzenesulfonyl chloride in an inert solvent like, for example, tetrahydrofuran or toluene or dichloromethane optionally in the presence of a suitable base like, for example, triethylamine or pyridine or N,N-dimethylpyridin-4-amine at temperatures ranging from −40° C. to the boiling point of the solvent, for example.

Compounds of formula I′ can be synthesized by reacting compounds of general formula II with a compound of general formula IV with R^{1b}, R^{1c}, V, Q and A as defined supra to give compounds of general formula I. The amino group present in IV displaces LG in compounds of general formula II to form amines of general formula I′ or I.

Compounds of general formula II can be reacted with amines of formula IV in which PG represents a protective group or a hydrogen atom optionally in the presence of an acid like, for example, hydrochloric acid in an inert solvent like, for example, ethanol or 1,4-dioxane at temperatures ranging from room temperature to the boiling point of the solvent, for example, to give compounds of general formula I′ or I.

Compounds of general formula I′ or I can also be built by Ullmann-type coupling reactions in the presence of suitable catalysts, such as, for example, copper based catalysts like copper(II)diacetate or copper(I) chloride in the presence of a suitable base, like for example, caesium carbonate starting from compounds of general formula IV. Optionally, suitable ligands like N,N-dimethylglycine or phenyl hydrogen pyrrolidin-2-ylphosphonate can be added. The reaction can be performed at temperatures ranging from −40° C. to the boiling point of the solvent, for example. In a similar way, palladium catalysed amination reactions can be employed to form compounds of general formula I′ or I from compounds of general formulae IV with II; for a contemporary review on such aminations see e.g. David S. Surry and Stephen L Buchwald, Chem. Sci. 2011, 2, 27, and the literature cited therein.

Compounds of general formula I, I′, II, III and IV in which R^{1a}, R^{1b}, R^{1c}, R^{2a} and/or R^{2b} represent a halogen atom such as, for example, a chlorine, bromine or iodine atom, can be further modified via coupling reactions such as, for example Ullmann-, Negishi-, Suzuki- or Sonogashira-type coupling reactions.

Said coupling reactions are performed in the presence of suitable catalysts, such as, for example, copper- or palladium based catalysts like, for example, copper(II)diacetate, copper(I) chloride, Palladium (II) acetate, tetrakis(triphenylphosphine)palladium (0), bis(triphenylphosphine)palladium (II) chloride or (1,1,-bis(diphenylphosphino) ferrocene)-dichloropalladium (II) and optionally suitable additives such as, for example, phosphines like, for example, P(oTol)₃ or triphenylphosphine and, and optionally with a suitable base, such as, for example, potassium carbonate, sodium 2-methylpropan-2-olate, tetrabutylammonium fluoride or tribasic potassium phosphate in a suitable solvent, such as, for example, tetrahydrofuran.

Examples of such coupling reactions may be found in the textbook entitled “Metal-Catalyzed Cross-Coupling Reactions”, Armin de Meijere (Editor), Frangois Diederich (Editor) September 2004, Wiley Interscience ISBN: 978-3-527-30518-6.

Compounds of general formulae I, I′, II, III and IV in which R^{1a}, R^{1b}, R^{1c}, R^{2a} and/or R^{2b} represent a halogen atom such as a fluorine, chlorine, bromine or iodine atom, can also be further modified via substitution reactions. Said halogen atoms in R^{1a}, R^{1b}, R^{1c}, R^{2a} and/or R^{2b} can be substituted by nucleophiles like primary or secondary amines, alkoxides, thiolates or carbon anion bearing groups to add secondary or tertiary amines, ethers, thioethers or carbon attached groups. The reactions are performed in inert solvents like tetrahydrofuran. Furthermore, residues in compounds of formulae I, I′, II, III and IV can be optionally modified using, for example, oxidation-, reduction-, substitution- or elimination-reactions and conditions that are well known to a person skilled in the art of organic synthesis. For example, thioethers can be oxidized using oxidation reagents like 3-chlorobenzenecarboperoxoic acid, oxone or dimethyldioxirane in inert solvents like dichloromethane or acetone, respectively. Depending on the stoichiometric ratio of oxidation reagent to the aforementioned compounds sulfoxides or sulfones or mixtures thereof will be obtained.

Further, the compounds of formula I of the present invention can be converted to any salt as described herein, by any method which is known to the person skilled in the art. Similarly, any salt of a compound of formula I of the present invention can be converted into the free compound, by any method which is known to the person skilled in the art.

The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallisation. In some cases, impurities may be removed by stirring using a suitable solvent. In some cases, the compounds may be purified by chromatography, particularly flash chromatography, using for example pre-packed silica gel cartridges, e.g. from Separtis such as Isolute® Flash silica gel or Isolute® Flash NH₂ silica gel in combination with a suitable chromatographic system such as an Isolera system (Biotage) and eluents such as, for example, gradients of hexane/ethyl acetate or dichloromethane/methanol. In some cases, the compounds may be purified by preparative HPLC using, for example, a Waters autopurifier equipped with a diode array detector and/or on-line electrospray ionisation mass spectrometer in combination with a suitable pre-packed reverse phase column and eluents such as, for example, gradients of water and acetonitrile which may contain additives such as trifluoroacetic acid, formic acid or aqueous ammonia.

### EXAMPLES

Chemical naming of the examples and intermediates was performed using ACD software by ACD/LABS (Name Batch version 12.01.)

### Example 1

### N-(7H-Pyrrolo[2,3-d]pyrimidin-4-yl)-1H-pyrazolo[3,4-b]pyridin-5-amine

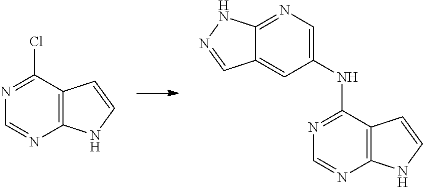 [SMILES: Clc1ncnc2[nH]ccc12 ; c1nc(Nc2cnc3[nH]ncc3c2)c2cc[nH]c2n1]

A mixture comprising 57.2 mg (373 μmol) 4-chloro-7H-pyrrolo[2,3-d]pyrimidine (CAS-No. 3680-69-1), 100 mg 1H-pyrazolo[3,4-b]pyridin-5-amine (CAS-No. 942185-01-5), 23.3 μL hydrochloric acid (4M) and 1.5 mL ethanol was heated under reflux for 6 hours. The solvent was removed to give 95.0 mg (100%) of the title compound.

¹H-NMR (DMSO-d6): δ=6.95 (1H), 7.46 (1H), 8.23 (1H), 8.30 (1H), 8.48 (1H), 8.68 (1H), 11.39 (1H), 12.74 (1H), 13.86 (1H) ppm.

### Example 2

### N-(7H-Pyrrolo[2,3-d]pyrimidin-4-yl)-1H-pyrazolo[3,4-c]pyridin-5-amine

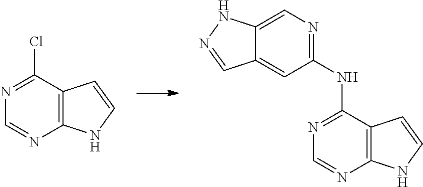 [SMILES: Clc1ncnc2[nH]ccc12 ; c1nc(Nc2cc3cn[nH]c3cn2)c2cc[nH]c2n1]

A mixture comprising 114.5 mg (745 μmol) 4-chloro-7H-pyrrolo[2,3-d]pyrimidine (CAS-No. 3680-69-1), 100 mg 1H-pyrazolo[3,4-c]pyridin-5-amine (CAS-No. 1049672-75-4), 93 μL hydrochloric acid (4M) and 5 mL ethanol was heated at 120° C. under microwave irradiation for 2 hours. The solvent was removed and the residue purified by chromatography to give 11.0 mg (6%) of the title compound.

¹H-NMR (DMSO-d6): δ=7.01 (1H), 7.20 (1H), 8.20 (1H), 8.34 (1H), 8.81-8.84 (2H), 9.86 (1H), 11.72 (1H), 13.43 (1H) ppm.

### Example 3

### N-(5-Methyl-7H-pyrrolo[2,3-d]pyrimidin-4-yl)-1H-pyrazolo[3,4-b]pyridin-5-amine

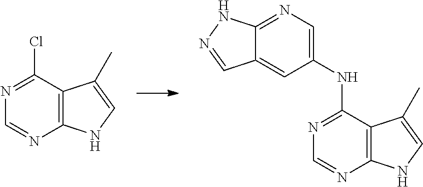 [SMILES: Cc1c[nH]c2ncnc(Cl)c12 ; Cc1c[nH]c2ncnc(Nc3cnc4[nH]ncc4c3)c12]

124.9 mg (745 μmol) 4-chloro-5-methyl-7H-pyrrolo[2,3-d]pyrimidine (CAS-No. 1618-36-6) were transformed in analogy to example 1 to give 198 mg (100%) of the title compound.

¹H-NMR (DMSO-d6): δ=2.52 (3H), 7.28 (1H), 8.17 (1H), 8.24 (1H), 8.42 (1H), 8.63 (1H), 9.75 (1H), 12.47 (1H), 13.87 (1H) ppm.

### Example 4

### N-(5-Methyl-7H-pyrrolo[2,3-d]pyrimidin-4-yl)-1H-pyrazolo[3,4-c]pyridin-5-amine

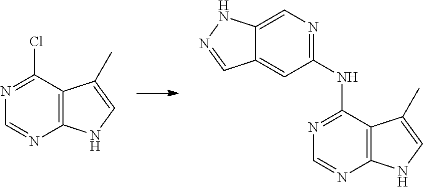 [SMILES: Cc1c[nH]c2ncnc(Cl)c12 ; Cc1c[nH]c2ncnc(Nc3cc4cn[nH]c4cn3)c12]

124.9 mg (745 μmol) 4-chloro-5-methyl-7H-pyrrolo[2,3-d]pyrimidine (CAS-No. 1618-36-6) were transformed in analogy to example 2 to give after working up and purification 26.0 mg (13%) of the title compound.

¹H-NMR (DMSO-d6): δ=2.53 (3H), 7.04 (1H), 8.12 (1H), 8.21 (1H), 8.33 (1H), 8.77 (1H), 8.81 (1H), 11.54 (1H), 13.45 (1H) ppm.

### Example 5

### N-(5-Ethyl-7H-pyrrolo[2,3-d]pyrimidin-4-yl)-1H-pyrazolo[3,4-b]pyridin-5-amine

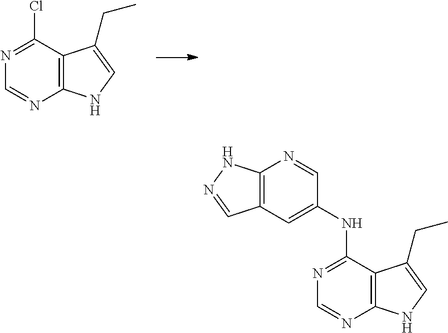 [SMILES: CCc1c[nH]c2ncnc(Cl)c12 ; CCc1c[nH]c2ncnc(Nc3cnc4[nH]ncc4c3)c12]

47 mg (259 μmol) 4-chloro-5-ethyl-7H-pyrrolo[2,3-d]pyrimidine (CAS-No. 1004992-44-2) were transformed in analogy to example 1 to give after working up and purification 45 mg (62%) of the title compound.

¹H-NMR (DMSO-d6): δ=1.28 (3H), 2.99 (2H), 7.27 (1H), 8.18 (1H), 8.24 (1H), 8.41 (1H), 8.62 (1H), 9.62 (1H), 12.51 (1H), 13.83 (1H) ppm.

### Example 6

### N-(5-Ethyl-7H-pyrrolo[2,3-d]pyrimidin-4-yl)-1H-pyrazolo[3,4-c]pyridin-5-amine

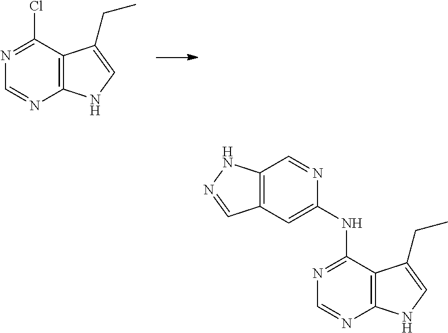 [SMILES: CCc1c[nH]c2ncnc(Cl)c12 ; CCc1c[nH]c2ncnc(Nc3cc4cn[nH]c4cn3)c12]

A mixture comprising 43 mg (237 μmol) 4-chloro-5-ethyl-7H-pyrrolo[2,3-d]pyrimidine (CAS-No. 1004992-44-2), 34.9 mg 1H-pyrazolo[3,4-c]pyridin-5-amine (CAS-No. 1049672-75-4), 17 μL hydrochloric acid (4M in dioxane) and 1.0 mL ethanol was heated in a sealed tube at 120° C. for 16 hours. The solvent was removed and the residue purified by chromatography to give 5.0 mg (8%) of the title compound.

¹H-NMR (DMSO-d6): δ=1.32 (3H), 2.96 (2H), 7.05 (1H), 8.01 (1H), 8.22 (1H), 8.35 (1H), 8.80 (1H), 8.82 (1H), 11.62 (1H), 13.48 (1H) ppm.

### Example 7

### N-(6-Ethyl-5-methyl-7H-pyrrolo[2,3-d]pyrimidin-4-yl)-1H-pyrazolo[3,4-b]pyridin-5-amine

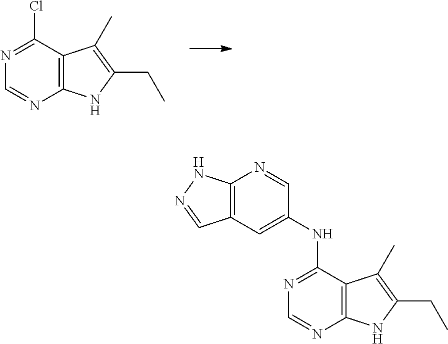 [SMILES: CCc1[nH]c2ncnc(Cl)c2c1C ; CCc1[nH]c2ncnc(Nc3cnc4[nH]ncc4c3)c2c1C]

A mixture comprising 50 mg (256 μmol) 4-chloro-6-ethyl-5-methyl-7H-pyrrolo[2,3-d]pyrimidine (prepared according to intermediate example 7a), 34.3 mg 1H-pyrazolo[3,4-b]pyridin-5-amine (CAS-No. 942185-01-5), 15.9 μL hydrochloric acid (4M in dioxane) and 0.8 mL ethanol was under microwave irradiation at 150° C. for 5 hours. Methanol was added, the precipitate filtered off, washed with methanol and diethyl ether and dried to give 56.5 mg (72%) of the title compound.

¹H-NMR (DMSO-d6): δ=1.20 (3H), 2.45 (3H), 2.71 (2H), 8.12 (1H), 8.22 (1H), 8.40 (1H), 8.62 (1H), 9.53 (1H), 12.39 (1H), 13.83 (1H) ppm.

### Example 7a

### 4-Chloro-6-ethyl-5-methyl-7H-pyrrolo[2,3-d]pyrimidine

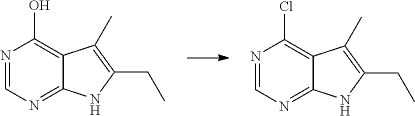

A mixture comprising 1.18 g (6.64 mmol) 6-ethyl-5-methyl-7H-pyrrolo[2,3-d]pyrimidin-4-ol (prepared according to intermediate example 7b) and 37.1 mL phosphorus oxychloride was heated at 100° C. for 1 hour. The reagent was removed and the residue purified by chromatography. The product was further purified by digestion with diethyl ether to give 855 mg (66%) of the title compound.

### Example 7b

### 6-Ethyl-5-methyl-7H-pyrrolo[2,3-d]pyrimidin-4-ol

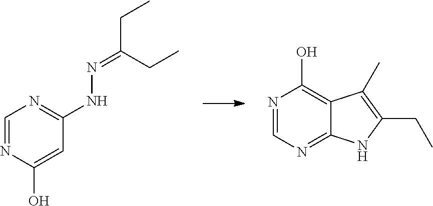

A mixture comprising 735 mg (3.78 mmol) 6-[2-(pentan-3-ylidene)hydrazino]pyrimidin-4-ol (prepared according to intermediate example 7c) and 20 mL 2-[2-(2-tert-butoxyethoxy)ethoxy]-2-methylpropane was heated at 250° C. for 2.5 hours. The solid was filtered off and washed with diethyl ether to give 477 mg (68%) of the title compound.

### Example 7c

### 6-[2-(Pentan-3-ylidene)hydrazino]pyrimidin-4-ol

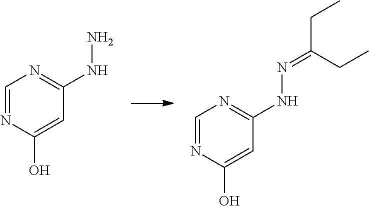

A mixture comprising 5.0 g (39.6 mmol) 6-hydrazinopyrimidin-4-ol/6-hydrazinopyrimidin-4(1H)-one (CAS-No: 29939-37-5), 5.12 g pentan-3-one and 80.8 mL ethanol was heated under reflux for 2 hours. After cooling to 3° C., the precipitated solid was filtered off and washed with diethyl ether to give 5.82 g (72%) of the title compound.

### Example 8

### N-(6-Ethyl-5-methyl-7H-pyrrolo[2,3-d]pyrimidin-4-yl)-1H-pyrazolo[3,4-c]pyridin-5-amine

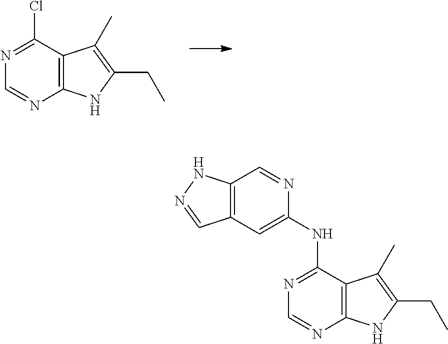 [SMILES: CCc1[nH]c2ncnc(Cl)c2c1C ; CCc1[nH]c2ncnc(Nc3cc4cn[nH]c4cn3)c2c1C]

50 mg (256 μmol) (256 μmol) 4-chloro-6-ethyl-5-methyl-7H-pyrrolo[2,3-d]pyrimidine (prepared according to intermediate example 7a) were transformed in analogy to example 7 using 1H-pyrazolo[3,4-c]pyridin-5-amine (CAS-No. 1049672-75-4) to give after working up and purification 15.4 mg (20%) of the title compound.

¹H-NMR (DMSO-d6): δ=1.19 (3H), 2.45 (3H), 2.66 (2H), 8.07 (1H), 8.20 (1H), 8.28 (1H), 8.75 (1H), 8.80 (1H), 11.51 (1H), 13.42 (1H) ppm.

### Example 9

### Ethyl 4-(1H-pyrazolo[3,4-b]pyridin-5-ylamino)-7H-pyrrolo[2,3-d]pyrimidine-6-carboxylate

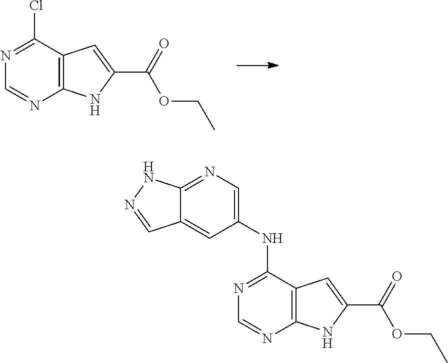

168 mg (745 μmol) ethyl 4-chloro-7H-pyrrolo[2,3-d]pyrimidine-6-carboxylate (CAS-No. 187725-00-4) were transformed in analogy to example 1 to give after working up and purification 204 mg (85%) of the title compound.

¹H-NMR (DMSO-d6): δ=1.34 (3H), 4.33 (2H), 7.58 (1H), 8.14 (1H), 8.36 (1H), 8.77 (2H), 9.86 (1H), 12.62 (1H), 13.59 (1H) ppm.

### Example 10

### Ethyl 4-(1H-pyrazolo[3,4-c]pyridin-5-ylamino)-7H-pyrrolo[2,3-d]pyrimidine-6-carboxylate

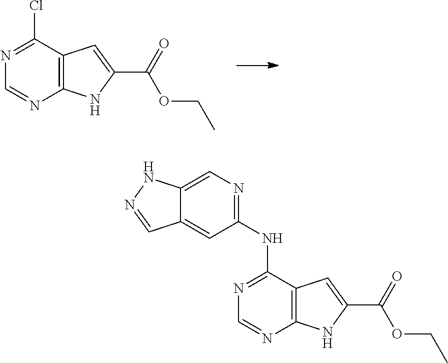

168 mg (745 μmol) ethyl 4-chloro-7H-pyrrolo[2,3-d]pyrimidine-6-carboxylate (CAS-No. 187725-00-4) were transformed in analogy to example 2 to give after working up and purification 220 mg (91%) of the title compound.

¹H-NMR (DMSO-d6): δ=1.35 (3H), 4.36 (2H), 8.11 (1H), 8.31 (1H), 8.37 (1H), 8.62 (1H), 8.99 (1H), 12.31 (1H), 13.47 (1H), 13.84 (1H) ppm.

### Example 11

### 4-(1H-Pyrazolo[3,4-c]pyridin-5-ylamino)-7H-pyrrolo[2,3-d]pyrimidine-6-carboxylic acid

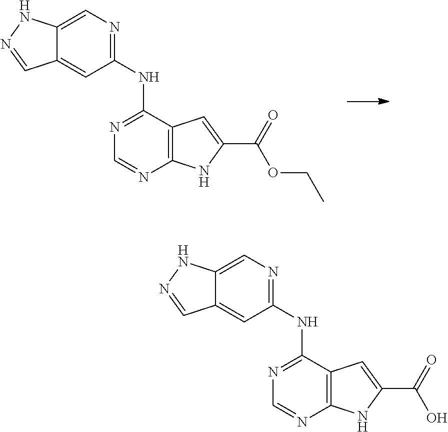 [SMILES: CCOC(=O)c1cc2c(Nc3cc4cn[nH]c4cn3)ncnc2[nH]1 ; O=C(O)c1cc2c(Nc3cc4cn[nH]c4cn3)ncnc2[nH]1]

A mixture comprising 1.00 g (3.09 mmol) ethyl 4-(1H-pyrazolo[3,4-c]pyridin-5-ylamino)-7H-pyrrolo[2,3-d]pyrimidine-6-carboxylate (prepared according to example 10), 30 mL dioxane, 10 mL ethanol and 37.1 mL lithium hydroxide solution (1M in water) was stirred at 23° C. overnight. The mixture was acidified with hydrochloric acid, the precipitate filtered off, washed with water and dried to give 820 mg (90%) of the title compound.

¹H-NMR (DMSO-d6): δ=8.23 (1H), 8.36 (2H), 8.64 (1H), 9.01 (1H), 12.92 (1H), 13.42 (1H), 13.92 (1H) ppm.

### Example 12

### Ethyl 5-bromo-4-(1H-pyrazolo[3,4-b]pyridin-5-ylamino)-7H-pyrrolo[2,3-d]pyrimidine-6-carboxylate

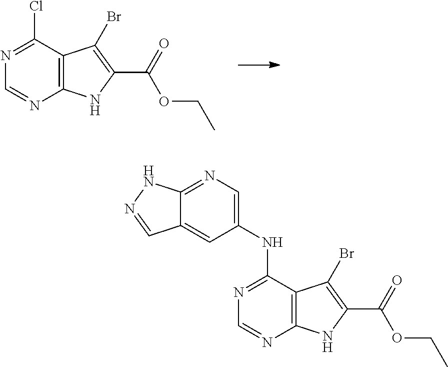

52 mg (171 μmol) ethyl 5-bromo-4-chloro-7H-pyrrolo[2,3-d]pyrimidine-6-carboxylate (prepared according to intermediate example 12a) were transformed in analogy to example 6 using 1H-pyrazolo[3,4-b]pyridin-5-amine (CAS-No. 942185-01-5) to give after working up and purification 17.0 mg (25%) of the title compound.

¹H-NMR (DMSO-d6): δ=1.35 (3H), 4.35 (2H), 8.15 (1H), 8.31 (1H), 8.55 (1H), 8.68 (1H), 8.73 (1H), 12.99 (1H), 13.63 (1H) ppm.

### Example 12a

### Ethyl 5-bromo-4-chloro-7H-pyrrolo[2,3-d]pyrimidine-6-carboxylate

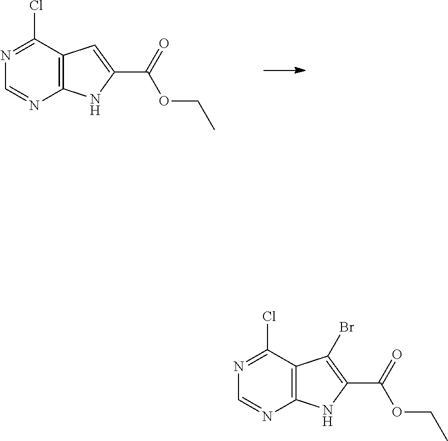 [SMILES: CCOC(=O)c1cc2c(Cl)ncnc2[nH]1 ; CCOC(=O)c1[nH]c2ncnc(Cl)c2c1Br]

A mixture comprising 1.01 g (4.45 mmol) ethyl 4-chloro-7H-pyrrolo[2,3-d]pyrimidine-6-carboxylate (CAS-No. 187725-00-4), 10 mL N,N-dimethylformamide and 832 mg N-bromosuccinimide was stirred at 23° C. for 16 hours. The mixture was poured into ice-cold water, the precipitate filtered off and dried to give 1.17 g (86%) of the title compound.

### Example 13

### Ethyl 5-bromo-4-(1H-pyrazolo[3,4-c]pyridin-5-ylamino)-7H-pyrrolo[2,3-d]pyrimidine-6-carboxylate

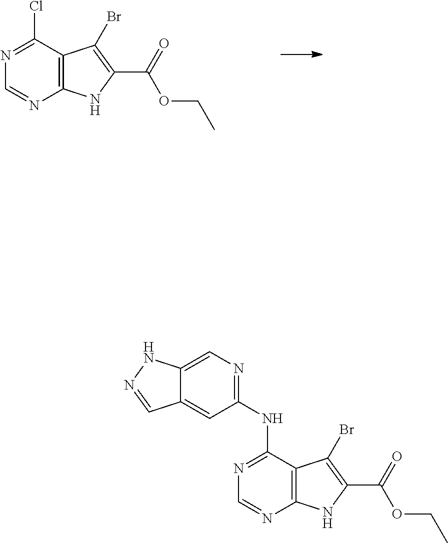 [SMILES: CCOC(=O)c1[nH]c2ncnc(Cl)c2c1Br ; CCOC(=O)c1[nH]c2ncnc(Nc3cc4cn[nH]c4cn3)c2c1Br]

49 mg (161 μmol) ethyl 5-bromo-4-chloro-7H-pyrrolo[2,3-d]pyrimidine-6-carboxylate (prepared according to intermediate example 12a) were transformed in analogy to example 6 to give after working up and purification 31.0 mg (65%) of the title compound.

¹H-NMR (DMSO-d6): δ=1.35 (3H), 4.36 (2H), 8.28 (1H), 8.55 (1H), 8.86 (1H), 8.90 (1H), 9.08 (1H), 13.14 (1H), 13.59 (1H) ppm.

### Example 14

### 5-Bromo-4-(1H-pyrazolo[3,4-c]pyridin-5-ylamino)-7H-pyrrolo[2,3-d]pyrimidine-6-carboxylic acid

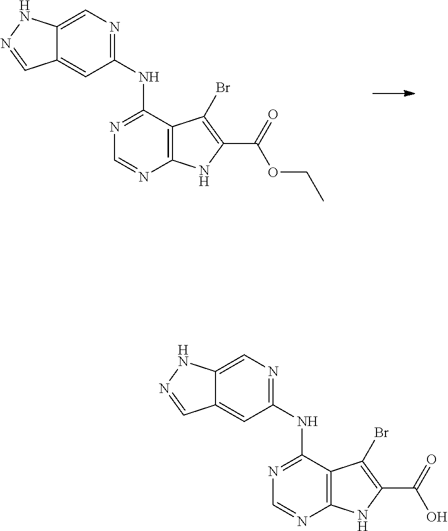 [SMILES: CCOC(=O)c1[nH]c2ncnc(Nc3cc4cn[nH]c4cn3)c2c1Br ; O=C(O)c1[nH]c2ncnc(Nc3cc4cn[nH]c4cn3)c2c1Br]

26 mg (65 μmol) ethyl 5-bromo-4-(1H-pyrazolo[3,4-c]pyridin-5-ylamino)-7H-pyrrolo[2,3-d]pyrimidine-6-carboxylate (prepared according to example 13) were transformed in analogy to example 11 to give after working up and purification 9.0 mg (37%) of the title compound.

¹H-NMR (DMSO-d6): δ=8.28 (1H), 8.53 (1H), 8.86 (1H), 8.87 (1H), 9.15 (1H), 13.03 (1H), 13.57 (1H) ppm.

### Example 15

### N-[5-(Trifluoromethyl)-7H-pyrrolo[2,3-d]pyrimidin-4-yl]-1H-pyrazolo[3,4-b]pyridin-5-amine

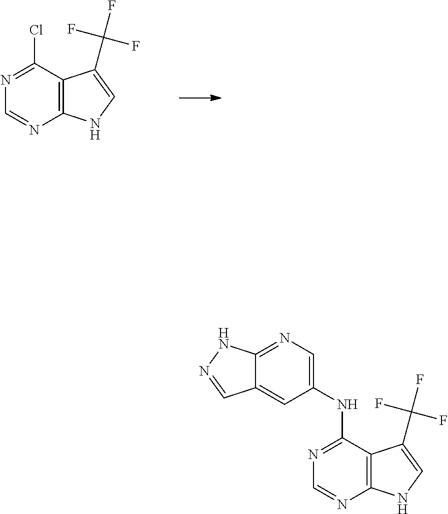 [SMILES: FC(F)(F)c1c[nH]c2ncnc(Cl)c12 ; FC(F)(F)c1c[nH]c2ncnc(Nc3cnc4[nH]ncc4c3)c12]

50 mg (226 μmol) 4-chloro-5-(trifluoromethyl)-7H-pyrrolo[2,3-d]pyrimidine (prepared according to intermediate example 15a) were transformed in analogy to example 2 using 1H-pyrazolo[3,4-b]pyridin-5-amine (CAS-No. 942185-01-5) to give after working up and purification 55 mg (76%) of the title compound.

¹H-NMR (DMSO-d6): δ=7.38 (1H), 8.16 (1H), 8.39 (1H), 8.69 (1H), 8.74 (1H), 10.12 (1H), 13.20 (1H), 13.63 (1H) ppm.

### Example 15a

### 4-Chloro-5-(trifluoromethyl)-7H-pyrrolo[2,3-d]pyrimidine

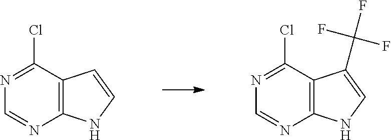 [SMILES: Clc1ncnc2[nH]ccc12 ; FC(F)(F)c1c[nH]c2ncnc(Cl)c12]

To a mixture comprising 940 mg (6.12 mmol) 4-chloro-7H-pyrrolo[2,3-d]pyrimidine (CAS-No. 3680-69-1), 2.87 g sodium trifluoromethylsulfinate, 26 mL dichloromethane and 10 mL water were slowly added at 0° C. 4.37 mL tert-butylhydroperoxide. The mixture was stirred at 23° C. for 3 days, dichloromethane and saturated sodium bicarbonate solution were added, the aqueous layer was extracted with dichloromethane and the combined organic layers dried over sodium sulfate. After filtration and removal of the solvent, the residue was purified by chromatography to give 247 mg (18%) of the title compound.

### Example 16

### N-[5-(Trifluoromethyl)-7H-pyrrolo[2,3-d]pyrimidin-4-yl]-1H-pyrazolo[3,4-c]pyridin-5-amine

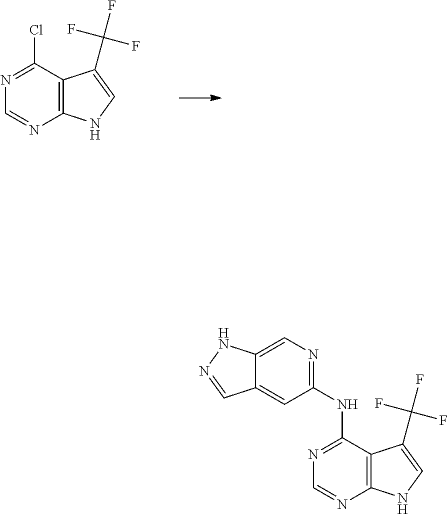 [SMILES: FC(F)(F)c1c[nH]c2ncnc(Cl)c12 ; FC(F)(F)c1c[nH]c2ncnc(Nc3cc4cn[nH]c4cn3)c12]

50 mg (226 μmol) 4-chloro-5-(trifluoromethyl)-7H-pyrrolo[2,3-d]pyrimidine (prepared according to intermediate example 15a) were transformed in analogy to example 2 to give after working up and purification 55.0 mg (69%) of the title compound.

¹H-NMR (DMSO-d6): δ=7.96 (1H), 8.34 (1H), 8.37 (1H), 8.65 (1H), 8.99 (1H), 12.24 (1H), 13.77 (1H), 13.90 (1H) ppm.

### Example 17

### [4-(1H-Pyrazolo[3,4-b]pyridin-5-ylamino)-7H-pyrrolo[2,3-d]pyrimidin-5-yl]methanol

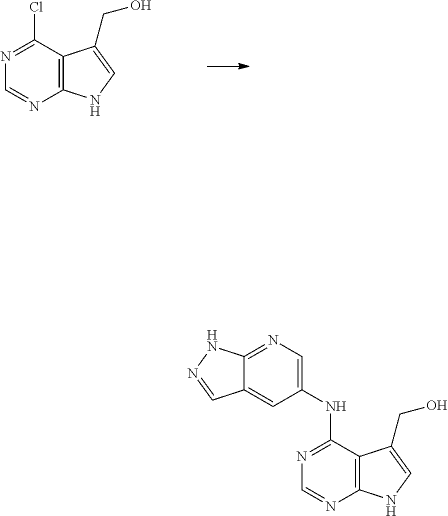 [SMILES: OCc1c[nH]c2ncnc(Cl)c12 ; OCc1c[nH]c2ncnc(Nc3cnc4[nH]ncc4c3)c12]

50 mg (272 μmol) (4-chloro-7H-pyrrolo[2,3-d]pyrimidin-5-yl)methanol (purchased from FCH Group Company, Ukraine) were transformed in analogy to example 2 using 1H-pyrazolo[3,4-b]pyridin-5-amine (CAS-No. 942185-01-5) to give after working up and purification 24.0 mg (28%) of the title compound.

¹H-NMR (DMSO-d6): δ=4.78 (2H), 6.42 (1H), 7.18 (1H), 8.12 (1H), 8.29 (1H), 8.58 (1H), 8.83 (1H), 10.09 (1H), 11.63 (1H), 13.52 (1H) ppm.

### Example 18

### N,N-Dimethyl-4-(1H-pyrazolo[3,4-c]pyridin-5-ylamino)-7H-pyrrolo[2,3-d]pyrimidine-6-carboxamide

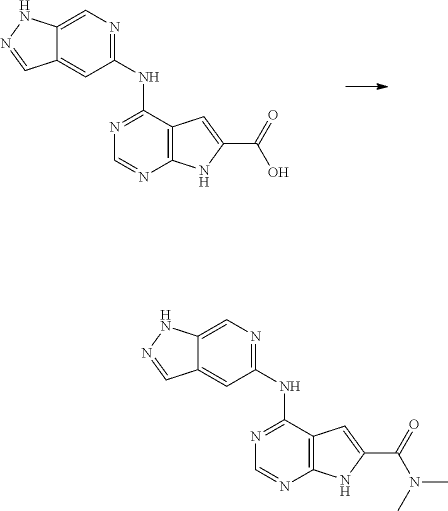 [SMILES: O=C(O)c1cc2c(Nc3cc4cn[nH]c4cn3)ncnc2[nH]1 ; CN(C)C(=O)c1cc2c(Nc3cc4cn[nH]c4cn3)ncnc2[nH]1]

A mixture comprising 50 mg (169 μmol) 4-(1H-Pyrazolo[3,4-c]pyridin-5-ylamino)-7H-pyrrolo[2,3-d]pyrimidine-6-carboxylic acid (prepared according to example 11), 2 mL dimethylsulfoxide, 88 μL N-ethyl-N-isopropylpropan-2-amine, 51 μL N-methylmethanamine and 302 μL 2,4,6-tripropyl-1,3,5,2,4,6-trioxatriphosphinane 2,4,6-trioxide solution (50% in ethyl acetate) was stirred at 23° C. overnight. The solvents were removed and the residue purified by chromatography to give 11 mg (20%) of the title compound.

¹H-NMR (DMSO-d6): δ=3.34 (6H), 7.56 (1H), 8.23 (1H), 8.41 (1H), 8.82-8.89 (2H), 10.18 (1H), 12.16 (1H), 13.47 (1H) ppm.

### Example 19

### [4-(1H-Pyrazolo[3,4-c]pyridin-5-ylamino)-7H-pyrrolo[2,3-d]pyrimidin-6-yl](pyrrolidin-1-yl)methanone

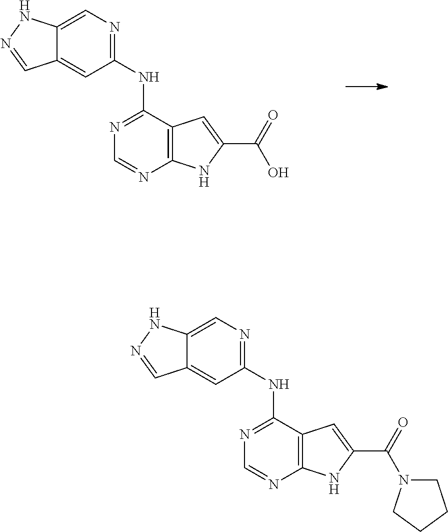 [SMILES: O=C(O)c1cc2c(Nc3cc4cn[nH]c4cn3)ncnc2[nH]1 ; O=C(c1cc2c(Nc3cc4cn[nH]c4cn3)ncnc2[nH]1)N1CCCC1]

50 mg (169 μmol) 4-(1H-Pyrazolo[3,4-c]pyridin-5-ylamino)-7H-pyrrolo[2,3-d]pyrimidine-6-carboxylic acid (prepared according to example 11) were transformed in analogy to example 18 using pyrrolidine to give after working up and purification 4.2 mg (7%) of the title compound.

¹H-NMR (DMSO-d6): δ=1.89 (2H), 2.00 (2H), 3.54 (2H), 3.81 (2H), 7.72 (1H), 8.22 (1H), 8.42 (1H), 8.85 (1H), 8.87 (1H), 10.20 (1H), 12.12 (1H), 13.48 (1H) ppm.

### Example 20

### Piperidin-1-yl[4-(1H-pyrazolo[3,4-c]pyridin-5-ylamino)-7H-pyrrolo[2,3-d]pyrimidin-6-yl]methanone

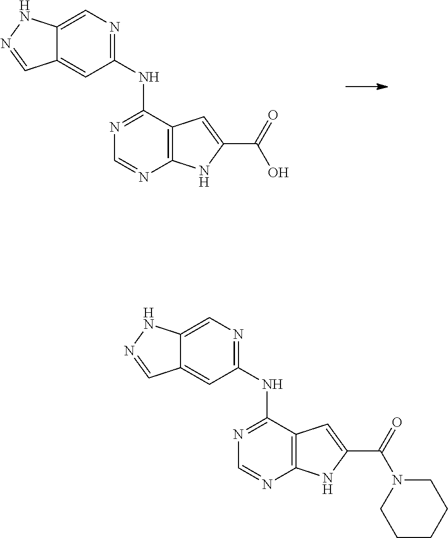 [SMILES: O=C(O)c1cc2c(Nc3cc4cn[nH]c4cn3)ncnc2[nH]1 ; O=C(c1cc2c(Nc3cc4cn[nH]c4cn3)ncnc2[nH]1)N1CCCCC1]

50 mg (169 μmol) 4-(1H-Pyrazolo[3,4-c]pyridin-5-ylamino)-7H-pyrrolo[2,3-d]pyrimidine-6-carboxylic acid (prepared according to example 11) were transformed in analogy to example 18 using piperidine to give after working up and purification 6.4 mg (10%) of the title compound.

¹H-NMR (DMSO-d6): δ=1.55-1.64 (4H), 1.68 (2H), 3.68 (4H), 7.39 (1H), 8.22 (1H), 8.40 (1H), 8.83-8.86 (2H), 10.16 (1H), 12.15 (1H), 13.47 (1H) ppm.

### Example 21

### Morpholin-4-yl[4-(1H-pyrazolo[3,4-c]pyridin-5-ylamino)-7H-pyrrolo[2,3-d]pyrimidin-6-yl]methanone

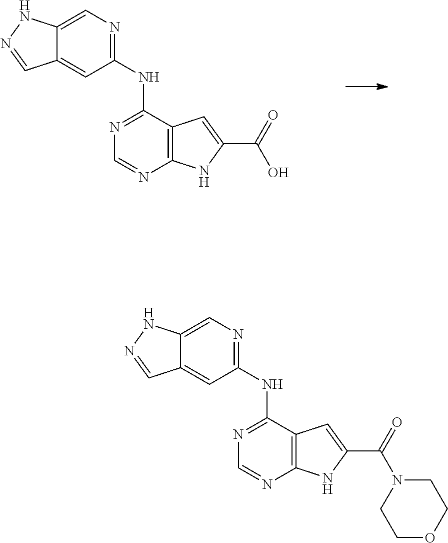 [SMILES: O=C(O)c1cc2c(Nc3cc4cn[nH]c4cn3)ncnc2[nH]1 ; O=C(c1cc2c(Nc3cc4cn[nH]c4cn3)ncnc2[nH]1)N1CCOCC1]

50 mg (169 μmol) 4-(1H-Pyrazolo[3,4-c]pyridin-5-ylamino)-7H-pyrrolo[2,3-d]pyrimidine-6-carboxylic acid (prepared according to example 11) were transformed in analogy to example 18 using morpholine to give after working up and purification 11.0 mg (18%) of the title compound.

¹H-NMR (DMSO-d6): δ=3.62-3.80 (8H), 7.46 (1H), 8.22 (1H), 8.42 (1H), 8.85 (2H), 10.15 (1H), 12.23 (1H), 13.46 (1H) ppm.

### Example 22

### N-[2-(Dimethylamino)ethyl]-4-(1H-pyrazolo[3,4-c]pyridin-5-ylamino)-7H-pyrrolo[2,3-d]pyrimidine-6-carboxamide

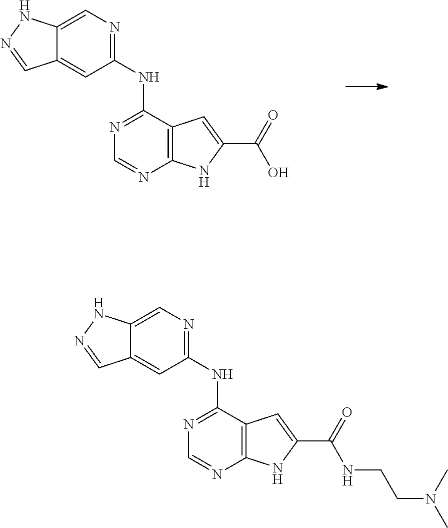 [SMILES: O=C(O)c1cc2c(Nc3cc4cn[nH]c4cn3)ncnc2[nH]1 ; CN(C)CCNC(=O)c1cc2c(Nc3cc4cn[nH]c4cn3)ncnc2[nH]1]

50 mg (169 μmol) 4-(1H-Pyrazolo[3,4-c]pyridin-5-ylamino)-7H-pyrrolo[2,3-d]pyrimidine-6-carboxylic acid (prepared according to example 11) were transformed in analogy to example 18 using N,N-dimethylethane-1,2-diamine to give after working up and purification 10.0 mg (16%) of the title compound.

¹H-NMR (DMSO-d6): δ=2.19 (6H), 2.41 (2H), 3.37 (2H), 7.49 (1H), 8.07 (1H), 8.22 (1H), 8.40 (1H), 8.73 (1H), 8.85 (1H), 10.13 (1H), 12.16 (1H), 13.48 (1H) ppm.

### Example 23

### (RS)—N-(Propan-2-yl)-4-(1H-pyrazolo[3,4-b]pyridin-5-ylamino)-5,6,7,8-tetrahydro[1]benzothieno[2,3-d]pyrimidine-7-carboxamide

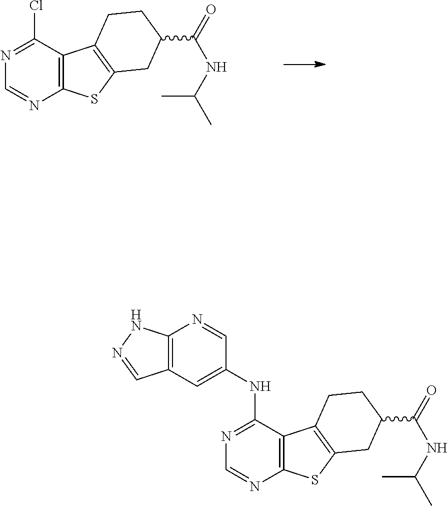 [SMILES: CC(C)NC(=O)C1CCc2c(sc3ncnc(Cl)c23)C1 ; CC(C)NC(=O)C1CCc2c(sc3ncnc(Nc4cnc5[nH]ncc5c4)c23)C1]

100 mg (323 μmol) (RS)-4-chloro-N-isopropyl-5,6,7,8-tetrahydro[1]benzothieno[2,3-d]pyrimidine-7-carboxamide (prepared according to example 23a) were transformed in analogy to example 6 using 1H-pyrazolo[3,4-b]pyridin-5-amine (CAS-No. 942185-01-5) to give after working up and purification 23.5 mg (17%) of the title compound.

¹H-NMR (DMSO-d6): δ=1.00-1.11 (6H), 1.79 (1H), 2.05 (1H), 2.59 (1H), 2.90 (2H), 3.08 (1H), 3.26 (1H), 3.41 (1H), 3.85 (1H), 7.81 (1H), 8.11 (1H), 8.27 (1H), 8.33 (1H), 8.58 (1H), 13.58 (1H) ppm.

### Example 23a

### (RS)-4-Chloro-N-isopropyl-5,6,7,8-tetrahydro[1]benzothieno[2,3-d]pyrimidine-7-carboxamide

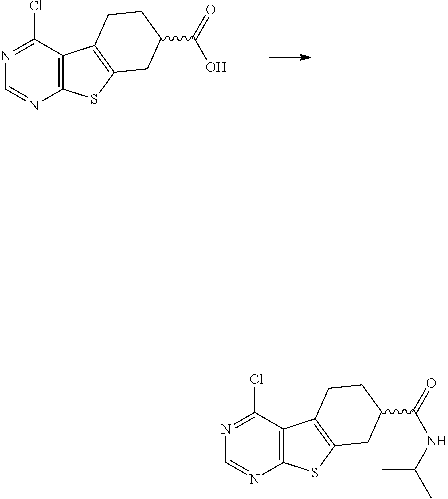 [SMILES: O=C(O)C1CCc2c(sc3ncnc(Cl)c23)C1 ; CC(C)NC(=O)C1CCc2c(sc3ncnc(Cl)c23)C1]

13.2 g (49.0 mmol) (RS)-4-chloro-5,6,7,8-tetrahydro[1]benzothieno[2,3-d]pyrimidine-7-carboxylic acid (prepared according to intermediate example 23b) were transformed in analogy to example 18 using propan-2-amine to give after working up and purification 11.4 g (71%) of the title compound.

### Example 23b

### (RS)-4-Chloro-5,6,7,8-tetrahydro[1]benzothieno[2,3-d]pyrimidine-7-carboxylic acid

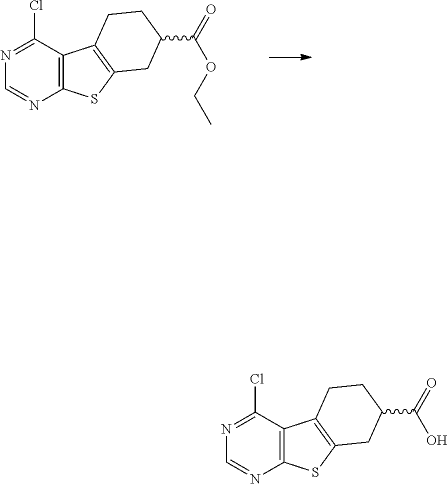 [SMILES: CCOC(=O)C1CCc2c(sc3ncnc(Cl)c23)C1 ; O=C(O)C1CCc2c(sc3ncnc(Cl)c23)C1]

20.0 g (37.4 mmol) (RS)-ethyl 4-chloro-5,6,7,8-tetrahydro[1]benzothieno[2,3-d]pyrimidine-7-carboxylate (prepared according to intermediate example 23c) were transformed in analogy to example 11 to give after working up and purification 17.2 g (95%) of the title compound.

### Example 23c

### (RS)-Ethyl 4-chloro-5,6,7,8-tetrahydro[1]benzothieno[2,3-d]pyrimidine-7-carboxylate

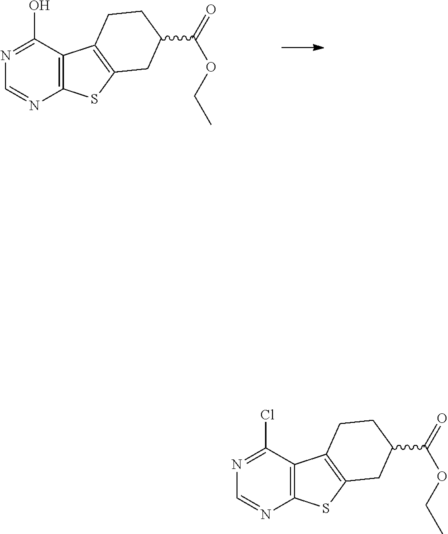 [SMILES: CCOC(=O)C1CCc2c(sc3ncnc(O)c23)C1 ; CCOC(=O)C1CCc2c(sc3ncnc(Cl)c23)C1]

A mixture comprising 195 g (700.6 mmol) (RS)-ethyl 4-hydroxy-5,6,7,8-tetrahydro[1]benzothieno[2,3-d]pyrimidine-7-carboxylate (prepared according to WO2005/10008), 1.92 L toluene, 195 mL N-ethyl-N-isopropylpropan-2-amine and 78.4 mL phosphorus oxychloride was heated at 80° C. overnight. The mixture was poured into sodium hydrogencarbonate solution and extracted with ethyl acetate. The organic layer was washed with brine and dried over sodium sulphate. After filtration and removal of the solvent the residue was crystallized from diisopropyl ether to give 120 g (58%) of the title compound.

### Example 24

### (RS)—N-(Propan-2-yl)-4-(1H-pyrazolo[3,4-c]pyridin-5-ylamino)-5,6,7,8-tetrahydro[1]benzothieno[2,3-d]pyrimidine-7-carboxamide

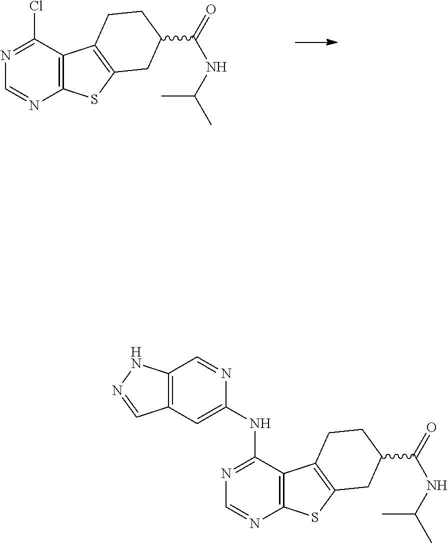 [SMILES: CC(C)NC(=O)C1CCc2c(sc3ncnc(Cl)c23)C1 ; CC(C)NC(=O)C1CCc2c(sc3ncnc(Nc4cc5cn[nH]c5cn4)c23)C1]

100 mg (323 μmol) (RS)-4-chloro-N-isopropyl-5,6,7,8-tetrahydro[1]benzothieno[2,3-d]pyrimidine-7-carboxamide (prepared according to example 23a) were transformed in analogy to example 6 to give after working up and purification 33.3 mg (24%) of the title compound.

¹H-NMR (DMSO-d6): δ=1.01-1.10 (6H), 1.80 (1H), 2.07 (1H), 2.58 (1H), 2.90 (2H), 3.05-3.45 (2H), 3.84 (1H), 7.84 (1H), 8.21 (1H), 8.36 (1H), 8.50 (1H), 8.63 (1H), 8.82 (1H), 13.54 (1H) ppm.

### Example 25

### (RS)-(4-Methylpiperazin-1-yl)[4-(1H-pyrazolo[3,4-b]pyridin-5-ylamino)-5,6,7,8-tetrahydro[1]benzothieno[2,3-d]pyrimidin-7-yl]methanone

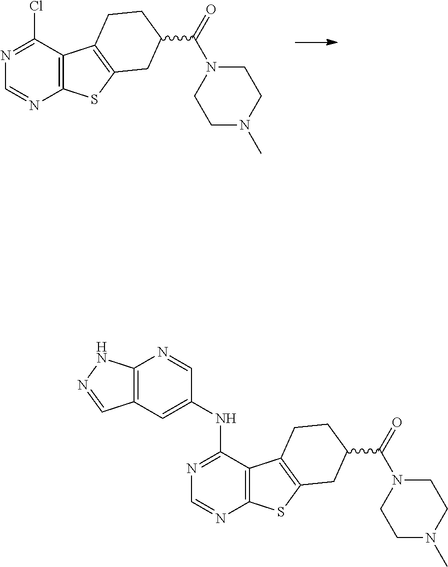 [SMILES: CN1CCN(C(=O)C2CCc3c(sc4ncnc(Cl)c34)C2)CC1 ; CN1CCN(C(=O)C2CCc3c(sc4ncnc(Nc5cnc6[nH]ncc6c5)c34)C2)CC1]

70 mg (200 μmol) (RS)-(4-chloro-5,6,7,8-tetrahydro[1]benzothieno[2,3-d]pyrimidin-7-yl)(4-methylpiperazin-1-yl)methanone (prepared according to intermediate example 25a) were transformed in analogy to example 7 to give after working up and purification 38.0 mg (38%) of the title compound.

¹H-NMR (DMSO-d6): δ=1.81 (1H), 2.04 (1H), 2.21 (3H), 2.30 (2H), 2.37 (2H), 2.84-3.06 (2H), 3.13-3.29 (3H), 3.50 (2H), 3.57 (2H), 8.13 (1H), 8.30 (1H), 8.36 (2H), 8.60 (1H), 13.61 (1H) ppm.

### Example 25a

### (RS)-(4-Chloro-5,6,7,8-tetrahydro[1]benzothieno[2,3-d]pyrimidin-7-yl)(4-methylpiperazin-1-yl)methanone

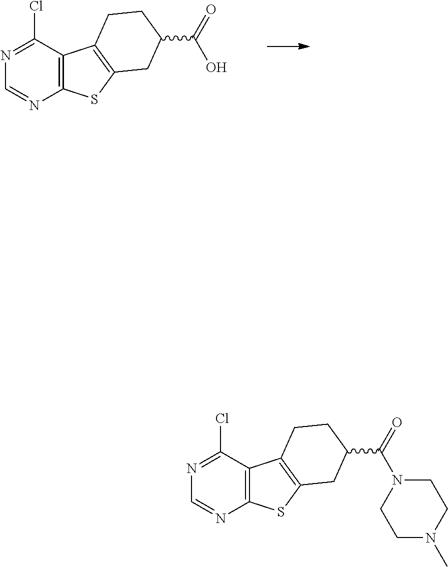 [SMILES: O=C(O)C1CCc2c(sc3ncnc(Cl)c23)C1 ; CN1CCN(C(=O)C2CCc3c(sc4ncnc(Cl)c34)C2)CC1]

500 mg (1.861 mmol) (RS)-4-chloro-5,6,7,8-tetrahydro[1]benzothieno[2,3-d]pyrimidine-7-carboxylic acid (prepared according to intermediate example 23b) were transformed in analogy to example 18 using 1-methylpiperazine to give after working up and purification 204 mg (28%) of the title compound.

### Example 26

### (RS)-(4-Methylpiperazin-1-yl)[4-(1H-pyrazolo[3,4-c]pyridin-5-ylamino)-5,6,7,8-tetrahydro[1]benzothieno[2,3-d]pyrimidin-7-yl]methanone

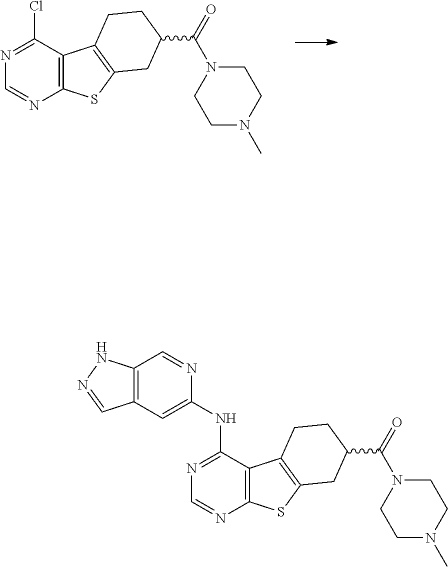 [SMILES: CN1CCN(C(=O)C2CCc3c(sc4ncnc(Cl)c34)C2)CC1 ; CN1CCN(C(=O)C2CCc3c(sc4ncnc(Nc5cc6cn[nH]c6cn5)c34)C2)CC1]

70 mg (200 μmol) (RS)-(4-chloro-5,6,7,8-tetrahydro[1]benzothieno[2,3-d]pyrimidin-7-yl)(4-methylpiperazin-1-yl)methanone (prepared according to intermediate example 25a) were transformed in analogy to example 7 using 1H-pyrazolo[3,4-c]pyridin-5-amine to give after working up and purification 14.6 mg (16%) of the title compound.

¹H-NMR (DMSO-d6): δ=1.81 (1H), 2.10 (1H), 2.19 (3H), 2.25-2.38 (4H), 2.86-3.04 (2H), 3.16 (1H), 3.27 (2H), 3.50 (2H), 3.56 (2H), 8.24 (1H), 8.33 (1H), 8.53 (1H), 8.68 (1H), 8.84 (1H), 13.55 (1H) ppm.

### Example 27

### (RS)—N,N-Dimethyl-4-(1H-pyrazolo[3,4-b]pyridin-5-ylamino)-5,6,7,8-tetrahydro[1]benzothieno[2,3-d]pyrimidine-7-carboxamide

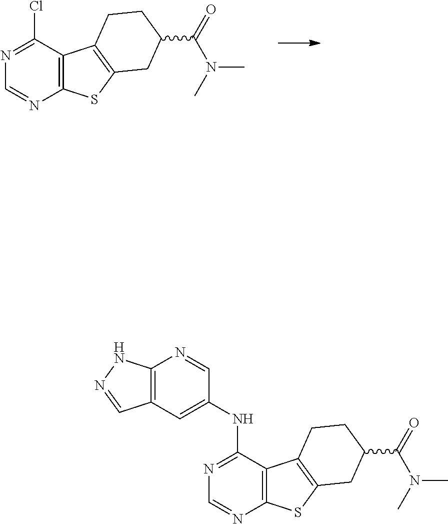 [SMILES: CN(C)C(=O)C1CCc2c(sc3ncnc(Cl)c23)C1 ; CN(C)C(=O)C1CCc2c(sc3ncnc(Nc4cnc5[nH]ncc5c4)c23)C1]

60 mg (203 μmol) (RS)-4-chloro-N,N-dimethyl-5,6,7,8-tetrahydro[1]benzothieno[2,3-d]pyrimidine-7-carboxamide (prepared according to intermediate example 27a) were transformed in analogy to example 6 using 1H-pyrazolo[3,4-b]pyridin-5-amine to give after working up and purification 10.6 mg (13%) of the title compound.

¹H-NMR (DMSO-d6): δ=1.76 (1H), 2.05 (1H), 2.85 (3H), 2.87-2.98 (2H), 3.07 (3H), 3.10-3.23 (2H), 3.31 (1H), 8.11 (1H), 8.27 (1H), 8.34 (2H), 8.58 (1H), 13.58 (1H) ppm.

### Example 27a

### (RS)-4-Chloro-N,N-dimethyl-5,6,7,8-tetrahydro[1]benzothieno[2,3-d]pyrimidine-7-carboxamide

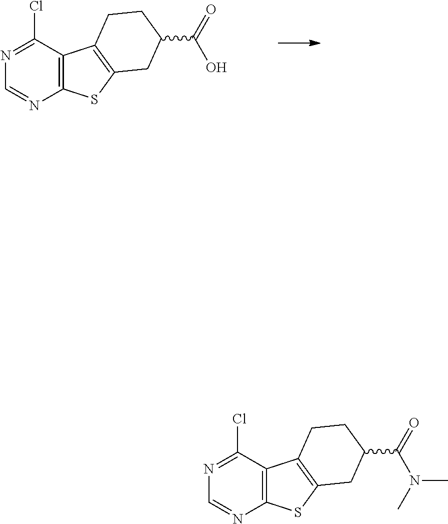 [SMILES: O=C(O)C1CCc2c(sc3ncnc(Cl)c23)C1 ; CN(C)C(=O)C1CCc2c(sc3ncnc(Cl)c23)C1]

500 mg (1.861 mmol) (RS)-4-chloro-5,6,7,8-tetrahydro[1]benzothieno[2,3-d]pyrimidine-7-carboxylic acid (prepared according to intermediate example 23b) were transformed in analogy to example 18 using N-methylmethanamine to give after working up and purification 350 mg (64%) of the title compound.

### Example 28

### (RS)—N,N-Dimethyl-4-(1H-pyrazolo[3,4-c]pyridin-5-ylamino)-5,6,7,8-tetrahydro[1]benzothieno[2,3-d]pyrimidine-7-carboxamide

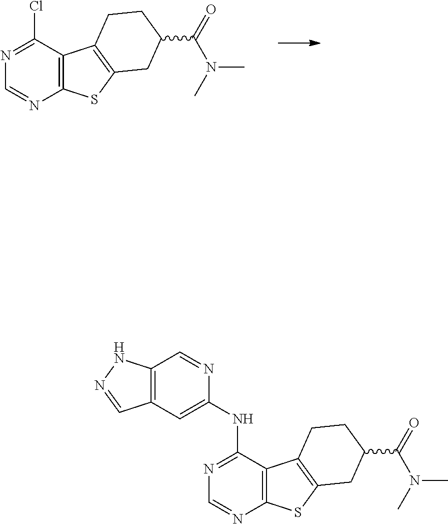 [SMILES: CN(C)C(=O)C1CCc2c(sc3ncnc(Cl)c23)C1 ; CN(C)C(=O)C1CCc2c(sc3ncnc(Nc4cc5cn[nH]c5cn4)c23)C1]

60 mg (203 μmol) (RS)-4-chloro-N,N-dimethyl-5,6,7,8-tetrahydro[1]benzothieno[2,3-d]pyrimidine-7-carboxamide (prepared according to intermediate example 27a) were transformed in analogy to example 6 to give after working up and purification 24.7 mg (29%) of the title compound.

¹H-NMR (DMSO-d6): δ=1.76 (1H), 2.10 (1H), 2.85 (3H), 2.86-2.97 (2H), 3.07 (3H), 3.11 (1H), 3.24 (2H), 8.21 (1H), 8.32 (1H), 8.51 (1H), 8.66 (1H), 8.81 (1H), 13.53 (1H) ppm.

### Example 29

### (RS)—N-(Propan-2-yl)-4-(1H-pyrazolo[3,4-b]pyridin-5-ylamino)-6,7,8,9-tetrahydro-5H-pyrimido[4,5-b]indole-6-carboxamide

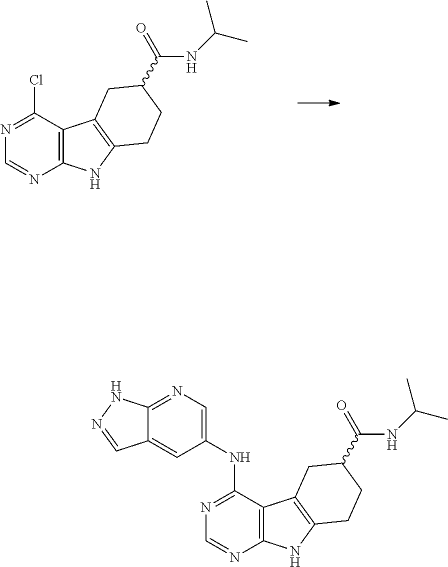 [SMILES: CC(C)NC(=O)C1CCc2[nH]c3ncnc(Cl)c3c2C1 ; CC(C)NC(=O)C1CCc2[nH]c3ncnc(Nc4cnc5[nH]ncc5c4)c3c2C1]

60 mg (205 μmol) (RS)-4-chloro-N-isopropyl-6,7,8,9-tetrahydro-5H-pyrimido[4,5-b]indole-6-carboxamide (prepared according to intermediate example 29a) were transformed in analogy to example 6 using 1H-pyrazolo[3,4-b]pyridin-5-amine to give after working up and purification 58.7 mg (70%) of the title compound.

¹H-NMR (DMSO-d6): δ=1.04 (6H), 1.77 (1H), 1.95 (1H), 2.51 (1H), 2.68 (1H), 2.94 (1H), 3.09 (1H), 3.36 (1H), 3.84 (1H), 7.75 (1H), 8.03 (1H), 8.06 (1H), 8.20 (1H), 8.33 (1H), 8.60 (1H), 11.42 (1H), 13.50 (1H) ppm.

### Example 29a

### (RS)-4-Chloro-N-isopropyl-6,7,8,9-tetrahydro-5H-pyrimido[4,5-b]indole-6-carboxamide

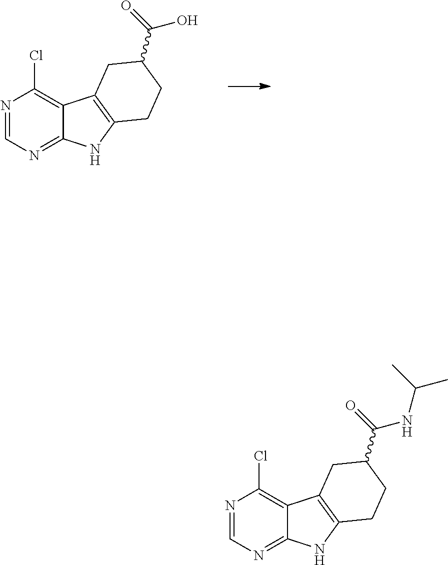 [SMILES: O=C(O)C1CCc2[nH]c3ncnc(Cl)c3c2C1 ; CC(C)NC(=O)C1CCc2[nH]c3ncnc(Cl)c3c2C1]

550 mg (2.19 mmol) (RS)-4-chloro-6,7,8,9-tetrahydro-5H-pyrimido[4,5-b]indole-6-carboxylic acid (prepared according to intermediate example 29b) were transformed in analogy to example 18 using propan-2-amine to give after working up and purification 526.7 mg (82%) of the title compound.

### Example 29b

### (RS)-4-Chloro-6,7,8,9-tetrahydro-5H-pyrimido[4,5-b]indole-6-carboxylic acid

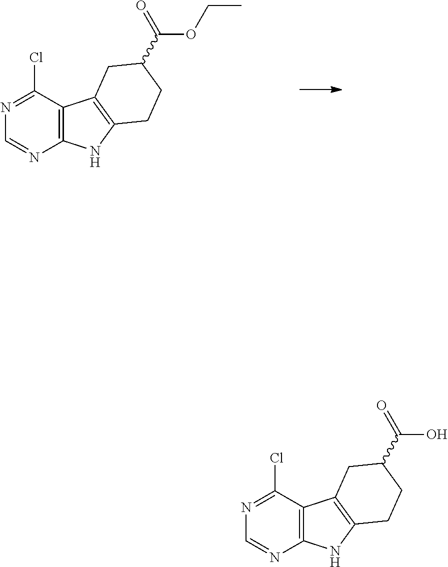 [SMILES: CCOC(=O)C1CCc2[nH]c3ncnc(Cl)c3c2C1 ; O=C(O)C1CCc2[nH]c3ncnc(Cl)c3c2C1]

4.56 g (16.3 mmol) (RS)-ethyl 4-chloro-6,7,8,9-tetrahydro-5H-pyrimido[4,5-b]indole-6-carboxylate (prepared according to intermediate example 29c) were transformed in analogy to example 11 to give after working up and purification 3.57 g (83%) of the title compound.

### Example 29c

### (RS)-Ethyl 4-chloro-6,7,8,9-tetrahydro-5H-pyrimido[4,5-b]indole-6-carboxylate

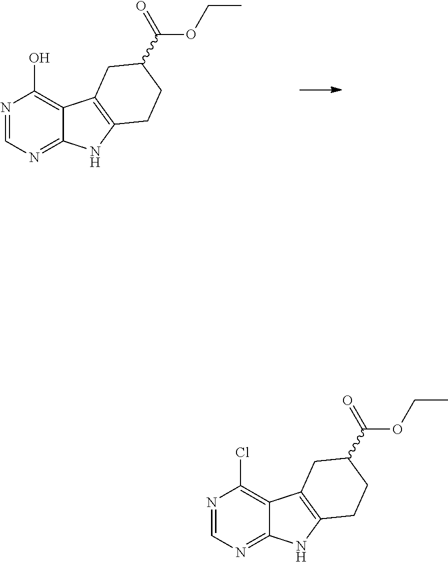 [SMILES: CCOC(=O)C1CCc2[nH]c3ncnc(O)c3c2C1 ; CCOC(=O)C1CCc2[nH]c3ncnc(Cl)c3c2C1]

A mixture comprising 412 mg (1.58 mmol) (RS)-ethyl 4-hydroxy-6,7,8,9-tetrahydro-5H-pyrimido[4,5-b]indole-6-carboxylate (prepared according to intermediate example 29d) and 8.82 mL phosphorus oxychloride was heated at 100° C. for 1 hour. The reagent was removed and the residue purified by chromatography to give 321.6 mg (73%) of the title compound.

### Example 29d

### (RS)-Ethyl 4-hydroxy-6,7,8,9-tetrahydro-5H-pyrimido[4,5-b]indole-6-carboxylate

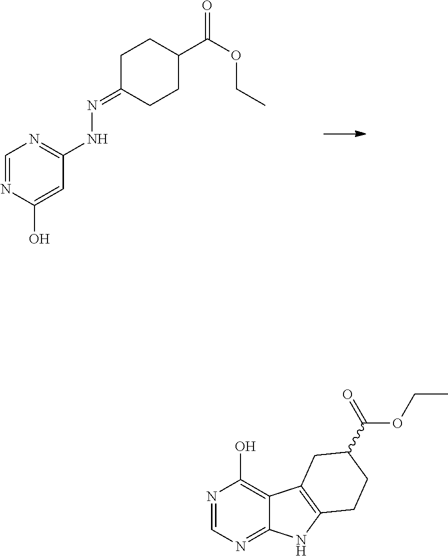 [SMILES: CCOC(=O)C1CCC(=NNc2cc(O)ncn2)CC1 ; CCOC(=O)C1CCc2[nH]c3ncnc(O)c3c2C1]

A mixture comprising 3.17 g (11.37 mmol) ethyl 4-[(6-hydroxypyrimidin-4-yl)hydrazono]cyclohexanecarboxylate (prepared according to intermediate example 29e) and 20 mL xylol was heated at 250° C. under microwave irradiation for 5 hours. The solid was filtered off and washed with diethyl ether to give 2.83 g (95%) of the title compound.

### Example 29e

### Ethyl 4-[(6-hydroxypyrimidin-4-yl)hydrazono]cyclohexanecarboxylate

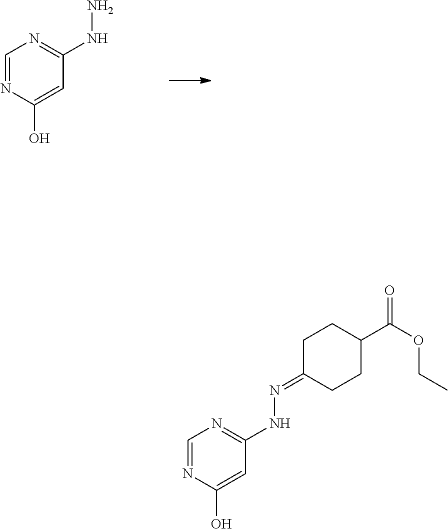 [SMILES: NNc1cc(O)ncn1 ; CCOC(=O)C1CCC(=NNc2cc(O)ncn2)CC1]

A mixture comprising 4.29 g (34.0 mmol) 6-hydrazinopyrimidin-4-ol/6-hydrazinopyrimidin-4(1H)-one (CAS-No: 29939-37-5), 8.69 g ethyl 4-oxocyclohexanecarboxylate and 69 mL ethanol was heated under reflux for 1.5 h. After cooling to 3° C., the precipitated solid was filtered off and washed with diethyl ether to give 6.83 g (72%) of the title compound.

### Example 30

### (RS)—N,N-Dimethyl-4-(1H-pyrazolo[3,4-b]pyridin-5-ylamino)-6,7,8,9-tetrahydro-5H-pyrimido[4,5-b]indole-6-carboxamide

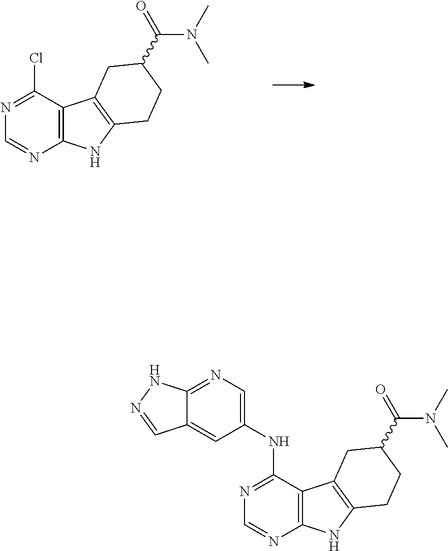 [SMILES: CN(C)C(=O)C1CCc2[nH]c3ncnc(Cl)c3c2C1 ; CN(C)C(=O)C1CCc2[nH]c3ncnc(Nc4cnc5[nH]ncc5c4)c3c2C1]

80 mg (287 μmol) (RS)-4-chloro-N,N-dimethyl-6,7,8,9-tetrahydro-5H-pyrimido[4,5-b]indole-6-carboxamide (prepared according to intermediate example 27a) were transformed in analogy to example 7 to give after working up and purification 73.3 mg (64%) of the title compound.

¹H-NMR (DMSO-d6): δ=1.79 (1H), 1.99 (1H), 2.74-2.95 (3H), 2.85 (3H), 3.01-3.19 (2H), 3.09 (3H), 8.15 (1H), 8.23 (1H), 8.36 (1H), 8.59 (1H), 9.75 (1H), 12.56 (1H), 13.84 (1H) ppm.

### Example 31

### (RS)—N,N-Dimethyl-4-(1H-pyrazolo[3,4-c]pyridin-5-ylamino)-6,7,8,9-tetrahydro-5H-pyrimido[4,5-b]indole-6-carboxamide

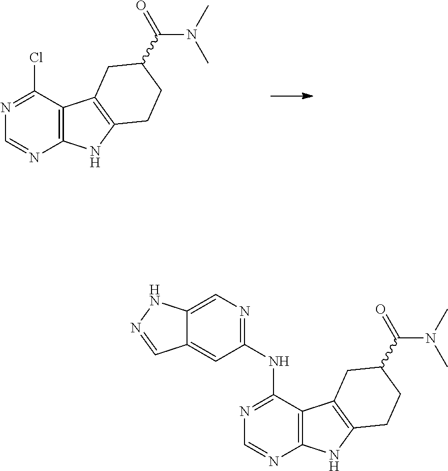 [SMILES: CN(C)C(=O)C1CCc2[nH]c3ncnc(Cl)c3c2C1 ; CN(C)C(=O)C1CCc2[nH]c3ncnc(Nc4cc5cn[nH]c5cn4)c3c2C1]

80 mg (287 μmol) (RS)-4-chloro-N,N-dimethyl-6,7,8,9-tetrahydro-5H-pyrimido[4,5-b]indole-6-carboxamide (prepared according to intermediate example 27a) were transformed in analogy to example 7 using 1H-pyrazolo[3,4-c]pyridin-5-amine to give after working up and purification 9.4 mg (8%) of the title compound.

¹H-NMR (DMSO-d6): δ=1.77 (1H), 1.98 (1H), 2.67-2.84 (2H), 2.89 (3H), 2.95-3.14 (3H), 3.10 (3H), 7.95 (1H), 8.19 (1H), 8.28 (1H), 8.72 (1H), 8.78 (1H), 11.54 (1H), 13.42 (1H) ppm.

### Example 32

### (RS)-(4-Methylpiperazin-1-yl)[4-(1H-pyrazolo[3,4-b]pyridin-5-ylamino)-6,7,8,9-tetrahydro-5H-pyrimido[4,5-b]indol-6-yl]methanone

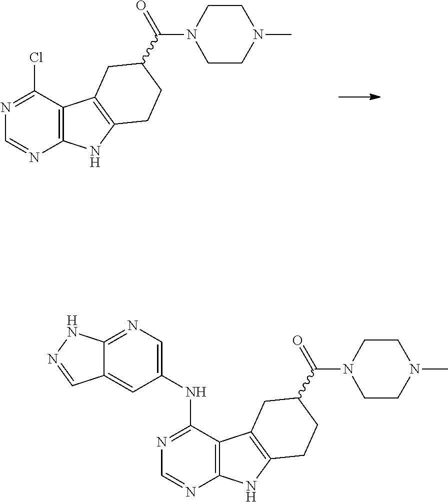 [SMILES: CN1CCN(C(=O)C2CCc3[nH]c4ncnc(Cl)c4c3C2)CC1 ; CN1CCN(C(=O)C2CCc3[nH]c4ncnc(Nc5cnc6[nH]ncc6c5)c4c3C2)CC1]

60 mg (180 μmol) (RS)-(4-chloro-6,7,8,9-tetrahydro-5H-pyrimido[4,5-b]indol-6-yl)(4-methylpiperazin-1-yl)methanone (prepared according to intermediate example 32a) were transformed in analogy to example 7 to give after working up and purification 10.4 mg (13%) of the title compound.

¹H-NMR (DMSO-d6): δ=1.81 (1H), 1.95 (1H), 2.25 (3H), 2.29-2.46 (3H), 2.67-2.85 (2H), 2.92-3.13 (3H), 3.33-3.38 (1H), 3.39-3.69 (4H), 8.07 (1H), 8.09 (1H), 8.20 (1H), 8.39 (1H), 8.64 (1H), 11.44 (1H), 13.50 (1H) ppm.

### Example 32a

### (RS)-(4-Chloro-6,7,8,9-tetrahydro-5H-pyrimido[4,5-b]indol-6-yl)(4-methylpiperazin-1-yl)methanone

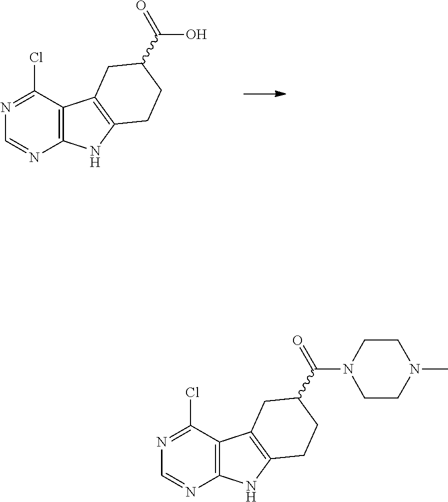 [SMILES: O=C(O)C1CCc2[nH]c3ncnc(Cl)c3c2C1 ; CN1CCN(C(=O)C2CCc3[nH]c4ncnc(Cl)c4c3C2)CC1]

500 mg (1.99 mmol) (RS)-4-chloro-6,7,8,9-tetrahydro-5H-pyrimido[4,5-b]indole-6-carboxylic acid (prepared according to intermediate example 29b) were transformed in analogy to example 18 using 1-methylpiperazine to give after working up and purification 411 mg (62%) of the title compound.

### Example 33

### (RS)-(4-Methylpiperazin-1-yl)[4-(1H-pyrazolo[3,4-c]pyridin-5-ylamino)-6,7,8,9-tetrahydro-5H-pyrimido[4,5-b]indol-6-yl]methanone

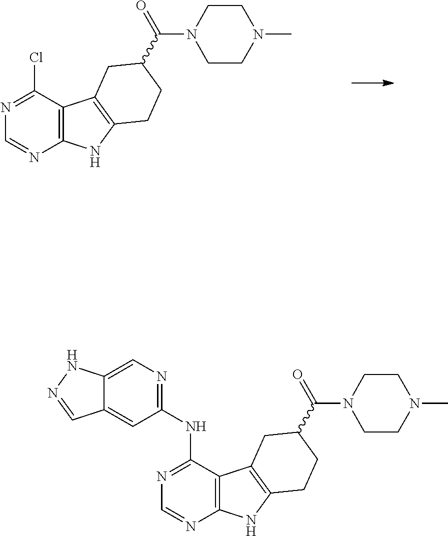 [SMILES: CN1CCN(C(=O)C2CCc3[nH]c4ncnc(Cl)c4c3C2)CC1 ; CN1CCN(C(=O)C2CCc3[nH]c4ncnc(Nc5cc6cn[nH]c6cn5)c4c3C2)CC1]

60 mg (180 μmol) (RS)-(4-chloro-6,7,8,9-tetrahydro-5H-pyrimido[4,5-b]indol-6-yl)(4-methylpiperazin-1-yl)methanone (prepared according to intermediate example 32a) were transformed in analogy to example 7 using 1H-pyrazolo[3,4-c]pyridin-5-amine to give after working up and purification 4.0 mg (5%) of the title compound.

¹H-NMR (DMSO-d6): δ=1.78 (1H), 1.96 (1H), 2.20 (3H), 2.24-2.39 (4H), 2.65-2.86 (2H), 2.99-3.17 (3H), 3.46-3.63 (4H), 7.98 (1H), 8.20 (1H), 8.28 (1H), 8.70 (1H), 8.79 (1H), 11.56 (1H), 13.45 (1H) ppm.

### Example 34

### {(7S)-4-[(6-Methoxy-1H-pyrazolo[3,4-b]pyridin-5-yl)amino]-5,6,7,8-tetrahydro[1]benzothieno[2,3-d]pyrimidin-7-yl)}(morpholin-4-yl)methanone

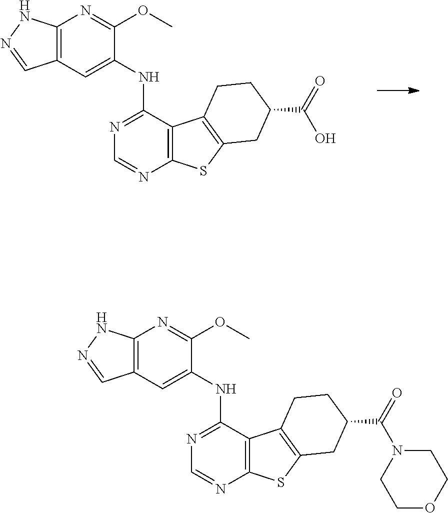 [SMILES: COc1nc2[nH]ncc2cc1Nc1ncnc2sc3c(c12)CC[C@H](C(=O)O)C3 ; COc1nc2[nH]ncc2cc1Nc1ncnc2sc3c(c12)CC[C@H](C(=O)N1CCOCC1)C3]

100 mg (252 μmol) (7S)-4-[(6-methoxy-1H-pyrazolo[3,4-b]pyridin-5-yl)amino]-5,6,7,8-tetrahydro[1]benzothieno[2,3-d]pyrimidine-7-carboxylic acid (prepared according to intermediate example 34a) were transformed in analogy to example 18 using morpholine to give after working up and purification 43.5 mg (35%) of the title compound.

¹H-NMR (DMSO-d6): δ=1.86 (1H), 2.12 (1H), 2.86-3.05 (2H), 3.12-3.29 (3H), 3.45-3.66 (8H), 4.02 (3H), 8.01 (1H), 8.09 (1H), 8.41 (1H), 8.82 (1H), 13.37 (1H) ppm.

### Example 34a

### (7S)-4-[(6-Methoxy-1H-pyrazolo[3,4-b]pyridin-5-yl)amino]-5,6,7,8-tetrahydro[1]benzothieno[2,3-d]pyrimidine-7-carboxylic acid

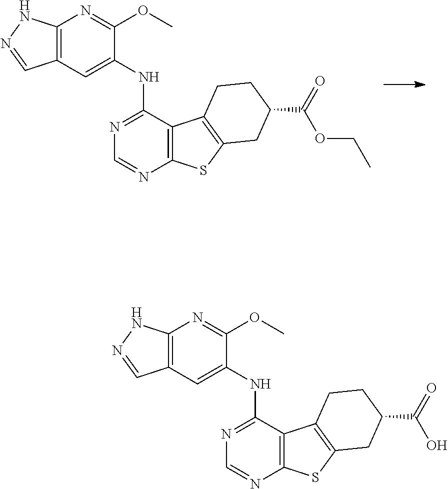 [SMILES: CCOC(=O)[C@H]1CCc2c(sc3ncnc(Nc4cc5cn[nH]c5nc4OC)c23)C1 ; COc1nc2[nH]ncc2cc1Nc1ncnc2sc3c(c12)CC[C@H](C(=O)O)C3]

2.60 g (6.13 mmol) ethyl (7S)-4-[(6-methoxy-1H-pyrazolo[3,4-b]pyridin-5-yl)amino]-5,6,7,8-tetrahydro[1]benzothieno[2,3-d]pyrimidine-7-carboxylate (prepared according to intermediate example 34b) were transformed in analogy to example 11 to give after working up and purification 2.36 g (97%) of the title compound.

### Example 34b

### (7S)-4-[(6-Methoxy-1H-pyrazolo[3,4-b]pyridin-5-yl)amino]-5,6,7,8-tetrahydro[1]benzothieno[2,3-d]pyrimidine-7-carboxylate

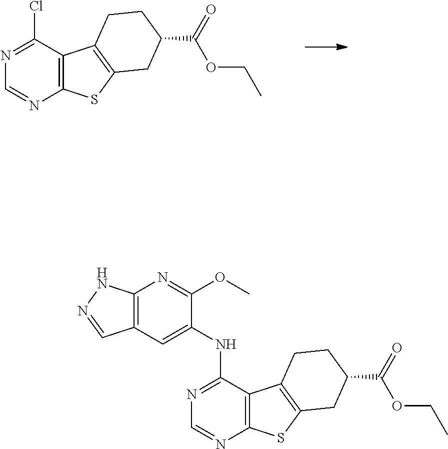 [SMILES: CCOC(=O)[C@H]1CCc2c(sc3ncnc(Cl)c23)C1 ; CCOC(=O)[C@H]1CCc2c(sc3ncnc(Nc4cc5cn[nH]c5nc4OC)c23)C1]

A mixture comprising 2.38 g (8.01 mmol) ethyl (7S)-4-chloro-5,6,7,8-tetrahydro[1]benzothieno[2,3-d]pyrimidine-7-carboxylate (prepared according to intermediate example 34c), 1.32 g 6-methoxy-1H-pyrazolo[3,4-b]pyridin-5-amine (prepared according to intermediate example 34f) and 25 mL ethanol was heated at reflux overnight. 1.2 mL triethylamine were added and the precipitate purified by recrystallization from ethanol to give 2.60 g (76%) of the title compound.

### Example 34c

### Ethyl (7S)-4-chloro-5,6,7,8-tetrahydro[1]benzothieno[2,3-d]pyrimidine-7-carboxylate

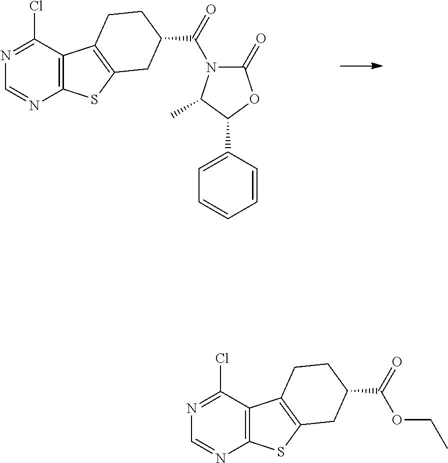 [SMILES: C[C@H]1[C@@H](c2ccccc2)OC(=O)N1C(=O)[C@H]1CCc2c(sc3ncnc(Cl)c23)C1 ; CCOC(=O)[C@H]1CCc2c(sc3ncnc(Cl)c23)C1]

A mixture comprising 27.6 g (64.6 mmol) (4S,5R)-3-{[(7S)-4-chloro-5,6,7,8-tetrahydro[1]benzothieno[2,3-d]pyrimidin-7-yl]carbonyl}-4-methyl-5-phenyl-1,3-oxazolidin-2-one (prepared according to intermediate example 34d), 830 mL ethanol and 24.4 mL titanium(4+) tetraethanolate was refluxed for 20 hours. 1.4 L ethyl acetate and 18 mL water were added and the mixture was stirred for 30 minutes. Silica gel was added and stirring was continued for 10 minutes. The mixture was filtered through celite, the solvents were removed and the residue was purified by chromatography to give 18.8 g (93%) of the title compound.

### Example 34d

### (4S,5R)-3-{[(7S)-4-Chloro-5,6,7,8-tetrahydro[1]benzothieno[2,3-d]pyrimidin-7-yl]carbonyl}-4-methyl-5-phenyl-1,3-oxazolidin-2-one (A) and (4S,5R)-3-{[(7R)-4-chloro-5,6,7,8-tetrahydro[1]benzothieno[2,3-d]pyrimidin-7-yl]carbonyl}-4-methyl-5-phenyl-1,3-oxazolidin-2-one (B)

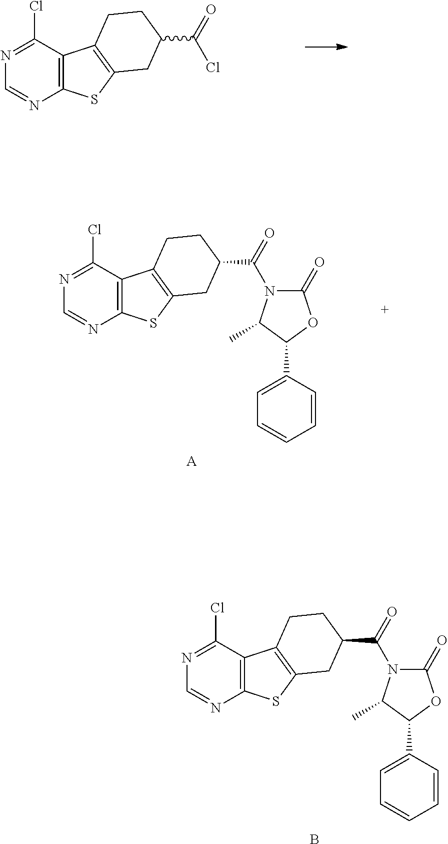

To a solution of 26.8 g (4S,5R)-4-methyl-5-phenyl-1,3-oxazolidin-2-one in 428 mL tetrahydrofurane were added 70 mL n-butyllithium (2.5 M in hexane) at −78° C. and the mixture was stirred at −60° C. for 1 hour. A solution of 45.8 g (159 mmol) 4-chloro-5,6,7,8-tetrahydro[1]benzothieno[2,3-d]pyrimidine-7(RS)-carbonyl chloride (prepared according to intermediate example 34e) in 428 mL tetrahydrofurane was added and stirring was continued at −70° C. for 1 hour. The mixture was poured into water, tetrahydrofurane was removed, the precipitate was filtered off, washed with water and resolved in dichloromethane. The organic layer was dried over sodium sulphate followed by addition of acetonitrile. The dichloromethane was removed, the precipitate filtered, washed with acetonitrile and diethylether to give 27.6 g (38%) of the title compound A. From the mother liquor a second precipitate was obtained on standing overnight to give 25.5 g (35%) of the title compound B.

### Example 34e

### 4-Chloro-5,6,7,8-tetrahydro[1]benzothieno[2,3-d]pyrimidine-7(RS)-carbonyl chloride

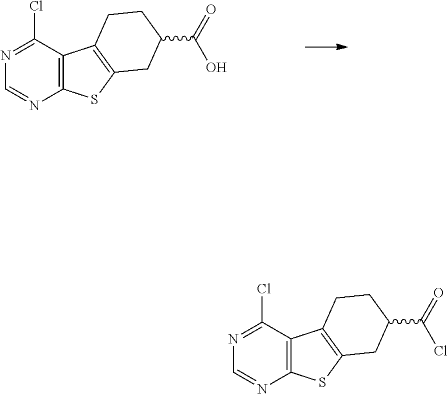 [SMILES: O=C(O)C1CCc2c(sc3ncnc(Cl)c23)C1 ; O=C(Cl)C1CCc2c(sc3ncnc(Cl)c23)C1]

A mixture comprising 42.87 g (159 mmol) 4-chloro-5,6,7,8-tetrahydro[1]benzothieno[2,3-d]pyrimidine-7-carboxylic acid (prepared according to intermediate example 23b) and 349 mL thionyl chloride was heated at 100° C. for 3 hours. The reagent was removed to give the title compound that was used without further purification.

### Example 34f

### 6-Methoxy-1H-pyrazolo[3,4-b]pyridin-5-amine

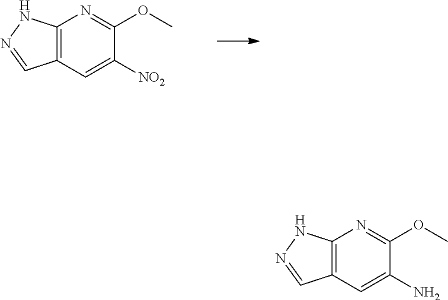

A mixture comprising 4.00 g (20.6 mmol) 6-methoxy-5-nitro-1H-pyrazolo[3,4-b]pyridine (prepared according to intermediate example 34g), 120 mL ethanol, 120 mL tetrahydrofurane and 1.10 g palladium on charcoal (10%) was stirred at 23° C. under an atmosphere of hydrogen overnight. The catalyst and solvents were removed to give 3.26 g (96%) of the title compound.

### Example 34g

### 6-Methoxy-5-nitro-1H-pyrazolo[3,4-b]pyridine

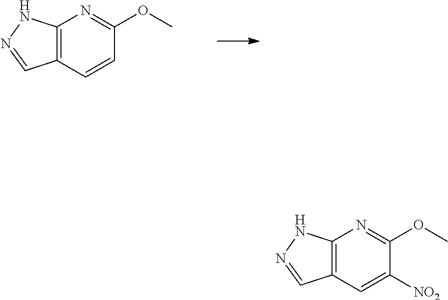

At 0° C. 2.2 mL fuming nitric acid was carefully added to 6.8 mL acetic anhydride. 1.00 g (mmol) 6-methoxy-1H-pyrazolo[3,4-b]pyridine (CAS-No. 1260664-24-1) was added in portions, the mixture was stirred at 10° C. for 22 hours and poured on ice. Sodium hydrocarbonate was carefully added until a pH between 2 and 3 was obtained and the mixture was extracted with dichloromethane. The organic layer was washed with brine and dried over sodium sulphate. After filtration and removal of the solvent, the residue was purified by chrystallization to give 640 mg (39%) of the title compound.

### Example 35

### Azetidin-1-yl{(7S)-4-[(6-methoxy-1H-pyrazolo[3,4-b]pyridin-5-yl)amino]-5,6,7,8-tetrahydro[1]benzothieno[2,3-d]pyrimidin-7-yl}methanone

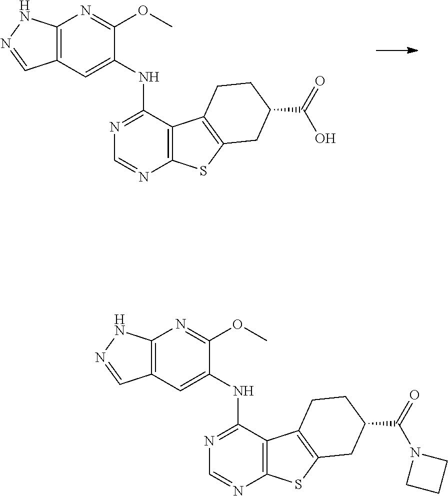 [SMILES: COc1nc2[nH]ncc2cc1Nc1ncnc2sc3c(c12)CC[C@H](C(=O)O)C3 ; COc1nc2[nH]ncc2cc1Nc1ncnc2sc3c(c12)CC[C@H](C(=O)N1CCC1)C3]

100 mg (252 μmol) (7S)-4-[(6-methoxy-1H-pyrazolo[3,4-b]pyridin-5-yl)amino]-5,6,7,8-tetrahydro[1]benzothieno[2,3-d]pyrimidine-7-carboxylic acid (prepared according to intermediate example 34a) were transformed in analogy to example 18 using azetidine to give after working up and purification 44.8 mg (39%) of the title compound.

¹H-NMR (DMSO-d6): δ=1.80 (1H), 2.11 (1H), 2.17-2.27 (2H), 2.76 (1H), 2.90 (2H), 3.13 (1H), 3.24 (1H), 3.88 (2H), 4.02 (3H), 4.24 (2H), 8.01 (1H), 8.09 (1H), 8.41 (1H), 8.83 (1H), 13.35 (1H) ppm.

### Example 36

### [(2R,6S)-2,6-Dimethylmorpholin-4-yl]{(7S)-4-[(6-methoxy-1H-pyrazolo[3,4-b]pyridin-5-yl)amino]-5,6,7,8-tetrahydro[1]benzothieno[2,3-d]pyrimidin-7-yl}methanone

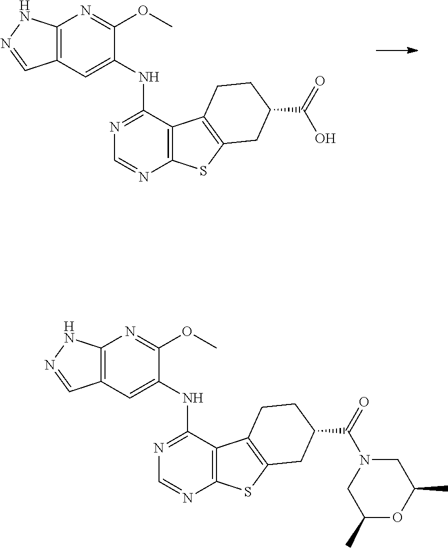 [SMILES: COc1nc2[nH]ncc2cc1Nc1ncnc2sc3c(c12)CC[C@H](C(=O)O)C3 ; COc1nc2[nH]ncc2cc1Nc1ncnc2sc3c(c12)CC[C@H](C(=O)N1C[C@@H](C)O[C@@H](C)C1)C3]

100 mg (252 μmol) (7S)-4-[(6-methoxy-1H-pyrazolo[3,4-b]pyridin-5-yl)amino]-5,6,7,8-tetrahydro[1]benzothieno[2,3-d]pyrimidine-7-carboxylic acid (prepared according to intermediate example 34a) were transformed in analogy to example 18 using (2R,6S)-2,6-dimethylmorpholine to give after working up and purification 27.6 mg (21%) of the title compound.

¹H-NMR (DMSO-d6): δ=1.07-1.15 (6H), 1.85 (1H), 2.09 (1H), 2.27 (1H), 2.77 (1H), 2.87-3.06 (2H), 3.23 (3H), 3.44 (1H), 3.52 (1H), 3.99 (1H), 4.01 (3H), 4.31 (1H), 8.01 (1H), 8.08 (1H), 8.40 (1H), 8.81 (1H), 13.36 (1H) ppm.

### Example 37

### (7S)-4-[(6-Methoxy-1H-pyrazolo[3,4-b]pyridin-5-yl)amino]-N-methyl-N-(propan-2-yl)-5,6,7,8-tetrahydro[1]benzothieno[2,3-d]pyrimidine-7-carboxamide

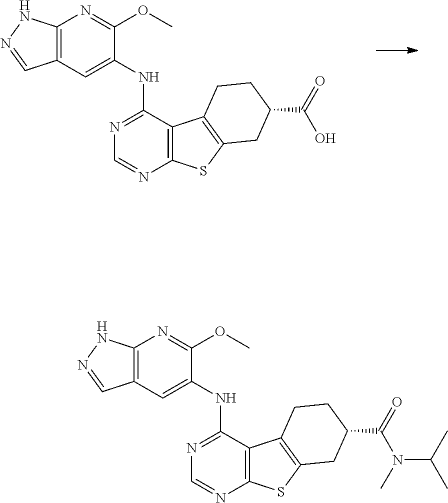 [SMILES: COc1nc2[nH]ncc2cc1Nc1ncnc2sc3c(c12)CC[C@H](C(=O)O)C3 ; COc1nc2[nH]ncc2cc1Nc1ncnc2sc3c(c12)CC[C@H](C(=O)N(C)C(C)C)C3]

100 mg (252 μmol) (7S)-4-[(6-methoxy-1H-pyrazolo[3,4-b]pyridin-5-yl)amino]-5,6,7,8-tetrahydro[1]benzothieno[2,3-d]pyrimidine-7-carboxylic acid (prepared according to intermediate example 34a) were transformed in analogy to example 18 using N-methylpropan-2-amine to give after working up and purification 57.8 mg (48%) of the title compound.

¹H-NMR (DMSO-d6): δ=1.05+1.18 (6H), 1.84 (1H), 2.10 (1H), 2.71+2.91 (3H), 2.85-3.06 (2H), 3.07-3.25 (3H), 4.02 (3H), 4.29+4.72 (1H), 8.02 (1H), 8.08 (1H), 8.40-8.42 (1H), 8.82+8.86 (1H), 13.33 (1H) ppm.

### Example 38

### (7S)-4-[(6-Methoxy-1H-pyrazolo[3,4-b]pyridin-5-yl)amino]-N-methyl-N-propyl-5,6,7,8-tetrahydro[1]benzothieno[2,3-d]pyrimidine-7-carboxamide

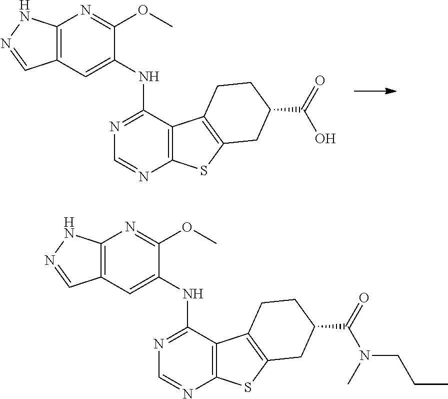 [SMILES: COc1nc2[nH]ncc2cc1Nc1ncnc2sc3c(c12)CC[C@H](C(=O)O)C3 ; CCCN(C)C(=O)[C@H]1CCc2c(sc3ncnc(Nc4cc5cn[nH]c5nc4OC)c23)C1]

100 mg (252 μmol) (7S)-4-[(6-methoxy-1H-pyrazolo[3,4-b]pyridin-5-yl)amino]-5,6,7,8-tetrahydro[1]benzothieno[2,3-d]pyrimidine-7-carboxylic acid (prepared according to intermediate example 34a) were transformed in analogy to example 18 using N-methylpropan-1-amine to give after working up and purification 66.0 mg (55%) of the title compound.

¹H-NMR (DMSO-d6): δ=0.83+0.87 (3H), 1.49+1.57 (2H), 1.84 (1H), 2.10 (1H), 2.85+3.08 (3H), 2.88-3.03 (2H), 3.12-3.43 (5H), 4.02 (3H), 8.01 (1H), 8.08 (1H), 8.41 (1H), 8.82+8.86 (1H), 13.35 (1H) ppm.

### Example 39

### (7S)—N-Ethyl-4-[(6-methoxy-1H-pyrazolo[3,4-b]pyridin-5-yl)amino]-N-methyl-5,6,7,8-tetrahydro[1]benzothieno[2,3-d]pyrimidine-7-carboxamide

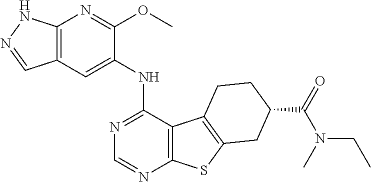 [SMILES: CCN(C)C(=O)[C@H]1CCc2c(sc3ncnc(Nc4cc5cn[nH]c5nc4OC)c23)C1]

100 mg (252 μmol) (7S)-4-[(6-methoxy-1H-pyrazolo[3,4-b]pyridin-5-yl)amino]-5,6,7,8-tetrahydro[1]benzothieno[2,3-d]pyrimidine-7-carboxylic acid (prepared according to intermediate example 34a) were transformed in analogy to example 18 using N-methylethanamine to give after working up and purification 51.3 mg (44%) of the title compound.

¹H-NMR (DMSO-d6): δ=1.03+1.15 (3H), 1.84 (1H), 2.10 (1H), 2.85+3.07 (3H), 2.86-3.02 (2H), 3.10-3.52 (5H), 4.02 (3H), 8.01 (1H), 8.08 (1H), 8.41 (1H), 8.83+8.85 (1H), 13.35 (1H) ppm.

### Example 40

### {(7S)-4-[(6-Methoxy-1H-pyrazolo[3,4-b]pyridin-5-yl)amino]-5,6,7,8-tetrahydro[1]benzothieno[2,3-d]pyrimidin-7-yl)}[(1S,4S)-2-oxa-5-azabicyclo[2.2.1]hept-5-yl]methanone

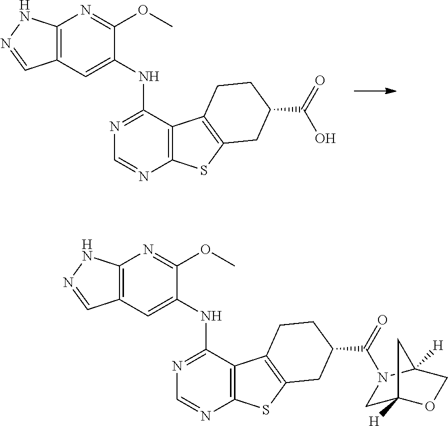 [SMILES: COc1nc2[nH]ncc2cc1Nc1ncnc2sc3c(c12)CC[C@H](C(=O)O)C3 ; COc1nc2[nH]ncc2cc1Nc1ncnc2sc3c(c12)CC[C@H](C(=O)N1C[C@@H]2C[C@H]1CO2)C3]

100 mg (252 μmol) (7S)-4-[(6-methoxy-1H-pyrazolo[3,4-b]pyridin-5-yl)amino]-5,6,7,8-tetrahydro[1]benzothieno[2,3-d]pyrimidine-7-carboxylic acid (prepared according to intermediate example 34a) were transformed in analogy to example 18 using (1S,4S)-2-oxa-5-azabicyclo[2.2.1]heptane to give after working up and purification 53.7 mg (42%) of the title compound.

¹H-NMR (DMSO-d6): δ=1.75-1.92 (3H), 2.12 (1H), 2.80-3.03 (3H), 3.08-3.25 (3H), 3.53+3.65 (1H), 3.58+3.72 (1H), 3.76 (1H), 4.01 (3H), 4.61+4.66 (1H), 4.77+4.87 (1H), 8.01 (1H), 8.07 (1H), 8.40+8.41 (1H), 8.80+8.85 (1H), 13.35 (1H) ppm.

### Example 41

### {(7S)-4-[(6-Methoxy-1H-pyrazolo[3,4-b]pyridin-5-yl)amino]-5,6,7,8-tetrahydro[1]benzothieno[2,3-d]pyrimidin-7-yl)}[(1R,4R)-2-oxa-5-azabicyclo[2.2.1]hept-5-yl]methanone

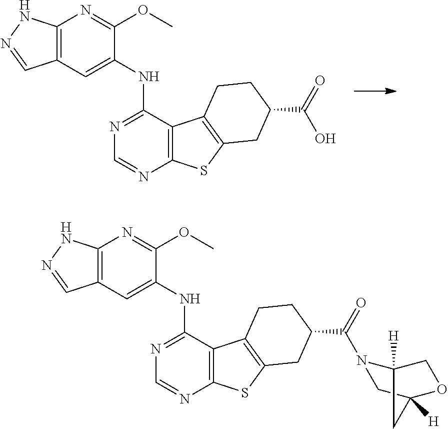 [SMILES: COc1nc2[nH]ncc2cc1Nc1ncnc2sc3c(c12)CC[C@H](C(=O)O)C3 ; COc1nc2[nH]ncc2cc1Nc1ncnc2sc3c(c12)CC[C@H](C(=O)N1C[C@H]2C[C@@H]1CO2)C3]

100 mg (252 μmol) (7S)-4-[(6-methoxy-1H-pyrazolo[3,4-b]pyridin-5-yl)amino]-5,6,7,8-tetrahydro[1]benzothieno[2,3-d]pyrimidine-7-carboxylic acid (prepared according to intermediate example 34a) were transformed in analogy to example 18 using (1R,4R)-2-oxa-5-azabicyclo[2.2.1]heptane to give after working up and purification 38.1 mg (30%) of the title compound.

¹H-NMR (DMSO-d6): δ=1.75-1.96 (3H), 2.16 (1H), 2.77-3.25 (5H), 3.51+3.65 (1H), 3.60+3.73 (1H), 3.74 (2H), 4.01 (3H), 4.61+4.67 (1H), 4.78+4.86 (1H), 8.01 (1H), 8.09 (1H), 8.42 (1H), 8.86+8.88 (1H), 13.35 (1H) ppm.

### Example 42

### {(7S)-4-[(6-Methoxy-1H-pyrazolo[3,4-b]pyridin-5-yl)amino]-5,6,7,8-tetrahydro[1]benzothieno[2,3-d]pyrimidin-7-yl)}(4-methylpiperazin-1-yl)methanone

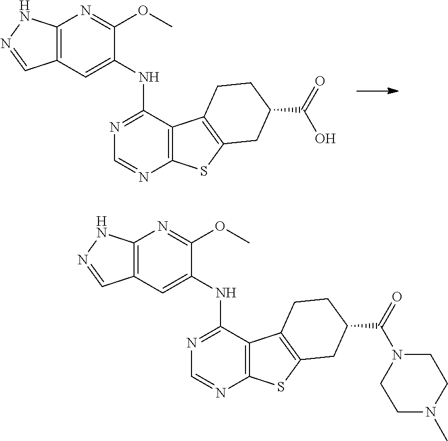 [SMILES: COc1nc2[nH]ncc2cc1Nc1ncnc2sc3c(c12)CC[C@H](C(=O)O)C3 ; COc1nc2[nH]ncc2cc1Nc1ncnc2sc3c(c12)CC[C@H](C(=O)N1CCN(C)CC1)C3]

100 mg (252 μmol) (7S)-4-[(6-methoxy-1H-pyrazolo[3,4-b]pyridin-5-yl)amino]-5,6,7,8-tetrahydro[1]benzothieno[2,3-d]pyrimidine-7-carboxylic acid (prepared according to intermediate example 34a) were transformed in analogy to example 18 using 1-methylpiperazine to give after working up and purification 80.2 mg (63%) of the title compound.

¹H-NMR (DMSO-d6): δ=1.85 (1H), 2.10 (1H), 2.19 (3H), 2.25-2.38 (4H), 2.85-3.01 (2H), 3.14-3.25 (3H), 3.50 (2H), 3.58 (2H), 4.02 (3H), 8.01 (1H), 8.08 (1H), 8.41 (1H), 8.83 (1H), 13.35 (1H) ppm.

### Example 43

### [4-(Dimethylamino)piperidin-1-yl]{(7S)-4-[(6-methoxy-1H-pyrazolo[3,4-b]pyridin-5-yl)amino]-5,6,7,8-tetrahydro[1]benzothieno[2,3-d]pyrimidin-7-yl}methanone

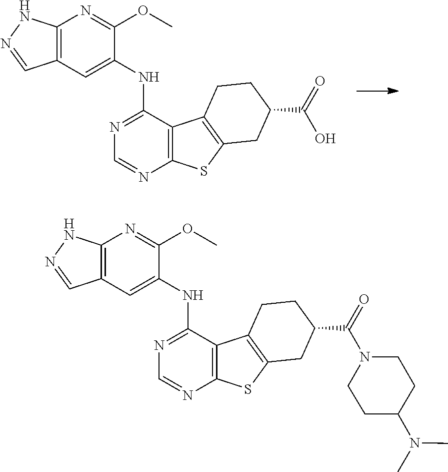 [SMILES: COc1nc2[nH]ncc2cc1Nc1ncnc2sc3c(c12)CC[C@H](C(=O)O)C3 ; COc1nc2[nH]ncc2cc1Nc1ncnc2sc3c(c12)CC[C@H](C(=O)N1CCC(N(C)C)CC1)C3]

100 mg (252 μmol) (7S)-4-[(6-methoxy-1H-pyrazolo[3,4-b]pyridin-5-yl)amino]-5,6,7,8-tetrahydro[1]benzothieno[2,3-d]pyrimidine-7-carboxylic acid (prepared according to intermediate example 34a) were transformed in analogy to example 18 using N,N-dimethylpiperidin-4-amine to give after working up and purification 65.0 mg (48%) of the title compound.

¹H-NMR (DMSO-d6): δ=1.21 (1H), 1.35 (1H), 1.73-1.90 (3H), 2.10 (1H), 2.20 (6H), 2.36 (1H), 2.61 (1H), 2.84-3.24 (6H), 4.02 (3H), 4.06 (1H), 4.41 (1H), 8.02 (1H), 8.08 (1H), 8.41 (1H), 8.83 (1H), 13.34 (1H) ppm.

### Example 44

### {(7S)-4-[(6-Methoxy-1H-pyrazolo[3,4-b]pyridin-5-yl)amino]-5,6,7,8-tetrahydro[1]benzothieno[2,3-d]pyrimidin-7-yl)}[(3R)-3-methylmorpholin-4-yl]methanone

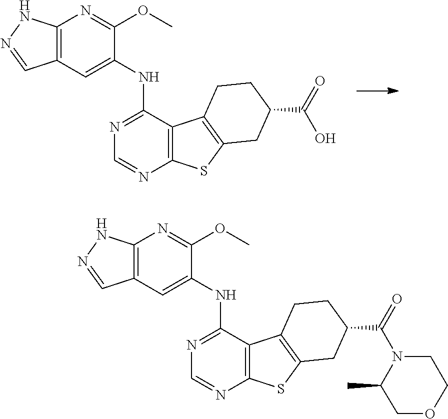 [SMILES: COc1nc2[nH]ncc2cc1Nc1ncnc2sc3c(c12)CC[C@H](C(=O)O)C3 ; COc1nc2[nH]ncc2cc1Nc1ncnc2sc3c(c12)CC[C@H](C(=O)N1CCOC[C@H]1C)C3]

100 mg (252 μmol) (7S)-4-[(6-methoxy-1H-pyrazolo[3,4-b]pyridin-5-yl)amino]-5,6,7,8-tetrahydro[1]benzothieno[2,3-d]pyrimidine-7-carboxylic acid (prepared according to intermediate example 34a) were transformed in analogy to example 18 using (3R)-3-methylmorpholine to give after working up and purification 51.1 mg (40%) of the title compound.

¹H-NMR (DMSO-d6): δ=1.15+1.31 (3H), 1.90 (1H), 2.09 (1H), 2.85-3.89 (11H), 4.02 (3H), 4.14+4.42 (1H), 8.01 (1H), 8.08 (1H), 8.41 (1H), 8.82 (1H), 13.35 (1H) ppm.

### Example 45

### {(7S)-4-[(6-Methoxy-1H-pyrazolo[3,4-b]pyridin-5-yl)amino]-5,6,7,8-tetrahydro[1]benzothieno[2,3-d]pyrimidin-7-yl)}[(3S)-3-methyl morpholin-4-yl]methanone

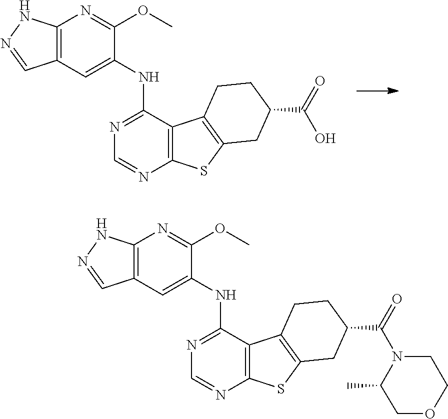 [SMILES: COc1nc2[nH]ncc2cc1Nc1ncnc2sc3c(c12)CC[C@H](C(=O)O)C3 ; COc1nc2[nH]ncc2cc1Nc1ncnc2sc3c(c12)CC[C@H](C(=O)N1CCOC[C@@H]1C)C3]

100 mg (252 μmol) (7S)-4-[(6-methoxy-1H-pyrazolo[3,4-b]pyridin-5-yl)amino]-5,6,7,8-tetrahydro[1]benzothieno[2,3-d]pyrimidine-7-carboxylic acid (prepared according to intermediate example 34a) were transformed in analogy to example 18 using (3S)-3-methylmorpholine to give after working up and purification 83.0 mg (65%) of the title compound.

¹H-NMR (DMSO-d6): δ=1.16+1.29 (3H), 1.83 (1H), 2.10 (1H), 2.84-3.90 (11H), 4.02 (3H), 4.13+4.43 (1H), 8.02 (1H), 8.08 (1H), 8.42 (1H), 8.85 (1H), 13.35 (1H) ppm.

### Example 46

### (7S)—N-Ethyl-N-(propan-2-yl)-4-(1H-pyrazolo[3,4-c]pyridin-5-ylamino)-5,6,7,8-tetrahydro[1]benzothieno[2,3-d]pyrimidine-7-carboxamide

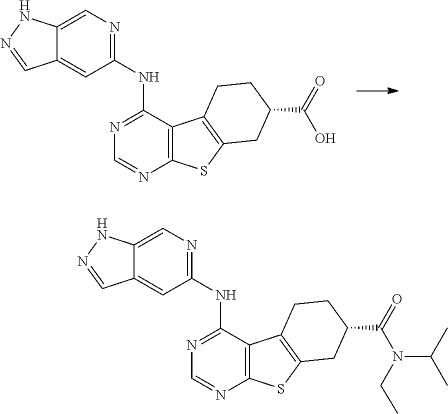 [SMILES: O=C(O)[C@H]1CCc2c(sc3ncnc(Nc4cc5cn[nH]c5cn4)c23)C1 ; CCN(C(=O)[C@H]1CCc2c(sc3ncnc(Nc4cc5cn[nH]c5cn4)c23)C1)C(C)C]

100 mg (273 μmol) (7S)-4-(1H-pyrazolo[3,4-c]pyridin-5-ylamino)-5,6,7,8-tetrahydro[1]benzothieno[2,3-d]pyrimidine-7-carboxylic acid (prepared according to intermediate example 46a) were transformed in analogy to example 18 using N-ethylpropan-2-amine to give after working up and purification 18.6 mg (15%) of the title compound.

¹H-NMR (DMSO-d6): δ=1.04-1.23 (9H), 1.84 (1H), 2.07 (1H), 2.85-36 (7H), 4.24+4.55 (1H), 8.24 (1H), 8.33 (1H), 8.54 (1H), 8.69 (1H), 8.84 (1H), 13.54 (1H) ppm.

### Example 46a

### (7S)-4-(1H-Pyrazolo[3,4-c]pyridin-5-ylamino)-5,6,7,8-tetrahydro[1]benzothieno[2,3-d]pyrimidine-7-carboxylic acid

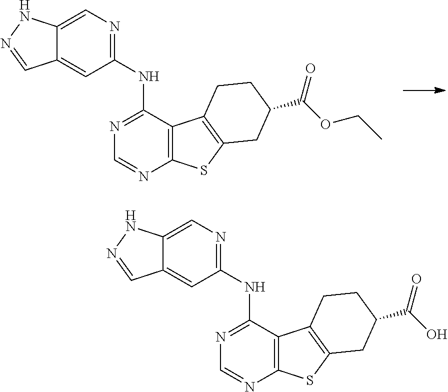 [SMILES: CCOC(=O)[C@H]1CCc2c(sc3ncnc(Nc4cc5cn[nH]c5cn4)c23)C1 ; O=C(O)[C@H]1CCc2c(sc3ncnc(Nc4cc5cn[nH]c5cn4)c23)C1]

1.79 g (4.53 mmol) ethyl (7S)-4-(1H-pyrazolo[3,4-c]pyridin-5-ylamino)-5,6,7,8-tetrahydro[1]benzothieno[2,3-d]pyrimidine-7-carboxylate (prepared according to intermediate example 46b) were transformed in analogy to example 11 to give after working up and purification 1.53 g (88%) of the title compound.

### Example 46b

### Ethyl (7S)-4-(1H-pyrazolo[3,4-c]pyridin-5-ylamino)-5,6,7,8-tetrahydro[1]benzothieno[2,3-d]pyrimidine-7-carboxylate

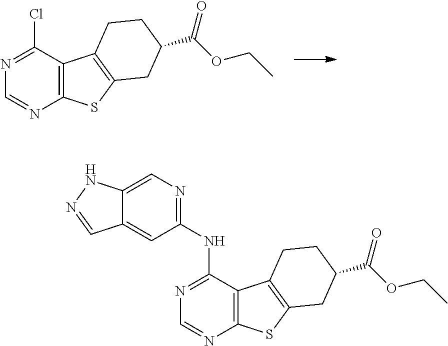 [SMILES: CCOC(=O)[C@H]1CCc2c(sc3ncnc(Cl)c23)C1 ; CCOC(=O)[C@H]1CCc2c(sc3ncnc(Nc4cc5cn[nH]c5cn4)c23)C1]

A mixture comprising 1.20 g (4.04 mmol) ethyl (7S)-4-chloro-5,6,7,8-tetrahydro[1]benzothieno[2,3-d]pyrimidine-7-carboxylate (prepared according to intermediate example 34c), 515 mg 1H-pyrazolo[3,4-c]pyridin-5-amine (CAS-No. 1049672-75-4) and 36 mL ethanol was heated at 150° C. under microwave irradiation. The precipitate was washed with ethanol and diethylether to give 618 mg (39%) of the title compound.

### Example 47

### (7S)—N-(2-Hydroxy-2-methylpropyl)-N-methyl-4-(1H-pyrazolo[3,4-c]pyridin-5-ylamino)-5,6,7,8-tetrahydro[1]benzothieno[2,3-d]pyrimidine-7-carboxamide

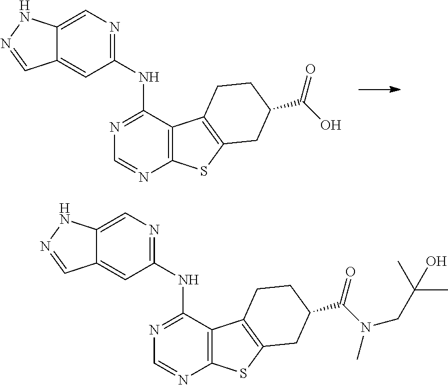

100 mg (273 μmol) (7S)-4-(1H-pyrazolo[3,4-c]pyridin-5-ylamino)-5,6,7,8-tetrahydro[1]benzothieno[2,3-d]pyrimidine-7-carboxylic acid (prepared according to intermediate example 46a) were transformed in analogy to example 18 using 2-methyl-1-(methylamino)propan-2-ol to give after working up and purification 38.9 mg (30%) of the title compound.

¹H-NMR (DMSO-d6): δ=1.05-1.15 (6H), 1.81 (1H), 2.08+2.16 (1H), 2.84-3.01 (2H), 2.95+3.20 (3H), 3.14-3.44 (5H), 4.49+4.56 (1H), 8.24 (1H), 8.34 (1H), 8.53 (1H), 8.68 (1H), 8.84 (1H), 13.53 (1H) ppm.

### Example 48

### (7S)—N-Methyl-4-(1H-pyrazolo[3,4-c]pyridin-5-ylamino)-N-(3,3,3-trifluoropropyl)-5,6,7,8-tetrahydro[1]benzothieno[2,3-d]pyrimidine-7-carboxamide

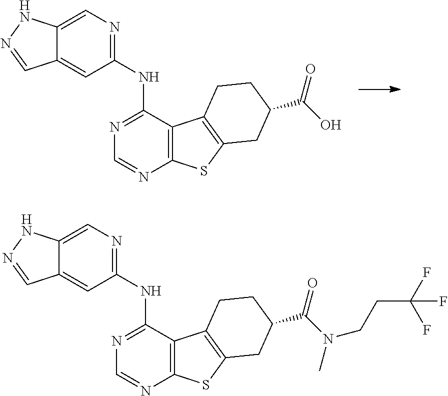 [SMILES: O=C(O)[C@H]1CCc2c(sc3ncnc(Nc4cc5cn[nH]c5cn4)c23)C1 ; CN(CCC(F)(F)F)C(=O)[C@H]1CCc2c(sc3ncnc(Nc4cc5cn[nH]c5cn4)c23)C1]

100 mg (273 μmol) (7S)-4-(1H-pyrazolo[3,4-c]pyridin-5-ylamino)-5,6,7,8-tetrahydro[1]benzothieno[2,3-d]pyrimidine-7-carboxylic acid (prepared according to intermediate example 46a) were transformed in analogy to example 18 using 3,3,3-trifluoro-N-methylpropan-1-amine to give after working up and purification 37.6 mg (28%) of the title compound.

¹H-NMR (DMSO-d6): δ=1.80 (1H), 2.12 (1H), 2.53 (2H), 2.87+3.12 (3H), 2.93 (2H), 3.05-3.72 (5H), 8.24 (1H), 8.34 (1H), 8.54 (1H), 8.68 (1H), 8.84 (1H), 13.54 (1H) ppm.

### Example 49

### (7S)—N-(2-Methoxyethyl)-4-[(6-methoxy-1H-pyrazolo[3,4-b]pyridin-5-yl)amino]-N-propyl-5,6,7,8-tetrahydro[1]benzothieno[2,3-d]pyrimidine-7-carboxamide

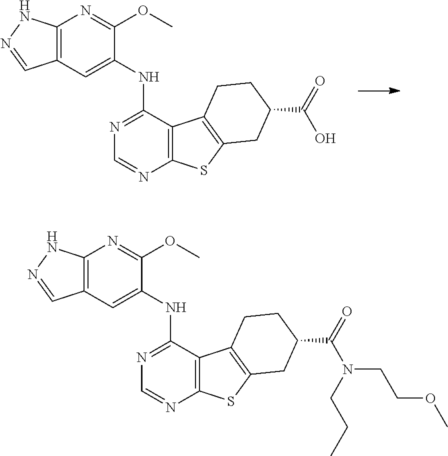 [SMILES: COc1nc2[nH]ncc2cc1Nc1ncnc2sc3c(c12)CC[C@H](C(=O)O)C3 ; CCCN(CCOC)C(=O)[C@H]1CCc2c(sc3ncnc(Nc4cc5cn[nH]c5nc4OC)c23)C1]

50 mg (136 μmol) (7S)-4-[(6-methoxy-1H-pyrazolo[3,4-b]pyridin-5-yl)amino]-5,6,7,8-tetrahydro[1]benzothieno[2,3-d]pyrimidine-7-carboxylic acid (prepared according to intermediate example 34a) were transformed in analogy to example 18 using N-(2-methoxyethyl)propan-1-amine to give after working up and purification 6.9 mg (9%) of the title compound.

¹H-NMR (DMSO-d6): δ=0.80-0.89 (3H), 1.41-1.62 (2H), 1.85 (1H), 2.09 (1H), 2.85-3.03 (2H), 3.07-3.66 (12H), 4.02 (3H), 8.02 (1H), 8.10 (1H), 8.41 (1H), 8.84 (1H), 13.36 (1H) ppm.

### Example 50

### (7S)-4-[(6-Hydroxy-1H-pyrazolo[3,4-b]pyridin-5-yl)amino]-N-(2-methoxyethyl)-N-propyl-5,6,7,8-tetrahydro[1]benzothieno[2,3-d]pyrimidine-7-carboxamide

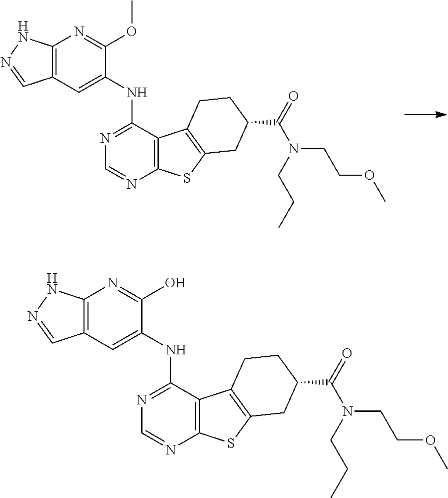 [SMILES: CCCN(CCOC)C(=O)[C@H]1CCc2c(sc3ncnc(Nc4cc5cn[nH]c5nc4OC)c23)C1 ; CCCN(CCOC)C(=O)[C@H]1CCc2c(sc3ncnc(Nc4cc5cn[nH]c5nc4O)c23)C1]

A mixture comprising 31.1 mg (63 μmol) (7S)—N-(2-methoxyethyl)-4-[(6-methoxy-1H-pyrazolo[3,4-b]pyridin-5-yl)amino]-N-propyl-5,6,7,8-tetrahydro[1]benzothieno[2,3-d]pyrimidine-7-carboxamide (prepared according to example 49), 0.7 mL ethanol and 15.7 μL hydrochloric acid (4M in dioxane) was heated under microwave irradiation at 140° C. for two hours. 100 μL triethylamine were added and the mixture purified by chromatography to give 15.1 mg (47%) of the title compound.

¹H-NMR (DMSO-d6): δ=0.80-0.88 (3H), 1.44-1.61 (2H), 1.86 (1H), 2.07 (1H), 2.84-3.04 (2H), 3.07-3.66 (12H), 8.09 (1H), 8.55 (1H), 8.88 (1H), 8.96 (1H), 12.40 (1H), 13.05 (1H) ppm.

### Example 51

### (7S)—N—Butyl-4-[(6-methoxy-1H-pyrazolo[3,4-b]pyridin-5-yl)amino]-N-methyl-5,6,7,8-tetrahydro[1]benzothieno[2,3-d]pyrimidine-7-carboxamide

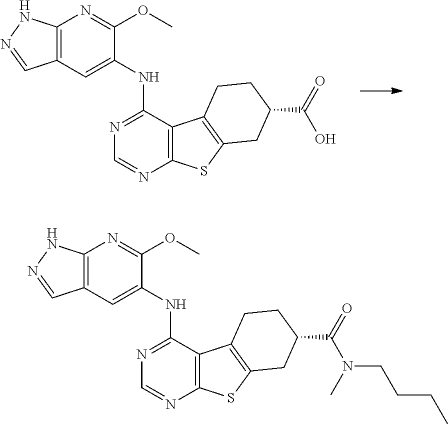 [SMILES: COc1nc2[nH]ncc2cc1Nc1ncnc2sc3c(c12)CC[C@H](C(=O)O)C3 ; CCCCN(C)C(=O)[C@H]1CCc2c(sc3ncnc(Nc4cc5cn[nH]c5nc4OC)c23)C1]

50 mg (148 μmol) (7S)-4-[(6-methoxy-1H-pyrazolo[3,4-b]pyridin-5-yl)amino]-5,6,7,8-tetrahydro[1]benzothieno[2,3-d]pyrimidine-7-carboxylic acid (prepared according to intermediate example 34a) were transformed in analogy to example 18 using N-methylbutan-1-amine to give after working up and purification 17.1 mg (24%) of the title compound.

¹H-NMR (DMSO-d6): δ=0.86-0.95 (3H), 1.27 (2H), 1.40-1.59 (2H), 1.83 (1H), 2.09 (1H), 2.85+3.07 (3H), 2.93 (2H), 3.03-3.45 (5H), 4.02 (3H), 8.02 (1H), 8.10 (1H), 8.41 (1H), 8.81+8.85 (1H), 13.38 (1H) ppm.

### Example 52

### (7S)—N-Butyl-4-[(6-hydroxy-1H-pyrazolo[3,4-b]pyridin-5-yl)amino]-N-methyl-5,6,7,8-tetrahydro[1]benzothieno[2,3-d]pyrimidine-7-carboxamide

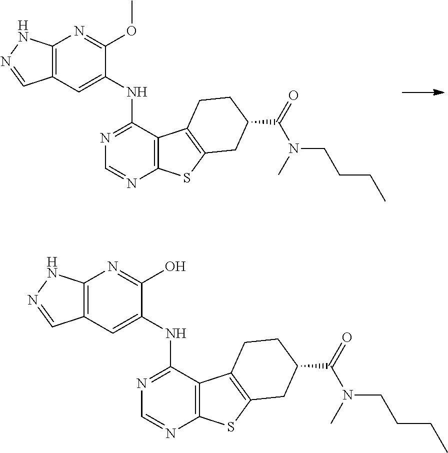 [SMILES: CCCCN(C)C(=O)[C@H]1CCc2c(sc3ncnc(Nc4cc5cn[nH]c5nc4OC)c23)C1 ; CCCCN(C)C(=O)[C@H]1CCc2c(sc3ncnc(Nc4cc5cn[nH]c5nc4O)c23)C1]

42.5 mg (91 μmol) (7S)—N-butyl-4-[(6-methoxy-1H-pyrazolo[3,4-b]pyridin-5-yl)amino]-N-methyl-5,6,7,8-tetrahydro[1]benzothieno[2,3-d]pyrimidine-7-carboxamide (prepared according to example 51) were transformed in analogy to example 50 to give after working up and purification 6.9 mg (16%) of the title compound.

¹H-NMR (DMSO-d6): δ=0.89 (3H), 1.26 (2H), 1.40-1.59 (2H), 1.85 (1H), 2.10 (1H), 2.85+3.07 (3H), 2.86-3.45 (7H), 8.08 (1H), 8.55 (1H), 8.89 (1H), 8.96 (1H), 12.46 (1H), 13.04 (1H) ppm.

### Example 53

### (7S)-4-[(6-Methoxy-1H-pyrazolo[3,4-b]pyridin-5-yl)amino]-N,N-dimethyl-5,6,7,8-tetrahydro[1]benzothieno[2,3-d]pyrimidine-7-carboxamide

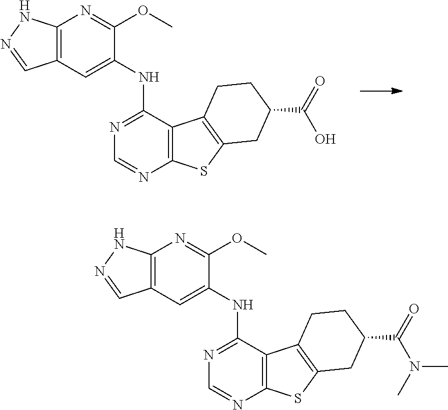 [SMILES: COc1nc2[nH]ncc2cc1Nc1ncnc2sc3c(c12)CC[C@H](C(=O)O)C3 ; COc1nc2[nH]ncc2cc1Nc1ncnc2sc3c(c12)CC[C@H](C(=O)N(C)C)C3]

50 mg (169 μmol) (7S)-4-[(6-methoxy-1H-pyrazolo[3,4-b]pyridin-5-yl)amino]-5,6,7,8-tetrahydro[1]benzothieno[2,3-d]pyrimidine-7-carboxylic acid (prepared according to intermediate example 34a) were transformed in analogy to example 18 using N-methylmethanamine to give after working up and purification 32.7 mg (43%) of the title compound.

¹H-NMR (DMSO-d6): δ=1.82 (1H), 2.12 (1H), 2.87 (3H), 2.93 (2H), 3.10 (3H), 3.13-3.28 (3H), 4.01 (3H), 8.02 (1H), 8.10 (1H), 8.41 (1H), 8.83 (1H), 13.38 (1H) ppm.

### Example 54

### (7S)-4-[(6-Hydroxy-1H-pyrazolo[3,4-b]pyridin-5-yl)amino]-N,N-dimethyl-5,6,7,8-tetrahydro[1]benzothieno[2,3-d]pyrimidine-7-carboxamide

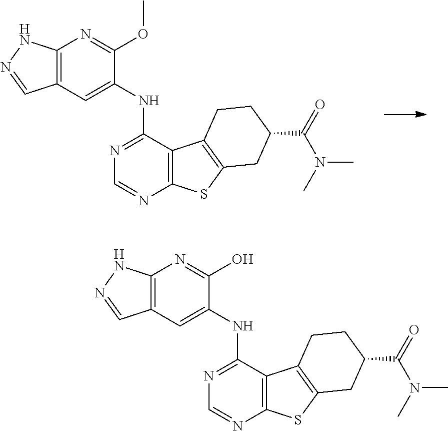 [SMILES: COc1nc2[nH]ncc2cc1Nc1ncnc2sc3c(c12)CC[C@H](C(=O)N(C)C)C3 ; CN(C)C(=O)[C@H]1CCc2c(sc3ncnc(Nc4cc5cn[nH]c5nc4O)c23)C1]

31 mg (73 μmol) (7S)-4-[(6-methoxy-1H-pyrazolo[3,4-b]pyridin-5-yl)amino]-N,N-dimethyl-5,6,7,8-tetrahydro[1]benzothieno[2,3-d]pyrimidine-7-carboxamide (prepared according to example 53) were transformed in analogy to example 50 to give after working up and purification 10.1 mg (32%) of the title compound.

¹H-NMR (DMSO-d6): δ=1.82 (1H), 2.12 (1H), 2.87 (3H), 2.90-3.02 (2H), 3.10 (3H), 3.16-3.26 (3H), 8.07 (1H), 8.55 (1H), 8.90 (1H), 8.95 (1H), 12.47 (1H), 13.03 (1H) ppm.

### Example 55

### Azetidin-1-yl[(7S)-4-(1H-pyrazolo[3,4-c]pyridin-5-ylamino)-5,6,7,8-tetrahydro[1]benzothieno[2,3-d]pyrimidin-7-yl]methanone

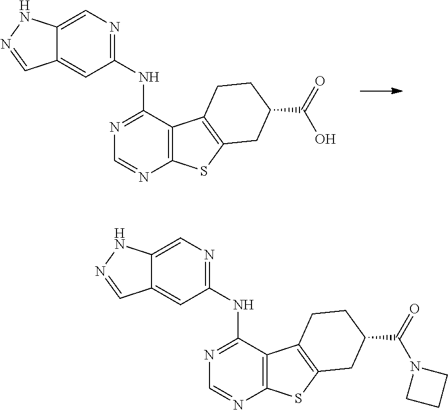 [SMILES: O=C(O)[C@H]1CCc2c(sc3ncnc(Nc4cc5cn[nH]c5cn4)c23)C1 ; O=C([C@H]1CCc2c(sc3ncnc(Nc4cc5cn[nH]c5cn4)c23)C1)N1CCC1]

78 mg (213 μmol) (7S)-4-(1H-pyrazolo[3,4-c]pyridin-5-ylamino)-5,6,7,8-tetrahydro[1]benzothieno[2,3-d]pyrimidine-7-carboxylic acid (prepared according to intermediate example 46a) were transformed in analogy to example 18 using aziridine to give after working up and purification 63.1 mg (69%) of the title compound.

¹H-NMR (DMSO-d6): δ=1.78 (1H), 2.10 (1H), 2.21 (2H), 2.72 (1H), 2.80-2.98 (2H), 3.14-3.28 (2H), 3.88 (2H), 4.24 (2H), 8.24 (1H), 8.34 (1H), 8.53 (1H), 8.68 (1H), 8.84 (1H), 13.50 (1H) ppm.

### Example 56

### (7S)—N, N-Bis(2-methoxyethyl)-4-(1H-pyrazolo[3,4-c]pyridin-5-ylamino)-5,6,7,8-tetrahydro[1]benzothieno[2,3-d]pyrimidine-7-carboxamide

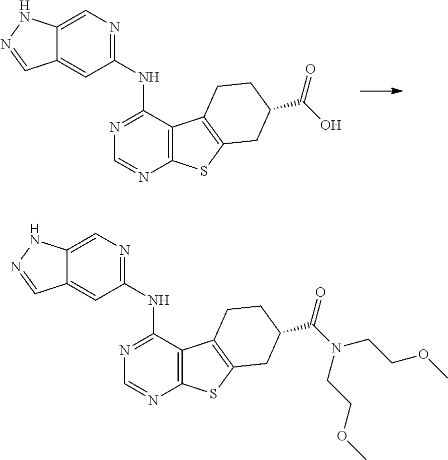 [SMILES: O=C(O)[C@H]1CCc2c(sc3ncnc(Nc4cc5cn[nH]c5cn4)c23)C1 ; COCCN(CCOC)C(=O)[C@H]1CCc2c(sc3ncnc(Nc4cc5cn[nH]c5cn4)c23)C1]

100 mg (273 μmol) (7S)-4-(1H-pyrazolo[3,4-c]pyridin-5-ylamino)-5,6,7,8-tetrahydro[1]benzothieno[2,3-d]pyrimidine-7-carboxylic acid (prepared according to intermediate example 46a) were transformed in analogy to example 18 using 2-methoxy-N-(2-methoxyethyl)ethanamine to give after working up and purification 42.6 mg (11%) of the title compound.

¹H-NMR (DMSO-d6): δ=1.81 (1H), 2.07 (1H), 2.92 (2H), 3.11-3.67 (11H), 3.25 (3H), 3.26 (3H), 8.24 (1H), 8.35 (1H), 8.53 (1H), 8.68 (1H), 8.84 (1H), 13.55 (1H) ppm.

### Example 57

### (7S)—N-(2-Methoxyethyl)-N-methyl-4-(1H-pyrazolo[3,4-c]pyridin-5-ylamino)-5,6,7,8-tetrahydro[1]benzothieno[2,3-d]pyrimidine-7-carboxamide

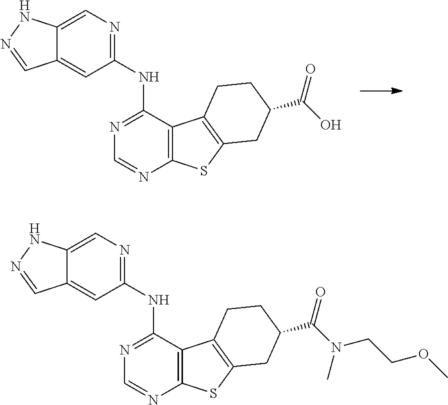 [SMILES: O=C(O)[C@H]1CCc2c(sc3ncnc(Nc4cc5cn[nH]c5cn4)c23)C1 ; COCCN(C)C(=O)[C@H]1CCc2c(sc3ncnc(Nc4cc5cn[nH]c5cn4)c23)C1]

100 mg (273 μmol) (7S)-4-(1H-pyrazolo[3,4-c]pyridin-5-ylamino)-5,6,7,8-tetrahydro[1]benzothieno[2,3-d]pyrimidine-7-carboxylic acid (prepared according to intermediate example 46a) were transformed in analogy to example 18 using 2-methoxy-N-methylethanamine to give after working up and purification 25.3 mg (21%) of the title compound.

¹H-NMR (DMSO-d6): δ=1.80 (1H), 2.11 (1H), 2.87+3.12 (3H), 2.93 (2H), 3.08-3.64 (7H), 3.25+3.27 (3H), 8.24 (1H), 8.35 (1H), 8.54 (1H), 8.69 (1H), 8.84 (1H), 13.55 (1H) ppm.

### Example 58

### (7S)—N-Ethyl-N-methyl-4-(1H-pyrazolo[3,4-c]pyridin-5-ylamino)-5,6,7,8-tetrahydro[1]benzothieno[2,3-d]pyrimidine-7-carboxamide

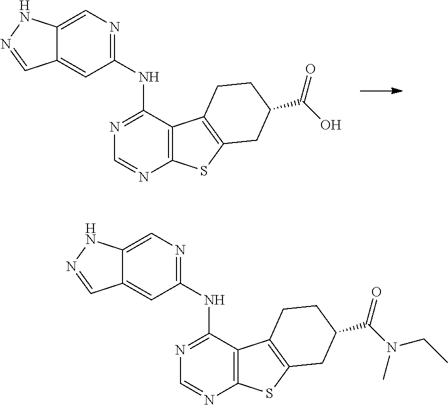 [SMILES: O=C(O)[C@H]1CCc2c(sc3ncnc(Nc4cc5cn[nH]c5cn4)c23)C1 ; CCN(C)C(=O)[C@H]1CCc2c(sc3ncnc(Nc4cc5cn[nH]c5cn4)c23)C1]

100 mg (273 μmol) (7S)-4-(1H-pyrazolo[3,4-c]pyridin-5-ylamino)-5,6,7,8-tetrahydro[1]benzothieno[2,3-d]pyrimidine-7-carboxylic acid (prepared according to intermediate example 46a) were transformed in analogy to example 18 using N-methylethanamine to give after working up and purification 30.0 mg (25%) of the title compound.

¹H-NMR (DMSO-d6): δ=1.03+1.15 (3H), 1.80 (1H), 1.78 (1H), 2.10 (1H), 2.84+3.06 (3H), 2.93 (2H), 3.08 (1H), 3.22-3.49 (3H), 8.24 (1H), 8.35 (1H), 8.54 (1H), 8.69 (1H), 8.84 (1H), 13.55 (1H) ppm.

### Example 59

### (7S)—N,N-Dimethyl-4-(1H-pyrazolo[3,4-b]pyridin-5-ylamino)-5,6,7,8-tetrahydro[1]benzothieno[2,3-d]pyrimidine-7-carboxamide

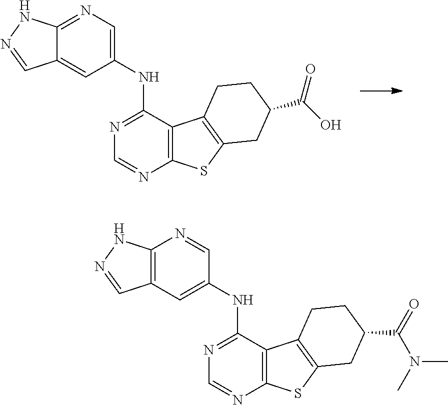 [SMILES: O=C(O)[C@H]1CCc2c(sc3ncnc(Nc4cnc5[nH]ncc5c4)c23)C1 ; CN(C)C(=O)[C@H]1CCc2c(sc3ncnc(Nc4cnc5[nH]ncc5c4)c23)C1]

50 mg (169 μmol) (7S)-4-(1H-pyrazolo[3,4-b]pyridin-5-ylamino)-5,6,7,8-tetrahydro[1]benzothieno[2,3-d]pyrimidine-7-carboxylic acid (prepared according to intermediate example 59a) were transformed in analogy to example 18 using N-methylmethanamine to give after working up and purification 30.0 mg (43%) of the title compound.

¹H-NMR (DMSO-d6): δ=1.78 (1H), 2.08 (1H), 2.87 (3H), 2.93 (2H), 3.09 (3H), 3.13-3.36 (3H), 8.13 (1H), 8.30 (1H), 8.36 (2H), 8.61 (1H), 13.60 (1H) ppm.

### Example 59a

### (7S)-4-(1H-Pyrazolo[3,4-b]pyridin-5-ylamino)-5,6,7,8-tetrahydro[1]benzothieno[2,3-d]pyrimidine-7-carboxylic acid

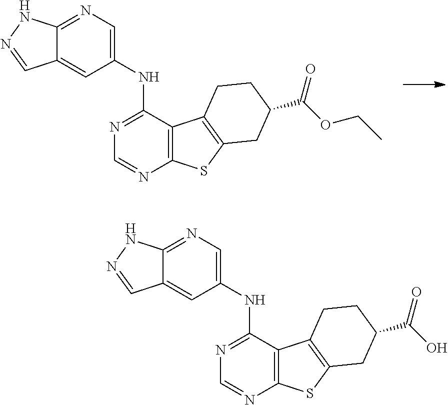 [SMILES: CCOC(=O)[C@H]1CCc2c(sc3ncnc(Nc4cnc5[nH]ncc5c4)c23)C1 ; O=C(O)[C@H]1CCc2c(sc3ncnc(Nc4cnc5[nH]ncc5c4)c23)C1]

5.13 g (13.0 mmol) ethyl (7S)-4-(1H-pyrazolo[3,4-b]pyridin-5-ylamino)-5,6,7,8-tetrahydro[1]benzothieno[2,3-d]pyrimidine-7-carboxylate (prepared according to intermediate example 59b) were transformed in analogy to example 11 to give after working up and purification 4.93 g (98%) of the title compound.

### Example 59b

### Ethyl (7S)-4-(1H-pyrazolo[3,4-b]pyridin-5-ylamino)-5,6,7,8-tetrahydro[1]benzothieno[2,3-d]pyrimidine-7-carboxylate

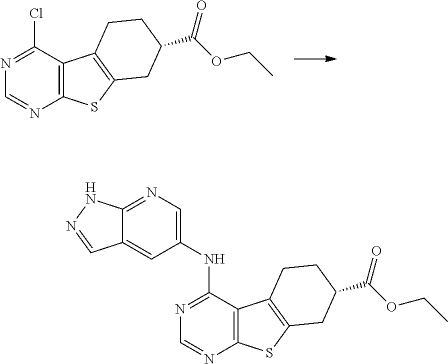 [SMILES: CCOC(=O)[C@H]1CCc2c(sc3ncnc(Cl)c23)C1 ; CCOC(=O)[C@H]1CCc2c(sc3ncnc(Nc4cnc5[nH]ncc5c4)c23)C1]

5.00 g (16.8 mmol) ethyl (7S)-4-chloro-5,6,7,8-tetrahydro[1]benzothieno[2,3-d]pyrimidine-7-carboxylate (prepared according to intermediate example 34c) were transformed in analogy to intermediate example 34b using 1H-pyrazolo[3,4-b]pyridin-5-amine (CAS-No. 942185-01-5) to give after working up and purification 5.05 g (72%) of the title compound.

### Example 60

### (7S)—N,N-Dimethyl-4-(1H-pyrazolo[3,4-c]pyridin-5-ylamino)-5,6,7,8-tetrahydro[1]benzothieno[2,3-d]pyrimidine-7-carboxamide

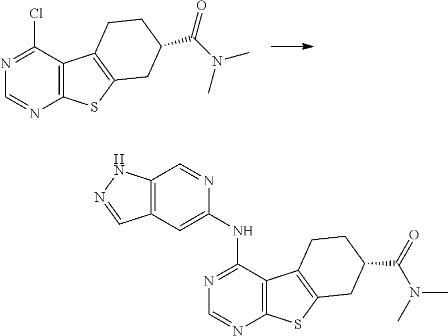 [SMILES: CN(C)C(=O)[C@H]1CCc2c(sc3ncnc(Cl)c23)C1 ; CN(C)C(=O)[C@H]1CCc2c(sc3ncnc(Nc4cc5cn[nH]c5cn4)c23)C1]

89 mg (301 μmol) (7S)-4-chloro-N,N-dimethyl-5,6,7,8-tetrahydro[1]benzothieno[2,3-d]pyrimidine-7-carboxamide (prepared according to intermediate example 60a) were transformed in analogy to intermediate example 46b to give after working up and purification 46.4 mg (37%) of the title compound.

¹H-NMR (DMSO-d6): δ=1.79 (1H), 2.12 (1H), 2.87 (3H), 2.93 (2H), 3.09 (3H), 3.15 (1H), 3.27 (2H), 8.24 (1H), 8.34 (1H), 8.54 (1H), 8.69 (1H), 8.84 (1H), 13.53 (1H) ppm.

### Example 60a

### (7S)-4-Chloro-N,N-dimethyl-5,6,7,8-tetrahydro[1]benzothieno[2,3-d]pyrimidine-7-carboxamide

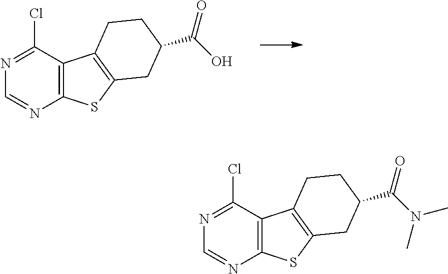 [SMILES: O=C(O)[C@H]1CCc2c(sc3ncnc(Cl)c23)C1 ; CN(C)C(=O)[C@H]1CCc2c(sc3ncnc(Cl)c23)C1]

2.64 g (9.82 mmol) (7S)-4-chloro-5,6,7,8-tetrahydro[1]benzothieno[2,3-d]pyrimidine-7-carboxylic acid (prepared according to intermediate example 60b) were transformed in analogy to example 18 using N-methylmethanamine to give after working up and purification 2.74 g (94%) of the title compound.

### Example 60b

### (7S)-4-Chloro-5,6,7,8-tetrahydro[1]benzothieno[2,3-d]pyrimidine-7-carboxylic acid

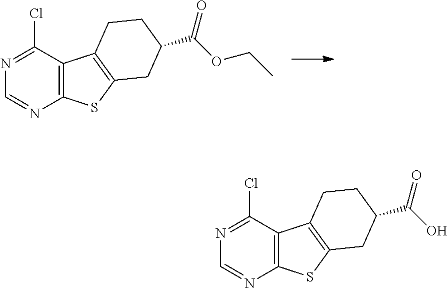 [SMILES: CCOC(=O)[C@H]1CCc2c(sc3ncnc(Cl)c23)C1 ; O=C(O)[C@H]1CCc2c(sc3ncnc(Cl)c23)C1]

5.00 g (16.8 mmol) ethyl (7S)-4-chloro-5,6,7,8-tetrahydro[1]benzothieno[2,3-d]pyrimidine-7-carboxylate (prepared according to intermediate example 34c) were transformed in analogy to example 11 to give after working up and purification 4.35 g (96%) of the title compound.

### Example 61

### [5-Bromo-4-(1H-pyrazolo[3,4-b]pyridin-5-ylamino)-7H-pyrrolo[2,3-d]pyrimidin-6-yl](piperidin-1-yl)methanone

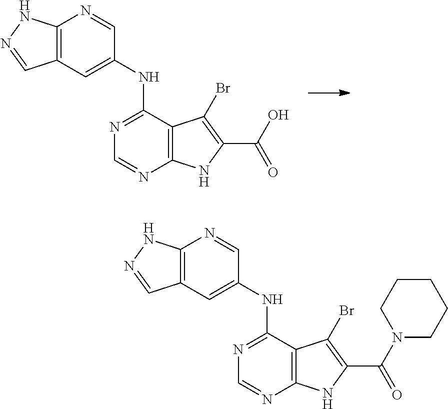 [SMILES: O=C(O)c1[nH]c2ncnc(Nc3cnc4[nH]ncc4c3)c2c1Br ; O=C(c1[nH]c2ncnc(Nc3cnc4[nH]ncc4c3)c2c1Br)N1CCCCC1]

100 mg (267 μmol) 5-bromo-4-(1H-pyrazolo[3,4-b]pyridin-5-ylamino)-7H-pyrrolo[2,3-d]pyrimidine-6-carboxylic acid (prepared according to intermediate example 61a) were transformed in analogy to example 18 using piperidine to give after working up and purification 20.0 mg (16%) of the title compound.

¹H-NMR (DMSO-d6): δ=1.56 (4H), 1.63 (2H), 3.61 (4H), 8.13 (1H), 8.25 (1H), 8.46 (1H), 8.51 (1H), 8.66 (1H), 12.69 (1H), 13.59 (1H) ppm.

### Example 61a

### 5-Bromo-4-(1H-pyrazolo[3,4-b]pyridin-5-ylamino)-7H-pyrrolo[2,3-d]pyrimidine-6-carboxylic acid

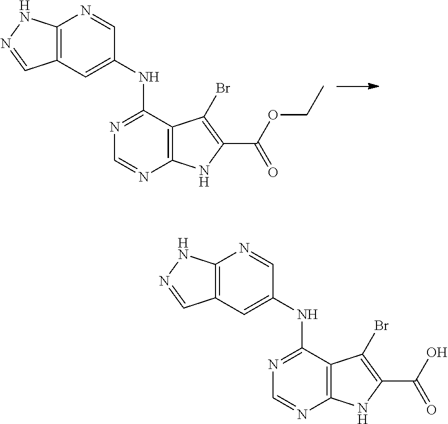 [SMILES: CCOC(=O)c1[nH]c2ncnc(Nc3cnc4[nH]ncc4c3)c2c1Br ; O=C(O)c1[nH]c2ncnc(Nc3cnc4[nH]ncc4c3)c2c1Br]

1.71 g (4.26 mmol) ethyl 5-bromo-4-(1H-pyrazolo[3,4-b]pyridin-5-ylamino)-7H-pyrrolo[2,3-d]pyrimidine-6-carboxylate (prepared according to example 12) were transformed in analogy to example 11 to give after working up and purification 1.68 g (99%) of the title compound.

### Example 62

### 5-Bromo-N-[2-(dimethylamino)ethyl]-4-[(6-methoxy-1H-pyrazolo[3,4-b]pyridin-5-yl)amino]-N-methyl-7H-pyrrolo[2,3-d]pyrimidine-6-carboxamide

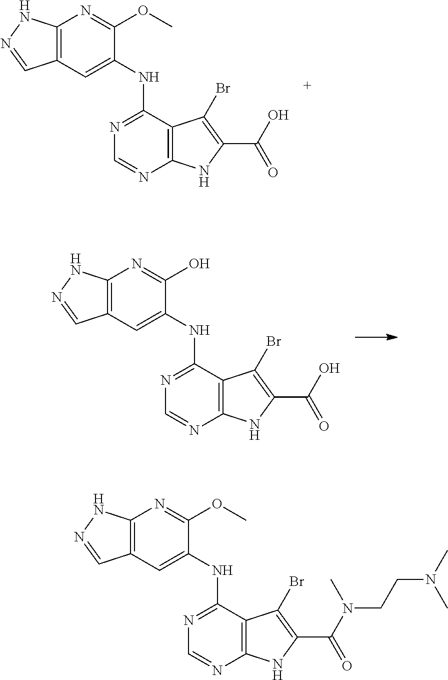 [SMILES: COc1nc2[nH]ncc2cc1Nc1ncnc2[nH]c(C(=O)O)c(Br)c12 ; O=C(O)c1[nH]c2ncnc(Nc3cc4cn[nH]c4nc3O)c2c1Br ; COc1nc2[nH]ncc2cc1Nc1ncnc2[nH]c(C(=O)N(C)CCN(C)C)c(Br)c12]

200 mg (495 μmol) of a mixture comprising 5-bromo-4-[(6-methoxy-1H-pyrazolo[3,4-b]pyridin-5-yl)amino]-7H-pyrrolo[2,3-d]pyrimidine-6-carboxylic acid and 5-bromo-4-[(6-hydroxy-1H-pyrazolo[3,4-b]pyridin-5-yl)amino]-7H-pyrrolo[2,3-d]pyrimidine-6-carboxylic acid (prepared according to intermediate example 62a) were transformed in analogy to example 18 using N,N,N′-trimethylethane-1,2-diamine to give after working up and purification 31.7 mg (12%) of the title compound.

¹H-NMR (DMSO-d6): δ=2.10-2.25 (6H), 3.03 (3H), 3.35-3.63 (4H), 4.12 (3H), 7.97-8.07 (1H), 8.44 (1H), 8.65-8.75 (1H), 9.28 (1H), 13.05-13.56 (1H) ppm.

### Example 62a

### 5-Bromo-4-[(6-methoxy-1H-pyrazolo[3,4-b]pyridin-5-yl)amino]-7H-pyrrolo[2,3-d]pyrimidine-6-carboxylic acid (A) and 5-bromo-4-[(6-hydroxy-1H-pyrazolo[3,4-b]pyridin-5-yl)amino]-7H-pyrrolo[2,3-d]pyrimidine-6-carboxylic acid (B)

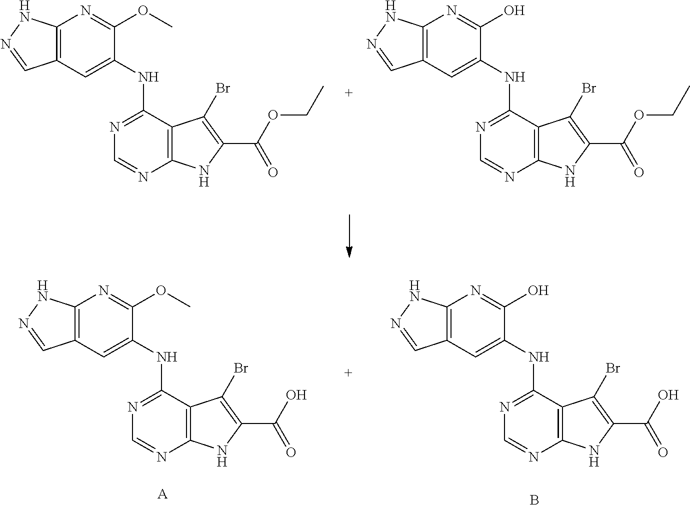

1.65 g (3.81 mmol) of a mixture comprising ethyl 5-bromo-4-[(6-methoxy-1H-pyrazolo[3,4-b]pyridin-5-yl)amino]-7H-pyrrolo[2,3-d]pyrimidine-6-carboxylate and ethyl 5-bromo-4-[(6-hydroxy-1H-pyrazolo[3,4-b]pyridin-5-yl)amino]-7H-pyrrolo[2,3-d]pyrimidine-6-carboxylate (prepared according to intermediate example 62b) were transformed in analogy to example 11 to give after working up and purification 1.52 g (50%) a mixture of title compounds A and B that was not separated.

### Example 62b

### Ethyl 5-bromo-4-[(6-methoxy-1H-pyrazolo[3,4-b]pyridin-5-yl)amino]-7H-pyrrolo[2,3-d]pyrimidine-6-carboxylate (A) and ethyl 5-bromo-4-[(6-hydroxy-1H-pyrazolo[3,4-b]pyridin-5-yl)amino]-7H-pyrrolo[2,3-d]pyrimidine-6-carboxylate (B)

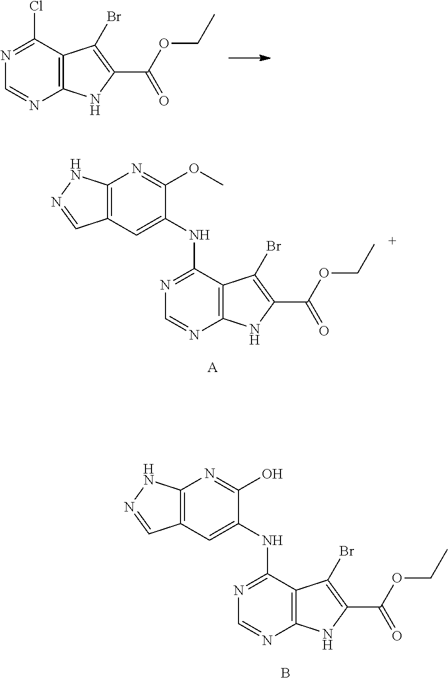

1.94 g (6.36 mmol) ethyl 5-bromo-4-chloro-7H-pyrrolo[2,3-d]pyrimidine-6-carboxylate (prepared according to intermediate example 61c) were transformed in analogy to example 6 using 6-methoxy-1H-pyrazolo[3,4-b]pyridin-5-amine (prepared according to intermediate example 34f) to give after working up and purification 1.65 g (60%) of a mixture of title compounds A and B that was not separated.

### Example 63

### 5-Bromo-N-[2-(dimethylamino)ethyl]-4-[(6-hydroxy-1H-pyrazolo[3,4-b]pyridin-5-yl)amino]-N-methyl-7H-pyrrolo[2,3-d]pyrimidine-6-carboxamide

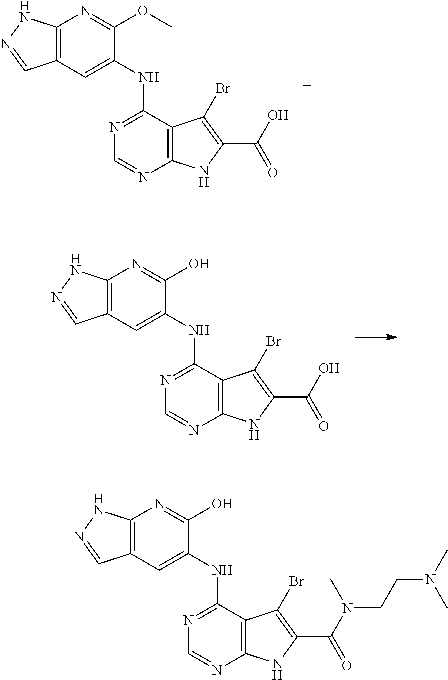 [SMILES: COc1nc2[nH]ncc2cc1Nc1ncnc2[nH]c(C(=O)O)c(Br)c12 ; O=C(O)c1[nH]c2ncnc(Nc3cc4cn[nH]c4nc3O)c2c1Br ; CN(C)CCN(C)C(=O)c1[nH]c2ncnc(Nc3cc4cn[nH]c4nc3O)c2c1Br]

200 mg (495 μmol) of a mixture comprising 5-bromo-4-[(6-methoxy-1H-pyrazolo[3,4-b]pyridin-5-yl)amino]-7H-pyrrolo[2,3-d]pyrimidine-6-carboxylic acid and 5-bromo-4-[(6-hydroxy-1H-pyrazolo[3,4-b]pyridin-5-yl)amino]-7H-pyrrolo[2,3-d]pyrimidine-6-carboxylic acid (prepared according to intermediate example 62a) were transformed in analogy to example 18 using N,N,N′-trimethylethane-1,2-diamine to give after working up and purification 68.6 mg (28%) of the title compound.

¹H-NMR (DMSO-d6): δ=2.10 (3H), 2.26 (3H), 3.02 (3H), 3.34-3.70 (4H), 8.09 (1H), 8.16 (1H), 8.47 (1H), 9.04 (1H), 9.26-9.40 (1H), 11.78-12.63 (1H), 12.70-13.24 (1H) ppm.

### Example 64

### {5-Bromo-4-[(6-methoxy-1H-pyrazolo[3,4-b]pyridin-5-yl)amino]-7H-pyrrolo[2,3-d]pyrimidin-6-yl}[4-(dimethylamino)piperidin-1-yl]methanone

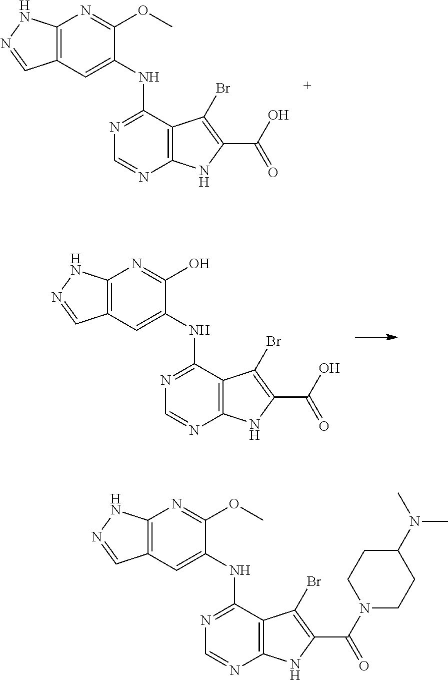 [SMILES: COc1nc2[nH]ncc2cc1Nc1ncnc2[nH]c(C(=O)O)c(Br)c12 ; O=C(O)c1[nH]c2ncnc(Nc3cc4cn[nH]c4nc3O)c2c1Br ; COc1nc2[nH]ncc2cc1Nc1ncnc2[nH]c(C(=O)N3CCC(N(C)C)CC3)c(Br)c12]

200 mg (495 μmol) of a mixture comprising 5-bromo-4-[(6-methoxy-1H-pyrazolo[3,4-b]pyridin-5-yl)amino]-7H-pyrrolo[2,3-d]pyrimidine-6-carboxylic acid and 5-bromo-4-[(6-hydroxy-1H-pyrazolo[3,4-b]pyridin-5-yl)amino]-7H-pyrrolo[2,3-d]pyrimidine-6-carboxylic acid (prepared according to intermediate example 62a) were transformed in analogy to example 18 using N,N-dimethylpiperidin-4-amine to give after working up and purification 32.2 mg (12%) of the title compound.

¹H-NMR (DMSO-d6): δ=1.39 (2H), 1.79 (2H), 2.17 (6H), 2.37 (1H), 2.82-3.31 (4H), 4.10 (3H), 8.03 (1H), 8.39 (1H), 8.64 (1H), 9.28 (1H), 13.33 (1H) ppm.

### Example 65

### {5-Bromo-4-[(6-hydroxy-1H-pyrazolo[3,4-b]pyridin-5-yl)amino]-7H-pyrrolo[2,3-d]pyrimidin-6-yl)}[4-(dimethylamino)piperidin-1-yl]methanone

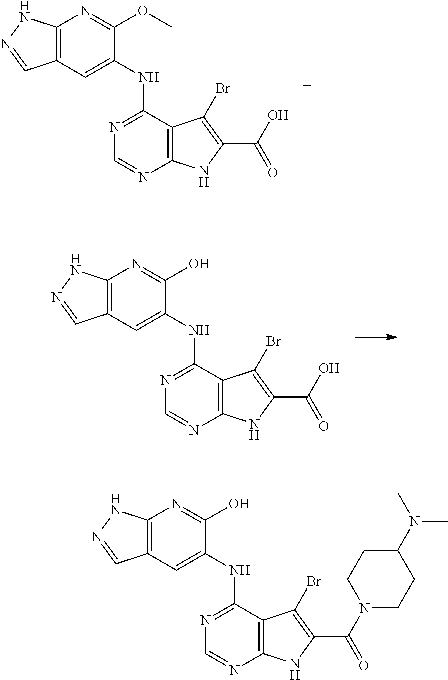 [SMILES: COc1nc2[nH]ncc2cc1Nc1ncnc2[nH]c(C(=O)O)c(Br)c12 ; O=C(O)c1[nH]c2ncnc(Nc3cc4cn[nH]c4nc3O)c2c1Br ; CN(C)C1CCN(C(=O)c2[nH]c3ncnc(Nc4cc5cn[nH]c5nc4O)c3c2Br)CC1]

200 mg (495 μmol) of a mixture comprising 5-bromo-4-[(6-methoxy-1H-pyrazolo[3,4-b]pyridin-5-yl)amino]-7H-pyrrolo[2,3-d]pyrimidine-6-carboxylic acid and 5-bromo-4-[(6-hydroxy-1H-pyrazolo[3,4-b]pyridin-5-yl)amino]-7H-pyrrolo[2,3-d]pyrimidine-6-carboxylic acid (prepared according to intermediate example 62a) were transformed in analogy to example 18 using N,N-dimethylpiperidin-4-amine to give after working up and purification 32.2 mg (12%) of the title compound.

¹H-NMR (DMSO-d6): δ=1.39 (2H), 1.79 (2H), 2.18 (6H), 2.39 (1H), 2.83-3.69 (4H), 8.08 (1H), 8.46 (1H), 9.02 (1H), 9.30 (1H), 12.26 (1H), 13.05 (1H) ppm.

### Example 66

### {5-Bromo-4-[(6-methoxy-1H-pyrazolo[3,4-b]pyridin-5-yl)amino]-7H-pyrrolo[2,3-d]pyrimidin-6-yl)}[(3R)-3-methylmorpholin-4-yl]methanone

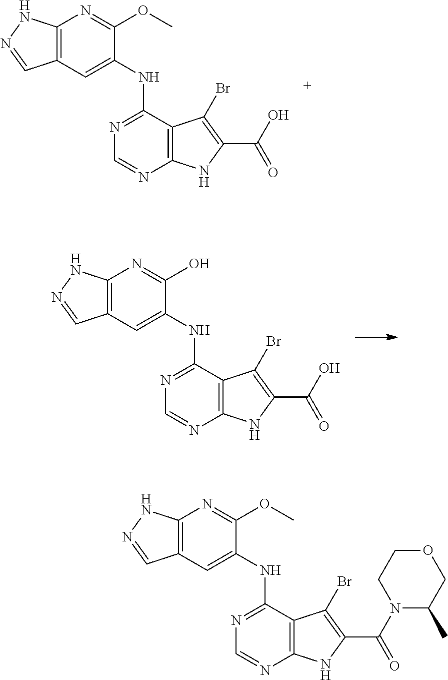 [SMILES: COc1nc2[nH]ncc2cc1Nc1ncnc2[nH]c(C(=O)O)c(Br)c12 ; O=C(O)c1[nH]c2ncnc(Nc3cc4cn[nH]c4nc3O)c2c1Br ; COc1nc2[nH]ncc2cc1Nc1ncnc2[nH]c(C(=O)N3CCOC[C@H]3C)c(Br)c12]

200 mg (495 μmol) of a mixture comprising 5-bromo-4-[(6-methoxy-1H-pyrazolo[3,4-b]pyridin-5-yl)amino]-7H-pyrrolo[2,3-d]pyrimidine-6-carboxylic acid and 5-bromo-4-[(6-hydroxy-1H-pyrazolo[3,4-b]pyridin-5-yl)amino]-7H-pyrrolo[2,3-d]pyrimidine-6-carboxylic acid (prepared according to intermediate example 62a) were transformed in analogy to example 18 using (3R)-3-methylmorpholine to give after working up and purification 9.6 mg (4%) of the title compound.

¹H-NMR (DMSO-d6): δ=1.28 (3H), 3.19-3.73 (6H), 3.88 (1H), 4.11 (3H), 8.03 (1H), 8.44 (1H), 8.66 (1H), 9.26 (1H), 12.73 (1H), 13.33 (1H) ppm.

### Example 67

### {5-Bromo-4-[(6-methoxy-1H-pyrazolo[3,4-b]pyridin-5-yl)amino]-7H-pyrrolo[2,3-d]pyrimidin-6-yl}(morpholin-4-yl)methanone

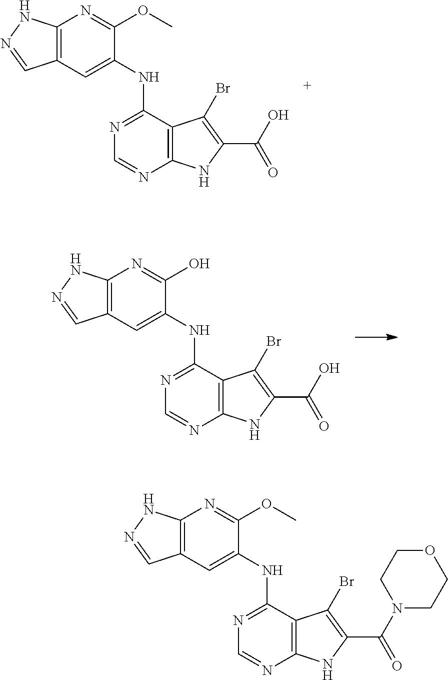 [SMILES: COc1nc2[nH]ncc2cc1Nc1ncnc2[nH]c(C(=O)O)c(Br)c12 ; O=C(O)c1[nH]c2ncnc(Nc3cc4cn[nH]c4nc3O)c2c1Br ; COc1nc2[nH]ncc2cc1Nc1ncnc2[nH]c(C(=O)N3CCOCC3)c(Br)c12]

200 mg (495 μmol) of a mixture comprising 5-bromo-4-[(6-methoxy-1H-pyrazolo[3,4-b]pyridin-5-yl)amino]-7H-pyrrolo[2,3-d]pyrimidine-6-carboxylic acid and 5-bromo-4-[(6-hydroxy-1H-pyrazolo[3,4-b]pyridin-5-yl)amino]-7H-pyrrolo[2,3-d]pyrimidine-6-carboxylic acid (prepared according to intermediate example 62a) were transformed in analogy to example 18 using (3R)-3-methylmorpholine to give after working up and purification 28.0 mg (12%) of the title compound.

¹H-NMR (DMSO-d6): δ=3.40-3.76 (8H), 4.11 (3H), 8.03 (1H), 8.44 (1H), 8.67 (1H), 9.25 (1H), 12.74 (1H), 13.34 (1H) ppm.

### Example 68

### {5-bromo-4-[(6-hydroxy-1H-pyrazolo[3,4-b]pyridin-5-yl)amino]-7H-pyrrolo[2,3-d]pyrimidin-6-yl)}(morpholin-4-yl)methanone

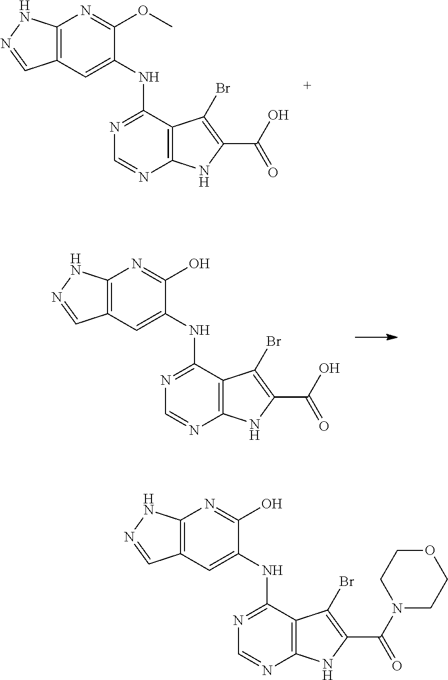 [SMILES: COc1nc2[nH]ncc2cc1Nc1ncnc2[nH]c(C(=O)O)c(Br)c12 ; O=C(O)c1[nH]c2ncnc(Nc3cc4cn[nH]c4nc3O)c2c1Br ; O=C(c1[nH]c2ncnc(Nc3cc4cn[nH]c4nc3O)c2c1Br)N1CCOCC1]

200 mg (495 μmol) of a mixture comprising 5-bromo-4-[(6-methoxy-1H-pyrazolo[3,4-b]pyridin-5-yl)amino]-7H-pyrrolo[2,3-d]pyrimidine-6-carboxylic acid and 5-bromo-4-[(6-hydroxy-1H-pyrazolo[3,4-b]pyridin-5-yl)amino]-7H-pyrrolo[2,3-d]pyrimidine-6-carboxylic acid (prepared according to intermediate example 62a) were transformed in analogy to example 18 using (3R)-3-methylmorpholine to give after working up and purification 59.0 mg (26%) of the title compound.

¹H-NMR (DMSO-d6): δ=3.41-3.72 (8H), 8.08 (1H), 8.47 (1H), 9.03 (1H), 9.32 (1H), 12.41 (1H), 13.04 (1H) ppm.

### Example 69

### [5-Bromo-4-(1H-pyrazolo[3,4-c]pyridin-5-ylamino)-7H-pyrrolo[2,3-d]pyrimidin-6-yl][4-(dimethylamino)piperidin-1-yl]methanone

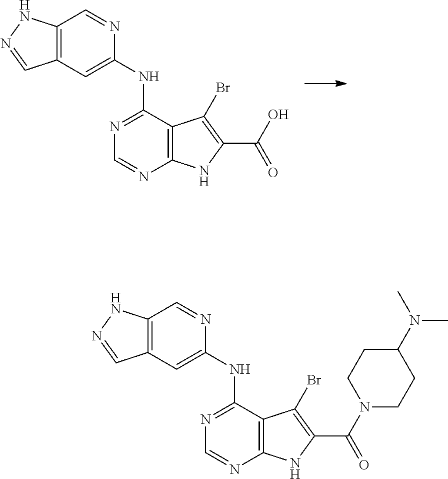 [SMILES: O=C(O)c1[nH]c2ncnc(Nc3cc4cn[nH]c4cn3)c2c1Br ; CN(C)C1CCN(C(=O)c2[nH]c3ncnc(Nc4cc5cn[nH]c5cn4)c3c2Br)CC1]

100 mg (267 μmol) 5-bromo-4-(1H-pyrazolo[3,4-c]pyridin-5-ylamino)-7H-pyrrolo[2,3-d]pyrimidine-6-carboxylic acid (prepared according to example 14) were transformed in analogy to example 18 using N,N-dimethylpiperidin-4-amine to give after working up and purification 20.9 mg (15%) of the title compound.

¹H-NMR (DMSO-d6): δ=1.40 (2H), 1.80 (2H), 2.19 (6H), 2.26 (1H), 2.40 (1H), 2.85-3.72 (3H), 8.26 (1H), 8.49 (1H), 8.72 (1H), 8.85 (1H), 8.87 (1H), 12.74 (1H), 13.55 (1H) ppm.

### Example 70

### [5-Bromo-4-(1H-pyrazolo[3,4-c]pyridin-5-ylamino)-7H-pyrrolo[2,3-d]pyrimidin-6-yl][(3R)-3-methylmorpholin-4-yl]methanone

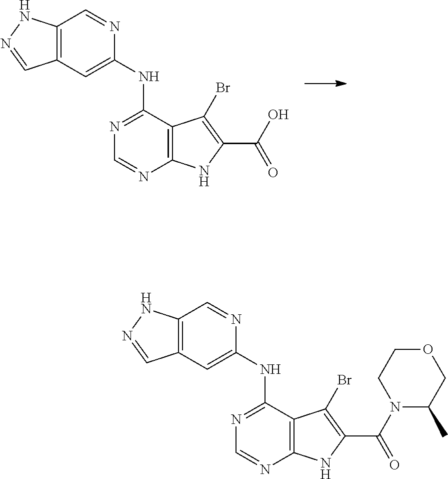 [SMILES: O=C(O)c1[nH]c2ncnc(Nc3cc4cn[nH]c4cn3)c2c1Br ; C[C@@H]1COCCN1C(=O)c1[nH]c2ncnc(Nc3cc4cn[nH]c4cn3)c2c1Br]

100 mg (267 μmol) 5-bromo-4-(1H-pyrazolo[3,4-c]pyridin-5-ylamino)-7H-pyrrolo[2,3-d]pyrimidine-6-carboxylic acid (prepared according to intermediate example 14) were transformed in analogy to example 18 using (3R)-3-methylmorpholine to give after working up and purification 17.1 mg (13%) of the title compound.

¹H-NMR (DMSO-d6): δ=1.29 (3H), 3.42 (2H), 3.64 (2H), 3.87 (1H), 8.28 (1H), 8.50 (1H), 8.78 (1H), 8.88 (2H), 12.98 (1H) ppm.

### Example 71

### [5-Bromo-4-(1H-pyrazolo[3,4-c]pyridin-5-ylamino)-7H-pyrrolo[2,3-d]pyrimidin-6-yl](morpholin-4-yl)methanone

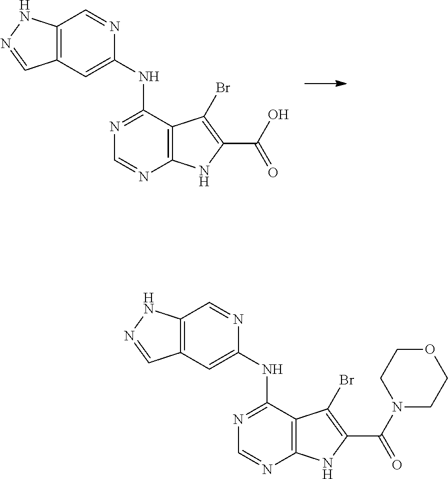 [SMILES: O=C(O)c1[nH]c2ncnc(Nc3cc4cn[nH]c4cn3)c2c1Br ; O=C(c1[nH]c2ncnc(Nc3cc4cn[nH]c4cn3)c2c1Br)N1CCOCC1]

100 mg (267 μmol) 5-bromo-4-(1H-pyrazolo[3,4-c]pyridin-5-ylamino)-7H-pyrrolo[2,3-d]pyrimidine-6-carboxylic acid (prepared according to intermediate example 14) were transformed in analogy to example 18 using morpholine to give after working up and purification 27.8 mg (23%) of the title compound.

¹H-NMR (DMSO-d6): δ=3.30-3.75 (8H), 8.26 (1H), 8.50 (1H), 8.74 (1H), 8.85 (1H), 8.87 (1H), 12.85 (1H), 13.56 (1H) ppm.

### Example 72

### [5-Bromo-4-(1H-pyrazolo[3,4-c]pyridin-5-ylamino)-7H-pyrrolo[2,3-d]pyrimidin-6-yl](piperidin-1-yl)methanone

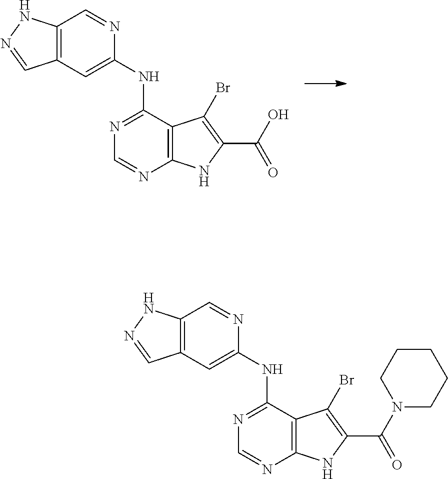 [SMILES: O=C(O)c1[nH]c2ncnc(Nc3cc4cn[nH]c4cn3)c2c1Br ; O=C(c1[nH]c2ncnc(Nc3cc4cn[nH]c4cn3)c2c1Br)N1CCCCC1]

100 mg (267 μmol) 5-bromo-4-(1H-pyrazolo[3,4-c]pyridin-5-ylamino)-7H-pyrrolo[2,3-d]pyrimidine-6-carboxylic acid (prepared according to intermediate example 14) were transformed in analogy to example 18 using piperidine to give after working up and purification 21.0 mg (17%) of the title compound.

¹H-NMR (DMSO-d6): δ=1.56 (4H), 1.63 (2H), 3.37 (2H), 3.61 (2H), 8.29 (1H), 8.50 (1H), 8.75 (1H), 8.89 (1H), 8.93 (1H), 12.96 (1H) ppm.

### Example 73

### [5-Bromo-4-(1H-pyrazolo[3,4-b]pyridin-5-ylamino)-7H-pyrrolo[2,3-d]pyrimidin-6-yl][(3R)-3-methylmorpholin-4-yl]methanone

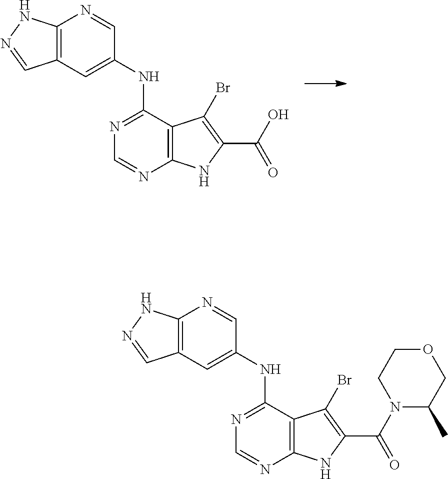 [SMILES: O=C(O)c1[nH]c2ncnc(Nc3cnc4[nH]ncc4c3)c2c1Br ; C[C@@H]1COCCN1C(=O)c1[nH]c2ncnc(Nc3cnc4[nH]ncc4c3)c2c1Br]

100 mg (267 μmol) 5-bromo-4-(1H-pyrazolo[3,4-b]pyridin-5-ylamino)-7H-pyrrolo[2,3-d]pyrimidine-6-carboxylic acid (prepared according to intermediate example 61a) were transformed in analogy to example 18 using (3R)-3-methylmorpholine to give after working up and purification 23.1 mg (17%) of the title compound.

¹H-NMR (DMSO-d6): δ=1.28 (3H), 3.21-3.49 (4H), 3.53-3.75 (2H), 3.87 (1H), 8.13 (1H), 8.26 (1H), 8.47 (1H), 8.51 (1H), 8.66 (1H), 12.74 (1H), 13.60 (1H) ppm.

### Example 74

### [5-Bromo-4-(1H-pyrazolo[3,4-b]pyridin-5-ylamino)-7H-pyrrolo[2,3-d]pyrimidin-6-yl](morpholin-4-yl)methanone

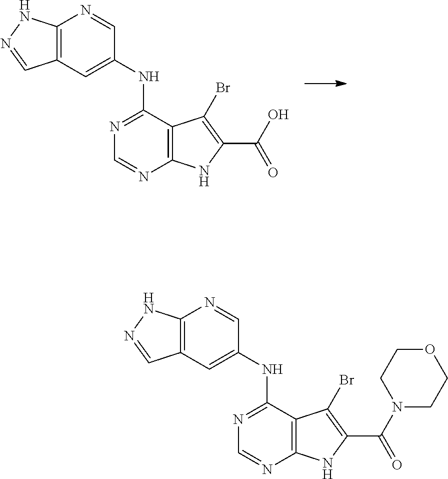 [SMILES: O=C(O)c1[nH]c2ncnc(Nc3cnc4[nH]ncc4c3)c2c1Br ; O=C(c1[nH]c2ncnc(Nc3cnc4[nH]ncc4c3)c2c1Br)N1CCOCC1]

100 mg (267 μmol) 5-bromo-4-(1H-pyrazolo[3,4-b]pyridin-5-ylamino)-7H-pyrrolo[2,3-d]pyrimidine-6-carboxylic acid (prepared according to intermediate example 61a) were transformed in analogy to example 18 using morpholine to give after working up and purification 33.2 mg (26%) of the title compound.

¹H-NMR (DMSO-d6): δ=3.24-3.70 (8H), 8.13 (1H), 8.26 (1H), 8.49 (1H), 8.51 (1H), 8.66 (1H), 12.73 (1H), 13.60 (1H) ppm.

### Example 75

### [5-Bromo-4-(1H-pyrazolo[3,4-b]pyridin-5-ylamino)-7H-pyrrolo[2,3-d]pyrimidin-6-yl][(3S)-3-methy morpholin-4-yl]methanone

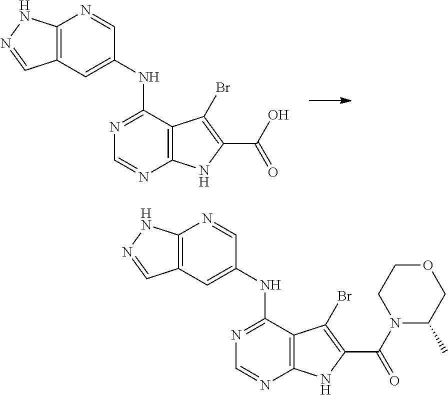 [SMILES: O=C(O)c1[nH]c2ncnc(Nc3cnc4[nH]ncc4c3)c2c1Br ; C[C@H]1COCCN1C(=O)c1[nH]c2ncnc(Nc3cnc4[nH]ncc4c3)c2c1Br]

100 mg (267 μmol) 5-bromo-4-(1H-pyrazolo[3,4-b]pyridin-5-ylamino)-7H-pyrrolo[2,3-d]pyrimidine-6-carboxylic acid (prepared according to intermediate example 61a) were transformed in analogy to example 18 using (3S)-3-methylmorpholine to give after working up and purification 21.9 mg (17%) of the title compound.

¹H-NMR (DMSO-d6): δ=1.28 (3H), 3.20-3.48 (4H), 3.55-3.74 (2H), 3.87 (1H), 8.13 (1H), 8.26 (1H), 8.47 (1H), 8.51 (1H), 8.66 (1H), 12.75 (1H), 13.60 (1H) ppm.

### Example 76

### N-[2-(Dimethylamino)-2-oxoethyl]-4-(1H-pyrazolo[3,4-c]pyridin-5-ylamino)-7H-pyrrolo[2,3-d]pyrimidine-6-carboxamide

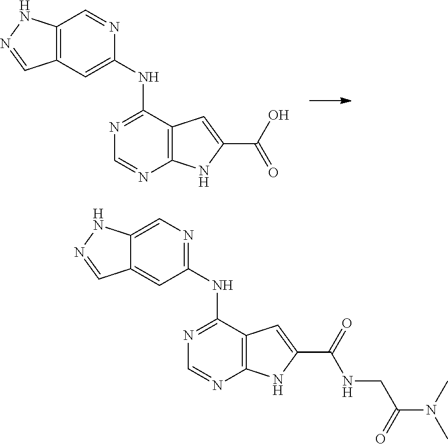

100 mg (339 μmol) 4-(1H-pyrazolo[3,4-c]pyridin-5-ylamino)-7H-pyrrolo[2,3-d]pyrimidine-6-carboxylic acid (prepared according to example 11) were transformed in analogy to example 18 using N,N-dimethylglycinamide to give after working up and purification 5.0 mg (4%) of the title compound.

¹H-NMR (DMSO-d6): δ=2.87 (3H), 3.01 (3H), 3.10 (1H), 4.15 (2H), 7.59 (1H), 8.19 (1H), 8.22 (1H), 8.41 (1H), 8.75 (1H), 8.86 (1H), 10.22 (1H), 13.46 (1H) ppm.

### Example 77

### N-[2-(Dimethylamino)ethyl]-N-methyl-4-(1H-pyrazolo[3,4-c]pyridin-5-ylamino)-7H-pyrrolo[2,3-d]pyrimidine-6-carboxamide

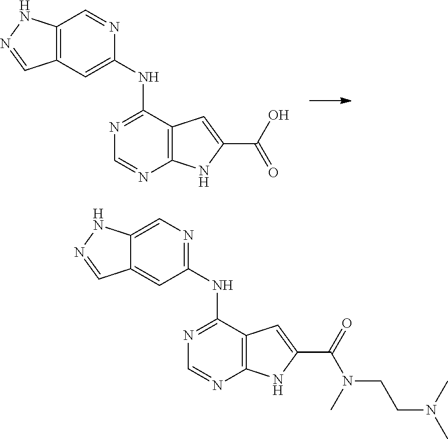

100 mg (339 μmol) 4-(1H-pyrazolo[3,4-c]pyridin-5-ylamino)-7H-pyrrolo[2,3-d]pyrimidine-6-carboxylic acid (prepared according to example 11) were transformed in analogy to example 18 using N,N,N′-trimethylethane-1,2-diamine to give after working up and purification 29.0 mg (23%) of the title compound.

¹H-NMR (DMSO-d6): δ=2.18 (6H), 2.93-3.39 (2H), 3.30 (3H), 3.62 (2H), 7.53 (1H), 8.22 (1H), 8.41 (1H), 8.84 (2H), 10.14 (1H), 12.15 (1H), 13.47 (1H) ppm.

### Example 78

### N-{2-[Benzyl(methyl)amino]ethyl}-N-methyl-4-(1H-pyrazolo[3,4-c]pyridin-5-ylamino)-7H-pyrrolo[2,3-d]pyrimidine-6-carboxamide

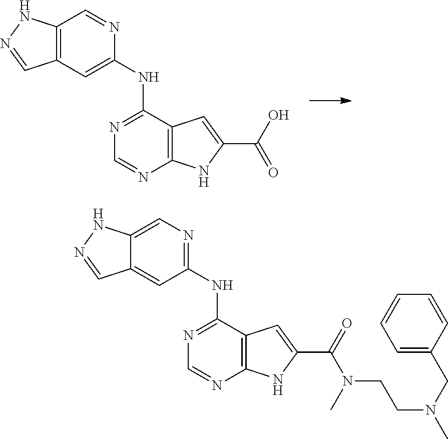

50 mg (169 μmol) 4-(1H-pyrazolo[3,4-c]pyridin-5-ylamino)-7H-pyrrolo[2,3-d]pyrimidine-6-carboxylic acid (prepared according to example 11) were transformed in analogy to example 18 using N-benzyl-N,N′-dimethylethane-1,2-diamine to give after working up and purification 5.0 mg (6%) of the title compound.

¹H-NMR (DMSO-d6): δ=2.17 (3H), 2.60 (2H), 3.30 (3H), 3.52 (2H), 3.68 (2H), 7.16-7.34 (5H), 7.60 (1H), 8.23 (1H), 8.41 (1H), 8.85 (2H), 10.19 (1H), 12.17 (1H), 13.49 (1H) ppm.

### Example 79

### 2-(2-Phenylethyl)-N-(1H-pyrazolo[3,4-c]pyridin-5-yl)[1,3]thiazolo[5,4-d]pyrimidin-7-amine

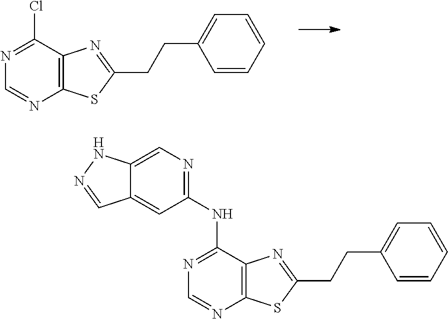 [SMILES: Clc1ncnc2sc(CCc3ccccc3)nc12 ; c1ccc(CCc2nc3c(Nc4cc5cn[nH]c5cn4)ncnc3s2)cc1]

40 mg (145 μmol) 7-chloro-2-(2-phenylethyl)[1,3]thiazolo[5,4-d]pyrimidine (prepared according to intermediate example 79a) were transformed in analogy to intermediate example 46b to give after working up and purification 9.4 mg (16%) of the title compound.

¹H-NMR (DMSO-d6): δ=3.18 (2H), 3.49 (2H), 7.17-7.36 (5H), 8.26 (1H), 8.60 (1H), 8.62 (1H), 8.89 (1H), 9.26 (1H), 13.60 (1H) ppm.

### Example 79a

### 7-Chloro-2-(2-phenylethyl)[1,3]thiazolo[5,4-d]pyrimidine

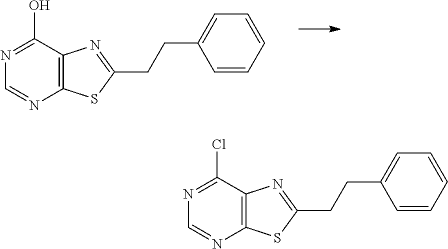 [SMILES: Oc1ncnc2sc(CCc3ccccc3)nc12 ; Clc1ncnc2sc(CCc3ccccc3)nc12]

485 mg (1.89 mmol) 2-(2-Phenylethyl)[1,3]thiazolo[5,4-d]pyrimidin-7-ol (prepared according to intermediate example 79b) were transformed in analogy to intermediate example 7a to give after working up and purification 226 mg (43%) of the title compound.

### Example 79b

### 2-(2-Phenylethyl)[1,3]thiazolo[5,4-d]pyrimidin-7-ol

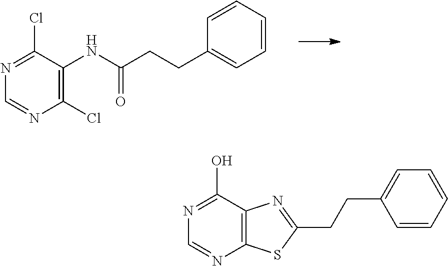 [SMILES: O=C(CCc1ccccc1)Nc1c(Cl)ncnc1Cl ; Oc1ncnc2sc(CCc3ccccc3)nc12]

A mixture comprising 789 mg (2.66 mmol) N-(4,6-dichloropyrimidin-5-yl)-3-phenylpropanamide (prepared according to intermediate example 79c), 7.5 mL ethanol, 203 mg thiourea and 35.7 μL formic acid was heated at 90° C. for 12 hours. The formed precipitate was washed with ethanol and diethylether and purified by chromatography to give 230 mg (34%) of the title compound.

### Example 79c

### N-(4,6-Dichloropyrimidin-5-yl)-3-phenylpropanamide

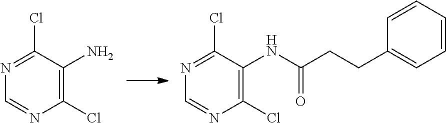 [SMILES: Nc1c(Cl)ncnc1Cl ; O=C(CCc1ccccc1)Nc1c(Cl)ncnc1Cl]

A mixture comprising 1.00 g (6.10 mmol) 4,6-dichloropyrimidin-5-amine (CAS-No. 5413-85-4), 4 mL tetrahydrofurane and 1.82 mL 3-phenylpropanoyl chloride was heated at 70° C. overnight. Dichloromethane and methanol were added the solvents removed and the residue was purified by chromatography to give 794 mg (44%) of the title compound.

### Example 80

### (7S)—N-(Propan-2-yl)-4-(1H-pyrazolo[3,4-c]pyridin-5-ylamino)-5,6,7,8-tetrahydro[1]benzothieno[2,3-d]pyrimidine-7-carboxamide

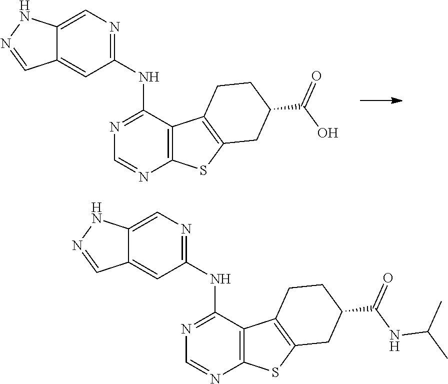

100 mg (273 μmol) (7S)-4-(1H-pyrazolo[3,4-c]pyridin-5-ylamino)-5,6,7,8-tetrahydro[1]benzothieno[2,3-d]pyrimidine-7-carboxylic acid (prepared according to intermediate example 46a) were transformed in analogy to example 18 using propan-2-amine to give after working up and purification 27.7 mg (24%) of the title compound.

¹H-NMR (DMSO-d6): δ=1.07 (6H), 1.83 (1H), 2.11 (1H), 2.61 (1H), 2.93 (2H), 3.15 (1H), 3.25 (1H), 3.87 (1H), 7.84 (1H), 8.24 (1H), 8.39 (1H), 8.53 (1H), 8.66 (1H), 8.85 (1H), 13.55 (1H) ppm.

### Example 81

### (7S)—N-Methyl-N-propyl-4-(1H-pyrazolo[3,4-c]pyridin-5-ylamino)-5,6,7,8-tetrahydro[1]benzothieno[2,3-d]pyrimidine-7-carboxamide

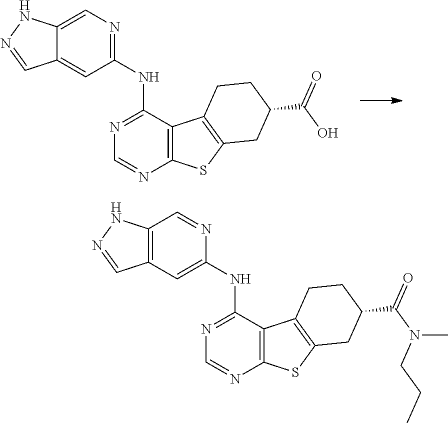 [SMILES: O=C(O)[C@H]1CCc2c(sc3ncnc(Nc4cc5cn[nH]c5cn4)c23)C1 ; CCCN(C)C(=O)[C@H]1CCc2c(sc3ncnc(Nc4cc5cn[nH]c5cn4)c23)C1]

100 mg (273 μmol) (7S)-4-(1H-pyrazolo[3,4-c]pyridin-5-ylamino)-5,6,7,8-tetrahydro[1]benzothieno[2,3-d]pyrimidine-7-carboxylic acid (prepared according to intermediate example 46a) were transformed in analogy to example 18 using N-methylpropan-1-amine to give after working up and purification 32.4 mg (27%) of the title compound.

¹H-NMR (DMSO-d6): δ=0.83+0.87 (3H), 1.44-1.62 (2H), 1.81 (1H), 2.10 (1H), 2.85+3.07 (3H), 2.86-3.01 (2H), 3.09-3.41 (5H), 8.24 (1H), 8.34 (1H), 8.53 (1H), 8.68 (1H), 8.83 (1H), 13.56 (1H) ppm.

### Example 82

### 1-{[(7S)-4-(1H-Pyrazolo[3,4-c]pyridin-5-ylamino)-5,6,7,8-tetrahydro[1]benzothieno[2,3-d]pyrimidin-7-yl]carbonyl}azetidine-3-carbonitrile

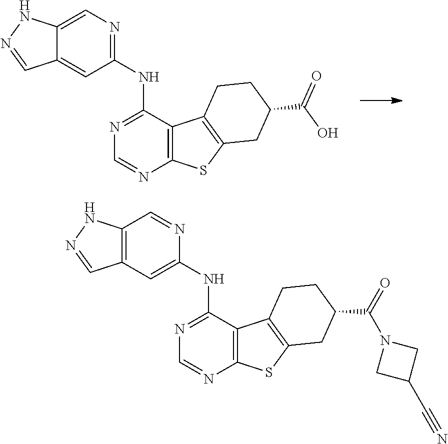

70 mg (191 μmol) (7S)-4-(1H-pyrazolo[3,4-c]pyridin-5-ylamino)-5,6,7,8-tetrahydro[1]benzothieno[2,3-d]pyrimidine-7-carboxylic acid (prepared according to intermediate example 46a) were transformed in analogy to example 18 using azetidine-3-carbonitrile to give after working up and purification 70.2 mg (81%) of the title compound.

¹H-NMR (DMSO-d6): δ=1.77 (1H), 2.12 (1H), 2.68-3.02 (3H), 3.12-3.42 (2H), 3.81 (1H), 4.05 (1H), 4.18 (1H), 4.51 (2H), 8.24 (1H), 8.36 (1H), 8.53 (1H), 8.67 (1H), 8.84 (1H), 13.58 (1H) ppm.

### Example 83

### 2-Oxa-6-azaspiro[3.3]hept-6-yl[(7S)-4-(1H-pyrazolo[3,4-c]pyridin-5-ylamino)-5,6,7,8-tetrahydro[1]benzothieno[2,3-d]pyrimidin-7-yl]methanone

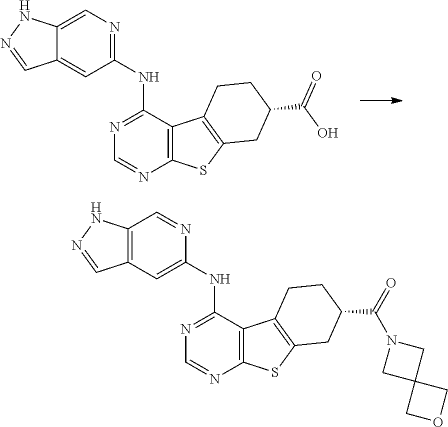

75 mg (205 μmol) (7S)-4-(1H-pyrazolo[3,4-c]pyridin-5-ylamino)-5,6,7,8-tetrahydro[1]benzothieno[2,3-d]pyrimidine-7-carboxylic acid (prepared according to intermediate example 46a) were transformed in analogy to example 18 using 2-oxa-6-azaspiro[3.3]heptane to give after working up and purification 4.3 mg (4%) of the title compound.

¹H-NMR (DMSO-d6): δ=1.76 (1H), 2.10 (1H), 2.71 (1H), 2.82-2.95 (2H), 3.15-3.29 (2H), 4.05 (2H), 4.41 (2H), 4.68 (4H), 8.24 (1H), 8.36 (1H), 8.53 (1H), 8.67 (1H), 8.84 (1H), 13.53 (1H) ppm.

### Example 84

### [(3R)-3-(Dimethylamino)pyrrolidin-1-yl][(7S)-4-(1H-pyrazolo[3,4-c]pyridin-5-ylamino)-5,6,7,8-tetrahydro[1]benzothieno[2,3-d]pyrimidin-7-yl]methanone

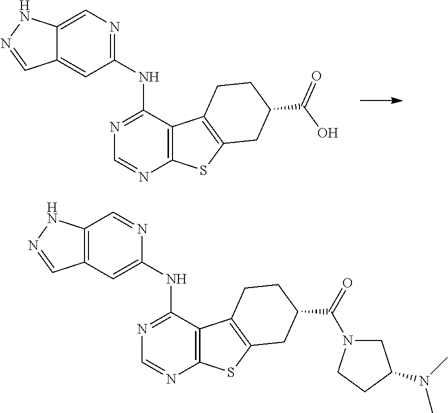

100 mg (273 μmol) (7S)-4-(1H-pyrazolo[3,4-c]pyridin-5-ylamino)-5,6,7,8-tetrahydro[1]benzothieno[2,3-d]pyrimidine-7-carboxylic acid (prepared according to intermediate example 46a) were transformed in analogy to example 18 using (3R)—N,N-dimethylpyrrolidin-3-amine to give after working up and purification 95.1 mg (72%) of the title compound.

¹H-NMR (DMSO-d6): δ=1.60-1.84 (2H), 1.99-2.20 (9H), 2.59-2.74 (1H), 2.92-3.05 (3H), 3.17-3.65 (4H), 3.77+3.86 (1H), 8.24 (1H), 8.34 (1H), 8.53 (1H), 8.69 (1H), 8.84 (1H), 13.49 (1H) ppm.

### Example 85

### [(3S)-3-(Dimethylamino)pyrrolidin-1-yl][(7S)-4-(1H-pyrazolo[3,4-c]pyridin-5-ylamino)-5,6,7,8-tetrahydro[1]benzothieno[2,3-d]pyrimidin-7-yl]methanone

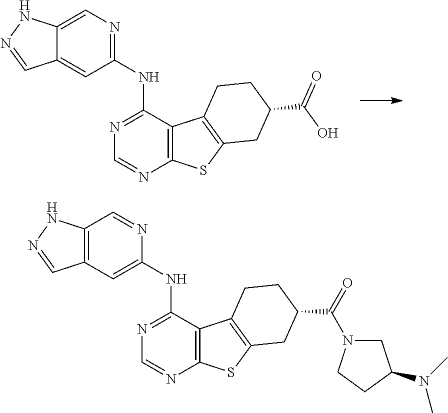

100 mg (273 μmol) (7S)-4-(1H-pyrazolo[3,4-c]pyridin-5-ylamino)-5,6,7,8-tetrahydro[1]benzothieno[2,3-d]pyrimidine-7-carboxylic acid (prepared according to intermediate example 46a) were transformed in analogy to example 18 using (3S)—N,N-dimethylpyrrolidin-3-amine to give after working up and purification 103 mg (78%) of the title compound.

¹H-NMR (DMSO-d6): δ=1.58-1.83 (2H), 1.98-2.21 (9H), 2.61+2.72 (1H), 2.89-3.04 (3H), 3.18-3.46 (4H), 3.73+3.81 (1H), 8.24 (1H), 8.34 (1H), 8.54 (1H), 8.69 (1H), 8.83 (1H), 13.48 (1H) ppm.

### Example 86

### Morpholin-4-yl [(7S)-4-(1H-pyrazolo[3,4-c]pyridin-5-ylamino)-5,6,7,8-tetrahydro[1]benzothieno[2,3-d]pyrimidin-7-yl]methanone

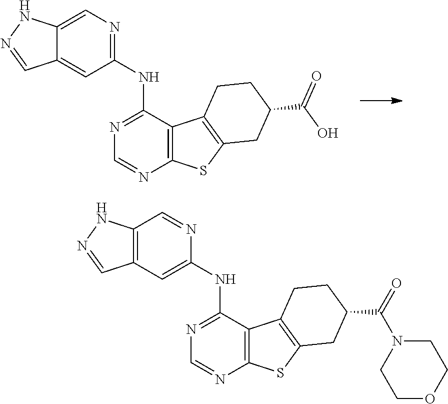

100 mg (273 μmol) (7S)-4-(1H-pyrazolo[3,4-c]pyridin-5-ylamino)-5,6,7,8-tetrahydro[1]benzothieno[2,3-d]pyrimidine-7-carboxylic acid (prepared according to intermediate example 46a) were transformed in analogy to example 18 using morpholine to give after working up and purification 67.8 mg (54%) of the title compound.

¹H-NMR (DMSO-d6): δ=1.81 (1H), 2.11 (1H), 2.87-3.04 (2H), 3.10-3.41 (3H), 3.45-3.66 (8H), 8.24 (1H), 8.34 (1H), 8.54 (1H), 8.69 (1H), 8.83 (1H), 13.55 (1H) ppm.

### Example 87

### [(3S)-3-Methylmorpholin-4-yl][(7S)-4-(1H-pyrazolo[3,4-c]pyridin-5-ylamino)-5,6,7,8-tetrahydro[1]benzothieno[2,3-d]pyrimidin-7-yl]methanone

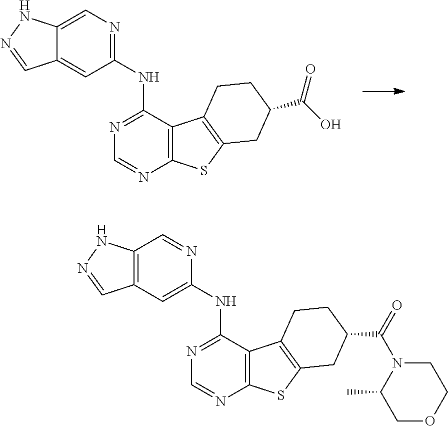 [SMILES: O=C(O)[C@H]1CCc2c(sc3ncnc(Nc4cc5cn[nH]c5cn4)c23)C1 ; C[C@H]1COCCN1C(=O)[C@H]1CCc2c(sc3ncnc(Nc4cc5cn[nH]c5cn4)c23)C1]

100 mg (273 μmol) (7S)-4-(1H-pyrazolo[3,4-c]pyridin-5-ylamino)-5,6,7,8-tetrahydro[1]benzothieno[2,3-d]pyrimidine-7-carboxylic acid (prepared according to intermediate example 46a) were transformed in analogy to example 18 using (3S)-3-methylmorpholine to give after working up and purification 24.1 mg (19%) of the title compound.

¹H-NMR (DMSO-d6): δ=1.16+1.29 (3H), 1.79 (1H), 2.10 (1H), 2.82-4.46 (12H), 8.24 (1H), 8.32 (1H), 8.53 (1H), 8.68 (1H), 8.83 (1H), 13.58 (1H) ppm.

### Example 88

### [(3R)-3-Methylmorpholin-4-yl][(7S)-4-(1H-pyrazolo[3,4-c]pyridin-5-ylamino)-5,6,7,8-tetrahydro[1]benzothieno[2,3-d]pyrimidin-7-yl]methanone

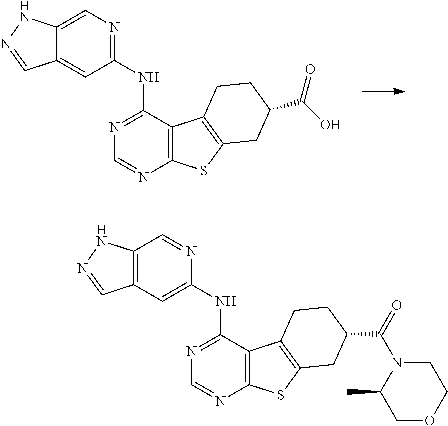 [SMILES: O=C(O)[C@H]1CCc2c(sc3ncnc(Nc4cc5cn[nH]c5cn4)c23)C1 ; C[C@@H]1COCCN1C(=O)[C@H]1CCc2c(sc3ncnc(Nc4cc5cn[nH]c5cn4)c23)C1]

100 mg (273 μmol) (7S)-4-(1H-pyrazolo[3,4-c]pyridin-5-ylamino)-5,6,7,8-tetrahydro[1]benzothieno[2,3-d]pyrimidine-7-carboxylic acid (prepared according to intermediate example 46a) were transformed in analogy to example 18 using (3R)-3-methylmorpholine to give after working up and purification 27.4 mg (21%) of the title compound.

¹H-NMR (DMSO-d6): δ=1.15+1.31 (3H), 1.87 (1H), 2.10 (1H), 2.86-4.45 (12H), 8.24 (1H), 8.34 (1H), 8.54 (1H), 8.69 (1H), 8.83 (1H), 13.53 (1H) ppm.

### Example 89

### [(2R,6S)-2,6-Dimethylmorpholin-4-yl][(7S)-4-(1H-pyrazolo[3,4-c]pyridin-5-ylamino)-5,6,7,8-tetrahydro[1]benzothieno[2,3-d]pyrimidin-7-yl]methanone

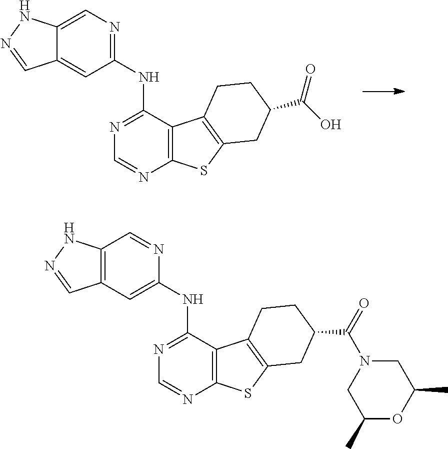 [SMILES: O=C(O)[C@H]1CCc2c(sc3ncnc(Nc4cc5cn[nH]c5cn4)c23)C1 ; C[C@@H]1CN(C(=O)[C@H]2CCc3c(sc4ncnc(Nc5cc6cn[nH]c6cn5)c34)C2)C[C@H](C)O1]

100 mg (273 μmol) (7S)-4-(1H-pyrazolo[3,4-c]pyridin-5-ylamino)-5,6,7,8-tetrahydro[1]benzothieno[2,3-d]pyrimidine-7-carboxylic acid (prepared according to intermediate example 46a) were transformed in analogy to example 18 using (2R,6S)-2,6-dimethylmorpholine to give after working up and purification 13.1 mg (10%) of the title compound.

¹H-NMR (DMSO-d6): δ=1.10 (6H), 1.81 (1H), 2.10 (1H), 2.26 (1H), 2.76 (1H), 2.87-3.03 (2H), 3.11-3.60 (5H), 3.95 (1H), 4.31 (1H), 8.24 (1H), 8.34 (1H), 8.54 (1H), 8.69 (1H), 8.84 (1H), 13.54 (1H) ppm.

### Example 90

### (4-Methylpiperazin-1-yl)[(7S)-4-(1H-pyrazolo[3,4-c]pyridin-5-ylamino)-5,6,7,8-tetrahydro[1]benzothieno[2,3-d]pyrimidin-7-yl]methanone

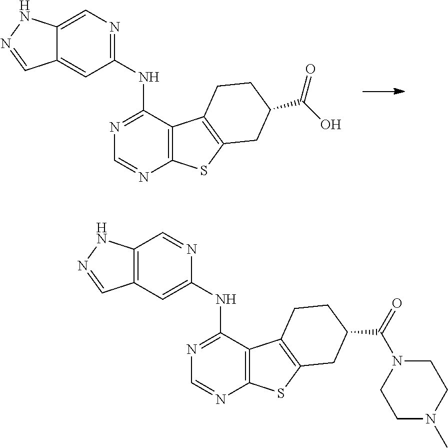 [SMILES: O=C(O)[C@H]1CCc2c(sc3ncnc(Nc4cc5cn[nH]c5cn4)c23)C1 ; CN1CCN(C(=O)[C@H]2CCc3c(sc4ncnc(Nc5cc6cn[nH]c6cn5)c34)C2)CC1]

100 mg (273 μmol) (7S)-4-(1H-pyrazolo[3,4-c]pyridin-5-ylamino)-5,6,7,8-tetrahydro[1]benzothieno[2,3-d]pyrimidine-7-carboxylic acid (prepared according to intermediate example 46a) were transformed in analogy to example 18 using 1-methylpiperazine to give after working up and purification 66.5 mg (52%) of the title compound.

¹H-NMR (DMSO-d6): δ=1.80 (1H), 2.10 (1H), 2.19 (3H), 2.27 (2H), 2.34 (2H), 2.82-3.03 (2H), 3.10-3.40 (3H), 3.50 (2H), 3.56 (2H), 8.24 (1H), 8.33 (1H), 8.53 (1H), 8.68 (1H), 8.84 (1H), 13.55 (1H) ppm.

### Example 91

### [4-(Dimethylamino)piperidin-1-yl][(7S)-4-(1H-pyrazolo[3,4-c]pyridin-5-ylamino)-5,6,7,8-tetrahydro[1]benzothieno[2,3-d]pyrimidin-7-yl]methanone

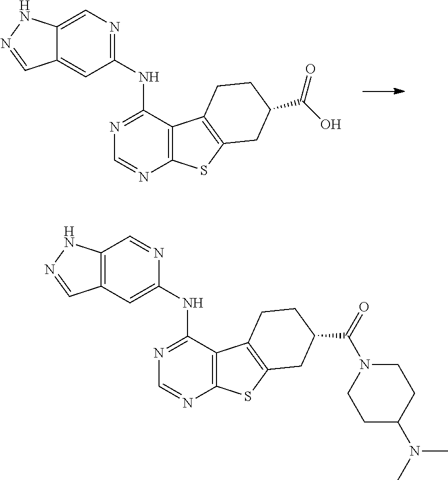 [SMILES: O=C(O)[C@H]1CCc2c(sc3ncnc(Nc4cc5cn[nH]c5cn4)c23)C1 ; CN(C)C1CCN(C(=O)[C@H]2CCc3c(sc4ncnc(Nc5cc6cn[nH]c6cn5)c34)C2)CC1]

100 mg (273 μmol) (7S)-4-(1H-pyrazolo[3,4-c]pyridin-5-ylamino)-5,6,7,8-tetrahydro[1]benzothieno[2,3-d]pyrimidine-7-carboxylic acid (prepared according to intermediate example 46a) were transformed in analogy to example 18 using N,N-dimethylpiperidin-4-amine to give after working up and purification 39.5 mg (29%) of the title compound.

¹H-NMR (DMSO-d6): δ=1.20 (1H), 1.35 (1H), 1.71-1.87 (3H), 2.10 (1H), 2.17 (6H), 2.32 (1H), 2.61 (1H), 2.84-3.37 (6H), 4.03 (1H), 4.40 (1H), 8.24 (1H), 8.33 (1H), 8.53 (1H), 8.69 (1H), 8.84 (1H), 13.56 (1H) ppm.

### Example 92

### (7S)—N-Ethyl-N-methyl-4-(1H-pyrazolo[3,4-b]pyridin-5-ylamino)-5,6,7,8-tetrahydro[1]benzothieno[2,3-d]pyrimidine-7-carboxamide

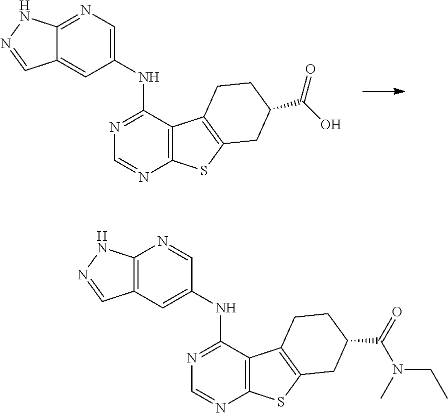 [SMILES: O=C(O)[C@H]1CCc2c(sc3ncnc(Nc4cnc5[nH]ncc5c4)c23)C1 ; CCN(C)C(=O)[C@H]1CCc2c(sc3ncnc(Nc4cnc5[nH]ncc5c4)c23)C1]

80 mg (218 μmol) (7S)-4-(1H-pyrazolo[3,4-b]pyridin-5-ylamino)-5,6,7,8-tetrahydro[1]benzothieno[2,3-d]pyrimidine-7-carboxylic acid (prepared according to intermediate example 59a) were transformed in analogy to example 18 using N-methylethanamine to give after working up and purification 69.3 mg (89%) of the title compound.

¹H-NMR (DMSO-d6): δ=1.03+1.15 (3H), 1.81 (1H), 2.05 (1H), 2.84+3.06 (3H), 2.86-3.02 (2H), 3.08-3.39 (4H), 3.44 (1H), 8.13 (1H), 8.30 (1H), 8.34 (1H), 8.36 (1H), 8.61 (1H), 13.59 (1H) ppm.

### Example 93

### (7S)—N-Methyl-N-propyl-4-(1H-pyrazolo[3,4-b]pyridin-5-ylamino)-5,6,7,8-tetrahydro[1]benzothieno[2,3-d]pyrimidine-7-carboxamide

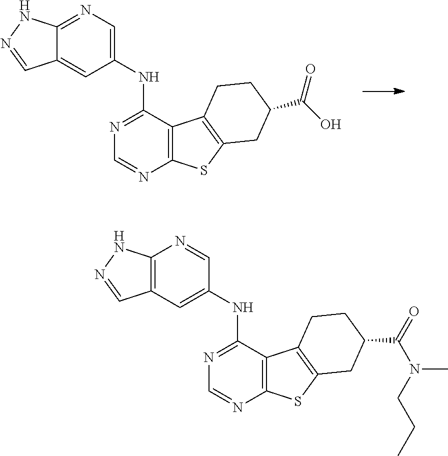 [SMILES: O=C(O)[C@H]1CCc2c(sc3ncnc(Nc4cnc5[nH]ncc5c4)c23)C1 ; CCCN(C)C(=O)[C@H]1CCc2c(sc3ncnc(Nc4cnc5[nH]ncc5c4)c23)C1]

80 mg (218 μmol) (7S)-4-(1H-pyrazolo[3,4-b]pyridin-5-ylamino)-5,6,7,8-tetrahydro[1]benzothieno[2,3-d]pyrimidine-7-carboxylic acid (prepared according to intermediate example 59a) were transformed in analogy to example 18 using N-methylpropan-1-amine to give after working up and purification 71.6 mg (74%) of the title compound.

¹H-NMR (DMSO-d6): δ=0.83+0.87 (3H), 1.44-1.62 (2H), 1.81 (1H), 2.05 (1H), 2.85+3.07 (3H), 2.87-3.02 (2H), 3.08-3.40 (5H), 8.13 (1H), 8.30 (1H), 8.34 (1H), 8.36 (1H), 8.61 (1H), 13.60 (1H) ppm.

### Example 94

### (7S)—N-Methyl-4-(1H-pyrazolo[3,4-b]pyridin-5-ylamino)-N-(3,3,3-trifluoropropyl)-5,6,7,8-tetrahydro[1]benzothieno[2,3-d]pyrimidine-7-carboxamide

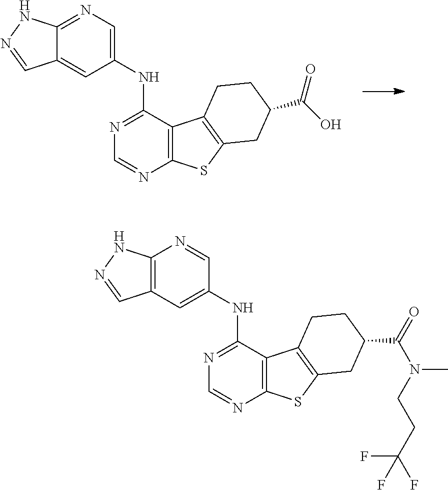 [SMILES: O=C(O)[C@H]1CCc2c(sc3ncnc(Nc4cnc5[nH]ncc5c4)c23)C1 ; CN(CCC(F)(F)F)C(=O)[C@H]1CCc2c(sc3ncnc(Nc4cnc5[nH]ncc5c4)c23)C1]

70 mg (191 μmol) (7S)-4-(1H-pyrazolo[3,4-b]pyridin-5-ylamino)-5,6,7,8-tetrahydro[1]benzothieno[2,3-d]pyrimidine-7-carboxylic acid (prepared according to intermediate example 59a) were transformed in analogy to example 18 using 3,3,3-trifluoro-N-methylpropan-1-amine to give after working up and purification 57.1 mg (91%) of the title compound.

¹H-NMR (DMSO-d6): δ=1.79 (1H), 2.06 (1H), 2.43-2.60 (2H), 2.87+3.12 (3H), 2.93 (2H), 3.13-3.70 (5H), 8.13 (1H), 8.24-8.47 (3H), 8.60 (1H), 13.59 (1H) ppm.

### Example 95

### (7S)—N-Methyl-N-(propan-2-yl)-4-(1H-pyrazolo[3,4-b]pyridin-5-ylamino)-5,6,7,8-tetrahydro[1]benzothieno[2,3-d]pyrimidine-7-carboxamide

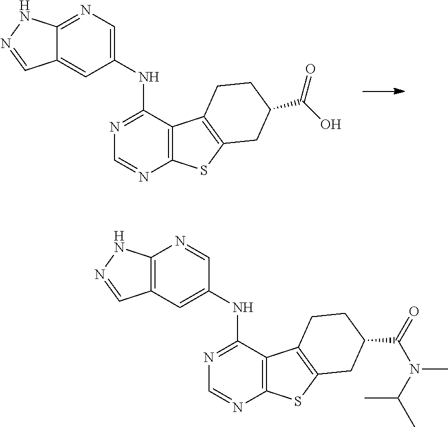 [SMILES: O=C(O)[C@H]1CCc2c(sc3ncnc(Nc4cnc5[nH]ncc5c4)c23)C1 ; CC(C)N(C)C(=O)[C@H]1CCc2c(sc3ncnc(Nc4cnc5[nH]ncc5c4)c23)C1]

80 mg (218 μmol) (7S)-4-(1H-pyrazolo[3,4-b]pyridin-5-ylamino)-5,6,7,8-tetrahydro[1]benzothieno[2,3-d]pyrimidine-7-carboxylic acid (prepared according to intermediate example 59a) were transformed in analogy to example 18 using N-methylpropan-2-amine to give after working up and purification 55.6 mg (57%) of the title compound.

¹H-NMR (DMSO-d6): δ=1.05+1.18 (6H), 1.81 (1H), 2.04 (1H), 2.71+2.90 (3H), 2.84-3.35 (5H), 4.26+4.72 (1H), 8.13 (1H), 8.24-8.45 (1H), 8.30 (1H), 8.36 (1H), 8.61 (1H), 13.60 (1H) ppm.

### Example 96

### (7S)—N-(2-Methoxyethyl)-N-methyl-4-(1H-pyrazolo[3,4-b]pyridin-5-ylamino)-5,6,7,8-tetrahydro[1]benzothieno[2,3-d]pyrimidine-7-carboxamide

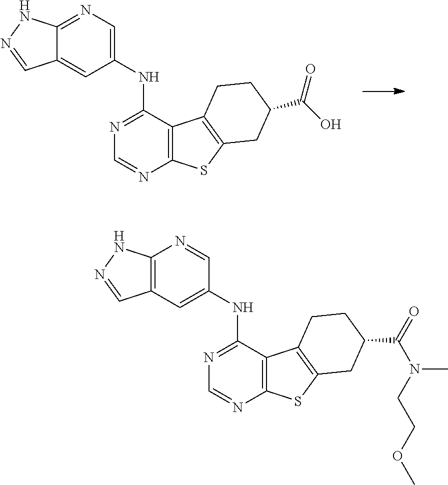 [SMILES: O=C(O)[C@H]1CCc2c(sc3ncnc(Nc4cnc5[nH]ncc5c4)c23)C1 ; COCCN(C)C(=O)[C@H]1CCc2c(sc3ncnc(Nc4cnc5[nH]ncc5c4)c23)C1]

300 mg (819 μmol) (7S)-4-(1H-pyrazolo[3,4-b]pyridin-5-ylamino)-5,6,7,8-tetrahydro[1]benzothieno[2,3-d]pyrimidine-7-carboxylic acid (prepared according to intermediate example 59a) were transformed in analogy to example 18 using 2-methoxy-N-methylethanamine to give after working up and purification 241 mg (64%) of the title compound.

¹H-NMR (DMSO-d6): δ=1.80 (1H), 2.06 (1H), 2.87+3.12 (3H), 2.93 (2H), 3.14-3.34 (3H), 3.25+3.28 (3H), 3.41-3.53 (3H), 3.59 (1H), 8.13 (1H), 8.22-8.42 (1H), 8.30 (1H), 8.36 (1H), 8.61 (1H), 13.60 (1H) ppm.

### Example 97

### Azetidin-1-yl[(7S)-4-(1H-pyrazolo[3,4-b]pyridin-5-ylamino)-5,6,7,8-tetrahydro[1]benzothieno[2,3-d]pyrimidin-7-yl]methanone

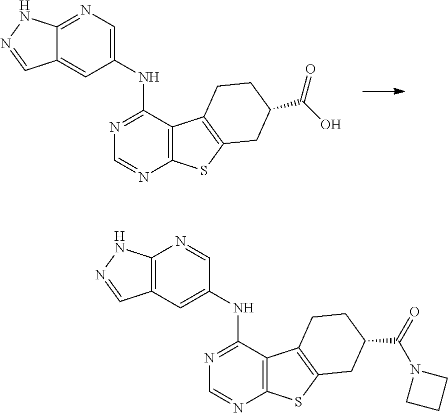 [SMILES: O=C(O)[C@H]1CCc2c(sc3ncnc(Nc4cnc5[nH]ncc5c4)c23)C1 ; O=C([C@H]1CCc2c(sc3ncnc(Nc4cnc5[nH]ncc5c4)c23)C1)N1CCC1]

80 mg (218 μmol) (7S)-4-(1H-pyrazolo[3,4-b]pyridin-5-ylamino)-5,6,7,8-tetrahydro[1]benzothieno[2,3-d]pyrimidine-7-carboxylic acid (prepared according to intermediate example 59a) were transformed in analogy to example 18 using azetidine to give after working up and purification 47.2 mg (51%) of the title compound.

¹H-NMR (DMSO-d6): δ=1.77 (1H), 2.06 (1H), 2.22 (2H), 2.74 (1H), 2.90 (2H), 3.17 (1H), 3.32 (1H), 3.88 (2H), 4.17-4.32 (2H), 8.13 (1H), 8.30 (1H), 8.34 (1H), 8.36 (1H), 8.61 (1H), 13.60 (1H) ppm.

### Example 98

### 2-Oxa-6-azaspiro[3.3]hept-6-yl[(7S)-4-(1H-pyrazolo[3,4-b]pyridin-5-ylamino)-5,6,7,8-tetrahydro[1]benzothieno[2,3-d]pyrimidin-7-yl]methanone

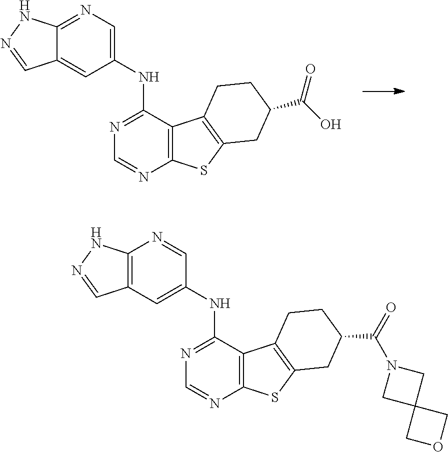 [SMILES: O=C(O)[C@H]1CCc2c(sc3ncnc(Nc4cnc5[nH]ncc5c4)c23)C1 ; O=C([C@H]1CCc2c(sc3ncnc(Nc4cnc5[nH]ncc5c4)c23)C1)N1CC2(COC2)C1]

75 mg (205 μmol) (7S)-4-(1H-pyrazolo[3,4-b]pyridin-5-ylamino)-5,6,7,8-tetrahydro[1]benzothieno[2,3-d]pyrimidine-7-carboxylic acid (prepared according to intermediate example 59a) were transformed in analogy to example 18 using 2-oxa-6-azaspiro[3.3]heptane to give after working up and purification 17.2 mg (18%) of the title compound.

¹H-NMR (DMSO-d6): δ=1.75 (1H), 2.05 (1H), 2.73 (1H), 2.89 (2H), 3.15 (1H), 3.29 (1H), 4.05 (2H), 4.38 (1H), 4.44 (1H), 4.67 (4H), 8.13 (1H), 8.30 (1H), 8.33 (1H), 8.36 (1H), 8.60 (1H), 13.59 (1H) ppm.

### Example 99

### 1-{[(7S)-4-(1H-Pyrazolo[3,4-b]pyridin-5-ylamino)-5,6,7,8-tetrahydro[1]benzothieno[2,3-d]pyrimidin-7-yl]carbonyl}azetidine-3-carbonitrile

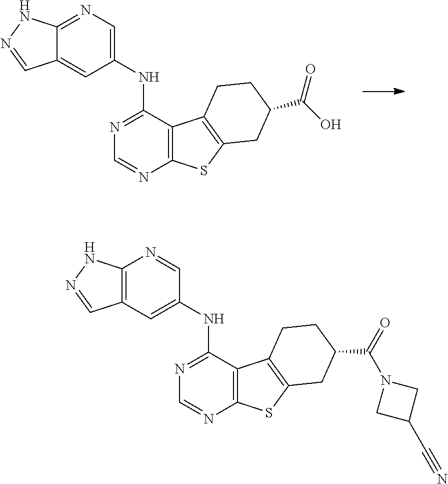 [SMILES: O=C(O)[C@H]1CCc2c(sc3ncnc(Nc4cnc5[nH]ncc5c4)c23)C1 ; N#CC1CN(C(=O)[C@H]2CCc3c(sc4ncnc(Nc5cnc6[nH]ncc6c5)c34)C2)C1]

70 mg (191 μmol) (7S)-4-(1H-pyrazolo[3,4-b]pyridin-5-ylamino)-5,6,7,8-tetrahydro[1]benzothieno[2,3-d]pyrimidine-7-carboxylic acid (prepared according to intermediate example 59a) were transformed in analogy to example 18 using azetidine-3-carbonitrile to give after working up and purification 45.80 mg (53%) of the title compound.

¹H-NMR (DMSO-d6): δ=1.76 (1H), 2.08 (1H), 2.70-2.99 (3H), 3.15 (1H), 3.28 (1H), 3.81 (1H), 4.04 (1H), 4.18 (1H), 4.41-4.61 (2H), 8.13 (1H), 8.30 (1H), 8.36 (2H), 8.60 (1H), 13.61 (1H) ppm.

### Example 100

### [(3S)-3-(Dimethylamino)pyrrolidin-1-yl][(7S)-4-(1H-pyrazolo[3,4-b]pyridin-5-ylamino)-5,6,7,8-tetrahydro[1]benzothieno[2,3-d]pyrimidin-7-yl]methanone

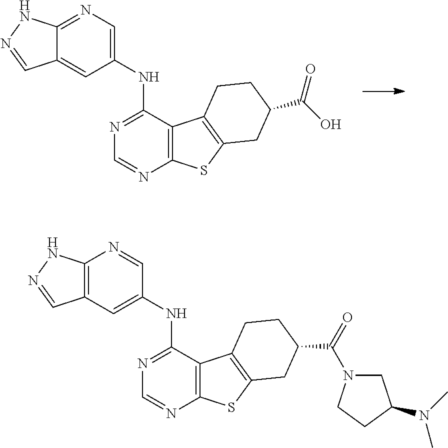 [SMILES: O=C(O)[C@H]1CCc2c(sc3ncnc(Nc4cnc5[nH]ncc5c4)c23)C1 ; CN(C)[C@H]1CCN(C(=O)[C@H]2CCc3c(sc4ncnc(Nc5cnc6[nH]ncc6c5)c34)C2)C1]

70 mg (191 μmol) (7S)-4-(1H-pyrazolo[3,4-b]pyridin-5-ylamino)-5,6,7,8-tetrahydro[1]benzothieno[2,3-d]pyrimidine-7-carboxylic acid (prepared according to intermediate example 59a) were transformed in analogy to example 18 using (3S)—N,N-dimethylpyrrolidin-3-amine to give after working up and purification 60.2 mg (65%) of the title compound.

¹H-NMR (DMSO-d6): δ=1.55-1.88 (2H), 1.96-2.13 (2H), 2.16 (6H), 2.54-2.76 (1H), 2.86-3.04 (4H), 3.11-3.28 (2H), 3.51-3.82 (3H), 8.13 (1H), 8.30 (1H), 8.36 (2H), 8.60 (1H), 13.62 (1H) ppm.

### Example 101

### [(3R)-3-(Dimethylamino)pyrrolidin-1-yl][(7S)-4-(1H-pyrazolo[3,4-b]pyridin-5-ylamino)-5,6,7,8-tetrahydro[1]benzothieno[2,3-d]pyrimidin-7-yl]methanone

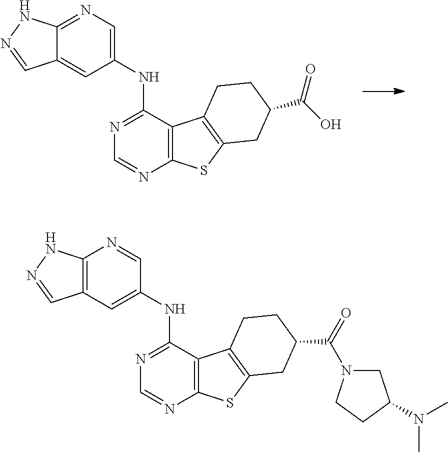 [SMILES: O=C(O)[C@H]1CCc2c(sc3ncnc(Nc4cnc5[nH]ncc5c4)c23)C1 ; CN(C)[C@@H]1CCN(C(=O)[C@H]2CCc3c(sc4ncnc(Nc5cnc6[nH]ncc6c5)c34)C2)C1]

75 mg (205 μmol) (7S)-4-(1H-pyrazolo[3,4-b]pyridin-5-ylamino)-5,6,7,8-tetrahydro[1]benzothieno[2,3-d]pyrimidine-7-carboxylic acid (prepared according to intermediate example 59a) were transformed in analogy to example 18 using (3R)—N,N-dimethylpyrrolidin-3-amine to give after working up and purification 28.7 mg (29%) of the title compound.

¹H-NMR (DMSO-d6): δ=1.59-1.84 (2H), 1.97-2.14 (2H), 2.16 (6H), 2.59-2.75 (1H), 2.91-3.06 (3H), 3.12-3.64 (5H), 3.79+3.89 (1H), 8.13 (1H), 8.29 (1H), 8.33 (1H), 8.36 (1H), 8.60 (1H), 13.60 (1H) ppm.

### Example 102

### Morpholin-4-yl[(7S)-4-(1H-pyrazolo[3,4-b]pyridin-5-ylamino)-5,6,7,8-tetrahydro[1]benzothieno[2,3-d]pyrimidin-7-yl]methanone

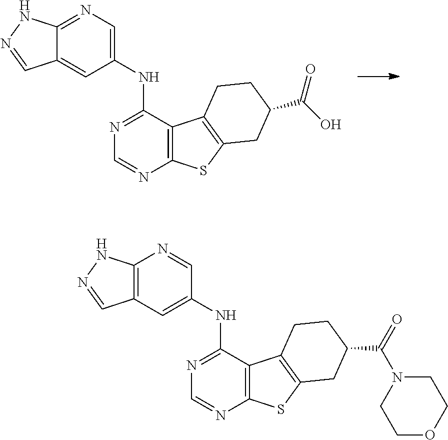 [SMILES: O=C(O)[C@H]1CCc2c(sc3ncnc(Nc4cnc5[nH]ncc5c4)c23)C1 ; O=C([C@H]1CCc2c(sc3ncnc(Nc4cnc5[nH]ncc5c4)c23)C1)N1CCOCC1]

80 mg (218 μmol) (7S)-4-(1H-pyrazolo[3,4-b]pyridin-5-ylamino)-5,6,7,8-tetrahydro[1]benzothieno[2,3-d]pyrimidine-7-carboxylic acid (prepared according to intermediate example 59a) were transformed in analogy to example 18 using morpholine to give after working up and purification 69.3 mg (69%) of the title compound.

¹H-NMR (DMSO-d6): δ=1.82 (1H), 2.06 (1H), 2.87-3.03 (2H), 3.19 (2H), 3.32 (1H), 3.44-3.66 (8H), 8.13 (1H), 8.30 (1H), 8.34 (1H), 8.36 (1H), 8.61 (1H), 13.60 (1H) ppm.

### Example 103

### [(3R)-3-Methylmorpholin-4-yl][(7S)-4-(1H-pyrazolo[3,4-b]pyridin-5-ylamino)-5,6,7,8-tetrahydro[1]benzothieno[2,3-d]pyrimidin-7-yl]methanone

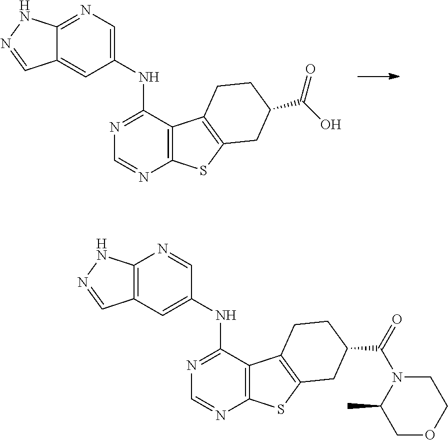 [SMILES: O=C(O)[C@H]1CCc2c(sc3ncnc(Nc4cnc5[nH]ncc5c4)c23)C1 ; C[C@@H]1COCCN1C(=O)[C@H]1CCc2c(sc3ncnc(Nc4cnc5[nH]ncc5c4)c23)C1]

80 mg (218 μmol) (7S)-4-(1H-pyrazolo[3,4-b]pyridin-5-ylamino)-5,6,7,8-tetrahydro[1]benzothieno[2,3-d]pyrimidine-7-carboxylic acid (prepared according to intermediate example 59a) were transformed in analogy to example 18 using (3R)-3-methylmorpholine to give after working up and purification 64.2 mg (62%) of the title compound.

¹H-NMR (DMSO-d6): δ=1.16+1.31 (3H), 1.87 (1H), 2.03 (1H), 2.84-4.47 (12H), 8.13 (1H), 8.30 (1H), 8.33 (1H), 8.36 (1H), 8.60 (1H), 13.60 (1H) ppm.

### Example 104

### [(3S)-3-Methylmorpholin-4-yl][(7S)-4-(1H-pyrazolo[3,4-b]pyridin-5-ylamino)-5,6,7,8-tetrahydro[1]benzothieno[2,3-d]pyrimidin-7-yl]methanone

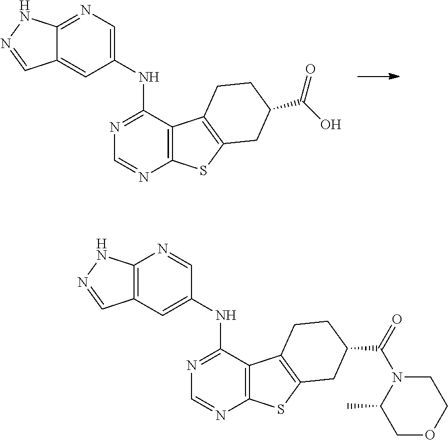 [SMILES: O=C(O)[C@H]1CCc2c(sc3ncnc(Nc4cnc5[nH]ncc5c4)c23)C1 ; C[C@H]1COCCN1C(=O)[C@H]1CCc2c(sc3ncnc(Nc4cnc5[nH]ncc5c4)c23)C1]

80 mg (218 μmol) (7S)-4-(1H-pyrazolo[3,4-b]pyridin-5-ylamino)-5,6,7,8-tetrahydro[1]benzothieno[2,3-d]pyrimidine-7-carboxylic acid (prepared according to intermediate example 59a) were transformed in analogy to example 18 using (3S)-3-methylmorpholine to give after working up and purification 75.3 mg (73%) of the title compound.

¹H-NMR (DMSO-d6): δ=1.15+1.29 (3H), 1.79 (1H), 2.03 (1H), 2.84-4.47 (12H), 8.13 (1H), 8.30 (1H), 8.33 (1H), 8.36 (1H), 8.60 (1H), 13.61 (1H) ppm.

### Example 105

### [(2R,6S)-2,6-Dimethylmorpholin-4-yl][(7S)-4-(1H-pyrazolo[3,4-b]pyridin-5-ylamino)-5,6,7,8-tetrahydro[1]benzothieno[2,3-d]pyrimidin-7-yl]methanone

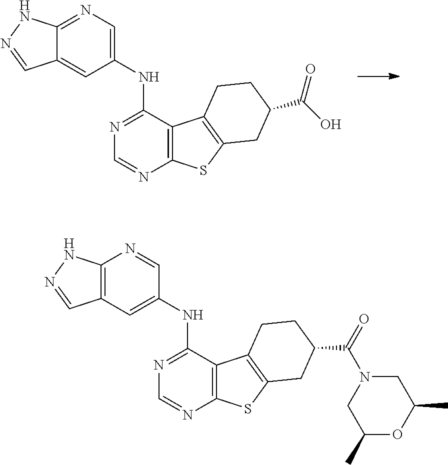 [SMILES: O=C(O)[C@H]1CCc2c(sc3ncnc(Nc4cnc5[nH]ncc5c4)c23)C1 ; C[C@@H]1CN(C(=O)[C@H]2CCc3c(sc4ncnc(Nc5cnc6[nH]ncc6c5)c34)C2)C[C@H](C)O1]

80 mg (218 μmol) (7S)-4-(1H-pyrazolo[3,4-b]pyridin-5-ylamino)-5,6,7,8-tetrahydro[1]benzothieno[2,3-d]pyrimidine-7-carboxylic acid (prepared according to intermediate example 59a) were transformed in analogy to example 18 using (2R,6S)-2,6-dimethylmorpholine to give after working up and purification 74.0 mg (69%) of the title compound.

¹H-NMR (DMSO-d6): δ=1.10 (6H), 1.81 (1H), 2.05 (1H), 2.27 (1H), 2.77 (1H), 2.83-3.05 (2H), 3.14-3.58 (5H), 3.95 (1H), 4.31 (1H), 8.13 (1H), 8.30 (1H), 8.36 (2H), 8.60 (1H), 13.62 (1H) ppm.

### Example 106

### (4-Methylpiperazin-1-yl)[(7S)-4-(1H-pyrazolo[3,4-b]pyridin-5-ylamino)-5,6,7,8-tetrahydro[1]benzothieno[2,3-d]pyrimidin-7-yl]methanone

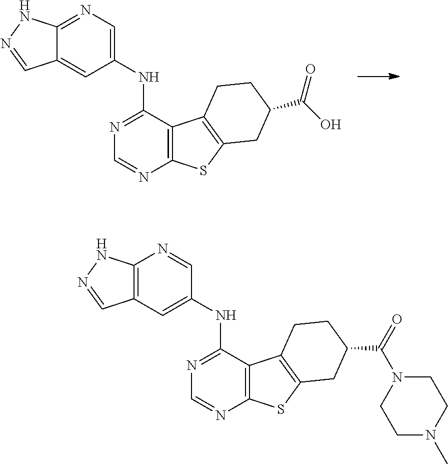 [SMILES: O=C(O)[C@H]1CCc2c(sc3ncnc(Nc4cnc5[nH]ncc5c4)c23)C1 ; CN1CCN(C(=O)[C@H]2CCc3c(sc4ncnc(Nc5cnc6[nH]ncc6c5)c34)C2)CC1]

80 mg (218 μmol) (7S)-4-(1H-pyrazolo[3,4-b]pyridin-5-ylamino)-5,6,7,8-tetrahydro[1]benzothieno[2,3-d]pyrimidine-7-carboxylic acid (prepared according to intermediate example 59a) were transformed in analogy to example 18 using 1-methylpiperazine to give after working up and purification 58.0 mg (56%) of the title compound.

¹H-NMR (DMSO-d6): δ=1.82 (1H), 2.04 (1H), 2.19 (3H), 2.27 (2H), 2.34 (2H), 2.84-3.02 (2H), 3.12-3.43 (3H), 3.45-3.61 (4H), 8.13 (1H), 8.23-8.46 (3H), 8.60 (1H), 13.61 (1H) ppm.

### Example 107

### [4-(Dimethylamino)piperidin-1-yl][(7S)-4-(1H-pyrazolo[3,4-b]pyridin-5-ylamino)-5,6,7,8-tetrahydro[1]benzothieno[2,3-d]pyrimidin-7-yl]methanone

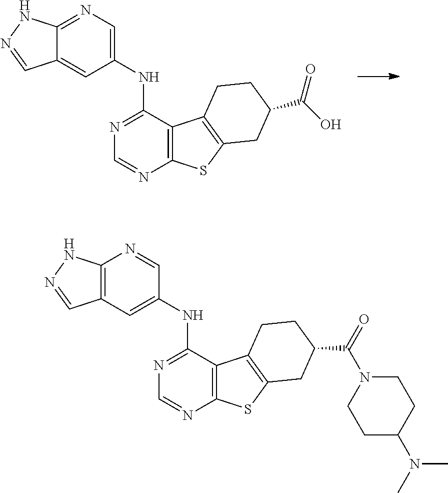 [SMILES: O=C(O)[C@H]1CCc2c(sc3ncnc(Nc4cnc5[nH]ncc5c4)c23)C1 ; CN(C)C1CCN(C(=O)[C@H]2CCc3c(sc4ncnc(Nc5cnc6[nH]ncc6c5)c34)C2)CC1]

80 mg (218 μmol) (7S)-4-(1H-pyrazolo[3,4-b]pyridin-5-ylamino)-5,6,7,8-tetrahydro[1]benzothieno[2,3-d]pyrimidine-7-carboxylic acid (prepared according to intermediate example 59a) were transformed in analogy to example 18 using N,N-dimethylpiperidin-4-amine to give after working up and purification 36.4 mg (33%) of the title compound.

¹H-NMR (DMSO-d6): δ=1.20 (1H), 1.34 (1H), 1.70-1.89 (3H), 2.04 (1H), 2.17 (6H), 2.24-2.40 (2H), 2.61 (1H), 2.85-3.12 (3H), 3.21 (2H), 4.02 (1H), 4.40 (1H), 8.13 (1H), 8.30 (1H), 8.34 (1H), 8.36 (1H), 8.61 (1H), 13.61 (1H) ppm.

### Example 108

### 1-({(7S)-4-[(6-Methoxy-1H-pyrazolo[3,4-b]pyridin-5-yl)amino]-5,6,7,8-tetrahydro[1]benzothieno[2,3-d]pyrimidin-7-yl}carbonyl)azetidine-3-carbonitrile

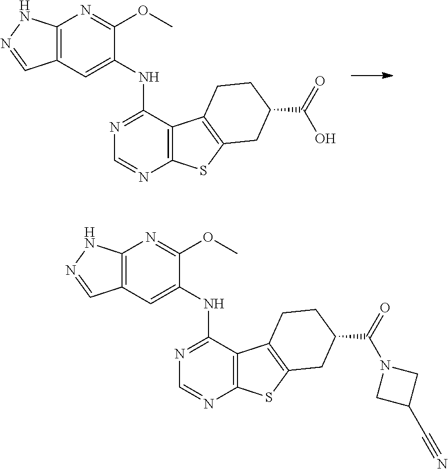 [SMILES: COc1nc2[nH]ncc2cc1Nc1ncnc2sc3c(c12)CC[C@H](C(=O)O)C3 ; COc1nc2[nH]ncc2cc1Nc1ncnc2sc3c(c12)CC[C@H](C(=O)N1CC(C#N)C1)C3]

100 mg (252 μmol) (7S)-4-[(6-methoxy-1H-pyrazolo[3,4-b]pyridin-5-yl)amino]-5,6,7,8-tetrahydro[1]benzothieno[2,3-d]pyrimidine-7-carboxylic acid (prepared according to intermediate example 34a) were transformed in analogy to example 18 using azetidine-3-carbonitrile to give after working up and purification 99.3 mg (81%) of the title compound.

¹H-NMR (DMSO-d6): δ=1.80 (1H), 2.14 (1H), 2.77 (1H), 2.83-3.01 (2H), 3.13 (1H), 3.25 (1H), 3.81 (1H), 4.01 (3H), 4.04 (1H), 4.18 (1H), 4.44-4.60 (2H), 8.00 (1H), 8.09 (1H), 8.41 (1H), 8.82 (1H), 13.35 (1H) ppm.

### Example 109

### {(7S)-4-[(6-Methoxy-1H-pyrazolo[3,4-b]pyridin-5-yl)amino]-5,6,7,8-tetrahydro[1]benzothieno[2,3-d]pyrimidin-7-yl)}(2-oxa-6-azaspiro[3.3]hept-6-yl)methanone

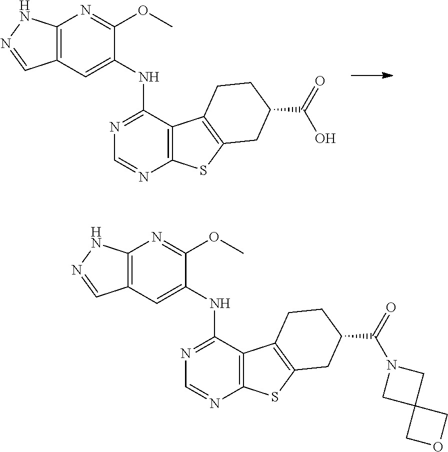 [SMILES: COc1nc2[nH]ncc2cc1Nc1ncnc2sc3c(c12)CC[C@H](C(=O)O)C3 ; COc1nc2[nH]ncc2cc1Nc1ncnc2sc3c(c12)CC[C@H](C(=O)N1CC2(COC2)C1)C3]

50 mg (126 μmol) (7S)-4-[(6-methoxy-1H-pyrazolo[3,4-b]pyridin-5-yl)amino]-5,6,7,8-tetrahydro[1]benzothieno[2,3-d]pyrimidine-7-carboxylic acid (prepared according to intermediate example 34a) were transformed in analogy to example 18 using 2-oxa-6-azaspiro[3.3]heptane to give after working up and purification 4.5 mg (7%) of the title compound.

¹H-NMR (DMSO-d6): δ=1.78 (1H), 2.10 (1H), 2.74 (1H), 2.82-2.96 (2H), 3.11 (1H), 3.24 (1H), 4.02 (3H), 4.06 (2H), 4.41 (2H), 4.64-4.73 (4H), 8.01 (1H), 8.07 (1H), 8.41 (1H), 8.83 (1H), 13.35 (1H) ppm.

### Example 110

### [(3R)-3-(Dimethylamino)pyrrolidin-1-yl]{(7S)-4-[(6-methoxy-1H-pyrazolo[3,4-b]pyridin-5-yl)amino]-5,6,7,8-tetrahydro[1]benzothieno[2,3-d]pyrimidin-7-yl}methanone

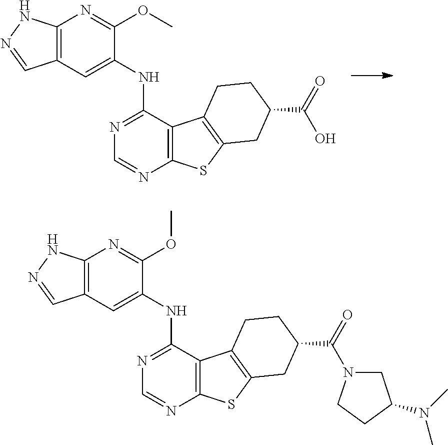

100 mg (252 μmol) (7S)-4-[(6-methoxy-1H-pyrazolo[3,4-b]pyridin-5-yl)amino]-5,6,7,8-tetrahydro[1]benzothieno[2,3-d]pyrimidine-7-carboxylic acid (prepared according to intermediate example 34a) were transformed in analogy to example 18 using (3R)—N,N-dimethylpyrrolidin-3-amine to give after working up and purification 70.4 mg (54%) of the title compound.

¹H-NMR (DMSO-d6): δ=1.58-1.89 (2H), 1.97-2.22 (2H), 2.16 (6H), 2.55-3.67 (9H), 3.78+3.88 (1H), 4.01 (3H), 8.02 (1H), 8.10 (1H), 8.40 (1H), 8.81+8.83 (1H), 13.37 (1H) ppm.

### Example 111

### [(3S)-3-(Dimethylamino)pyrrolidin-1-yl]{(7S)-4-[(6-methoxy-1H-pyrazolo[3,4-b]pyridin-5-yl)amino]-5,6,7,8-tetrahydro[1]benzothieno[2,3-d]pyrimidin-7-yl}methanone

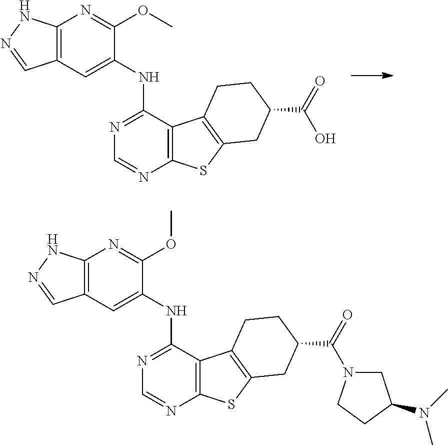

100 mg (252 μmol) (7S)-4-[(6-methoxy-1H-pyrazolo[3,4-b]pyridin-5-yl)amino]-5,6,7,8-tetrahydro[1]benzothieno[2,3-d]pyrimidine-7-carboxylic acid (prepared according to intermediate example 34a) were transformed in analogy to example 18 using (3S)—N,N-dimethylpyrrolidin-3-amine to give after working up and purification 94.9 mg (73%) of the title compound.

¹H-NMR (DMSO-d6): δ=1.56-1.88 (2H), 1.97-2.23 (2H), 2.17 (6H), 2.53-3.86 (10H), 4.01 (3H), 8.01 (1H), 8.08 (1H), 8.40 (1H), 8.83+8.85 (1H), 13.34 (1H) ppm.

### Example 112

### (7S)—N-Ethyl-4-[(6-hydroxy-1H-pyrazolo[3,4-b]pyridin-5-yl)amino]-N-methyl-5,6,7,8-tetrahydro[1]benzothieno[2,3-d]pyrimidine-7-carboxamide

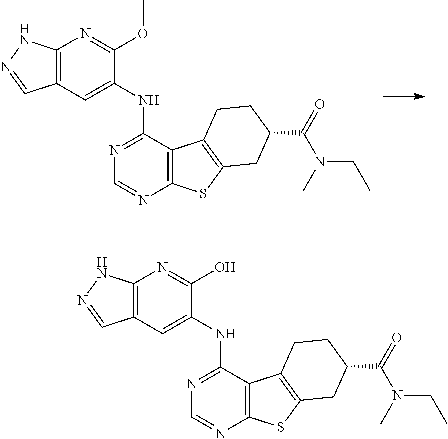 [SMILES: CCN(C)C(=O)[C@H]1CCc2c(sc3ncnc(Nc4cc5cn[nH]c5nc4OC)c23)C1 ; CCN(C)C(=O)[C@H]1CCc2c(sc3ncnc(Nc4cc5cn[nH]c5nc4O)c23)C1]

18.5 mg (42 μmol) (7S)—N-ethyl-4-[(6-methoxy-1H-pyrazolo[3,4-b]pyridin-5-yl)amino]-N-methyl-5,6,7,8-tetrahydro[1]benzothieno[2,3-d]pyrimidine-7-carboxamide (prepared according to example 39) were transformed in analogy to intermediate example 50 to give after working up and purification 6.8 mg (36%) of the title compound.

¹H-NMR (DMSO-d6): δ=1.02+1.14 (3H), 1.85 (1H), 2.09 (1H), 2.84+3.07 (3H), 2.86-3.03 (2H), 3.09-3.53 (5H), 8.07 (1H), 8.33 (2H), 8.55 (1H), 8.86 (1H), 8.95 (1H) ppm.

### Example 113

### (7S)-4-[(6-Hydroxy-1H-pyrazolo[3,4-b]pyridin-5-yl)amino]-N-methyl-N-propyl-5,6,7,8-tetrahydro[1]benzothieno[2,3-d]pyrimidine-7-carboxamide

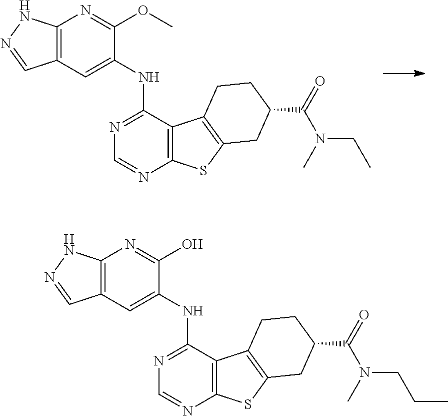 [SMILES: CCN(C)C(=O)[C@H]1CCc2c(sc3ncnc(Nc4cc5cn[nH]c5nc4OC)c23)C1 ; CCCN(C)C(=O)[C@H]1CCc2c(sc3ncnc(Nc4cc5cn[nH]c5nc4O)c23)C1]

18.6 mg (41 μmol) (7S)-4-[(6-methoxy-1H-pyrazolo[3,4-b]pyridin-5-yl)amino]-N-methyl-N-propyl-5,6,7,8-tetrahydro[1]benzothieno[2,3-d]pyrimidine-7-carboxamide (prepared according to example 38) were transformed in analogy to intermediate example 50 to give after working up and purification 5.7 mg (30%) of the title compound.

¹H-NMR (DMSO-d6): δ=0.82+0.87 (3H), 1.42-1.64 (2H), 1.85 (1H), 2.09 (1H), 2.85+3.07 (3H), 2.86-3.04 (2H), 3.11-3.44 (5H), 8.09 (1H), 8.55 (1H), 8.88 (1H), 8.96 (1H), 12.49 (1H), 13.06 (1H) ppm.

### Example 114

### (7S)-4-[(6-Hydroxy-1H-pyrazolo[3,4-b]pyridin-5-yl)amino]-N-methyl-N-(propan-2-yl)-5,6,7,8-tetrahydro[1]benzothieno[2,3-d]pyrimidine-7-carboxamide

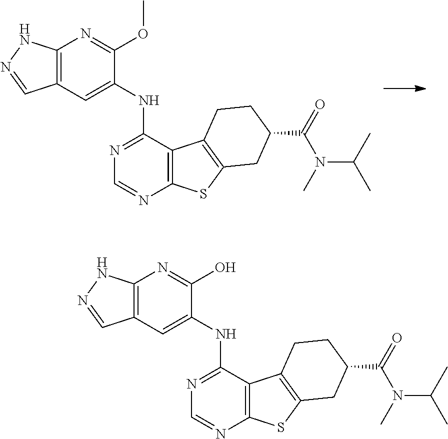 [SMILES: COc1nc2[nH]ncc2cc1Nc1ncnc2sc3c(c12)CC[C@H](C(=O)N(C)C(C)C)C3 ; CC(C)N(C)C(=O)[C@H]1CCc2c(sc3ncnc(Nc4cc5cn[nH]c5nc4O)c23)C1]

19.2 mg (43 μmol) (7S)-4-[(6-methoxy-1H-pyrazolo[3,4-b]pyridin-5-yl)amino]-N-methyl-N-(propan-2-yl)-5,6,7,8-tetrahydro[1]benzothieno[2,3-d]pyrimidine-7-carboxamide (prepared according to example 37) were transformed in analogy to intermediate example 50 to give after working up and purification 5.1 mg (26%) of the title compound.

¹H-NMR (DMSO-d6): δ=1.01-1.22 (6H), 1.84 (1H), 2.09 (1H), 2.70+2.91 (3H), 2.83-3.23 (5H), 4.29+4.71 (1H), 8.09 (1H), 8.55 (1H), 8.88 (1H), 8.96 (1H), 12.48 (1H), 13.07 (1H) ppm.

### Example 115

### {(7S)-4-[(6-Hydroxy-1H-pyrazolo[3,4-b]pyridin-5-yl)amino]-5,6,7,8-tetrahydro[1]benzothieno[2,3-d]pyrimidin-7-yl)}[(1S,4S)-2-oxa-5-azabicyclo[2.2.1]hept-5-yl]methanone

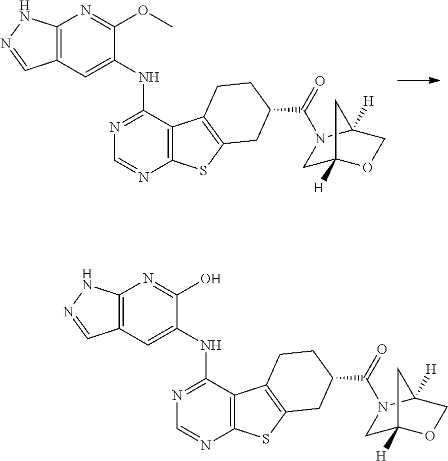 [SMILES: COc1nc2[nH]ncc2cc1Nc1ncnc2sc3c(c12)CC[C@H](C(=O)N1C[C@@H]2C[C@H]1CO2)C3 ; O=C([C@H]1CCc2c(sc3ncnc(Nc4cc5cn[nH]c5nc4O)c23)C1)N1C[C@@H]2C[C@H]1CO2]

18.4 mg (39 μmol) {(7S)-4-[(6-methoxy-1H-pyrazolo[3,4-b]pyridin-5-yl)amino]-5,6,7,8-tetrahydro[1]benzothieno[2,3-d]pyrimidin-7-yl}[(1S,4S)-2-oxa-5-azabicyclo[2.2.1]hept-5-yl]methanone (prepared according to example 40) were transformed in analogy to intermediate example 50 to give after working up and purification 2.8 mg (15%) of the title compound.

¹H-NMR (DMSO-d6): δ=1.73-1.91 (3H), 2.12 (1H), 2.82-3.80 (9H), 4.60+4.85 (1H), 4.76+4.88 (1H), 8.08 (1H), 8.55 (1H), 8.86+8.87 (1H), 8.95 (1H), 12.45 (1H), 13.06 (1H) ppm.

### Example 116

### {(7S)-4-[(6-Hydroxy-1H-pyrazolo[3,4-b]pyridin-5-yl)amino]-5,6,7,8-tetrahydro[1]benzothieno[2,3-d]pyrimidin-7-yl)}(4-methylpiperazin-1-yl)methanone

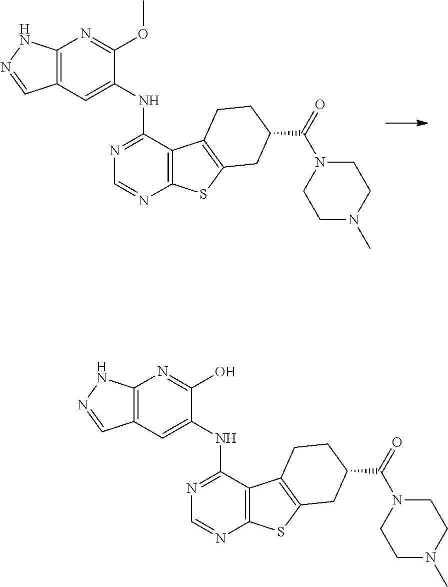 [SMILES: COc1nc2[nH]ncc2cc1Nc1ncnc2sc3c(c12)CC[C@H](C(=O)N1CCN(C)CC1)C3 ; CN1CCN(C(=O)[C@H]2CCc3c(sc4ncnc(Nc5cc6cn[nH]c6nc5O)c34)C2)CC1]

19.4 mg (41 μmol) {(7S)-4-[(6-methoxy-1H-pyrazolo[3,4-b]pyridin-5-yl)amino]-5,6,7,8-tetrahydro[1]benzothieno[2,3-d]pyrimidin-7-yl}(4-methylpiperazin-1-yl)methanone (prepared according to example 42) were transformed in analogy to intermediate example 50 to give after working up and purification 2.2 mg (11%) of the title compound.

¹H-NMR (DMSO-d6): δ=1.83 (1H), 2.10 (1H), 2.19 (3H), 2.24-2.38 (4H), 2.88 (1H), 3.00 (1H), 3.12-3.29 (3H), 3.50 (2H), 3.58 (2H), 8.08 (1H), 8.55 (1H), 8.86 (1H), 8.95 (1H), 12.40 (1H), 13.07 (1H) ppm.

### Example 117

### [4-(Dimethylamino)piperidin-1-yl]{(7S)-4-[(6-hydroxy-1H-pyrazolo[3,4-b]pyridin-5-yl)amino]-5,6,7,8-tetrahydro[1]benzothieno[2,3-d]pyrimidin-7-yl}methanone

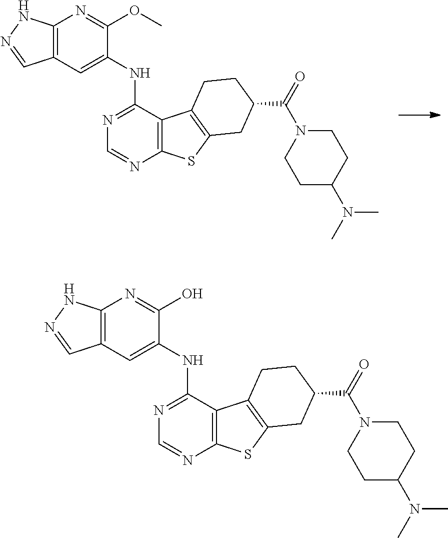 [SMILES: COc1nc2[nH]ncc2cc1Nc1ncnc2sc3c(c12)CC[C@H](C(=O)N1CCC(N(C)C)CC1)C3 ; CN(C)C1CCN(C(=O)[C@H]2CCc3c(sc4ncnc(Nc5cc6cn[nH]c6nc5O)c34)C2)CC1]

19.7 mg (39 μmol) [4-(dimethylamino)piperidin-1-yl]{(7S)-4-[(6-methoxy-1H-pyrazolo[3,4-b]pyridin-5-yl)amino]-5,6,7,8-tetrahydro[1]benzothieno[2,3-d]pyrimidin-7-yl}methanone (prepared according to example 43) were transformed in analogy to intermediate example 50 to give after working up and purification 5.5 mg (27%) of the title compound.

¹H-NMR (DMSO-d6): δ=1.22 (1H), 1.37 (1H), 1.74-1.90 (3H), 2.09 (1H), 2.22 (6H), 2.41 (1H), 2.61 (1H), 2.82-3.28 (6H), 4.07 (1H), 4.42 (1H), 8.09 (1H), 8.55 (1H), 8.86 (1H), 8.96 (1H), 12.43 (1H), 13.04 (1H) ppm.

### Example 118

### 5-Bromo-N-[3-(dimethylamino)propyl]-4-(1H-pyrazolo[3,4-c]pyridin-5-ylamino)-7H-pyrrolo[2,3-d]pyrimidine-6-carboxamide

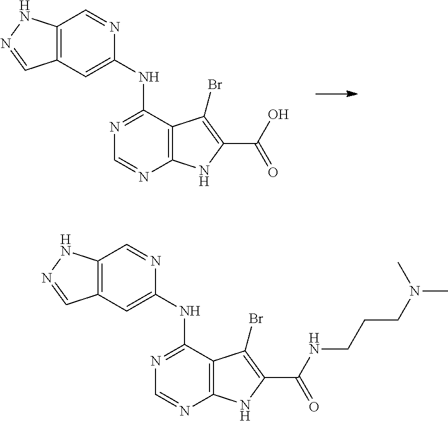 [SMILES: O=C(O)c1[nH]c2ncnc(Nc3cc4cn[nH]c4cn3)c2c1Br ; CN(C)CCCNC(=O)c1[nH]c2ncnc(Nc3cc4cn[nH]c4cn3)c2c1Br]

A mixture comprising 100 mg (267 μmol) 5-bromo-4-(1H-pyrazolo[3,4-c]pyridin-5-ylamino)-7H-pyrrolo[2,3-d]pyrimidine-6-carboxylic acid (prepared according to example 14), 1.85 mL dimethyl sulfoxide, 67 μL N,N-dimethylpropane-1,3-diamine, 140 μL N-ethyl-N-isopropylpropan-2-amine and 209 mg (1H-benzotriazol-1-yloxy)(tripyrrolidin-1-yl)phosphonium hexafluorophosphate was heated at 50° C. overnight. The crude mixture was purified by chromatography to give 20.5 mg (16%) of the title compound.

¹H-NMR (DMSO-d6): δ=1.68 (2H), 2.16 (6H), 2.32 (2H), 3.32 (2H), 8.22 (1H), 8.26 (1H), 8.49 (1H), 8.85 (1H), 8.89 (1H), 8.96 (1H), 13.56 (1H) ppm.

### Example 119

### 5-Bromo-N-[3-(dimethylamino)propyl]-N-methyl-4-(1H-pyrazolo[3,4-c]pyridin-5-ylamino)-7H-pyrrolo[2,3-d]pyrimidine-6-carboxamide

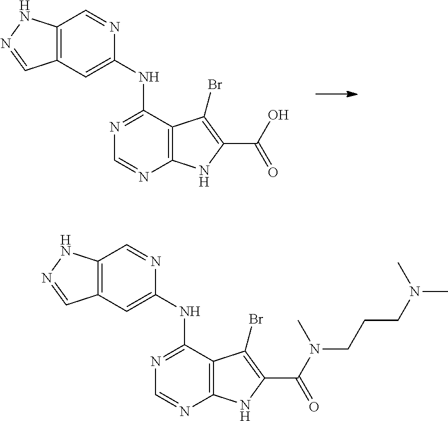 [SMILES: O=C(O)c1[nH]c2ncnc(Nc3cc4cn[nH]c4cn3)c2c1Br ; CN(C)CCCN(C)C(=O)c1[nH]c2ncnc(Nc3cc4cn[nH]c4cn3)c2c1Br]

100 mg (267 μmol) 5-bromo-4-(1H-pyrazolo[3,4-c]pyridin-5-ylamino)-7H-pyrrolo[2,3-d]pyrimidine-6-carboxylic acid (prepared according to example 14) were transformed in analogy to example 118 using N′-ethyl-N,N-dimethylethane-1,2-diamine to give after purification 27.0 mg (21%) of the title compound.

¹H-NMR (DMSO-d6): δ=1.71-1.88 (2H), 2.19 (2H), 2.31 (6H), 2.97 (3H), 3.40-3.54 (2H), 8.25 (1H), 8.48 (1H), 8.82 (1H), 8.84 (1H), 8.90 (1H), 13.54 (1H) ppm.

### Example 120

### 5-Bromo-N-[2-(dimethylamino)ethyl]-4-(1H-pyrazolo[3,4-c]pyridin-5-ylamino)-7H-pyrrolo[2,3-d]pyrimidine-6-carboxamide

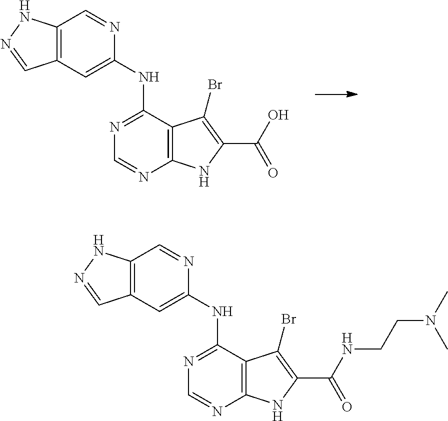 [SMILES: O=C(O)c1[nH]c2ncnc(Nc3cc4cn[nH]c4cn3)c2c1Br ; CN(C)CCNC(=O)c1[nH]c2ncnc(Nc3cc4cn[nH]c4cn3)c2c1Br]

100 mg (267 μmol) 5-bromo-4-(1H-pyrazolo[3,4-c]pyridin-5-ylamino)-7H-pyrrolo[2,3-d]pyrimidine-6-carboxylic acid (prepared according to example 14) were transformed in analogy to example 118 using N,N-dimethylethane-1,2-diamine to give after purification 5.0 mg (4%) of the title compound.

¹H-NMR (DMSO-d6): δ=2.21 (6H), 2.44 (2H), 3.40 (2H), 8.03 (1H), 8.26 (1H), 8.50 (1H), 8.85 (1H), 8.89 (1H), 8.97 (1H), 13.59 (1H) ppm.

### Example 121

### [5-Bromo-4-(1H-pyrazolo[3,4-c]pyridin-5-ylamino)-7H-pyrrolo[2,3-d]pyrimidin-6-yl][(3S)-3-methylmorpholin-4-yl]methanone

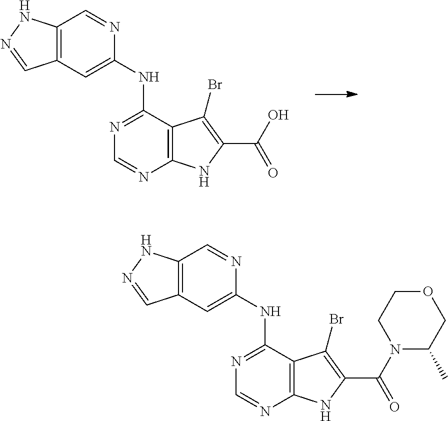 [SMILES: O=C(O)c1[nH]c2ncnc(Nc3cc4cn[nH]c4cn3)c2c1Br ; C[C@H]1COCCN1C(=O)c1[nH]c2ncnc(Nc3cc4cn[nH]c4cn3)c2c1Br]

100 mg (267 μmol) 5-bromo-4-(1H-pyrazolo[3,4-c]pyridin-5-ylamino)-7H-pyrrolo[2,3-d]pyrimidine-6-carboxylic acid (prepared according to example 14) were transformed in analogy to example 18 using (3S)-3-methylmorpholine to give after working up and purification 12.6 mg (10%) of the title compound.

¹H-NMR (DMSO-d6): δ=1.28 (3H), 3.34-3.91 (7H), 8.26 (2H), 8.49 (1H), 8.71 (1H), 8.85 (1H), 8.87 (1H) ppm.

### Example 122

### N-[2-(Dimethylamino)-2-oxoethyl]-N-methyl-7-(1H-pyrazolo[3,4-c]pyridin-5-ylamino)[1,3]thiazolo[5,4-d]pyrimidine-2-carboxamide

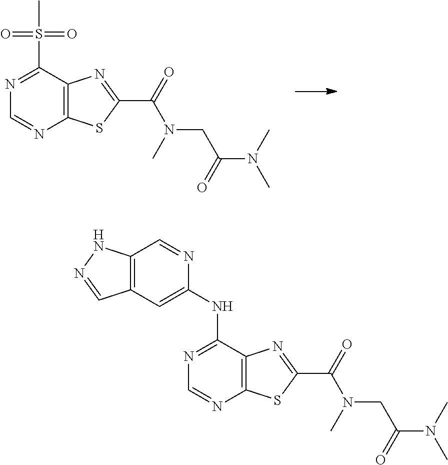 [SMILES: CN(C)C(=O)CN(C)C(=O)c1nc2c(S(C)(=O)=O)ncnc2s1 ; CN(C)C(=O)CN(C)C(=O)c1nc2c(Nc3cc4cn[nH]c4cn3)ncnc2s1]

A mixture comprising 100 mg (280 μmol) N-[2-(dimethylamino)-2-oxoethyl]-N-methyl-7-(methylsulfonyl)[1,3]thiazolo[5,4-d]pyrimidine-2-carboxamide of a dimethyl sulfoxide solution (prepared according to intermediate example 122a) and 37.5 mg 1H-pyrazolo[3,4-c]pyridin-5-amine was heated at 140° C. under microwave irradiation for four hours. The solvent was removed and the residue purified by chromatography to give 8.2 mg (7%) of the title compound.

¹H-NMR (DMSO-d6): δ=2.86+2.88 (3H), 3.00+3.09 (3H), 3.13+3.58 (3H), 4.39+5.02 (2H), 8.27+8.29 (1H), 8.55+8.71 (1H), 8.67+8.72 (1H), 8.91+8.92 (1H), 9.37+10.13 (1H), 13.64 (1H) ppm.

### Example 122a

### N-[2-(Dimethylamino)-2-oxoethyl]-N-methyl-7-(methylsulfonyl)[1,3]thiazolo[5,4-d]pyrimidine-2-carboxamide

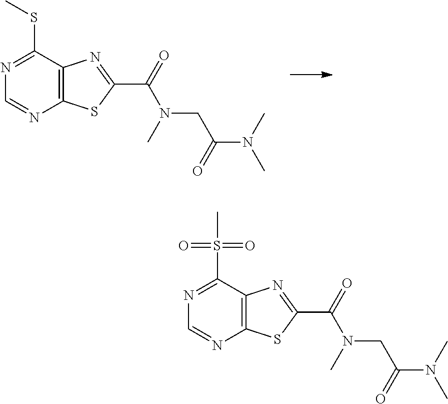

A mixture comprising 800 mg (2.46 mmol) N-[2-(dimethylamino)-2-oxoethyl]-N-methyl-7-(methylsulfanyl)[1,3]thiazolo[5,4-d]pyrimidine-2-carboxamide (prepared according to intermediate example 122b), 35 mL dichloromethane and 1.41 g 3-chlorobenzenecarboperoxoic acid (75%) was stirred at 23° C. for two hours. 22 mL dimethyl sulfoxide were added and the dichloromethane removed to give the title compound as a 0.1M solution in dimethyl sulfoxide.

### Example 122b

### N-[2-(Dimethylamino)-2-oxoethyl]-N-methyl-7-(methylsulfanyl)[1,3]thiazolo[5,4-d]pyrimidine-2-carboxamide

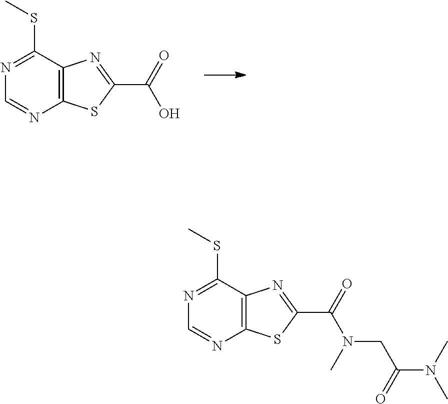 [SMILES: CSc1ncnc2sc(C(=O)O)nc12 ; CSc1ncnc2sc(C(=O)N(C)CC(=O)N(C)C)nc12]

1.00 g (4.40 mmol) 7-(methylsulfanyl)[1,3]thiazolo[5,4-d]pyrimidine-2-carboxylic acid (prepared according to intermediate example 122c) were transformed in analogy to example 18 using N,N,N²-trimethylglycinamide to give after working up and purification 1.03 g (72%) of the title compound.

### Example 122c

### 7-(Methylsulfanyl)[1,3]thiazolo[5,4-d]pyrimidine-2-carboxylic acid

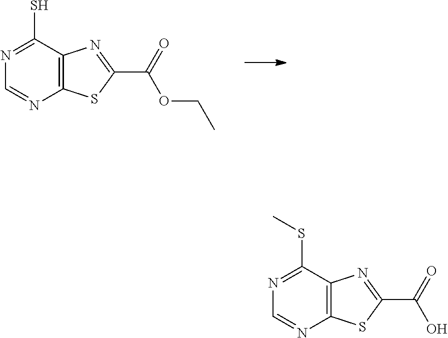

A mixture comprising 35.2 g (146 mmol) ethyl 7-sulfanyl[1,3]thiazolo[5,4-d]pyrimidine-2-carboxylate (prepared according to intermediate example 122d), 750 mL aqueous sodium hydroxide (2M), and 9.08 mL iodomethane was stirred at 23° C. overnight. The precipitate was collected and washed with ethanol and diethylether. 400 mL water were added and the pH adjusted to 2 by the addition of hydrochloric acid (4M). The precipitate was collected, washed with ethanol and diethylether and dried to give 14.28 g (43%) of the title compound.

### Example 122d

### Ethyl 7-sufanyl[1,3]thiazolo[5,4-d]pyrimidine-2-carboxylate

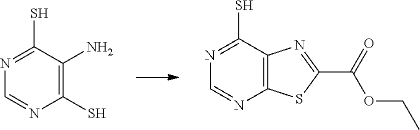

To a solution of 26.4 g (135 mmol) 5-aminopyrimidine-4,6-dithiol (prepared according to intermediate example 122e) in 720 mL pyridine were slowly added 20.5 mL ethyl chloro(oxo) acetate at 0° C. and the mixture was stirred at 23° C. for 4 hours. The solvent was removed and water was added. The precipitate was collected, crystallized from ethanol, washed with diethylether and dried to give 35.2 g that was used without further purification.

### Example 122e

### 5-Aminopyrimidine-4,6-dithiol

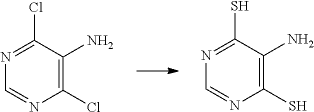 [SMILES: Nc1c(Cl)ncnc1Cl ; Nc1c(S)ncnc1S]

A mixture comprising 25 g (152 mmol) 4,6-dichloropyrimidin-5-amine (CAS-No: 5413-85-4), 45.2 g sulfanylsodium hydrate (1:1) and 600 mL water was heated under reflux for 3 hours. 16.9 g sulfanylsodium hydrate (1:1) were added and heating continued for 3 hours. The mixture was neutralized under cooling by the addition of conc. hydrochloric acid and concentrated. After the ph was adjusted between 2 to 3 by the addition of hydrochloric acid (2M) under cooling, the precipitate was collected, washed with cold water and dried to give 26.4 g (88%) of the title compound as hydrochloride.

### Example 123

### N,N-Dimethyl-7-(1H-pyrazolo[3,4-b]pyridin-5-ylamino)[1,3]thiazolo[5,4-d]pyrimidine-2-carboxamide

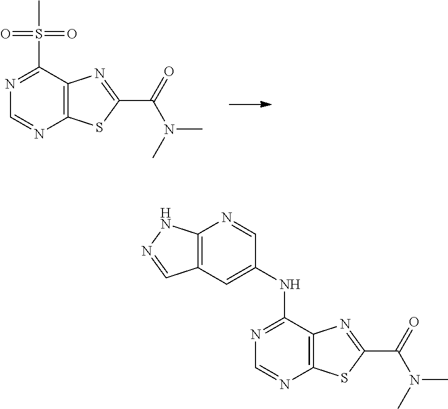 [SMILES: CN(C)C(=O)c1nc2c(S(C)(=O)=O)ncnc2s1 ; CN(C)C(=O)c1nc2c(Nc3cnc4[nH]ncc4c3)ncnc2s1]

100 mg (349 μmol) N,N-dimethyl-7-(methylsulfonyl)[1,3]thiazolo[5,4-d]pyrimidine-2-carboxamide (prepared according to intermediate example 123a) were transformed in analogy to example 122 using 1H-pyrazolo[3,4-b]pyridin-5-amine to give after working up and purification 4.4 mg (4%) of the title compound.

¹H-NMR (DMSO-d6): δ=3.09 (3H), 3.56 (3H), 8.17 (1H), 8.54 (1H), 8.55 (1H), 8.76 (1H), 10.29 (1H), 13.67 (1H) ppm.

### Example 123a

### N,N-Dimethyl-7-(methylsulfonyl)[1,3]thiazolo[5,4-d]pyrimidine-2-carboxamide

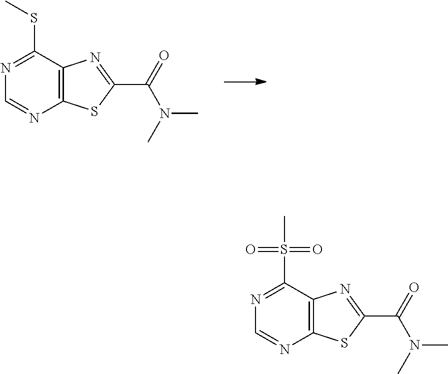 [SMILES: CSc1ncnc2sc(C(=O)N(C)C)nc12 ; CN(C)C(=O)c1nc2c(S(C)(=O)=O)ncnc2s1]

635 mg (2.50 mmol) N,N-dimethyl-7-(methylsulfanyl)[1,3]thiazolo[5,4-d]pyrimidine-2-carboxamide (prepared according to intermediate example 123b) were transformed in analogy to intermediate example 122a to give the title compound as a 0.1M solution in dimethyl sulfoxide.

### Example 123b

### N,N-Dimethyl-7-(methylsulfanyl)[1,3]thiazolo[5,4-d]pyrimidine-2-carboxamide

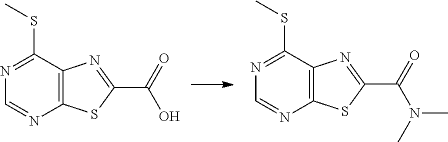 [SMILES: CSc1ncnc2sc(C(=O)O)nc12 ; CSc1ncnc2sc(C(=O)N(C)C)nc12]

1.00 g (4.40 mmol) 7-(methylsulfanyl)[1,3]thiazolo[5,4-d]pyrimidine-2-carboxylic acid (prepared according to intermediate example 122c) were transformed in analogy to example 18 using N-methylmethanamine to give after working up and purification 907 mg (81%) of the title compound.

### Example 124

### 5-Bromo-N-[2-(dimethylamino)ethyl]-4-(1H-pyrazolo[3,4-b]pyridin-5-ylamino)-7H-pyrrolo[2,3-d]pyrimidine-6-carboxamide

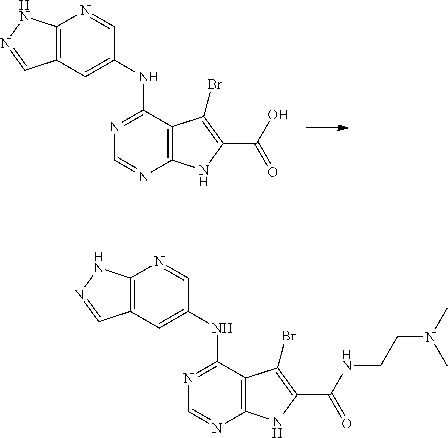 [SMILES: O=C(O)c1[nH]c2ncnc(Nc3cnc4[nH]ncc4c3)c2c1Br ; CN(C)CCNC(=O)c1[nH]c2ncnc(Nc3cnc4[nH]ncc4c3)c2c1Br]

100 mg (267 μmol) 5-bromo-4-(1H-pyrazolo[3,4-b]pyridin-5-ylamino)-7H-pyrrolo[2,3-d]pyrimidine-6-carboxylic acid (prepared according to intermediate example 61a) were transformed in analogy to example 118 using N,N-dimethylethane-1,2-diamine to give after working up and purification 1.8 mg (1%) of the title compound.

¹H-NMR (DMSO-d6): δ=2.21 (6H), 2.45 (2H), 3.40 (2H), 7.97 (1H), 8.14 (1H), 8.28 (1H), 8.56 (1H), 8.61 (1H), 8.67 (1H), 13.61 (1H) ppm.

### Example 125

### 5-Bromo-N-[3-(dimethylamino)propyl]-4-(1H-pyrazolo[3,4-b]pyridin-5-ylamino)-7H-pyrrolo[2,3-d]pyrimidine-6-carboxamide

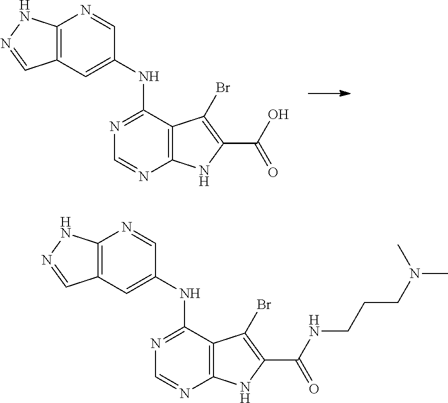 [SMILES: O=C(O)c1[nH]c2ncnc(Nc3cnc4[nH]ncc4c3)c2c1Br ; CN(C)CCCNC(=O)c1[nH]c2ncnc(Nc3cnc4[nH]ncc4c3)c2c1Br]

100 mg (267 μmol) 5-bromo-4-(1H-pyrazolo[3,4-b]pyridin-5-ylamino)-7H-pyrrolo[2,3-d]pyrimidine-6-carboxylic acid (prepared according to intermediate example 61a) were transformed in analogy to example 118 to give after working up and purification 18.8 mg (14%) of the title compound.

¹H-NMR (DMSO-d6): δ=1.68 (2H), 2.16 (6H), 2.33 (2H), 3.34 (2H), 8.14 (1H), 8.17 (1H), 8.28 (1H), 8.57 (1H), 8.60 (1H), 8.68 (1H), 13.61 (1H) ppm.

### Example 126

### Piperidin-1-yl[7-(1H-pyrazolo[3,4-b]pyridin-5-ylamino)[1,3]thiazolo[5,4-d]pyrimidin-2-yl]methanone

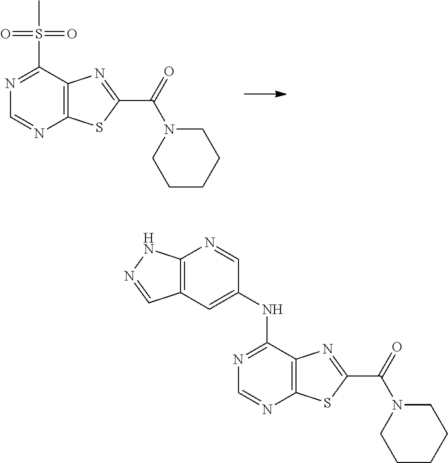 [SMILES: CS(=O)(=O)c1ncnc2sc(C(=O)N3CCCCC3)nc12 ; O=C(c1nc2c(Nc3cnc4[nH]ncc4c3)ncnc2s1)N1CCCCC1]

100 mg (306 μmol) [7-(methylsulfonyl)[1,3]thiazolo[5,4-d]pyrimidin-2-yl](piperidin-1-yl)methanone (prepared according to intermediate example 126a) were transformed in analogy to example 122 to give after working up and purification 32.7 mg (27%) of the title compound.

¹H-NMR (DMSO-d6): δ=1.44-1.75 (6H), 3.65 (2H), 4.15 (2H), 8.16 (1H), 8.53 (2H), 8.74 (1H), 10.28 (1H), 13.68 (1H) ppm.

### Example 126a

### [7-(Methylsulfonyl)[1,3]thiazolo[5,4-d]pyrimidin-2-yl](piperidin-1-yl)methanone

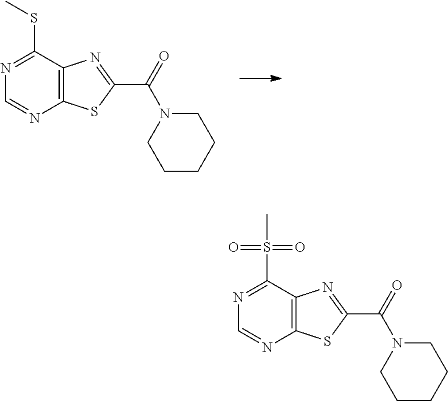 [SMILES: CSc1ncnc2sc(C(=O)N3CCCCC3)nc12 ; CS(=O)(=O)c1ncnc2sc(C(=O)N3CCCCC3)nc12]

100 mg (306 μmol) [7-(methylsulfanyl)[1,3]thiazolo[5,4-d]pyrimidin-2-yl](piperidin-1-yl)methanone (prepared according to intermediate example 126b) were transformed in analogy to intermediate example 122a to give the title compound as a 0.1M solution in dimethyl sulfoxide.

### Example 126b

### [7-(Methylsulfanyl)[1,3]thiazolo[5,4-d]pyrimidin-2-yl](piperidin-1-yl)methanone

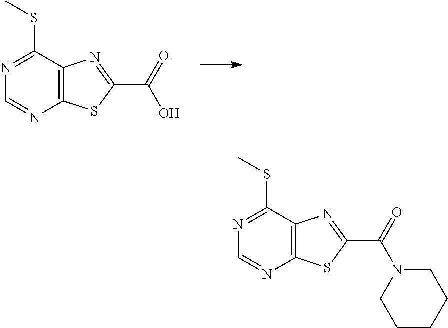 [SMILES: CSc1ncnc2sc(C(=O)O)nc12 ; CSc1ncnc2sc(C(=O)N3CCCCC3)nc12]

1.00 g (4.40 mmol) 7-(methylsulfanyl)[1,3]thiazolo[5,4-d]pyrimidine-2-carboxylic acid (prepared according to intermediate example 122c) were transformed in analogy to example 18 using piperidine to give after working up and purification 998 mg (77%) of the title compound.

### Example 127

### [5-Bromo-4-(1H-pyrazolo[3,4-b]pyridin-5-ylamino)-7H-pyrrolo[2,3-d]pyrimidin-6-yl][4-(dimethylamino)piperidin-1-yl]methanone

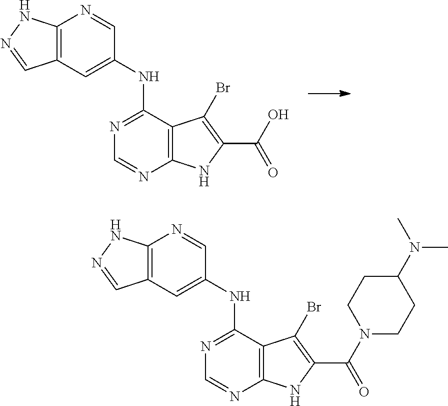 [SMILES: O=C(O)c1[nH]c2ncnc(Nc3cnc4[nH]ncc4c3)c2c1Br ; CN(C)C1CCN(C(=O)c2[nH]c3ncnc(Nc4cnc5[nH]ncc5c4)c3c2Br)CC1]

100 mg (267 μmol) 5-bromo-4-(1H-pyrazolo[3,4-b]pyridin-5-ylamino)-7H-pyrrolo[2,3-d]pyrimidine-6-carboxylic acid (prepared according to intermediate example 61a) were transformed in analogy to example 18 using N,N-dimethylpiperidin-4-amine to give after working up and purification 21.0 mg (15%) of the title compound.

¹H-NMR (DMSO-d6): δ=1.39 (2H), 1.80 (2H), 2.18 (6H), 2.38 (2H), 2.82-3.17 (2H), 3.62 (1H), 4.40 (1H), 8.13 (1H), 8.25 (1H), 8.46 (1H), 8.52 (1H), 8.66 (1H), 13.60 (1H) ppm.

### Example 128

### N-[2-(Dimethylamino)-2-oxoethyl]-N-methyl-7-(1H-pyrazolo[3,4-b]pyridin-5-ylamino)[1,3]thiazolo[5,4-d]pyrimidine-2-carboxamide

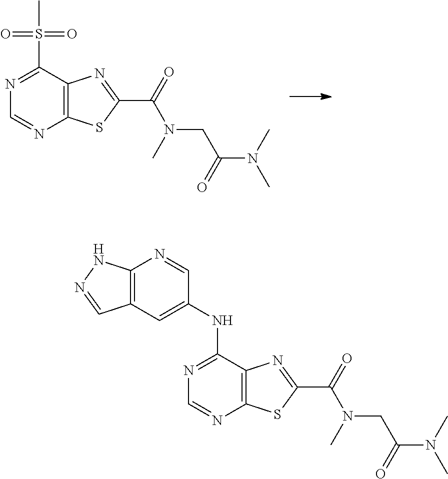 [SMILES: CN(C)C(=O)CN(C)C(=O)c1nc2c(S(C)(=O)=O)ncnc2s1 ; CN(C)C(=O)CN(C)C(=O)c1nc2c(Nc3cnc4[nH]ncc4c3)ncnc2s1]

100 mg (280 μmol) N-[2-(dimethylamino)-2-oxoethyl]-N-methyl-7-(methylsulfonyl)[1,3]thiazolo[5,4-d]pyrimidine-2-carboxamide (prepared according to intermediate example 122a) were transformed in analogy to example 122 to give after working up and purification 112.7 mg (93%) of the title compound.

¹H-NMR (DMSO-d6): δ=2.75+2.86 (3H), 3.00+3.01 (3H), 3.04+3.56 (3H), 4.40+5.21 (2H), 8.17 (1H), 8.55+8.62 (2H), 8.78 (1H), 10.20+10.31 (1H), 13.66 (1H) ppm.

### Example 129

### N-[3-(Dimethylamino)-3-oxopropyl]-N-methyl-7-(1H-pyrazolo[3,4-b]pyridin-5-ylamino)[1,3]thiazolo[5,4-d]pyrimidine-2-carboxamide

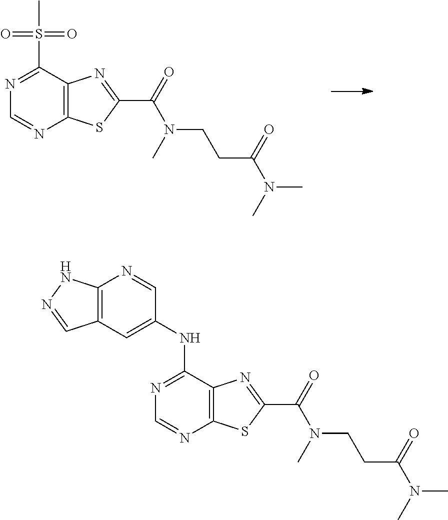 [SMILES: CN(C)C(=O)CCN(C)C(=O)c1nc2c(S(C)(=O)=O)ncnc2s1 ; CN(C)C(=O)CCN(C)C(=O)c1nc2c(Nc3cnc4[nH]ncc4c3)ncnc2s1]

95 mg (256 μmol) N-[3-(dimethylamino)-3-oxopropyl]-N-methyl-7-(methylsulfonyl)[1,3]thiazolo[5,4-d]pyrimidine-2-carboxamide (prepared according to intermediate example 129a) were transformed in analogy to example 122 to give after working up and purification 73.5 mg (64%) of the title compound.

¹H-NMR (DMSO-d6): δ=2.69+2.88 (2H), 2.79+2.83 (3H), 2.98+3.01 (3H), 3.12+3.58 (3H), 3.69+4.25 (2H), 8.17 (1H), 8.54+8.61 (1H), 8.55+8.80 (1H), 8.76+8.95 (1H), 10.29+10.35 (1H), 13.66 (1H) ppm.

### Example 129a

### N-[3-(Dimethylamino)-3-oxopropyl]-N-methyl-7-(methylsulfonyl)[1,3]thiazolo[5,4-d]pyrimidine-2-carboxamide

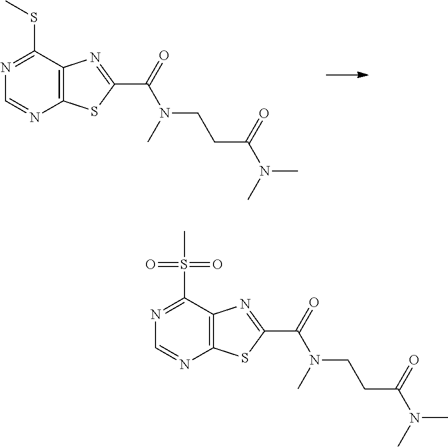 [SMILES: CSc1ncnc2sc(C(=O)N(C)CCC(=O)N(C)C)nc12 ; CN(C)C(=O)CCN(C)C(=O)c1nc2c(S(C)(=O)=O)ncnc2s1]

700 mg (2.06 mmol) N-[3-(dimethylamino)-3-oxopropyl]-N-methyl-7-(methylsulfanyl)[1,3]thiazolo[5,4-d]pyrimidine-2-carboxamide (prepared according to intermediate example 129b) were transformed in analogy to intermediate example 122a to give the title compound as a 0.1M solution in dimethyl sulfoxide.

### Example 129b

### N-[3-(Dimethylamino)-3-oxopropyl]-N-methyl-7-(methylsulfanyl)[1,3]thiazolo[5,4-d]pyrimidine-2-carboxamide

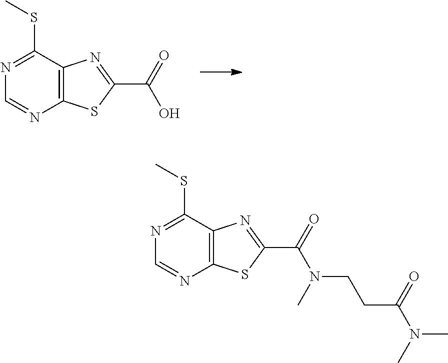 [SMILES: CSc1ncnc2sc(C(=O)O)nc12 ; CSc1ncnc2sc(C(=O)N(C)CCC(=O)N(C)C)nc12]

1.00 g (4.40 mmol) 7-(methylsulfanyl)[1,3]thiazolo[5,4-d]pyrimidine-2-carboxylic acid (prepared according to intermediate example 122c) were transformed in analogy to example 18 using N,N,N³-trimethyl-beta-alaninamide to give after working up and purification 759 mg (51%) of the title compound.

### Example 130

### N-[2-(Dimethylamino)-2-oxoethyl]-7-[(6-methoxy-1H-pyrazolo[3,4-b]pyridin-5-yl)amino]-N-methyl[1,3]thiazolo[5,4-d]pyrimidine-2-carboxamide

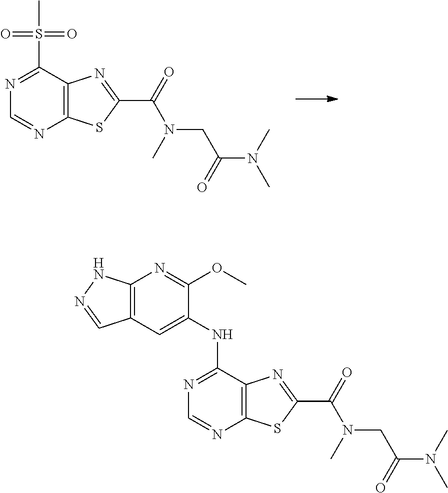 [SMILES: CN(C)C(=O)CN(C)C(=O)c1nc2c(S(C)(=O)=O)ncnc2s1 ; COc1nc2[nH]ncc2cc1Nc1ncnc2sc(C(=O)N(C)CC(=O)N(C)C)nc12]

100 mg (280 μmol) N-[2-(dimethylamino)-2-oxoethyl]-N-methyl-7-(methylsulfonyl)[1,3]thiazolo[5,4-d]pyrimidine-2-carboxamide (prepared according to intermediate example 122a) were transformed in analogy to example 122 using 6-methoxy-1H-pyrazolo[3,4-b]pyridin-5-amine to give after working up and purification 53.7 mg (41%) of the title compound.

¹H-NMR (DMSO-d6): δ=2.78+2.85 (3H), 2.98+3.01 (3H), 3.04+3.48 (3H), 3.92+3.96 (3H), 4.37+5.13 (2H), 8.02+8.04 (1H), 8.36+8.49 (1H), 8.51+8.53 (1H), 9.30+9.69 (1H), 13.46 (1H) ppm.

### Example 131

### 5-Bromo-N-[3-(dimethylamino)propyl]-4-[(6-methoxy-1H-pyrazolo[3,4-b]pyridin-5-yl)amino]-N-methyl-7H-pyrrolo[2,3-d]pyrimidine-6-carboxamide

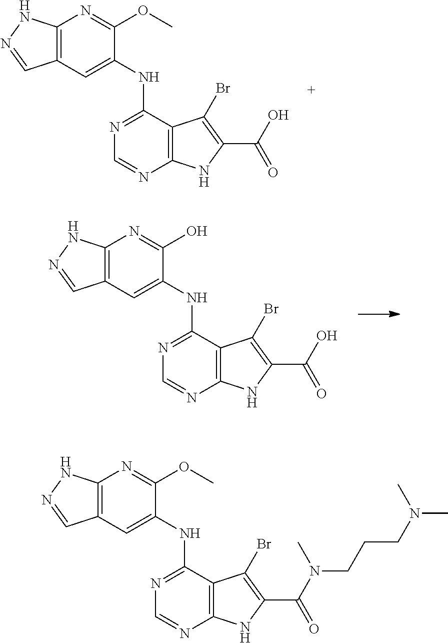 [SMILES: COc1nc2[nH]ncc2cc1Nc1ncnc2[nH]c(C(=O)O)c(Br)c12 ; O=C(O)c1[nH]c2ncnc(Nc3cc4cn[nH]c4nc3O)c2c1Br ; COc1nc2[nH]ncc2cc1Nc1ncnc2[nH]c(C(=O)N(C)CCCN(C)C)c(Br)c12]

175 mg (433 μmol) of a mixture comprising 5-bromo-4-[(6-methoxy-1H-pyrazolo[3,4-b]pyridin-5-yl)amino]-7H-pyrrolo[2,3-d]pyrimidine-6-carboxylic acid and 5-bromo-4-[(6-hydroxy-1H-pyrazolo[3,4-b]pyridin-5-yl)amino]-7H-pyrrolo[2,3-d]pyrimidine-6-carboxylic acid (prepared according to intermediate example 62a) were transformed in analogy to example 118 using N′-ethyl-N,N-dimethylethane-1,2-diamine to give after working up and purification 5.8 mg (2%) of the title compound.

¹H-NMR (DMSO-d6): δ=1.82 (2H), 2.19-2.41 (8H), 2.97 (3H), 3.44 (2H), 4.10 (3H), 8.03 (1H), 8.18 (1H), 8.43 (1H), 8.67+8.73 (1H), 9.30 (1H), 13.28 (1H) ppm.

¹H-NMR (DMSO-d6): δ=1.68 (2H), 2.16 (6H), 2.33 (2H), 3.34 (2H), 8.14 (1H), 8.17 (1H), 8.28 (1H), 8.57 (1H), 8.60 (1H), 8.68 (1H), 13.61 (1H) ppm.

### Example 132

### 5-Bromo-N-[3-(dimethylamino)propyl]-4-[(6-hydroxy-1H-pyrazolo[3,4-b]pyridin-5-yl)amino]-N-methyl-7H-pyrrolo[2,3-d]pyrimidine-6-carboxamide

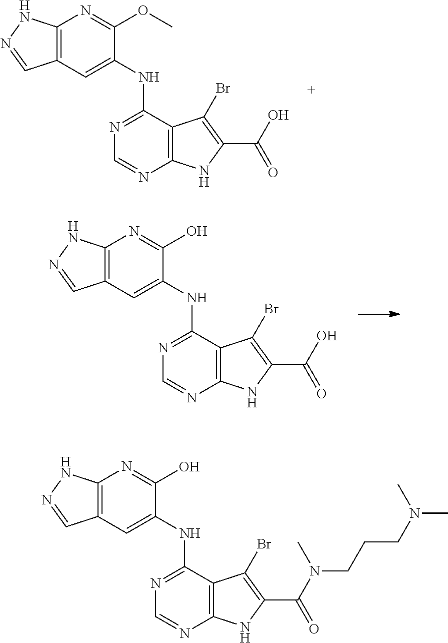 [SMILES: COc1nc2[nH]ncc2cc1Nc1ncnc2[nH]c(C(=O)O)c(Br)c12 ; O=C(O)c1[nH]c2ncnc(Nc3cc4cn[nH]c4nc3O)c2c1Br ; CN(C)CCCN(C)C(=O)c1[nH]c2ncnc(Nc3cc4cn[nH]c4nc3O)c2c1Br]

175 mg (433 μmol) of a mixture comprising 5-bromo-4-[(6-methoxy-1H-pyrazolo[3,4-b]pyridin-5-yl)amino]-7H-pyrrolo[2,3-d]pyrimidine-6-carboxylic acid and 5-bromo-4-[(6-hydroxy-1H-pyrazolo[3,4-b]pyridin-5-yl)amino]-7H-pyrrolo[2,3-d]pyrimidine-6-carboxylic acid (prepared according to intermediate example 62a) were transformed in analogy to example 118 using N′-ethyl-N,N-dimethylethane-1,2-diamine to give after working up and purification 19 mg (8%) of the title compound.

¹H-NMR (DMSO-d6): δ=1.80 (2H), 2.27 (6H), 2.43 (2H), 2.97 (3H), 3.42 (2H), 8.08 (1H), 8.45 (1H), 9.04 (1H), 9.35 (1H), 12.38 (1H), 13.02 (1H) ppm.

### Example 133

### {7-[(6-Methoxy-1H-pyrazolo[3,4-b]pyridin-5-yl)amino][1,3]thiazolo[5,4-d]pyrimidin-2-yl}(piperidin-1-yl)methanone

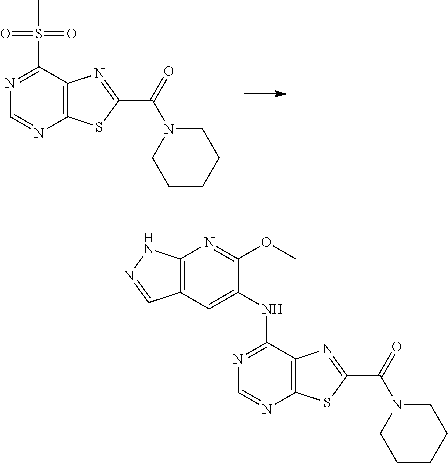 [SMILES: CS(=O)(=O)c1ncnc2sc(C(=O)N3CCCCC3)nc12 ; COc1nc2[nH]ncc2cc1Nc1ncnc2sc(C(=O)N3CCCCC3)nc12]

100 mg (306 μmol) [7-(methylsulfonyl)[1,3]thiazolo[5,4-d]pyrimidin-2-yl](piperidin-1-yl)methanone (prepared according to intermediate example 126a) were transformed in analogy to example 122 using 6-methoxy-1H-pyrazolo[3,4-b]pyridin-5-amine to give after working up and purification 51.9 mg (39%) of the title compound.

¹H-NMR (DMSO-d6): δ=1.46-1.73 (6H), 3.61 (2H), 3.80-4.24 (2H), 3.90 (3H), 8.01 (1H), 8.37 (1H), 8.49 (1H), 9.65 (1H), 13.45 (1H) ppm.

### Example 134

### {5-Bromo-4-[(6-hydroxy-1H-pyrazolo[3,4-b]pyridin-5-yl)amino]-7H-pyrrolo[2,3-d]pyrimidin-6-yl)}(piperidin-1-yl)methanone

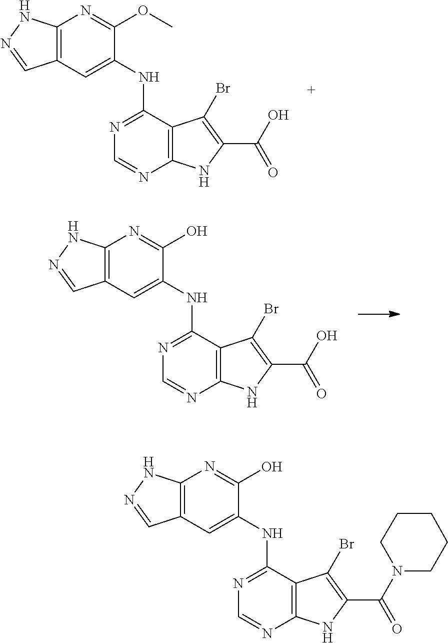 [SMILES: COc1nc2[nH]ncc2cc1Nc1ncnc2[nH]c(C(=O)O)c(Br)c12 ; O=C(O)c1[nH]c2ncnc(Nc3cc4cn[nH]c4nc3O)c2c1Br ; O=C(c1[nH]c2ncnc(Nc3cc4cn[nH]c4nc3O)c2c1Br)N1CCCCC1]

200 mg (495 μmol) of a mixture comprising 5-bromo-4-[(6-methoxy-1H-pyrazolo[3,4-b]pyridin-5-yl)amino]-7H-pyrrolo[2,3-d]pyrimidine-6-carboxylic acid and 5-bromo-4-[(6-hydroxy-1H-pyrazolo[3,4-b]pyridin-5-yl)amino]-7H-pyrrolo[2,3-d]pyrimidine-6-carboxylic acid (prepared according to intermediate example 62a) were transformed in analogy to example 18 using piperidine to give after working up and purification 9.1 mg (4%) of the title compound.

¹H-NMR (DMSO-d6): δ=1.47-1.69 (6H), 3.36-3.71 (4H), 8.09 (1H), 8.46 (1H), 9.02 (1H), 9.30 (1H), 12.39 (1H), 12.75 (1H), 13.03 (1H) ppm.

### Example 135

### {5-Bromo-4-[(6-methoxy-1H-pyrazolo[3,4-b]pyridin-5-yl)amino]-7H-pyrrolo[2,3-d]pyrimidin-6-yl)}[(3S)-3-methylmorpholin-4-yl]methanone

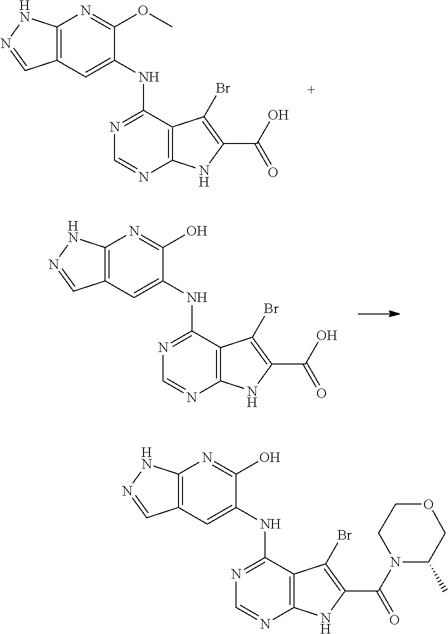 [SMILES: COc1nc2[nH]ncc2cc1Nc1ncnc2[nH]c(C(=O)O)c(Br)c12 ; O=C(O)c1[nH]c2ncnc(Nc3cc4cn[nH]c4nc3O)c2c1Br ; C[C@H]1COCCN1C(=O)c1[nH]c2ncnc(Nc3cc4cn[nH]c4nc3O)c2c1Br]

200 mg (495 μmol) of a mixture comprising 5-bromo-4-[(6-methoxy-1H-pyrazolo[3,4-b]pyridin-5-yl)amino]-7H-pyrrolo[2,3-d]pyrimidine-6-carboxylic acid and 5-bromo-4-[(6-hydroxy-1H-pyrazolo[3,4-b]pyridin-5-yl)amino]-7H-pyrrolo[2,3-d]pyrimidine-6-carboxylic acid (prepared according to intermediate example 62a) were transformed in analogy to example 18 using (3S)-3-methylmorpholine to give after working up and purification 19.4 mg (7%) of the title compound.

¹H-NMR (DMSO-d6): δ=1.28 (3H), 3.28-3.93 (7H), 4.10 (3H), 8.04 (1H), 8.44 (1H), 8.66 (1H), 9.26 (1H), 12.60 (1H), 13.30 (1H) ppm.

### Example 136

### (7S)-4-[(6-Methoxy-1H-pyrazolo[3,4-b]pyridin-5-yl)amino]-N-methyl-N-(3,3,3-trifluoropropyl)-5,6,7,8-tetrahydro[1]benzothieno[2,3-d]pyrimidine-7-carboxamide

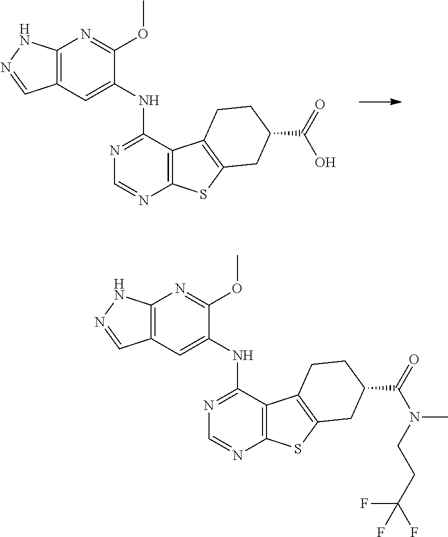 [SMILES: COc1nc2[nH]ncc2cc1Nc1ncnc2sc3c(c12)CC[C@H](C(=O)O)C3 ; COc1nc2[nH]ncc2cc1Nc1ncnc2sc3c(c12)CC[C@H](C(=O)N(C)CCC(F)(F)F)C3]

100 mg (252 μmol) (7S)-4-[(6-methoxy-1H-pyrazolo[3,4-b]pyridin-5-yl)amino]-5,6,7,8-tetrahydro[1]benzothieno[2,3-d]pyrimidine-7-carboxylic acid (prepared according to intermediate example 34a) were transformed in analogy to example 18 using 3,3,3-trifluoro-N-methylpropan-1-amine to give after working up and purification 94.0 mg (74%) of the title compound.

¹H-NMR (DMSO-d6): δ=1.82 (1H), 2.12 (1H), 2.41-2.66 (2H), 2.87+3.13 (3H), 2.92 (2H), 3.09-3.71 (5H), 4.02 (3H), 8.01 (1H), 8.08 (1H), 8.41 (1H), 8.81+8.84 (1H), 13.37 (1H) ppm.

### Example 137

### N-[3-(Dimethylamino)-3-oxopropyl]-N-methyl-7-(1H-pyrazolo[3,4-c]pyridin-5-ylamino)[1,3]thiazolo[5,4-d]pyrimidine-2-carboxamide

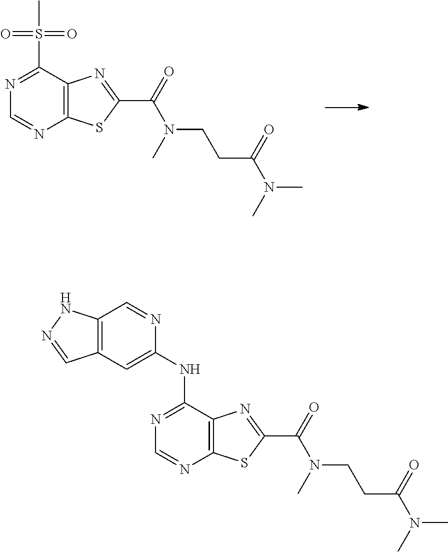 [SMILES: CN(C)C(=O)CCN(C)C(=O)c1nc2c(S(C)(=O)=O)ncnc2s1 ; CN(C)C(=O)CCN(C)C(=O)c1nc2c(Nc3cc4cn[nH]c4cn3)ncnc2s1]

95 mg (256 μmol) N-[3-(dimethylamino)-3-oxopropyl]-N-methyl-7-(methylsulfonyl)[1,3]thiazolo[5,4-d]pyrimidine-2-carboxamide (prepared according to intermediate example 129a) were transformed in analogy to example 122 using 1H-pyrazolo[3,4-c]pyridin-5-amine to give after working up and purification 4.0 mg (3%) of the title compound.

¹H-NMR (DMSO-d6): δ=2.74+3.00 (2H), 2.84 (3H), 2.94+3.00 (3H), 3.13+3.70 (3H), 3.73+4.19 (2H), 6.06 (2H), 6.80+6.83 (1H), 8.29+8.56 (1H), 9.12+9.13 (1H), 9.56+9.69 (1H) ppm.

### Example 138

### N-[3-(Dimethylamino)-3-oxopropyl]-7-[(6-methoxy-1H-pyrazolo[3,4-b]pyridin-5-yl)amino]-N-methyl[1,3]thiazolo[5,4-d]pyrimidine-2-carboxamide

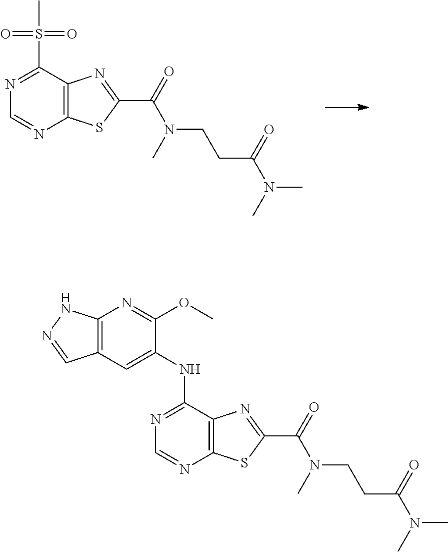 [SMILES: CN(C)C(=O)CCN(C)C(=O)c1nc2c(S(C)(=O)=O)ncnc2s1 ; COc1nc2[nH]ncc2cc1Nc1ncnc2sc(C(=O)N(C)CCC(=O)N(C)C)nc12]

95 mg (256 μmol) N-[3-(dimethylamino)-3-oxopropyl]-N-methyl-7-(methylsulfonyl)[1,3]thiazolo[5,4-d]pyrimidine-2-carboxamide (prepared according to intermediate example 129a) were transformed in analogy to example 122 using 6-methoxy-1H-pyrazolo[3,4-b]pyridin-5-amine to give after working up and purification 49.7 mg (41%) of the title compound.

¹H-NMR (DMSO-d6): δ=2.66+2.76 (2H), 2.72+2.82 (3H), 2.95+2.97 (3H), 3.08+3.31 (3H), 3.66+4.23 (2H), 3.92+3.95 (3H), 8.01+8.02 (1H), 8.38+8.52 (1H), 8.48+8.52 (1H), 9.40+9.62 (1H), 13.43 (1H) ppm.

### Example 139

### (7S)-4-[(6-Hydroxy-1H-pyrazolo[3,4-b]pyridin-5-yl)amino]-N-methyl-N-(3,3,3-trifluoropropyl)-5,6,7,8-tetrahydro[1]benzothieno[2,3-d]pyrimidine-7-carboxamide

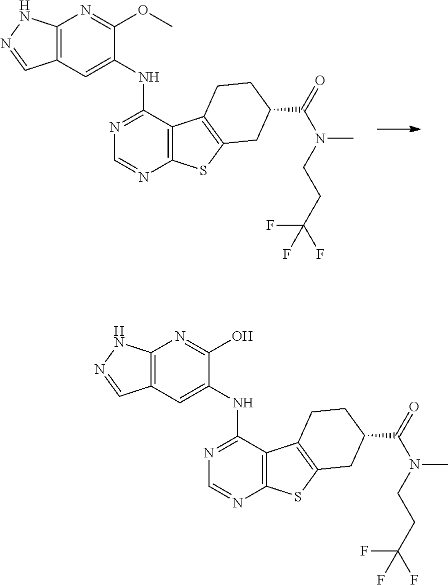 [SMILES: COc1nc2[nH]ncc2cc1Nc1ncnc2sc3c(c12)CC[C@H](C(=O)N(C)CCC(F)(F)F)C3 ; CN(CCC(F)(F)F)C(=O)[C@H]1CCc2c(sc3ncnc(Nc4cc5cn[nH]c5nc4O)c23)C1]

10 mg (20 μmol) (7S)-4-[(6-methoxy-1H-pyrazolo[3,4-b]pyridin-5-yl)amino]-N-methyl-N-(3,3,3-trifluoropropyl)-5,6,7,8-tetrahydro[1]benzothieno[2,3-d]pyrimidine-7-carboxamide (prepared according to example 136) were transformed in analogy to example 50 to give after working up and purification 9.0 mg (93%) of the title compound.

¹H-NMR (DMSO-d6): δ=1.81 (1H), 2.11 (1H), 2.39-2.62 (2H), 2.87+3.13 (3H), 2.92 (2H), 3.09-3.72 (5H), 7.49 (1H), 8.43 (1H), 8.63 (1H), 9.77 (1H), 11.68 (1H) ppm.

### Example 140

### 10 [3-(Dimethylamino)azetidin-1-yl][(7S)-4-(1H-pyrazolo[3,4-c]pyridin-5-ylamino)-5,6,7,8-tetrahydro[1]benzothieno[2,3-d]pyrimidin-7-yl]methanone

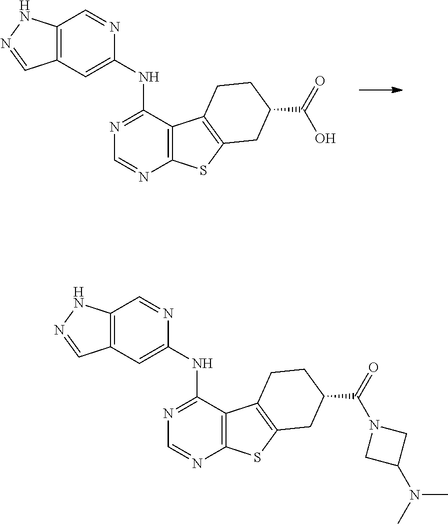 [SMILES: O=C(O)[C@H]1CCc2c(sc3ncnc(Nc4cc5cn[nH]c5cn4)c23)C1 ; CN(C)C1CN(C(=O)[C@H]2CCc3c(sc4ncnc(Nc5cc6cn[nH]c6cn5)c34)C2)C1]

50 mg (136 μmol) (7S)-4-(1H-pyrazolo[3,4-c]pyridin-5-ylamino)-5,6,7,8-tetrahydro[1]benzothieno[2,3-d]pyrimidine-7-carboxylic acid (prepared according to intermediate example 46a) were transformed in analogy to example 18 using N,N-dimethylazetidin-3-amine to give after working up and purification 44.6 mg (69%) of the title compound.

¹H-NMR (DMSO-d6): δ=1.77 (1H), 2.09 (6H), 2.11 (1H), 2.76 (1H), 2.91 (2H), 3.05 (1H), 3.15-3.30 (2H), 3.66 (1H), 3.88 (1H), 4.04 (1H), 4.24 (1H), 8.24 (1H), 8.35 (1H), 8.53 (1H), 8.68 (1H), 8.84 (1H), 13.51 (1H) ppm.

### Example 141

### [3-(Dimethylamino)azetidin-1-yl]{(7S)-4-[(6-methoxy-1H-pyrazolo[3,4-b]pyridin-5-yl)amino]-5,6,7,8-tetrahydro[1]benzothieno[2,3-d]pyrimidin-7-yl}methanone

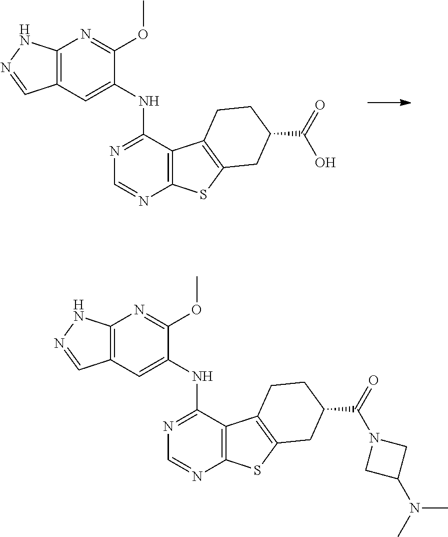 [SMILES: COc1nc2[nH]ncc2cc1Nc1ncnc2sc3c(c12)CC[C@H](C(=O)O)C3 ; COc1nc2[nH]ncc2cc1Nc1ncnc2sc3c(c12)CC[C@H](C(=O)N1CC(N(C)C)C1)C3]

50 mg (126 μmol) (7S)-4-[(6-methoxy-1H-pyrazolo[3,4-b]pyridin-5-yl)amino]-5,6,7,8-tetrahydro[1]benzothieno[2,3-d]pyrimidine-7-carboxylic acid (prepared according to intermediate example 34a) were transformed in analogy to example 18 using N,N-dimethylazetidin-3-amine to give after working up and purification 17.4 mg (27%) of the title compound.

¹H-NMR (DMSO-d6): δ=1.80 (1H), 2.10 (6H), 2.12 (1H), 2.79 (1H), 2.90 (2H), 2.97-3.30 (3H), 3.67 (1H), 3.88 (1H), 4.02 (3H), 4.05 (1H), 4.25 (1H), 8.01 (1H), 8.08 (1H), 8.41 (1H), 8.84 (1H), 13.34 (1H) ppm.

### Example 142

### [3-(Dimethylamino)azetidin-1-yl][(7S)-4-(1H-pyrazolo[3,4-b]pyridin-5-ylamino)-5,6,7,8-tetrahydro[1]benzothieno[2,3-d]pyrimidin-7-yl]methanone

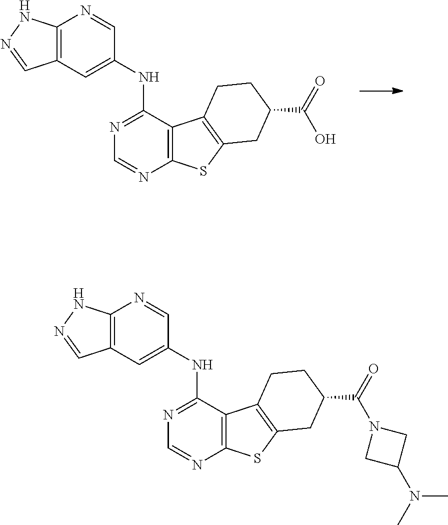 [SMILES: O=C(O)[C@H]1CCc2c(sc3ncnc(Nc4cnc5[nH]ncc5c4)c23)C1 ; CN(C)C1CN(C(=O)[C@H]2CCc3c(sc4ncnc(Nc5cnc6[nH]ncc6c5)c34)C2)C1]

50 mg (136 μmol) (7S)-4-(1H-pyrazolo[3,4-b]pyridin-5-ylamino)-5,6,7,8-tetrahydro[1]benzothieno[2,3-d]pyrimidine-7-carboxylic acid (prepared according to intermediate example 59a) were transformed in analogy to example 18 using N,N-dimethylazetidin-3-amine to give after working up and purification 47.5 mg (74%) of the title compound.

¹H-NMR (DMSO-d6): δ=1.78 (1H), 2.05 (1H), 2.08 (6H), 2.78 (1H), 2.91 (2H), 3.05 (1H), 3.16 (1H), 3.30 (1H), 3.66 (1H), 3.88 (1H), 4.02 (1H), 4.24 (1H), 8.13 (1H), 8.30 (1H), 8.34 (1H), 8.36 (1H), 8.61 (1H), 13.59 (1H) ppm.

### Example 143

### {(7S)-4-[(6-Methoxy-1H-pyrazolo[3,4-b]pyridin-5-yl)amino]-5,6,7,8-tetrahydro[1]benzothieno[2,3-d]pyrimidin-7-yl)}(2-oxa-6-azaspiro[3.3]hept-6-yl)methanone

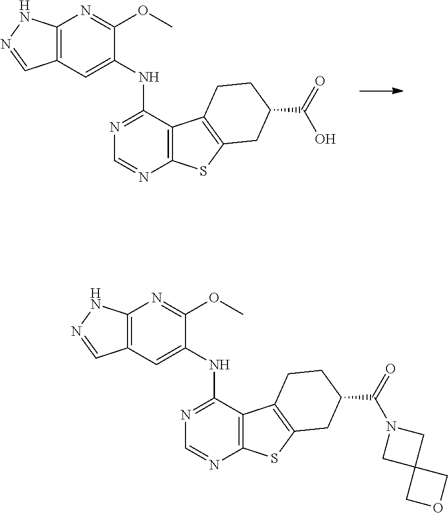 [SMILES: COc1nc2[nH]ncc2cc1Nc1ncnc2sc3c(c12)CC[C@H](C(=O)O)C3 ; COc1nc2[nH]ncc2cc1Nc1ncnc2sc3c(c12)CC[C@H](C(=O)N1CC2(COC2)C1)C3]

50 mg (126 μmol) (7S)-4-[(6-methoxy-1H-pyrazolo[3,4-b]pyridin-5-yl)amino]-5,6,7,8-tetrahydro[1]benzothieno[2,3-d]pyrimidine-7-carboxylic acid (prepared according to intermediate example 34a) were transformed in analogy to example 18 using 2-oxa-6-azaspiro[3.3]heptane to give after working up and purification 4.5 mg (7%) of the title compound.

¹H-NMR (DMSO-d6): δ=1.78 (1H), 2.11 (1H), 2.73 (1H), 2.88 (2H), 3.11 (1H), 3.24 (1H), 4.02 (3H), 4.06 (2H), 4.42 (2H), 4.68 (4H), 8.01 (1H), 8.07 (1H), 8.41 (1H), 8.83 (1H), 13.35 (1H) ppm.

Further, the compounds of formula I of the present invention can be converted to any salt as described herein, by any method which is known to the person skilled in the art. Similarly, any salt of a compound of formula I of the present invention can be converted into the free compound, by any method which is known to the person skilled in the art.

### Pharmaceutical Compositions of the Compounds of the Invention

This invention also relates to pharmaceutical compositions containing one or more compounds of the present invention. These compositions can be utilised to achieve the desired pharmacological effect by administration to a patient in need thereof. A patient, for the purpose of this invention, is a mammal, including a human, in need of treatment for the particular condition or disease. Therefore, the present invention includes pharmaceutical compositions that are comprised of a pharmaceutically acceptable carrier and a pharmaceutically effective amount of a compound, or salt thereof, of the present invention. A pharmaceutically acceptable carrier is preferably a carrier that is relatively non-toxic and innocuous to a patient at concentrations consistent with effective activity of the active ingredient so that any side effects ascribable to the carrier do not vitiate the beneficial effects of the active ingredient. A pharmaceutically effective amount of compound is preferably that amount which produces a result or exerts an influence on the particular condition being treated. The compounds of the present invention can be administered with pharmaceutically-acceptable carriers well known in the art using any effective conventional dosage unit forms, including immediate, slow and timed release preparations, orally, parenterally, topically, nasally, ophthalmically, optically, sublingually, rectally, vaginally, and the like.

For oral administration, the compounds can be formulated into solid or liquid preparations such as capsules, pills, tablets, troches, lozenges, melts, powders, solutions, suspensions, or emulsions, and may be prepared according to methods known to the art for the manufacture of pharmaceutical compositions. The solid unit dosage forms can be a capsule that can be of the ordinary hard- or soft-shelled gelatine type containing, for example, surfactants, lubricants, and inert fillers such as lactose, sucrose, calcium phosphate, and corn starch.

In another embodiment, the compounds of this invention may be tableted with conventional tablet bases such as lactose, sucrose and cornstarch in combination with binders such as acacia, corn starch or gelatine, disintegrating agents intended to assist the break-up and dissolution of the tablet following administration such as potato starch, alginic acid, corn starch, and guar gum, gum tragacanth, acacia, lubricants intended to improve the flow of tablet granulation and to prevent the adhesion of tablet material to the surfaces of the tablet dies and punches, for example talc, stearic acid, or magnesium, calcium or zinc stearate, dyes, colouring agents, and flavouring agents such as peppermint, oil of wintergreen, or cherry flavouring, intended to enhance the aesthetic qualities of the tablets and make them more acceptable to the patient. Suitable excipients for use in oral liquid dosage forms include dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above. Additional excipients, for example those sweetening, flavouring and colouring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived form fatty acids and hexitol anhydrides, for example, sorbitan monooleate, (4) condensation products of said partial esters with ethylene oxide, for example, polyoxyethylene sorbitan monooleate. The emulsions may also contain sweetening and flavouring agents.

Oily suspensions may be formulated by suspending the active ingredient in a vegetable oil such as, for example, arachis oil, olive oil, sesame oil or coconut oil, or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more colouring agents; one or more flavouring agents; and one or more sweetening agents such as sucrose or saccharin.

Syrups and elixirs may be formulated with sweetening agents such as, for example, glycerol, propylene glycol, sorbitol or sucrose. Such formulations may also contain a demulcent, and preservative, such as methyl and propyl parabens and flavouring and colouring agents.

The compounds of this invention may also be administered parenterally, that is, subcutaneously, intravenously, intraocularly, intrasynovially, intramuscularly, or interperitoneally, as injectable dosages of the compound in preferably a physiologically acceptable diluent with a pharmaceutical carrier which can be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,1-dioxolane-4-methanol, ethers such as poly(ethylene glycol) 400, an oil, a fatty acid, a fatty acid ester or, a fatty acid glyceride, or an acetylated fatty acid glyceride, with or without the addition of a pharmaceutically acceptable surfactant such as a soap or a detergent, suspending agent such as pectin, carbomers, methylcellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum and mineral oil. Suitable fatty acids include oleic acid, stearic acid, isostearic acid and myristic acid. Suitable fatty acid esters are, for example, ethyl oleate and isopropyl myristate. Suitable soaps include fatty acid alkali metal, ammonium, and triethanolamine salts and suitable detergents include cationic detergents, for example dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; non-ionic detergents, for example, fatty amine oxides, fatty acid alkanolamides, and poly(oxyethylene-oxypropylene)s or ethylene oxide or propylene oxide copolymers; and amphoteric detergents, for example, alkyl-beta-aminopropionates, and 2-alkylimidazoline quarternary ammonium salts, as well as mixtures.

The parenteral compositions of this invention will typically contain from about 0.5% to about 25% by weight of the active ingredient in solution. Preservatives and buffers may also be used advantageously. In order to minimise or eliminate irritation at the site of injection, such compositions may contain a non-ionic surfactant having a hydrophile-lipophile balance (HLB) preferably of from about 12 to about 17. The quantity of surfactant in such formulation preferably ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadeca-ethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, isotonic sodium chloride solutions and isotonic glucose solutions. In addition, sterile fixed oils are conventionally employed as solvents or suspending media. For this purpose, any bland, fixed oil may be employed including synthetic mono- or diglycerides. In addition, fatty acids such as oleic acid can be used in the preparation of injectables.

A composition of the invention may also be administered in the form of suppositories for rectal administration of the drug. These compositions can be prepared by mixing the drug with a suitable non-irritation excipient which is solid at ordinary temperatures but liquid at the rectal temperature and will therefore melt in the rectum to release the drug. Such materials are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, issued Jun. 11, 1991, incorporated herein by reference). Such patches may be constructed for continuous, pulsatile, or on demand delivery of pharmaceutical agents.

Controlled release formulations for parenteral administration include liposomal, polymeric microsphere and polymeric gel formulations that are known in the art.

It may be desirable or necessary to introduce the pharmaceutical composition to the patient via a mechanical delivery device. The construction and use of mechanical delivery devices for the delivery of pharmaceutical agents is well known in the art. Direct techniques for, for example, administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, issued Apr. 30, 1991.

The compositions of the invention can also contain other conventional pharmaceutically acceptable compounding ingredients, generally referred to as carriers or diluents, as necessary or desired. Conventional procedures for preparing such compositions in appropriate dosage forms can be utilized.

Such ingredients and procedures include those described in the following references, each of which is incorporated herein by reference: Powell, M. F. et al., “Compendium of Excipients for Parenteral Formulations” PDA Journal of Pharmaceutical Science & Technology 1998, 52(5), 238-311; Strickley, R. G “Parenteral Formulations of Small Molecule Therapeutics Marketed in the United States (1999)-Part-1” PDA Journal of Pharmaceutical Science & Technology 1999, 53(6), 324-349; and Nema, S. et al., “Excipients and Their Use in Injectable Products” PDA Journal of Pharmaceutical Science & Technology 1997, 51(4), 166-171.

Commonly used pharmaceutical ingredients that can be used as appropriate to formulate the composition for its intended route of administration include:

acidifying agents (examples include but are not limited to acetic acid, citric acid, fumaric acid, hydrochloric acid, nitric acid);
alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine);
adsorbents (examples include but are not limited to powdered cellulose and activated charcoal);
aerosol propellants (examples include but are not limited to carbon dioxide, CCl₂F₂, F₂ClC—CClF₂ and CClF₃) air displacement agents (examples include but are not limited to nitrogen and argon);
antifungal preservatives (examples include but are not limited to benzoic acid, butylparaben, ethylparaben, methylparaben, propylparaben, sodium benzoate);
antimicrobial preservatives (examples include but are not limited to benzalkonium chloride, benzethonium chloride, benzyl alcohol, cetylpyridinium chloride, chlorobutanol, phenol, phenylethyl alcohol, phenylmercuric nitrate and thimerosal);
antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite);
binding materials (examples include but are not limited to block polymers, natural and synthetic rubber, polyacrylates, polyurethanes, silicones, polysiloxanes and styrene-butadiene copolymers);
buffering agents (examples include but are not limited to potassium metaphosphate, dipotassium phosphate, sodium acetate, sodium citrate anhydrous and sodium citrate dihydrate)
carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection)
chelating agents (examples include but are not limited to edetate disodium and edetic acid)
colourants (examples include but are not limited to FD&C Red No. 3, FD&C Red No. 20, FD&C Yellow No. 6, FD&C Blue No. 2, D&C Green No. 5, D&C Orange No. 5, D&C Red No. 8, caramel and ferric oxide red);
clarifying agents (examples include but are not limited to bentonite);
emulsifying agents (examples include but are not limited to acacia, cetomacrogol, cetyl alcohol, glyceryl monostearate, lecithin, sorbitan monooleate, polyoxyethylene 50 monostearate);
encapsulating agents (examples include but are not limited to gelatin and cellulose acetate phthalate)
flavourants (examples include but are not limited to anise oil, cinnamon oil, cocoa, menthol, orange oil, peppermint oil and vanillin);
humectants (examples include but are not limited to glycerol, propylene glycol and sorbitol);
levigating agents (examples include but are not limited to mineral oil and glycerin);
oils (examples include but are not limited to arachis oil, mineral oil, olive oil, peanut oil, sesame oil and vegetable oil);
ointment bases (examples include but are not limited to lanolin, hydrophilic ointment, polyethylene glycol ointment, petrolatum, hydrophilic petrolatum, white ointment, yellow ointment, and rose water ointment);
penetration enhancers (transdermal delivery) (examples include but are not limited to monohydroxy or polyhydroxy alcohols, mono- or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas)
plasticizers (examples include but are not limited to diethyl phthalate and glycerol)
solvents (examples include but are not limited to ethanol, corn oil, cottonseed oil, glycerol, isopropanol, mineral oil, oleic acid, peanut oil, purified water, water for injection, sterile water for injection and sterile water for irrigation);
stiffening agents (examples include but are not limited to cetyl alcohol, cetyl esters wax, microcrystalline wax, paraffin, stearyl alcohol, white wax and yellow wax);
suppository bases (examples include but are not limited to cocoa butter and polyethylene glycols (mixtures));
surfactants (examples include but are not limited to benzalkonium chloride, nonoxynol 10, oxtoxynol 9, polysorbate 80, sodium lauryl sulfate and sorbitan mono-palmitate);
suspending agents (examples include but are not limited to agar, bentonite, carbomers, carboxymethylcellulose sodium, hydroxyethyl cellulose, hydroxypropyl cellulose, hydroxypropyl methylcellulose, kaolin, methylcellulose, tragacanth and veegum);
sweetening agents (examples include but are not limited to aspartame, dextrose, glycerol, mannitol, propylene glycol, saccharin sodium, sorbitol and sucrose);
tablet anti-adherents (examples include but are not limited to magnesium stearate and talc);
tablet binders (examples include but are not limited to acacia, alginic acid, carboxymethylcellulose sodium, compressible sugar, ethylcellulose, gelatin, liquid glucose, methylcellulose, non-crosslinked polyvinyl pyrrolidone, and pregelatinized starch);
tablet and capsule diluents (examples include but are not limited to dibasic calcium phosphate, kaolin, lactose, mannitol, microcrystalline cellulose, powdered cellulose, precipitated calcium carbonate, sodium carbonate, sodium phosphate, sorbitol and starch);
tablet coating agents (examples include but are not limited to liquid glucose, hydroxyethyl cellulose, hydroxypropyl cellulose, hydroxypropyl methylcellulose, methylcellulose, ethylcellulose, cellulose acetate phthalate and shellac);
tablet direct compression excipients (examples include but are not limited to dibasic calcium phosphate);
tablet disintegrants (examples include but are not limited to alginic acid, carboxymethylcellulose calcium, microcrystalline cellulose, polacrillin potassium, cross-linked polyvinylpyrrolidone, sodium alginate, sodium starch glycollate and starch);
tablet glidants (examples include but are not limited to colloidal silica, corn starch and talc);
tablet lubricants (examples include but are not limited to calcium stearate, magnesium stearate, mineral oil, stearic acid and zinc stearate);
tablet/capsule opaquants (examples include but are not limited to titanium dioxide);
tablet polishing agents (examples include but are not limited to carnuba wax and white wax);
thickening agents (examples include but are not limited to beeswax, cetyl alcohol and paraffin);
tonicity agents (examples include but are not limited to dextrose and sodium chloride);
viscosity increasing agents (examples include but are not limited to alginic acid, bentonite, carbomers, carboxymethylcellulose sodium, methylcellulose, polyvinyl pyrrolidone, sodium alginate and tragacanth); and
wetting agents (examples include but are not limited to heptadecaethylene oxycetanol, lecithins, sorbitol monooleate, polyoxyethylene sorbitol monooleate, and polyoxyethylene stearate).

Pharmaceutical compositions according to the present invention can be illustrated as follows:

Sterile IV Solution: A 5 mg/mL solution of the desired compound of this invention can be made using sterile, injectable water, and the pH is adjusted if necessary. The solution is diluted for administration to 1-2 mg/mL with sterile 5% dextrose and is administered as an IV infusion over about 60 minutes.

Lyophilised powder for IV administration: A sterile preparation can be prepared with (i) 100-1000 mg of the desired compound of this invention as a lyophilised powder, (ii) 32-327 mg/mL sodium citrate, and (iii) 300-3000 mg Dextran 40. The formulation is reconstituted with sterile, injectable saline or dextrose 5% to a concentration of 10 to 20 mg/mL, which is further diluted with saline or dextrose 5% to 0.2-0.4 mg/mL, and is administered either IV bolus or by IV infusion over 15-60 minutes.

Intramuscular suspension: The following solution or suspension can be prepared, for intramuscular injection:

50 mg/mL of the desired, water-insoluble compound of this invention
5 mg/mL sodium carboxymethylcellulose
4 mg/mL TWEEN 80
9 mg/mL sodium chloride
9 mg/mL benzyl alcohol

Hard Shell Capsules: A large number of unit capsules are prepared by filling standard two-piece hard galantine capsules each with 100 mg of powdered active ingredient, 150 mg of lactose, 50 mg of cellulose and 6 mg of magnesium stearate.

Soft Gelatin Capsules: A mixture of active ingredient in a digestible oil such as soybean oil, cottonseed oil or olive oil is prepared and injected by means of a positive displacement pump into molten gelatin to form soft gelatin capsules containing 100 mg of the active ingredient.

The capsules are washed and dried. The active ingredient can be dissolved in a mixture of polyethylene glycol, glycerin and sorbitol to prepare a water miscible medicine mix.

Tablets: A large number of tablets are prepared by conventional procedures so that the dosage unit is 100 mg of active ingredient, 0.2 mg. of colloidal silicon dioxide, 5 mg of magnesium stearate, 275 mg of microcrystalline cellulose, 11 mg. of starch, and 98.8 mg of lactose. Appropriate aqueous and non-aqueous coatings may be applied to increase palatability, improve elegance and stability or delay absorption.

Immediate Release Tablets/Capsules: These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques.

The drug compounds may be compressed with viscoelastic and thermoelastic sugars and polymers or effervescent components to produce porous matrices intended for immediate release, without the need of water.

### Combination Therapies

The term “combination” in the present invention is used as known to persons skilled in the art and may be present as a fixed combination, a non-fixed combination or kit-of-parts.

A “fixed combination” in the present invention is used as known to persons skilled in the art and is defined as a combination wherein the said first active ingredient and the said second active ingredient are present together in one unit dosage or in a single entity. One example of a “fixed combination” is a pharmaceutical composition wherein the said first active ingredient and the said second active ingredient are present in admixture for simultaneous administration, such as in a formulation. Another example of a “fixed combination” is a pharmaceutical combination wherein the said first active ingredient and the said second active ingredient are present in one unit without being in admixture.

A non-fixed combination or “kit-of-parts” in the present invention is used as known to persons skilled in the art and is defined as a combination wherein the said first active ingredient and the said second active ingredient are present in more than one unit. One example of a non-fixed combination or kit-of-parts is a combination wherein the said first active ingredient and the said second active ingredient are present separately. The components of the non-fixed combination or kit-of-parts may be administered separately, sequentially, simultaneously, concurrently or chronologically staggered.

The compounds of this invention can be administered as the sole pharmaceutical agent or in combination with one or more other pharmaceutical agents where the combination causes no unacceptable adverse effects. The present invention relates also to such combinations. For example, the compounds of this invention can be combined with known chemotherapeutic agents or anti-cancer agents, e.g. anti-hyper-proliferative or other indication agents, and the like, as well as with admixtures and combinations thereof. Other indication agents include, but are not limited to, anti-angiogenic agents, mitotic inhibitors, alkylating agents, anti-metabolites, DNA-intercalating antibiotics, growth factor inhibitors, cell cycle inhibitors, enzyme inhibitors, topoisomerase inhibitors, proteasome inhibitors, biological response modifiers, or anti-hormones.

The terms “chemotherapeutic agent” and anti-cancer agent”, include but are not limited to 131I-chTNT, abarelix, abiraterone, aclarubicin, aldesleukin, alemtuzumab, alitretinoin, altretamine, aminoglutethimide, amrubicin, amsacrine, anastrozole, arglabin, arsenic trioxide, asparaginase, azacitidine, basiliximab, BAY 80-6946, BAY 1000394, BAY 86-9766 (RDEA 119), belotecan, bendamustine, bevacizumab, bexarotene, bicalutamide, bisantrene, bleomycin, bortezomib, buserelin, busulfan, cabazitaxel, calcium folinate, calcium levofolinate, capecitabine, carboplatin, carmofur, carmustine, catumaxomab, celecoxib, celmoleukin, cetuximab, chlorambucil, chlormadinone, chlormethine, cisplatin, cladribine, clodronic acid, clofarabine, crisantaspase, cyclophosphamide, cyproterone, cytarabine, dacarbazine, dactinomycin, darbepoetin alfa, dasatinib, daunorubicin, decitabine, deforolimus, degarelix, denileukin diftitox, denosumab, deslorelin, dibrospidium chloride, docetaxel, doxifluridine, doxorubicin, doxorubicin+estrone, eculizumab, edrecolomab, elliptinium acetate, eltrombopag, endostatin, enocitabine, enzastaurin, epirubicin, epitiostanol, epoetin alfa, epoetin beta, eptaplatin, eribulin, erlotinib, estradiol, estramustine, etoposide, everolimus, exemestane, fadrozole, filgrastim, fludarabine, fluorouracil, flutamide, formestane, fotemustine, fulvestrant, gallium nitrate, ganirelix, gefitinib, gemcitabine, gemtuzumab, glutoxim, goserelin, histamine dihydrochloride, histrelin, hydroxycarbamide, I-125 seeds, ibandronic acid, ibritumomab tiuxetan, idarubicin, ifosfamide, imatinib, imiquimod, improsulfan, interferon alfa, interferon beta, interferon gamma, ipilimumab, irinotecan, ixabepilone, lanreotide, lapatinib, larotaxel, lenalidomide, lenograstim, lentinan, letrozole, leuprorelin, levamisole, lisuride, lobaplatin, lomustine, lonidamine, masoprocol, medroxyprogesterone, megestrol, melphalan, mepitiostane, mercaptopurine, methotrexate, methoxsalen, Methyl aminolevulinate, methyltestosterone, mifamurtide, miltefosine, miriplatin, mitobronitol, mitoguazone, mitolactol, mitomycin, mitotane, mitoxantrone, nedaplatin, nelarabine, nilotinib, nilutamide, nimotuzumab, nimustine, nitracrine, novolimus, ofatumumab, omeprazole, oprelvekin, oxaliplatin, p53 gene therapy, paclitaxel, palifermin, palladium-103 seed, pamidronic acid, panitumumab, pazopanib, pegaspargase, PEG-epoetin beta (methoxy PEG-epoetin beta), pegfilgrastim, peginterferon alfa-2b, pemetrexed, pentazocine, pentostatin, peplomycin, perfosfamide, perifosine, picibanil, pirarubicin, plerixafor, plicamycin, poliglusam, polyestradiol phosphate, polysaccharide-K, porfimer sodium, pralatrexate, prednimustine, procarbazine, quinagolide, raloxifene, raltitrexed, ranimustine, rapamycin, razoxane, regorafenib, risedronic acid, rituximab, romidepsin, romiplostim, sagopilone, sargramostim, selumetinib, sipuleucel-T, sizofiran, sobuzoxane, sodium glycididazole, sorafenib, streptozocin, sunitinib, talaporfin, tamibarotene, tamoxifen, tasonermin, teceleukin, tegafur, tegafur+gimeracil+oteracil, temoporfin, temozolomide, temsirolimus, teniposide, testosterone, tetrofosmin, thalidomide, thiotepa, thymalfasin, tioguanine, tocilizumab, topotecan, toremifene, tositumomab, trabectedin, trametinib, trastuzumab, treosulfan, tretinoin, triciribine, trilostane, triptorelin, trofosfamide, tryptophan, ubenimex, valrubicin, vandetanib, vapreotide, vemurafenib, vinblastine, vincristine, vindesine, vinflunine, vinorelbine, vorinostat, vorozole, yttrium-90 glass microspheres, zinostatin, zinostatin stimalamer, zoledronic acid, zorubicin, zotarolimus, ARRY-162, ARRY-300, ARRY-704, AS-703026, AZD-5363, AZD-8055, BEZ-235, BGT-226, BKM-120, BYL-719, CAL-101, CC-223, CH-5132799, E-6201, GDC-0032, GDC-0068, GDC-0623, GDC-0941, GDC-0973, GDC-0980, GSK-2110183, GSK-2126458, GSK-2141795, INK128, MK-2206, OSI-027, PF-04691502, PF-05212384, PX-866, RG-7167, RO-4987655, RO-5126766, TAK-733, UCN-01, WX-554, XL-147, XL-765, ZSTK-474.

The terms “chemotherapeutic agent” and anti-cancer agent”, also include protein therapeutics such as an interferon (e.g., interferon .alpha., .beta., or .gamma.) supraagonistic monoclonal antibodies, Tuebingen, TRP-1 protein vaccine, Colostrinin, anti-FAP antibody, YH-16, gemtuzumab, infliximab, cetuximab, trastuzumab, denileukin diftitox, rituximab, thymosin alpha 1, bevacizumab, mecasermin, mecasermin rinfabate, oprelvekin, natalizumab, rhMBL, MFE-CP1+ZD-2767-P, ABT-828, ErbB2-specific immunotoxin, SGN-35, MT-103, rinfabate, AS-1402, B43-genistein, L-19 based radioimmunotherapeutics, AC-9301, NY-ESO-1 vaccine, IMC-1C11, CT-322, rhCC10, r(m)CRP, MORAb-009, aviscumine, MDX-1307, Her-2 vaccine, APC-8024, NGR-hTNF, rhH1.3, IGN-311, Endostatin, volociximab, PRO-1762, lexatumumab, SGN-40, pertuzumab, EMD-273063, L19-IL-2 fusion protein, PRX-321, CNTO-328, MDX-214, tigapotide, CAT-3888, labetuzumab, alpha-particle-emitting radioisotope-llinked lintuzumab, EM-1421, HyperAcute vaccine, tucotuzumab celmoleukin, galiximab, HPV-16-E7, Javelin-prostate cancer, Javelin—melanoma, NY-ESO-1 vaccine, EGF vaccine, CYT-004-MelQbG10, WT1 peptide, oregovomab, ofatumumab, zalutumumab, cintredekin besudotox, WX-G250, Albuferon, aflibercept, denosumab, vaccine, CTP-37, efungumab, or 131-chTNT-1/B.

The terms “chemotherapeutic agent” and anti-cancer agent”, also include monoclonal antibodies useful as the protein therapeutic such as muromonab-CD3, abciximab, edrecolomab, daclizumab, gentuzumab, alemtuzumab, ibritumomab, cetuximab, bevicizumab, efalizumab, adalimumab, omalizumab, muromomab-CD3, rituximab, daclizumab, trastuzumab, palivizumab, basiliximab, and infliximab.

Generally, the use of cytotoxic and/or cytostatic agents in combination with a compound or composition of the present invention will serve to:
(1) yield better efficacy in reducing the growth of a tumor or even eliminate the tumor as compared to administration of either agent alone,
(2) provide for the administration of lesser amounts of the administered chemotherapeutic agents,
(3) provide for a chemotherapeutic treatment that is well tolerated in the patient with fewer deleterious pharmacological complications than observed with single agent chemotherapies and certain other combined therapies,
(4) provide for treating a broader spectrum of different cancer types in mammals, especially humans,
(5) provide for a higher response rate among treated patients,
(6) provide for a longer survival time among treated patients compared to standard chemotherapy treatments,
(7) provide a longer time for tumor progression, and/or
(8) yield efficacy and tolerability results at least as good as those of the agents used alone, compared to known instances where other cancer agent combinations produce antagonistic effects.

### Methods of Sensitizing Cells to Radiation

In a distinct embodiment of the present invention, a compound of the present invention may be used to sensitize a cell to radiation. That is, treatment of a cell with a compound of the present invention prior to radiation treatment of the cell renders the cell more susceptible to DNA damage and cell death than the cell would be in the absence of any treatment with a compound of the invention. In one aspect, the cell is treated with at least one compound of the invention.

Thus, the present invention also provides a method of killing a cell, wherein a cell is administered one or more compounds of the invention in combination with conventional radiation therapy.

The present invention also provides a method of rendering a cell more susceptible to cell death, wherein the cell is treated with one or more compounds of the invention prior to the treatment of the cell to cause or induce cell death. In one aspect, after the cell is treated with one or more compounds of the invention, the cell is treated with at least one compound, or at least one method, or a combination thereof, in order to cause DNA damage for the purpose of inhibiting the function of the normal cell or killing the cell.

In one embodiment, a cell is killed by treating the cell with at least one DNA damaging agent. That is, after treating a cell with one or more compounds of the invention to sensitize the cell to cell death, the cell is treated with at least one DNA damaging agent to kill the cell. DNA damaging agents useful in the present invention include, but are not limited to, chemotherapeutic agents (e.g., cisplatinum), ionizing radiation (X-rays, ultraviolet radiation), carcinogenic agents, and mutagenic agents.

In another embodiment, a cell is killed by treating the cell with at least one method to cause or induce DNA damage. Such methods include, but are not limited to, activation of a cell signalling pathway that results in DNA damage when the pathway is activated, inhibiting of a cell signalling pathway that results in DNA damage when the pathway is inhibited, and inducing a biochemical change in a cell, wherein the change results in DNA damage. By way of a non-limiting example, a DNA repair pathway in a cell can be inhibited, thereby preventing the repair of DNA damage and resulting in an abnormal accumulation of DNA damage in a cell.

In one aspect of the invention, a compound of the invention is administered to a cell prior to the radiation or other induction of DNA damage in the cell. In another aspect of the invention, a compound of the invention is administered to a cell concomitantly with the radiation or other induction of DNA damage in the cell. In yet another aspect of the invention, a compound of the invention is administered to a cell immediately after radiation or other induction of DNA damage in the cell has begun.

In another aspect, the cell is in vitro. In another embodiment, the cell is in vivo.

As mentioned supra, the compounds of the present invention have surprisingly been found to effectively inhibit MKNK1 and may therefore be used for the treatment or prophylaxis of diseases of uncontrolled cell growth, proliferation and/or survival, inappropriate cellular immune responses, or inappropriate cellular inflammatory responses, or diseases which are accompanied with uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses is mediated by MKNK1, such as, for example, haematological tumours, solid tumours, and/or metastases thereof, e.g. leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof.

In accordance with another aspect therefore, the present invention covers a compound of general formula I, or a stereoisomer, a tautomer, an N-oxide, a hydrate, a solvate, or a salt thereof, particularly a pharmaceutically acceptable salt thereof, or a mixture of same, as described and defined herein, for use in the treatment or prophylaxis of a disease, as mentioned supra.

Another particular aspect of the present invention is therefore the use of a compound of general formula I, described supra, or a stereoisomer, a tautomer, an N-oxide, a hydrate, a solvate, or a salt thereof, particularly a pharmaceutically acceptable salt thereof, or a mixture of same, for the prophylaxis or treatment of a disease.

Another particular aspect of the present invention is therefore the use of a compound of general formula I described supra for manufacturing a pharmaceutical composition for the treatment or prophylaxis of a disease.

The diseases referred to in the two preceding paragraphs are diseases of uncontrolled cell growth, proliferation and/or survival, inappropriate cellular immune responses, or inappropriate cellular inflammatory responses, or diseases which are accompanied with uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses is mediated by MKNK1, such as, for example, haematological tumours, solid tumours, and/or metastases thereof, e.g. leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof.

The term “inappropriate” within the context of the present invention, in particular in the context of “inappropriate cellular immune responses, or inappropriate cellular inflammatory responses”, as used herein, is to be understood as preferably meaning a response which is less than, or greater than normal, and which is associated with, responsible for, or results in, the pathology of said diseases.

Preferably, the use is in the treatment or prophylaxis of diseases, wherein the diseases are haemotological tumours, solid tumours and/or metastases thereof.

### Method of Treating Hyper-Proliferative Disorders

The present invention relates to a method for using the compounds of the present invention and compositions thereof, to treat mammalian hyper-proliferative disorders. Compounds can be utilized to inhibit, block, reduce, decrease, etc., cell proliferation and/or cell division, and/or produce apoptosis. This method comprises administering to a mammal in need thereof, including a human, an amount of a compound of this invention, or a pharmaceutically acceptable salt, isomer, polymorph, metabolite, hydrate, solvate or ester thereof; etc. which is effective to treat the disorder. Hyper-proliferative disorders include but are not limited, e.g., psoriasis, keloids, and other hyperplasias affecting the skin, benign prostate hyperplasia (BPH), solid tumours, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukaemias.

Examples of breast cancer include, but are not limited to invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypophtalmic glioma, cerebellar and cerebral astrocytoma, medulloblastoma, ependymoma, as well as neuroectodermal and pineal tumour.

Tumours of the male reproductive organs include, but are not limited to prostate and testicular cancer. Tumours of the female reproductive organs include, but are not limited to endometrial, cervical, ovarian, vaginal, and vulvar cancer, as well as sarcoma of the uterus.

Tumours of the digestive tract include, but are not limited to anal, colon, colorectal, oesophageal, gallbladder, gastric, pancreatic, rectal, small-intestine, and salivary gland cancers.

Tumours of the urinary tract include, but are not limited to bladder, penile, kidney, renal pelvis, ureter, urethral and human papillary renal cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal, hypopharyngeal, nasopharyngeal, oropharyngeal cancer, lip and oral cavity cancer and squamous cell.

Lymphomas include, but are not limited to AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

These disorders have been well characterized in humans, but also exist with a similar etiology in other mammals, and can be treated by administering pharmaceutical compositions of the present invention.

The term “treating” or “treatment” as stated throughout this document is used conventionally, e.g., the management or care of a subject for the purpose of combating, alleviating, reducing, relieving, improving the condition of, etc., of a disease or disorder, such as a carcinoma.

### Methods of Treating Kinase Disorders

The present invention also provides methods for the treatment of disorders associated with aberrant mitogen extracellular kinase activity, including, but not limited to stroke, heart failure, hepatomegaly, cardiomegaly, diabetes, Alzheimer's disease, cystic fibrosis, symptoms of xenograft rejections, septic shock or asthma.

Effective amounts of compounds of the present invention can be used to treat such disorders, including those diseases (e.g., cancer) mentioned in the Background section above. Nonetheless, such cancers and other diseases can be treated with compounds of the present invention, regardless of the mechanism of action and/or the relationship between the kinase and the disorder.

The phrase “aberrant kinase activity” or “aberrant serin threonin kinase activity,” includes any abnormal expression or activity of the gene encoding the kinase or of the polypeptide it encodes. Examples of such aberrant activity, include, but are not limited to, over-expression of the gene or polypeptide; gene amplification; mutations which produce constitutively-active or hyperactive kinase activity; gene mutations, deletions, substitutions, additions, etc.

The present invention also provides for methods of inhibiting a kinase activity, especially of mitogen extracellular kinase, comprising administering an effective amount of a compound of the present invention, including salts, polymorphs, metabolites, hydrates, solvates, prodrugs (e.g.: esters) thereof, and diastereoisomeric forms thereof. Kinase activity can be inhibited in cells (e.g., in vitro), or in the cells of a mammalian subject, especially a human patient in need of treatment.

### Dose and Administration

Based upon standard laboratory techniques known to evaluate compounds useful for the treatment of hyper-proliferative disorders and angiogenic disorders, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

Preferably, the diseases of said method are haematological tumours, solid tumour and/or metastases thereof.

The compounds of the present invention can be used in particular in therapy and prevention, i.e. prophylaxis, of tumour growth and metastases, especially in solid tumours of all indications and stages with or without pre-treatment of the tumour growth.

Methods of testing for a particular pharmacological or pharmaceutical property are well known to persons skilled in the art.

The example testing experiments described herein serve to illustrate the present invention and the invention is not limited to the examples given.

### Biological Assays

Examples were tested in selected biological assays one or more times. When tested more than once, data are reported as either average values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

Examples were synthesized one or more times. When synthesized more than once, data from biological assays represent average values or median values calculated utilizing data sets obtained from testing of one or more synthetic batch.

### MKNK1 Kinase Assay

MKNK1-inhibitory activity of compounds of the present invention was quantified employing the MKNK1 TR-FRET assay as described in the following paragraphs.

A recombinant fusion protein of Glutathione-S-Transferase (GST, N-terminally) and human full-length MKNK1 (amino acids 1-424 and T344D of accession number BAA 19885.1), expressed in insect cells using baculovirus expression system and purified via glutathione sepharose affinity chromatography, was purchased from Carna Biosciences (product no 02-145) and used as enzyme. As substrate for the kinase reaction the biotinylated peptide biotin-Ahx-IKKRKLTRRKSLKG (C-terminus in amide form) was used which can be purchased e.g. form the company Biosyntan (Berlin-Buch, Germany).

For the assay 50 nL of a 100 fold concentrated solution of the test compound in DMSO was pipetted into a black low volume 384 well microtiter plate (Greiner Bio-One, Frickenhausen, Germany), 2 μL of a solution of MKNK1 in aqueous assay buffer [50 mM HEPES pH 7.5, 5 mM MgCl₂, 1.0 mM dithiothreitol, 0.005% (v/v) Nonidet-P40 (Sigma)] was added and the mixture was incubated for 15 min at 22° C. to allow pre-binding of the test compounds to the enzyme before the start of the kinase reaction. Then the kinase reaction was started by the addition of 3 μL of a solution of adenosine-tri-phosphate (ATP, 16.7 μM=> final conc. in the 5 μL assay volume is 10 μM) and substrate (0.1 μM=> final conc. in the 5 μL assay volume is 0.06 μM) in assay buffer and the resulting mixture was incubated for a reaction time of 45 min at 22° C. The concentration of MKNK1 was adjusted depending of the activity of the enzyme lot and was chosen appropriate to have the assay in the linear range, typical concentrations were in the range of 0.05 μg/ml. The reaction was stopped by the addition of 5 μL of a solution of TR-FRET detection reagents (5 nM streptavidine-XL665 [Cisbio Bioassays, Codolet, France] and 1 nM anti-ribosomal protein S6 (pSer236)-antibody from Invitrogen [#44921G] and 1 nM LANCE EU-W1024 labeled ProteinG [Perkin-Elmer, product no. AD0071]) in an aqueous EDTA-solution (100 mM EDTA, 0.1% (w/v) bovine serum albumin in 50 mM HEPES pH 7.5).

The resulting mixture was incubated for 1 h at 22° C. to allow the formation of complex between the phosphorylated biotinylated peptide and the detection reagents. Subsequently the amount of phosphorylated substrate was evaluated by measurement of the resonance energy transfer from the Eu-chelate to the streptavidine-XL. Therefore, the fluorescence emissions at 620 nm and 665 nm after excitation at 350 nm were measured in a TR-FRET reader, e.g. a Rubystar (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Usually the test compounds were tested on the same microtiterplate in 11 different concentrations in the range of 20 μM to 0.1 nM (20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100 fold concentrated solutions in DMSO by serial 1:3.4 dilutions) in duplicate values for each concentration and IC₅₀ values were calculated by a 4 parameter fit using an in house software.

### MKNK1 Kinase High ATP Assay

MKNK1-inhibitory activity at high ATP of compounds of the present invention after their preincubation with MKNK1 was quantified employing the TR-FRET-based MKNK1 high ATP assay as described in the following paragraphs.

A recombinant fusion protein of Glutathione-S-Transferase (GST, N-terminally) and human full-length MKNK1 (amino acids 1-424 and T344D of accession number BAA 19885.1), expressed in insect cells using baculovirus expression system and purified via glutathione sepharose affinity chromatography, was purchased from Carna Biosciences (product no 02-145) and used as enzyme. As substrate for the kinase reaction the biotinylated peptide biotin-Ahx-IKKRKLTRRKSLKG (C-terminus in amide form) was used, which can be purchased e.g. from the company Biosyntan (Berlin-Buch, Germany).

For the assay 50 nL of a 100 fold concentrated solution of the test compound in DMSO was pipetted into a black low volume 384 well microtiter plate (Greiner Bio-One, Frickenhausen, Germany), 2 μL of a solution of MKNK1 in aqueous assay buffer [50 mM HEPES pH 7.5, 5 mM MgCl₂, 1.0 mM dithiothreitol, 0.005% (v/v) Nonidet-P40 (Sigma)] was added and the mixture was incubated for 15 min at 22° C. to allow pre-binding of the test compounds to the enzyme before the start of the kinase reaction. Then the kinase reaction was started by the addition of 3 μL of a solution of adenosine-tri-phosphate (ATP, 3.3 mM=> final conc. in the 5 μL assay volume is 2 mM) and substrate (0.1 μM=> final conc. in the 5 μL assay volume is 0.06 μM) in assay buffer and the resulting mixture was incubated for a reaction time of 30 min at 22° C. The concentration of MKNK1 was adjusted depending of the activity of the enzyme lot and was chosen appropriate to have the assay in the linear range, typical concentrations were in the range of 0.003 μg/mL. The reaction was stopped by the addition of 5 μL of a solution of TR-FRET detection reagents (5 nM streptavidine-XL665 [Cisbio Bioassays, Codolet, France] and 1 nM anti-ribosomal protein S6 (pSer236)-antibody from Invitrogen [#44921G] and 1 nM LANCE EU-W1024 labeled ProteinG [Perkin-Elmer, product no. AD0071]) in an aqueous EDTA-solution (100 mM EDTA, 0.1% (w/v) bovine serum albumin in 50 mM HEPES pH 7.5).

The resulting mixture was incubated for 1 h at 22° C. to allow the formation of complex between the phosphorylated biotinylated peptide and the detection reagents. Subsequently the amount of phosphorylated substrate was evaluated by measurement of the resonance energy transfer from the Eu-chelate to the streptavidine-XL. Therefore, the fluorescence emissions at 620 nm and 665 nm after excitation at 350 nm were measured in a TR-FRET reader, e.g. a Rubystar (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Usually the test compounds were tested on the same microtiterplate in 11 different concentrations in the range of 20 μM to 0.1 nM (e.g. 20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100 fold concentrated solutions in DMSO by serial dilutions, the exact concentrations may vary depending on the pipettor used) in duplicate values for each concentration and IC₅₀ values were calculated. Data are presented in Table 1.

TABLE 1

| | MKNK1 |
|---|---|
| Example | IC₅₀ [nM] |
| 1 | 28.1 |
| 2 | 23.8 |
| 3 | 25.9 |
| 4 | 4.2 |
| 5 | 12.2 |
| 6 | 7.3 |
| 7 | 28.9 |
| 8 | 11.5 |
| 9 | 3.0 |
| 10 | 6.6 |
| 11 | 24.1 |
| 12 | 5.0 |
| 13 | nd |
| 14 | 2.8 |
| 15 | 28.8 |
| 16 | 52.9 |
| 17 | 32.2 |
| 18 | 11.1 |
| 19 | 5.8 |
| 20 | 7.8 |
| 21 | 4.2 |
| 22 | 5.4 |
| 23 | 10.4 |
| 24 | 10.7 |
| 25 | 13.8 |
| 26 | 38.2 |
| 27 | 20.3 |
| 28 | 9.8 |
| 29 | 24.7 |
| 30 | 50.7 |
| 31 | 59.1 |
| 32 | 49.0 |
| 33 | 90.4 |
| 34 | 2.5 |
| 35 | 2.2 |
| 36 | 0.8 |
| 37 | 4.2 |
| 38 | 1.4 |
| 39 | 1.0 |
| 40 | 2.5 |
| 41 | 1.6 |
| 42 | 2.1 |
| 43 | 1.6 |
| 44 | 0.9 |
| 45 | 0.7 |
| 46 | 4.9 |
| 47 | 11.1 |
| 48 | 6.2 |
| 49 | 0.3 |
| 50 | 2.6 |
| 51 | 0.5 |
| 52 | 6.0 |
| 53 | 0.5 |
| 54 | 6.9 |
| 55 | 9.7 |
| 56 | 8.1 |
| 57 | 15.0 |
| 58 | 17.3 |
| 59 | 2.3 |
| 60 | 7.7 |
| 61 | 120.0 |
| 62 | 12.0 |
| 63 | 14.6 |
| 64 | 4.2 |
| 65 | 2.8 |
| 66 | 8.3 |
| 67 | 15.2 |
| 68 | 10.1 |
| 69 | 19.8 |
| 70 | 40.7 |
| 71 | 43.2 |
| 72 | 16.0 |
| 73 | 144.0 |
| 74 | 140.0 |
| 75 | 61.0 |
| 76 | 71.5 |
| 77 | 23.1 |
| 78 | 7.4 |
| 79 | 1350.0 |
| 80 | 23.9 |
| 81 | 16.1 |
| 82 | 3.2 |
| 83 | 6.9 |
| 84 | 3.6 |
| 85 | 4.5 |
| 86 | 7.1 |
| 87 | 10.5 |
| 88 | 8.0 |
| 89 | 5.9 |
| 90 | 5.2 |
| 91 | 3.1 |
| 92 | 15.4 |
| 93 | 13.5 |
| 94 | 8.2 |
| 95 | 18.0 |
| 96 | 18.8 |
| 97 | 47.6 |
| 98 | 15.8 |
| 99 | 14.5 |
| 100 | 3.7 |
| 101 | 4.6 |
| 102 | 27.0 |
| 103 | 35.8 |
| 104 | 28.7 |
| 105 | 18.1 |
| 106 | 20.4 |
| 107 | 13.3 |
| 108 | 0.7 |
| 109 | 0.7 |
| 110 | 0.4 |
| 111 | 1.3 |
| 112 | 4.6 |
| 113 | 3.3 |
| 114 | 4.6 |
| 115 | 6.9 |
| 116 | 2.1 |
| 117 | 1.4 |
| 118 | 8.1 |
| 119 | 67.7 |
| 120 | 14.5 |
| 121 | 30.1 |
| 122 | 237.0 |
| 123 | 230.0 |
| 124 | 65.9 |
| 125 | 29.2 |
| 126 | 171.0 |
| 127 | 76.8 |
| 128 | 621.0 |
| 129 | 4290.0 |
| 130 | 212.0 |
| 131 | 6.7 |
| 132 | 23.4 |
| 133 | 150.0 |
| 134 | 10.8 |
| 135 | 7.3 |
| 136 | 0.5 |
| 137 | 203.0 |
| 138 | 114.0 |
| 139 | 4.7 |
| 140 | 2.4 |
| 141 | 1.2 |
| 142 | 11.8 |
| 143 | 0.7 |

### MKNK 2 Kinase High ATP Assay

MKNK 2-inhibitory activity at high ATP of compounds of the present invention after their preincubation with MKNK 2 was quantified employing the TR-FRET-based MKNK 2 high ATP assay as described in the following paragraphs.

A recombinant fusion protein of Glutathione-S-Transferase (GST, N-terminally) and human full-length MKNK 2 (Genbank accession number NP_060042.2), expressed in insect cells using baculovirus expression system, purified via glutathione sepharose affinity chromatography, and activated in vitro with MAPK12, was purchased from Invitrogen (product no PV5608) and used as enzyme. As substrate for the kinase reaction the biotinylated peptide biotin-Ahx-IKKRKLTRRKSLKG (C-terminus in amide form) was used which can be purchased e.g. form the company Biosyntan (Berlin-Buch, Germany). For the assay 50 nl of a 100 fold concentrated solution of the test compound in DMSO was pipetted into a black low volume 384 well microtiter plate (Greiner Bio-One, Frickenhausen, Germany), 2 μl of a solution of MKNK 2 in aqueous assay buffer [50 mM HEPES pH 7.5, 5 mM MgCl₂, 1.0 mM dithiothreitol, 0.005% (v/v) Nonidet-P40 (G-Biosciences, St. Louis, USA)] was added and the mixture was incubated for 15 min at 22° C. to allow pre-binding of the test compounds to the enzyme before the start of the kinase reaction. Then the kinase reaction was started by the addition of 3 μl of a solution of adenosine-tri-phosphate (ATP, 3.3 mM=> final conc. in the 5 μl assay volume is 2 mM) and substrate (0.1 μM=> final conc. in the 5 μl assay volume is 0.06 μM) in assay buffer and the resulting mixture was incubated for a reaction time of 30 min at 22° C. The concentration of MKNK 2 was adjusted depending of the activity of the enzyme lot and was chosen appropriate to have the assay in the linear range, typical concentrations were in the range of 0.0045 μg/ml. The reaction was stopped by the addition of 5 μl of a solution of TR-FRET detection reagents (5 nM streptavidine-XL665 [Cisbio Bioassays, Codolet, France] and 1 nM anti-ribosomal protein S6 (pSer236)-antibody from Invitrogen [#44921G] and 1 nM LANCE EU-W1024 labeled ProteinG [Perkin-Elmer, product no. AD0071]) in an aqueous EDTA-solution (100 mM EDTA, 0.1% (w/v) bovine serum albumin in 50 mM HEPES pH 7.5).

The resulting mixture was incubated for 1 h at 22° C. to allow the formation of complex between the phosphorylated biotinylated peptide and the detection reagents. Subsequently the amount of phosphorylated substrate was evaluated by measurement of the resonance energy transfer from the Eu-chelate to the streptavidine-XL665. Therefore, the fluorescence emissions at 620 nm and 665 nm after excitation at 350 nm were measured in a TR-FRET reader, e.g. a Pherastar (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Usually the test compounds were tested on the same microtiterplate in 11 different concentrations in the range of 20 μM to 0.1 nM (e.g. 20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100 fold concentrated solutions in DMSO by serial dilutions, the exact concentrations may vary depending on the pipettor used) in duplicate values for each concentration and IC₅₀ values were calculated.

### EGFR Kinase Assay

EGFR inhibitory activity of compounds of the present invention was quantified employing the TR-FRET based EGFR assay as described in the following paragraphs.

Epidermal Growth Factor Receptor (EGFR) affinity purified from human carcinoma A431 cells (Sigma-Aldrich, #E3641) was used as kinase. As substrate for the kinase reaction the biotinylated peptide biotin-Ahx-AEEEEYFELVAKKK (C-terminus in amid form) was used which can be purchased e.g. form the company Biosynthan GmbH (Berlin-Buch, Germany).

For the assay 50 nL of a 100 fold concentrated solution of the test compound in DMSO was pipetted into a black low volume 384 well microtiter plate (Greiner Bio-One, Frickenhausen, Germany), 2 μL of a solution of EGFR in aqueous assay [50 mM Hepes/HCl pH 7.0, 1 mM MgCl₂, 5 mM MnCl₂, 0.5 mM activated sodium ortho-vanadate, 0.005% (v/v) Tween-20] were added and the mixture was incubated for 15 min at 22° C. to allow pre-binding of the test compounds to the enzyme before the start of the kinase reaction. Then the kinase reaction was started by the addition of 3 μL of a solution of adenosine-tri-phosphate (ATP, 16.7 μM=> final conc. in the 5 μL assay volume is 10 μM) and substrate (1.67 μM=> final conc. in the 5 μL assay volume is 1 μM) in assay buffer and the resulting mixture was incubated for a reaction time of 30 min at 22° C. The concentration of EGFR was adjusted depending of the activity of the enzyme lot and was chosen appropriate to have the assay in the linear range, typical concentration were in the range of 3 U/ml. The reaction was stopped by the addition of 5 μl of a solution of HTRF detection reagents (0.1 μM streptavidine-XL665 [Cis Biointernational] and 1 nM PT66-Tb-Chelate, an terbium-chelate labelled anti-phospho-tyrosine antibody from Cis Biointernational [instead of the PT66-Tb-chelate PT66-Eu-Cryptate from Perkin Elmer can also be used]) in an aqueous EDTA-solution (80 mM EDTA, 0.2% (w/v) bovine serum albumin in 50 mM HEPES pH 7.5).

The resulting mixture was incubated 1 h at 22° C. to allow the binding of the biotinylated phosphorylated peptide to the streptavidine-XL665 and the PT66-Eu-Chelate. Subsequently the amount of phosphorylated substrate was evaluated by measurement of the resonance energy transfer from the PT66-Eu-Chelate to the streptavidine-XL665. Therefore, the fluorescence emissions at 620 nm and 665 nm after excitation at 337 nm were measured in a HTRF reader, e.g. a Pherastar (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Usually the test compounds were tested on the same microtiterplate in 11 different concentrations in the range of 20 μM to 0.1 nM (e.g. 20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100 fold concentrated solutions in DMSO by serial dilutions, the exact concentrations may vary depending on the pipettor used) in duplicate values for each concentration and IC₅₀ values were calculated.

### CDK2/CycE Kinase Assay

CDK2/CycE inhibitory activity of compounds of the present invention can be quantified employing the CDK2/CycE TR-FRET assay as described in the following paragraphs.

Recombinant fusion proteins of GST and human CDK2 and of GST and human CycE, expressed in insect cells (Sf9) and purified by Glutathion-Sepharose affinity chromatography, can be purchased from ProQinase GmbH (Freiburg, Germany). As substrate for the kinase reaction biotinylated peptide biotin-Ttds-YISPLKSPYKISEG (C-terminus in amid form) can be used which can be purchased e.g. from the company JERINI peptide technologies (Berlin, Germany).

For the assay 50 nL of a 100 fold concentrated solution of the test compound in DMSO is pipetted into a black low volume 384 well microtiter plate (Greiner Bio-One, Frickenhausen, Germany), 2 μL of a solution of CDK2/CycE in aqueous assay buffer [50 mM Tris/HCl pH 8.0, 10 mM MgCl₂, 1.0 mM dithiothreitol, 0.1 mM sodium ortho-vanadate, 0.01% (v/v) Nonidet-P40 (Sigma)] are added and the mixture is incubated for 15 min at 22° C. to allow pre-binding of the test compounds to the enzyme before the start of the kinase reaction. Then the kinase reaction is started by the addition of 3 μL of a solution of adenosine-tri-phosphate (ATP, 16.7 μM=> final conc. in the 5 μL assay volume is 10 μM) and substrate (1.25 μM=> final conc. in the 5 μL assay volume is 0.75 μM) in assay buffer and the resulting mixture is incubated for a reaction time of 25 min at 22° C. The concentration of CDK2/CycE is adjusted depending of the activity of the enzyme lot and is chosen appropriate to have the assay in the linear range, typical concentrations ae in the range of 130 ng/ml. The reaction is stopped by the addition of 5 μL of a solution of TR-FRET detection reagents (0.2 μM streptavidine-XL665 [Cisbio Bioassays, Codolet, France] and 1 nM anti-RB(pSer807/pSer811)-antibody from BD Pharmingen [#558389] and 1.2 nM LANCE EU-W1024 labeled anti-mouse IgG antibody [Perkin-Elmer, product no. AD0077, as an alternative a Terbium-cryptate-labeled anti-mouse IgG antibody from Cisbio Bioassays can be used]) in an aqueous EDTA-solution (100 mM EDTA, 0.2% (w/v) bovine serum albumin in 100 mM HEPES/NaOH pH 7.0).

The resulting mixture is incubated 1 h at 22° C. to allow the formation of complex between the phosphorylated biotinylated peptide and the detection reagents. Subsequently the amount of phosphorylated substrate is evaluated by measurement of the resonance energy transfer from the Eu-chelate to the streptavidine-XL. Therefore, the fluorescence emissions at 620 nm and 665 nm after excitation at 350 nm is measured in a TR-FRET reader, e.g. a Rubystar (BMG Labtechnologies, Offenburg, Germany) or a Viewlux (Perkin-Elmer). The ratio of the emissions at 665 nm and at 622 nm is taken as the measure for the amount of phosphorylated substrate. The data are normalised (enzyme reaction without inhibitor=0% inhibition, all other assay components but no enzyme=100% inhibition). Usually the test compounds are tested on the same microtiterplate in 11 different concentrations in the range of 20 μM to 0.1 nM (20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100 fold concentrated solutions in DMSO by serial 1:3.4 dilutions) in duplicate values for each concentration and IC₅₀ values are calculated.

### PDGFRB Kinase Assay

PDGFRβ inhibitory activity of compounds of the present invention can be quantified employing the PDGFRβ HTRF assay as described in the following paragraphs.

As kinase, a GST-His fusion protein containing a C-terminal fragment of human PDGFRB (amino acids 561-1106, expressed in insect cells [SF9] and purified by affinity chromatography, purchased from Proqinase [Freiburg i. Brsg., Germany] is used. As substrate for the kinase reaction the biotinylated poly-Glu, Tyr (4:1) copolymer (#61GT0BLA) from Cis Biointernational (Marcoule, France) is used.

For the assay 50 nL of a 100 fold concentrated solution of the test compound in DMSO is pipetted into a black low volume 384 well microtiter plate (Greiner Bio-One, Frickenhausen, Germany), 2 μL of a solution of PDGFRB in aqueous assay buffer [50 mM HEPES/NaOH pH 7.5, 10 mM MgCl₂, 2.5 mM dithiothreitol, 0.01% (v/v) Triton-X100 (Sigma)] are added and the mixture was incubated for 15 min at 22° C. to allow pre-binding of the test compounds to the enzyme before the start of the kinase reaction. Then the kinase reaction is started by the addition of 3 μL of a solution of adenosine-tri-phosphate (ATP, 16.7 μM=> final conc. in the 5 μL assay volume is 10 μM) and substrate (2.27 μg/ml=> final conc. in the 5 μL assay volume is 1.36 μg/ml [30 nM]) in assay buffer and the resulting mixture is incubated for a reaction time of 25 min at 22° C. The concentration of PDGFRB in the assay is adjusted depending of the activity of the enzyme lot and is chosen appropriate to have the assay in the linear range, typical enzyme concentrations are in the range of about 125 μg/μL (final conc. in the 5 μL assay volume). The reaction is stopped by the addition of 5 μL of a solution of HTRF detection reagents (200 nM streptavidine-XLent [Cis Biointernational] and 1.4 nM PT66-Eu-Chelate, an europium-chelate labelled anti-phospho-tyrosine antibody from Perkin Elmer [instead of the PT66-Eu-chelate PT66-Tb-Cryptate from Cis Biointernational can also be used]) in an aqueous EDTA-solution (100 mM EDTA, 0.2% (w/v) bovine serum albumin in 50 mM HEPES/NaOH pH 7.5).

The resulting mixture is incubated 1 h at 22° C. to allow the binding of the biotinylated phosphorylated peptide to the streptavidine-XLent and the PT66-Eu-Chelate. Subsequently the amount of phosphorylated substrate is evaluated by measurement of the resonance energy transfer from the PT66-Eu-Chelate to the streptavidine-XLent. Therefore, the fluorescence emissions at 620 nm and 665 nm after excitation at 350 nm is measured in a HTRF reader, e.g. a Rubystar (BMG Labtechnologies, Offenburg, Germany) or a Viewlux (Perkin-Elmer). The ratio of the emissions at 665 nm and at 622 nm is taken as the measure for the amount of phosphorylated substrate. The data are normalised (enzyme reaction without inhibitor=0% inhibition, all other assay components but no enzyme=100% inhibition). Normally test compound are tested on the same microtiter plate at 10 different concentrations in the range of 20 μM to 1 nM (20 μM, 6.7 μM, 2.2 μM, 0.74 μM, 0.25 μM, 82 nM, 27 nM, 9.2 nM, 3.1 nM and 1 nM, dilution series prepared before the assay at the level of the 100 fold conc. stock solutions by serial 1:3 dilutions) in duplicate values for each concentration and IC₅₀ values are calculated.

### Fyn Kinase Assay

C-terminally His6-tagged human recombinant kinase domain of the human T-Fyn expressed in baculovirus infected insect cells (purchased from Invitrogen, P3042) is used as kinase. As substrate for the kinase reaction the biotinylated peptide biotin-KVEKIGEGTYGVV (C-terminus in amid form) is used which can be purchased e.g. form the company Biosynthan GmbH (Berlin-Buch, Germany).

For the assay 50 nL of a 100 fold concentrated solution of the test compound in DMSO is pipetted into a black low volume 384 well microtiter plate (Greiner Bio-One, Frickenhausen, Germany), 2 μL of a solution of T-Fyn in aqueous assay buffer [25 mM Tris/HCl pH 7.2, 25 mM MgCl₂, 2 mM dithiothreitol, 0.1% (w/v) bovine serum albumin, 0.03% (v/v) Nonidet-P40 (Sigma)]. are added and the mixture is incubated for 15 min at 22° C. to allow pre-binding of the test compounds to the enzyme before the start of the kinase reaction. Then the kinase reaction is started by the addition of 3 μL of a solution of adenosine-tri-phosphate (ATP, 16.7 μM=> final conc. in the 5 μL assay volume is 10 μM) and substrate (2 μM=> final conc. in the 5 μL assay volume is 1.2 μM) in assay buffer and the resulting mixture is incubated for a reaction time of 60 min at 22° C. The concentration of Fyn is adjusted depending of the activity of the enzyme lot and is chosen appropriate to have the assay in the linear range, typical concentration was 0.13 nM. The reaction is stopped by the addition of 5 μL of a solution of HTRF detection reagents (0.2 μM streptavidine-XL [Cisbio Bioassays, Codolet, France) and 0.66 nM PT66-Eu-Chelate, an europium-chelate labelled anti-phospho-tyrosine antibody from Perkin Elmer [instead of the PT66-Eu-chelate PT66-Tb-Cryptate from Cisbio Bioassays can also be used]) in an aqueous EDTA-solution (125 mM EDTA, 0.2% (w/v) bovine serum albumin in 50 mM HEPES/NaOH pH 7.0).

The resulting mixture is incubated 1 h at 22° C. to allow the binding of the biotinylated phosphorylated peptide to the streptavidine-XL and the PT66-Eu-Chelate. Subsequently the amount of phosphorylated substrate is evaluated by measurement of the resonance energy transfer from the PT66-Eu-Chelate to the streptavidine-XL. Therefore, the fluorescence emissions at 620 nm and 665 nm after excitation at 350 nm is measured in a HTRF reader, e.g. a Rubystar (BMG Labtechnologies, Offenburg, Germany) or a Viewlux (Perkin-Elmer). The ratio of the emissions at 665 nm and at 622 nm is taken as the measure for the amount of phosphorylated substrate. The data are normalised (enzyme reaction without inhibitor=0% inhibition, all other assay components but no enzyme=100% inhibition). Normally test compounds are tested on the same microtiter plate at 10 different concentrations in the range of 20 μM to 1 nM (20 μM, 6.7 μM, 2.2 μM, 0.74 μM, 0.25 μM, 82 nM, 27 nM, 9.2 nM, 3.1 nM and 1 nM, dilution series prepared before the assay at the level of the 100 fold conc. stock solutions by serial 1:3 dilutions) in duplicate values for each concentration and IC₅₀ values are calculated.

### Flt4 Kinase Assay

Flt4 inhibitory activity of compounds of the present invention can be quantified employing the Flt4 TR-FRET assay as described in the following paragraphs.

As kinase, a GST-His fusion protein containing a C-terminal fragment of human Flt4 (amino acids 799-1298, expressed in insect cells [SF9] and purified by affinity chromatography, purchased from Proqinase [Freiburg i. Brsg., Germany] is used. As substrate for the kinase reaction the biotinylated peptide Biotin-Ahx-GGEEEEYFELVKKKK (C-terminus in amide form, purchased from Biosyntan, Berlin-Buch, Germany) is used.

For the assay 50 nL of a 100 fold concentrated solution of the test compound in DMSO was pipetted into a black low volume 384 well microtiter plate (Greiner Bio-One, Frickenhausen, Germany), 2 μL of a solution of Flt4 in aqueous assay buffer [25 mM HEPES pH 7.5, 10 mM MgCl₂, 2 mM dithiothreitol, 0.01% (v/v) Triton-X100 (Sigma), 0.5 mM EGTA, and 5 mM β-phospho-glycerol] are added and the mixture is incubated for 15 min at 22° C. to allow pre-binding of the test compounds to the enzyme before the start of the kinase reaction.

Then the kinase reaction is started by the addition of 3 μL of a solution of adenosine-tri-phosphate (ATP, 16.7 μM=> final conc. in the 5 μL assay volume is 10 μM) and substrate (1.67 μM=> final conc. in the 5 μL assay volume is 1 μM) in assay buffer and the resulting mixture is incubated for a reaction time of 45 min at 22° C. The concentration of Flt4 in the assay is adjusted depending of the activity of the enzyme lot and was chosen appropriate to have the assay in the linear range, typical enzyme concentrations are in the range of about 120 μg/μL (final conc. in the 5 μL assay volume). The reaction is stopped by the addition of 5 μL of a solution of HTRF detection reagents (200 nM streptavidine-XL665 [Cis Biointernational] and 1 nM PT66-Tb-Cryptate, an terbium-cryptate labelled anti-phospho-tyrosine antibody from Cisbio Bioassays (Codolet, France) in an aqueous EDTA-solution (50 mM EDTA, 0.2% (w/v) bovine serum albumin in 50 mM HEPES pH 7.5).

The resulting mixture is incubated 1 h at 22° C. to allow the binding of the biotinylated phosphorylated peptide to the streptavidine-XL665 and the PT66-Tb-Cryptate. Subsequently the amount of phosphorylated substrate is evaluated by measurement of the resonance energy transfer from the PT66-Tb-Cryptate to the streptavidine-XL665. Therefore, the fluorescence emissions at 620 nm and 665 nm after excitation at 350 nm is measured in a HTRF reader, e.g. a Rubystar (BMG Labtechnologies, Offenburg, Germany) or a Viewlux (Perkin-Elmer). The ratio of the emissions at 665 nm and at 622 nm is taken as the measure for the amount of phosphorylated substrate. The data are normalised (enzyme reaction without inhibitor=0% inhibition, all other assay components but no enzyme=100% inhibition). Normally test compound are tested on the same microtiter plate at 10 different concentrations in the range of 20 μM to 1 nM (20 μM, 6.7 μM, 2.2 μM, 0.74 μM, 0.25 μM, 82 nM, 27 nM, 9.2 nM, 3.1 nM and 1 nM, dilution series prepared before the assay at the level of the 100 fold conc. stock solutions by serial 1:3 dilutions) in duplicate values for each concentration and IC₅₀ values are calculated.

### TrkA Kinase Assay

TrkA inhibitory activity of compounds of the present invention can be quantified employing the TrkA HTRF assay as described in the following paragraphs.

As kinase, a GST-His fusion protein containing a C-terminal fragment of human TrkA (amino acids 443-796, expressed in insect cells [SF9] and purified by affinity chromatography, purchased from Proqinase [Freiburg i. Brsg., Germany] is used. As substrate for the kinase reaction the biotinylated poly-Glu, Tyr (4:1) copolymer (#61GTOBLA) from Cis Biointernational (Marcoule, France) is used.

For the assay 50 nL of a 100 fold concentrated solution of the test compound in DMSO is pipetted into a black low volume 384 well microtiter plate (Greiner Bio-One, Frickenhausen, Germany), 2 μL of a solution of TrkA in aqueous assay buffer [8 mM MOPS/HCl pH 7.0, 10 mM MgCl₂, 1 mM dithiothreitol, 0.01% (v/v) NP-40 (Sigma), 0.2 mM EDTA] are added and the mixture was incubated for 15 min at 22° C. to allow pre-binding of the test compounds to the enzyme before the start of the kinase reaction. Then the kinase reaction is started by the addition of 3 μL of a solution of adenosine-tri-phosphate (ATP, 16.7 μM=> final conc. in the 5 μL assay volume is 10 μM) and substrate (2.27 μg/ml=> final conc. in the 5 μL assay volume is 1.36 μg/ml [30 nM]) in assay buffer and the resulting mixture is incubated for a reaction time of 60 min at 22° C. The concentration of TrkA in the assay is adjusted depending of the activity of the enzyme lot and is chosen appropriate to have the assay in the linear range, typical enzyme concentrations are in the range of about 20 μg/μL (final conc. in the 5 μL assay volume). The reaction is stopped by the addition of 5 μL of a solution of HTRF detection reagents (30 nM streptavidine-XL665 [Cis Biointernational] and 1.4 nM PT66-Eu-Chelate, an europium-chelate labelled anti-phospho-tyrosine antibody from Perkin Elmer [instead of the PT66-Eu-chelate PT66-Tb-Cryptate from Cis Biointernational can also be used]) in an aqueous EDTA-solution (100 mM EDTA, 0.2% (w/v) bovine serum albumin in 50 mM HEPES/NaOH pH 7.5).

The resulting mixture is incubated 1 h at 22° C. to allow the binding of the biotinylated phosphorylated peptide to the streptavidine-XL665 and the PT66-Eu-Chelate. Subsequently the amount of phosphorylated substrate is evaluated by measurement of the resonance energy transfer from the PT66-Eu-Chelate to the streptavidine-XL665. Therefore, the fluorescence emissions at 620 nm and 665 nm after excitation at 350 nm is measured in a HTRF reader, e.g. a Rubystar (BMG Labtechnologies, Offenburg, Germany) or a Viewlux (Perkin-Elmer). The ratio of the emissions at 665 nm and at 622 nm is taken as the measure for the amount of phosphorylated substrate. The data are normalised (enzyme reaction without inhibitor=0% inhibition, all other assay components but no enzyme=100% inhibition). Normally test compound are tested on the same microtiter plate at 10 different concentrations in the range of 20 μM to 1 nM (20 μM, 6.7 μM, 2.2 μM, 0.74 μM, 0.25 μM, 82 nM, 27 nM, 9.2 nM, 3.1 nM and 1 nM, dilution series prepared before the assay at the level of the 100 fold conc. stock solutions by serial 1:3 dilutions) in duplicate values for each concentration and IC₅₀ values are calculated.

AlphaScreen SureFire eIF4E Ser209 Phosphorylation Assay

The AlphaScreen SureFire eIF4E Ser209 phoshorylation assay can be used to measure the phosphorylation of endogenous eIF4E in cellular lysates. The AlphaScreen SureFire technology allows the detection of phosphorylated proteins in cellular lysates. In this assay, sandwich antibody complexes, which are only formed in the presence of the analyte (p-eIF4E Ser209), are captured by AlphaScreen donor and acceptor beads, bringing them into close proximity. The excitation of the donor bead provokes the release of singlet oxygen molecules that triggers a cascade of energy transfer in the Acceptor beads, resulting in the emission of light at 520-620 nm.

Surefire EIF4e Alphascreen in A549 Cells with 20% FCS Stimulation

For the assay the AlphaScreen SureFire p-eIF4E Ser209 10K Assay Kit and the AlphaScreen ProteinA Kit (for 1OK assay points) both from Perkin Elmer are used.

On day one 50.000 A549 cells are plated in a 96-well plate in 100-L per well in growth medium (DMEM/Hams' F12 with stable Glutamin, 10% FCS) and incubated at 37° C. After attachment of the cells, medium is changed to starving medium (DMEM, 0.1% FCS, without Glucose, with Glutamin, supplemented with 5 g/L Maltose). On day two, test compounds are serially diluted in 50 μL starving medium with a final DMSO concentration of 1% and are added to A549 cells in test plates at a final concentration range from as high 10 μM to as low 10 nM depending on the activities of the tested compounds. Treated cells are incubated at 37° C. for 2 h. 37 ul FCS is added to the wells (=final FCS concentration 20%) for 20 min. Then medium is removed and cells are lysed by adding 50 μL lysis buffer. Plates are then agitated on a plate shaker for 10 min. After 10 min lysis time, 41 L of the lysate is transferred to a 384 well plate (Proxiplate from Perkin Elmer) and 51 L Reaction Buffer plus Activation Buffer mix containing AlphaScreen Acceptor beads is added. Plates are sealed with TopSeal-A adhesive film, gently agitated on a plate shaker for 2 hours at room temperature. Afterwards 2 μL Dilution buffer with AlphaScreen Donor beads are added under subdued light and plates are sealed again with TopSeal-A adhesive film and covered with foil. Incubation takes place for further 2 h gently agitation at room temperature. Plates are then measured in an EnVision reader (Perkin Elmer) with the AlphaScreen program. Each data point (compound dilution) is measured as triplicate.

### Proliferation Assays

The tumor cell proliferation assay which can be used to test the compounds of the present invention involves a readout called Cell Titer-Glow® Luminescent Cell Viability Assay developed by Promega® (B. A. Cunningham, “A Growing Issue: Cell Proliferation Assays, Modern kits ease quantification of cell growth”, The Scientist 2001, 15(13), 26; S. P. Crouch et al., “The use of ATP bioluminescence as a measure of cell proliferation and cytotoxicity”, Journal of Immunological Methods 1993, 160, 81-88), that measures inhibition of cell proliferation. Generation of a luminescent signal corresponds to the amount of ATP present, which is directly proportional to the number of metabolically active (proliferating) cells.

### In Vitro Tumor Cell Proliferation Assay:

Cultivated tumour cells (MOLM-13 (human acute myeloid leukemia cells obtained from DSMZ #ACC 554), JJN-3 (human plasma cell leukemia cells obtained from DSMZ #ACC 541), Ramos (RA1) (human Burkitt's lymphoma cells obtained from ATCC #CRL-159)) are plated at a density of 2,500 cells/well (JJN-3), 3,000 cells/well (MOLM-13), 4,000 cells/well (Ramos (RA1)), in a 96-well multititer plate (Costar 3603 black/clear bottom) in 100 μL of their respective growth medium supplemented with 10% fetal calf serum. After 24 hours, the cells of one plate (zero-point plate) are measured for viability. Therefore, 70 μL/well CTG solution (Promega Cell Titer Glo solution (catalog #G755B and G756B)) is added to zero-point plate. The plates are mixed for two minutes on orbital shaker to ensure cell lysis and incubated for ten minutes at room temperature in the dark to stabilize luminescence signal. The samples are read on a VICTOR 3 plate reader. In parallel, serially test compounds are diluted in growth medium, and 50 μL of 3× dilutions/well are pipetted into the test plates (final concentrations: 0 μM, as well as in the range of 0.001-30 μM). The final concentration of the solvent dimethyl sulfoxide is 0.3-0.4%. The cells are incubated for 3 days in the presence of test substances. 105 μL/well CTG solution (Promega Cell Titer Glo solution (catalog #G755B and G756B)) is added to the test wells. The plates are mixed for 2 minutes on an orbital shaker to ensure cell lysis and incubated for 10 min at room temperature in the dark to stabilize luminescence signal. The samples are read on a VICTOR 3 plate reader. The change of cell number, in percent, is calculated by normalization of the measured values to the extinction values of the zero-point plate (=0%) and the extinction of the untreated (0 μm) cells (=100%).

| | | Cell | |
|---|---|---|---|
| | | number/ | |
| Cell line | Origin | well | Culture Medium |
| MOLM-13 (obtained | human acute | 3000 | RPMI 1640 with stable |
| from DSMZ # ACC | myeloid | | Glutamin with 10% |
| 554) | leukemia | | Fetal Bovine Serum |
| JJN-3 (obtained | human | 2500 | 45% Dulbecco's |
| from DSMZ # ACC | plasma cell | | Modified Eagle |
| 541) | leukemia | | Medium with stable |
| | | | Glutamin, 45% Iscove's |
| | | | Modified Dulbecco's |
| | | | Media with stable |
| | | | Glutamin and 10% |
| | | | Fetal Bovine Serum |
| Ramos (RA1) | human | 4000 | RPMI 1640 media |
| (obtained | Burkitt's | | with stable Glutamin |
| from ATCC | lymphoma | | with 10% Fetal Bovine |
| # CRL-159) | | | Serum |

Thus the compounds of the present invention effectively inhibit one or more kinases and are therefore suitable for the treatment or prophylaxis of diseases of uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses is mediated by MKNK, more particularly in which the diseases of uncontrolled cell growth, proliferation and/or survival, inappropriate cellular immune responses, or inappropriate cellular inflammatory responses are haemotological tumours, solid tumours and/or metastases thereof, e.g. leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof.

1. A compound of general formula I: 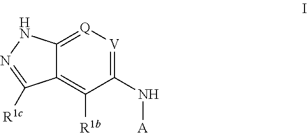
in which:
Q-V represents a group selected from: C(R^{1a})—N, N—C(R^{1a});
A represents a group selected from: 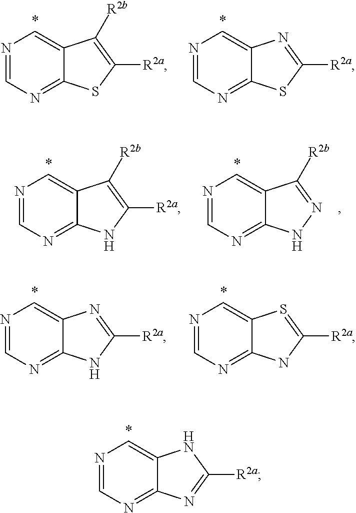
wherein * indicates the point of attachment of said groups to the rest of the molecule;
R^{1a} represents a hydrogen atom or a halogen atom or a group selected from: hydroxy-, cyano-, C₁-C₆-alkyl-, C₂-C₆-alkenyl-, C₂-C₆-alkynyl-, C₁-C₆-alkoxy-, halo-C₁-C₆-alkyl-, halo-C₁-C₆-alkoxy-, hydroxy-C₁-C₆-alkyl-, C₁-C₃-alkoxy-C₁-C₃-alkyl-, —N(R^{5b})R^{5c}, —SCF₃, —SF₅;
R^{1b} represents a hydrogen atom or a halogen atom or a group selected from: hydroxy-, cyano-, C₁-C₆-alkyl-, C₂-C₆-alkenyl-, C₂-C₆-alkynyl-, C₁-C₆-alkoxy-, halo-C₁-C₆-alkyl-, halo-C₁-C₆-alkoxy-, hydroxy-C₁-C₆-alkyl-, C₁-C₃-alkoxy-C₁-C₃-alkyl-, —N(R^{5b})R^{5c}, —SCF₃, —SF₅;
R^{1c} represents a hydrogen atom or a halogen atom or a group selected from: hydroxy-, cyano-, C₁-C₆-alkyl-, C₂-C₆-alkenyl-, C₂-C₆-alkynyl-, C₁-C₆-alkoxy-, halo-C₁-C₆-alkyl-, halo-C₁-C₆-alkoxy-, hydroxy-C₁-C₆-alkyl-, C₁-C₃-alkoxy-C₁-C₃-alkyl-, —N(R^{5b})R^{5c}, —SCF₃, —SF₅;
R^{2a} represents a hydrogen atom or a halogen atom or a group selected from:
C₁-C₆-alkyl-, C₃-C₆-cycloalkyl-, 3- to 10-membered heterocycloalkyl-, 4- to 10-membered heterocycloalkenyl-, aryl-, heteroaryl-, cyano-, —(CH₂)_{q}—U—(CH₂)ₚ—R^{3a}; wherein said C₁-C₆-alkyl-, C₃-C₆-cycloalkyl-, 3- to 10-membered heterocycloalkyl-, 4- to 10-membered heterocycloalkenyl-, aryl- or heteroaryl-group is optionally substituted, identically or differently, with 1, 2, 3, 4 or 5 R⁴ groups;
R^{2b} represents a hydrogen atom or a halogen atom or a group selected from:
C₁-C₆-alkyl-, C₃-C₆-cycloalkyl-, 3- to 10-membered heterocycloalkyl-, 4- to 10-membered heterocycloalkenyl-, aryl-, heteroaryl-, cyano-, —(CH₂)_{q}—U—(CH₂)ₚ—R^{3a}; wherein said C₁-C₆-alkyl-, C₃-C₆-cycloalkyl-, 3- to 10-membered heterocycloalkyl-, 4- to 10-membered heterocycloalkenyl-, aryl- or heteroaryl-group is optionally substituted, identically or differently, with 1, 2, 3, 4 or 5 R⁴ groups;
or
R^{2a} and R^{2b} together
represent —(CH₂)ᵣ-T-(CH₂)ₛ—
T represents a group selected from: U, —C[R^{6a}][(C(R^{6b})(R^{6c}))ₜ—U—R^{3a}];
U represents a single bond or a bivalent group selected from: —O—, —S—, —S(═O)—, —S(═O)₂—, —S(═O)—N(R^{3b})—, —N(R^{3c})—S(═O)-, —S(═O)₂—N(R^{3b})—, —N(R^{3c})—S(═O)₂—, —C(═O)—, —N(R^{3b})—, —C(═O)—O—, —O—C(═O)—, —C(═S)—O—, —O—C(═S)—, —C(═O)—N(R^{3b})—, —N(R^{3c})—C(═O)—, —N(R^{3c})—C(═O)—N(R^{3b})—, —O—C(═O)—N(R^{3b})—, —N(R^{3c})—C(═O)—O—;
R^{3a} represents a hydrogen atom or a group selected from: C₁-C₆-alkyl-, C₃-C₆-cycloalkyl-, 3- to 10-membered heterocycloalkyl-, 4- to 10-membered heterocycloalkenyl-, aryl-, heteroaryl-; wherein said C₁-C₆-alkyl-, C₃-C₆-cycloalkyl-, 3- to 10-membered heterocycloalkyl-, 4- to 10-membered heterocycloalkenyl-, aryl- or heteroaryl-group is optionally substituted, identically or differently, with 1, 2, 3, 4 or 5 R⁴ groups;
R^{3b} represents a hydrogen atom or a group selected from: C₁-C₆-alkyl-, C₃-C₆-cycloalkyl-, 3- to 10-membered heterocycloalkyl-, 4- to 10-membered heterocycloalkenyl-, aryl-, heteroaryl-; wherein said C₁-C₆-alkyl-, C₃-C₆-cycloalkyl-, 3- to 10-membered heterocycloalkyl-, 4- to 10-membered heterocycloalkenyl-, aryl- or heteroaryl-group is optionally substituted, identically or differently, with 1, 2, 3, 4 or 5 R⁴ groups;
R^{3c} represents a hydrogen atom or a group selected from: C₁-C₆-alkyl-, C₃-C₆-cycloalkyl-, 3- to 10-membered heterocycloalkyl-, 4- to 10-membered heterocycloalkenyl-, aryl-, heteroaryl-, wherein said C₁-C₆-alkyl-, C₃-C₆-cycloalkyl-, 3- to 10-membered heterocycloalkyl-, 4- to 10-membered heterocycloalkenyl-, aryl- or heteroaryl-group is optionally substituted, identically or differently, with 1, 2, 3, 4 or 5 R⁴ groups;
or
N(R^{3b})R^{3a} together
form a 3- to 10-membered heterocycloalkyl- or 4- to 10-membered heterocycloalkenyl-group, wherein said 3- to 10-membered heterocycloalkyl- or 4- to 10-membered heterocycloalkenyl-group is optionally substituted, identically or differently, with 1, 2, 3, 4 or 5 R⁴ groups;
R⁴ represents halo-, hydroxy-, oxo-(O═), cyano-, nitro-, C₁-C₆-alkyl-, C₂-C₆-alkenyl-, C₂-C₆-alkynyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R^{5c}—O—, —C(═O)—R^{5c}, —C(═O)—O—R^{5c}, —O—C(═O)—R^{5c}, —N(R^{5b})—C(═O)—R^{5c}, —N(R^{5c})—C(═O)—N(R^{5a})R^{5b}, —N(R^{5a})R^{5b}, —C(═O)—N(R^{5a})R^{5b}, R^{5c}—S—, R^{5c}—S(═O)—, R^{5c}—S(═O)₂—, —N(R^{5c})—S(═O)—R^{5b}, —S(═O)—N(R^{5a})R^{5b}, —N(R^{5c})—S(═O)₂—R^{5b}, —S(═O)₂—N(R^{5a})R^{5b}, —S(═O)═N(R^{5c})R^{5b}, —S(═O)═N(R^{5c})R^{5b} or —N═S(═O)(R^{5c})R^{5b};
R^{5a} represents a hydrogen atom or a C₁-C₆-alkyl-, C₃-C₆-cycloalkyl-, phenyl- or 3- to 10-membered heterocycloalkyl-group; wherein said C₁-C₆-alkyl-group is optionally substituted once with phenyl-;
R^{5b} represents a hydrogen atom, a C₁-C₆-alkyl-, a C₃-C₆-cycloalkyl- or a 3- to 10-membered heterocycloalkyl-group;
R^{5c} represents a hydrogen atom, a C₁-C₆-alkyl-, a C₃-C₆-cycloalkyl- or a 3- to 10-membered heterocycloalkyl-group;
or
N(R^{5a})R^{5b}
together form a 3- to 7-membered heterocycloalkyl-group;
R^{6a} represents a hydrogen atom or a group selected from: C₁-C₆-alkyl-, C₂-C₆-alkenyl-, C₂-C₆-alkynyl-; wherein said C₁-C₆-alkyl-, C₂-C₆-alkenyl- or C₂-C₆-alkynyl-group is optionally substituted, identically or differently, with 1, 2 or 3 R⁴ groups;
R^{6b} represents a hydrogen atom or a C₁-C₃-alkyl-group;
R^{6c} represents a hydrogen atom or a C₁-C₃-alkyl-group;
p represents an integer of 0, 1, 2 or 3;
q represents an integer of 0, 1, 2 or 3;
r represents an integer of 1, 2 or 3;
s represents an integer of 1, 2 or 3; and
t represents an integer of 0 or 1;
or a tautomer, an N-oxide, a hydrate, a solvate, or a salt thereof, or a mixture of same.

2. A compound according to claim 1, wherein
A represents a group selected from: 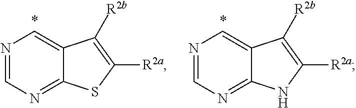
wherein * indicates the point of attachment of said groups to the rest of the molecule; or a tautomer, an N-oxide, a hydrate, a solvate, or a salt thereof, or a mixture of same.

3. A compound according to any one of claims 1 to 2, wherein:
Q-V represents C(R^{1a})—N and R^{1a} represents a hydrogen atom;
or
Q-V represents N—C(R^{1a}) and R^{1a} represents a hydrogen atom or a halogen atom or a group selected from: hydroxy-, cyano-, C₁-C₃-alkyl-, C₁-C₃-alkoxy-, halo-C₁-C₃-alkyl-, halo-C₁-C₃-alkoxy-, hydroxy-C₁-C₃-alkyl-, C₁-C₃-alkoxy-C₁-C₃-alkyl-, —N(R^{5b})R^{5c};
R^{1b} represents a hydrogen atom;
and
R^{1c} represents a hydrogen atom;
or a tautomer, an N-oxide, a hydrate, a solvate, or a salt thereof, or a mixture of same.

4. A compound according to any one of claims 1 to 3, wherein:
R^{2a} represents a hydrogen atom or a halogen atom or a group selected from: C₁-C₆-alkyl-, —(CH₂)_{q}—U—(CH₂)ₚ—R^{3a}; wherein said C₁-C₆-alkyl-group is optionally substituted, identically or differently, with 1, 2 or 3 R⁴ groups;
R^{2b} represents a hydrogen atom or a halogen atom or a group selected from: C₁-C₆-alkyl-, halo-C₁-C₃-alkyl-, —(CH₂)_{q}—U—(CH₂)ₚ—R^{3a};
wherein said C₁-C₆-alkyl-group is optionally substituted, identically or differently, with 1, 2 or 3 R⁴ groups,
with the proviso that said halo-C₁-C₃-alkyl-group does not contain more than 5 halogen atoms;
or
R^{2a} and R^{2b} together represent —(CH₂)₂-T-CH₂—;
or
R^{2a} and R^{2b} together represent —CH₂-T-(CH₂)₂—;
or a tautomer, an N-oxide, a hydrate, a solvate, or a salt thereof, or a mixture of same.

5. A compound according to any one of claims 1 to 4, wherein:
T represents —C(H)(U—R^{3a})—;
U represents a single bond or a bivalent group selected from: —C(═O)—, —N(R^{3b})—, —C(═O)—O—, —O—C(═O)—, —C(═O)—N(R^{3b})—, —N(R^{3c})—C(═O)—;
or a tautomer, an N-oxide, a hydrate, a solvate, or a salt thereof, or a mixture of same.

6. A compound according to any one of claims 1 to 5, wherein:
R^{3a} represents a hydrogen atom or a group selected from: C₁-C₆-alkyl-, C₃-C₆-cycloalkyl-, 3- to 10-membered heterocycloalkyl-; wherein said C₁-C₆-alkyl-, C₃-C₆-cycloalkyl-, 3- to 10-membered heterocycloalkyl-group is optionally substituted, identically or differently, with 1, 2 or 3 R⁴ groups;
R^{3b} represents a hydrogen atom or a C₁-C₆-alkyl-group;
or
N(R^{3b})R^{3a} together
form a 3- to 10-membered heterocycloalkyl-group, wherein said 3- to 10-membered heterocycloalkyl-group is optionally substituted, identically or differently, with 1 R⁴ group;
and
R^{3c} represents a hydrogen atom or a C₁-C₆-alkyl-;
or a tautomer, an N-oxide, a hydrate, a solvate, or a salt thereof, or a mixture of same.

7. A compound according to any one of claims 1 to 6, wherein:
R⁴ represents halo-, hydroxy-, cyano-, nitro-, C₁-C₃-alkyl-, halo-C₁-C₃-alkyl-, C₁-C₃-alkoxy-, halo-C₁-C₃-alkoxy-, hydroxy-C₁-C₃-alkyl-, C₁-C₃-alkoxy-C₁-C₃-alkyl-, R^{5c}—O, —C(═O)—R^{5c}, —C(═O)—O— R^{5c}—O—C(═O)—R^{5c}, —N(R^{5a})R^{5b}, —C(═O)—N(R^{5a})R^{5b}, R^{5c}—S(═O)₂—, —N(R⁵)—S(═O)₂R^{5b} or —S(═O)₂—N(R^{5a})R^{5b};
or a tautomer, an N-oxide, a hydrate, a solvate, or a salt thereof, or a mixture of same.

8. A compound according to any one of claims 1 to 7, wherein:
R^{5a} represents a hydrogen atom or a C₁-C₆-alkyl- or benzyl-group;
R^{5b} represents a hydrogen atom or a C₁-C₆-alkyl-group;
R^{5c} represents a hydrogen atom or a C₁-C₆-alkyl-group;
or a tautomer, an N-oxide, a hydrate, a solvate, or a salt thereof, or a mixture of same.

9. A compound according to any one of claims 1 to 8, wherein:
p represents an integer of 0 or 1;
q represents an integer of 0 or 1;
r represents an integer of 1 or 2;
s represents an integer of 1 or 2; and
t represents an integer of 0 or 1;
or a tautomer, an N-oxide, a hydrate, a solvate, or a salt thereof, or a mixture of same.

10. A compound according to claim 1, which is selected from the group consisting of:
N-(7H-pyrrolo[2,3-d]pyrimidin-4-yl)-1H-pyrazolo[3,4-b]pyridin-5-amine,
N-(7H-pyrrolo[2,3-d]pyrimidin-4-yl)-1H-pyrazolo[3,4-c]pyridin-5-amine,
N-(5-methyl-7H-pyrrolo[2,3-d]pyrimidin-4-yl)-1H-pyrazolo[3,4-b]pyridin-5-amine,
N-(5-methyl-7H-pyrrolo[2,3-d]pyrimidin-4-yl)-1H-pyrazolo[3,4-c]pyridin-5-amine,
N-(5-ethyl-7H-pyrrolo[2,3-d]pyrimidin-4-yl)-1H-pyrazolo[3,4-b]pyridin-5-amine,
N-(5-ethyl-7H-pyrrolo[2,3-d]pyrimidin-4-yl)-1H-pyrazolo[3,4-c]pyridin-5-amine,
N-(6-ethyl-5-methyl-7H-pyrrolo[2,3-d]pyrimidin-4-yl)-1H-pyrazolo[3,4-b]pyridin-5-amine,
N-(6-ethyl-5-methyl-7H-pyrrolo[2,3-d]pyrimidin-4-yl)-1H-pyrazolo[3,4-c]pyridin-5-amine,
ethyl 4-(1H-pyrazolo[3,4-b]pyridin-5-ylamino)-7H-pyrrolo[2,3-d]pyrimidine-6-carboxylate,
ethyl 4-(1H-pyrazolo[3,4-c]pyridin-5-ylamino)-7H-pyrrolo[2,3-d]pyrimidine-6-carboxylate,
4-(1H-pyrazolo[3,4-c]pyridin-5-ylamino)-7H-pyrrolo[2,3-d]pyrimidine-6-carboxylic acid,
ethyl 5-bromo-4-(1H-pyrazolo[3,4-b]pyridin-5-ylamino)-7H-pyrrolo[2,3-d]pyrimidine-6-carboxylate,
ethyl 5-bromo-4-(1H-pyrazolo[3,4-c]pyridin-5-ylamino)-7H-pyrrolo[2,3-d]pyrimidine-6-carboxylate,
5-bromo-4-(1H-pyrazolo[3,4-c]pyridin-5-ylamino)-7H-pyrrolo[2,3-d]pyrimidine-6-carboxylic acid,
N-[5-(trifluoromethyl)-7H-pyrrolo[2,3-d]pyrimidin-4-yl]-1H-pyrazolo[3,4-b]pyridin-5-amine,
N-[5-(trifluoromethyl)-7H-pyrrolo[2,3-d]pyrimidin-4-yl]-1H-pyrazolo[3,4-c]pyridin-5-amine,
[4-(1H-pyrazolo[3,4-b]pyridin-5-ylamino)-7H-pyrrolo[2,3-d]pyrimidin-5-yl]methanol,
N,N-dimethyl-4-(1H-pyrazolo[3,4-c]pyridin-5-ylamino)-7H-pyrrolo[2,3-d]pyrimidine-6-carboxamide,
[4-(1H-pyrazolo[3,4-c]pyridin-5-ylamino)-7H-pyrrolo[2,3-d]pyrimidin-6-yl](pyrrolidin-1-yl)methanone,
piperidin-1-yl[4-(1H-pyrazolo[3,4-c]pyridin-5-ylamino)-7H-pyrrolo[2,3-d]pyrimidin-6-yl]methanone,
morpholin-4-yl[4-(1H-pyrazolo[3,4-c]pyridin-5-ylamino)-7H-pyrrolo[2,3-d]pyrimidin-6-yl]methanone,
N-[2-(dimethylamino)ethyl]-4-(1H-pyrazolo[3,4-c]pyridin-5-ylamino)-7H-pyrrolo[2,3-d]pyrimidine-6-carboxamide,
(RS)—N-(propan-2-yl)-4-(1H-pyrazolo[3,4-b]pyridin-5-ylamino)-5,6,7,8-tetrahydro[1]benzothieno[2,3-d]pyrimidine-7-carboxamide,
(RS)—N-(propan-2-yl)-4-(1H-pyrazolo[3,4-c]pyridin-5-ylamino)-5,6,7,8-tetrahydro[1]benzothieno[2,3-d]pyrimidine-7-carboxamide,
(RS)-(4-methylpiperazin-1-yl)[4-(1H-pyrazolo[3,4-b]pyridin-5-ylamino)-5,6,7,8-tetrahydro[1]benzothieno[2,3-d]pyrimidin-7-yl]methanone,
(RS)-(4-methylpiperazin-1-yl)[4-(1H-pyrazolo[3,4-c]pyridin-5-ylamino)-5,6,7,8-tetrahydro[1]benzothieno[2,3-d]pyrimidin-7-yl]methanone,
(RS)—N,N-dimethyl-4-(1H-pyrazolo[3,4-b]pyridin-5-ylamino)-5,6,7,8-tetrahydro[1]benzothieno[2,3-d]pyrimidine-7-carboxamide,
(RS)—N,N-dimethyl-4-(1H-pyrazolo[3,4-c]pyridin-5-ylamino)-5,6,7,8-tetrahydro[1]benzothieno[2,3-d]pyrimidine-7-carboxamide,
(RS)—N-(propan-2-yl)-4-(1H-pyrazolo[3,4-b]pyridin-5-ylamino)-6,7,8,9-tetrahydro-5H-pyrimido[4,5-b]indole-6-carboxamide,
(RS)—N,N-dimethyl-4-(1H-pyrazolo[3,4-b]pyridin-5-ylamino)-6,7,8,9-tetrahydro-5H-pyrimido[4,5-b]indole-6-carboxamide,
(RS)—N,N-dimethyl-4-(1H-pyrazolo[3,4-c]pyridin-5-ylamino)-6,7,8,9-tetrahydro-5H-pyrimido[4,5-b]indole-6-carboxamide,
(RS)-(4-methylpiperazin-1-yl)[4-(1H-pyrazolo[3,4-b]pyridin-5-ylamino)-6,7,8,9-tetrahydro-5H-pyrimido[4,5-b]indol-6-yl]methanone,
(RS)-(4-methylpiperazin-1-yl)[4-(1H-pyrazolo[3,4-c]pyridin-5-ylamino)-6,7,8,9-tetrahydro-5H-pyrimido[4,5-b]indol-6-yl]methanone,
(7S)-4-[(6-methoxy-1H-pyrazolo[3,4-b]pyridin-5-yl)amino]-N-methyl-N-(3,3,3-trifluoropropyl)-5,6,7,8-tetrahydro[1]benzothieno[2,3-d]pyrimidine-7-carboxamide,
N-[3-(dimethylamino)-3-oxopropyl]-N-methyl-7-(1H-pyrazolo[3,4-c]pyridin-5-ylamino)[1,3]thiazolo[5,4-d]pyrimidine-2-carboxamide,
N-[3-(dimethylamino)-3-oxopropyl]-7-[(6-methoxy-1H-pyrazolo[3,4-b]pyridin-5-yl)amino]-N-methyl[1,3]thiazolo[5,4-d]pyrimidine-2-carboxamide,
(7S)-4-[(6-hydroxy-1H-pyrazolo[3,4-b]pyridin-5-yl)amino]-N-methyl-N-(3,3,3-trifluoropropyl)-5,6,7,8-tetrahydro[1]benzothieno[2,3-d]pyrimidine-7-carboxamide,
[3-(dimethylamino)azetidin-1-yl][(7S)-4-(1H-pyrazolo[3,4-c]pyridin-5-ylamino)-5,6,7,8-tetrahydro[1]benzothieno[2,3-d]pyrimidin-7-yl]methanone,
[3-(dimethylamino)azetidin-1-yl]{(7S)-4-[(6-methoxy-1H-pyrazolo[3,4-b]pyridin-5-yl)amino]-5,6,7,8-tetrahydro[1]benzothieno[2,3-d]pyrimidin-7-yl}methanone,
[3-(dimethylamino)azetidin-1-yl][(7S)-4-(1H-pyrazolo[3,4-b]pyridin-5-ylamino)-5,6,7,8-tetrahydro[1]benzothieno[2,3-d]pyrimidin-7-yl]methanone,
{(7S)-4-[(6-methoxy-1H-pyrazolo[3,4-b]pyridin-5-yl)amino]-5,6,7,8-tetrahydro[1]benzothieno[2,3-d]pyrimidin-7-y}(2-oxa-6-azaspiro[3.3]hept-6-yl)methanone,
{(7S)-4-[(6-methoxy-1H-pyrazolo[3,4-b]pyridin-5-yl)amino]-5,6,7,8-tetrahydro[1]benzothieno[2,3-d]pyrimidin-7-yl}(morpholin-4-yl)methanone,
azetidin-1-yl{(7S)-4-[(6-methoxy-1H-pyrazolo[3,4-b]pyridin-5-yl)amino]-5,6,7,8-tetrahydro[1]benzothieno[2,3-d]pyrimidin-7-yl}methanone,
[(2R,6S)-2,6-dimethylmorpholin-4-y]{(7S)-4-[(6-methoxy-1H-pyrazolo[3,4-b]pyridin-5-yl)amino]-5,6,7,8-tetrahydro[1]benzothieno[2,3-d]pyrimidin-7-yl}methanone,
(7S)-4-[(6-methoxy-1H-pyrazolo[3,4-b]pyridin-5-yl)amino]-N-methyl-N-(propan-2-yl)-5,6,7,8-tetrahydro[1]benzothieno[2,3-d]pyrimidine-7-carboxamide,
(7S)-4-[(6-methoxy-1H-pyrazolo[3,4-b]pyridin-5-yl)amino]-N-methyl-N-propyl-5,6,7,8-tetrahydro[1]benzothieno[2,3-d]pyrimidine-7-carboxamide,
(7S)—N-ethyl-4-[(6-methoxy-1H-pyrazolo[3,4-b]pyridin-5-yl)amino]-N-methyl-5,6,7,8-tetrahydro[1]benzothieno[2,3-d]pyrimidine-7-carboxamide,
{(7S)-4-[(6-methoxy-1H-pyrazolo[3,4-b]pyridin-5-yl)amino]-5,6,7,8-tetrahydro[1]benzothieno[2,3-d]pyrimidin-7-y}[(1S,4S)-2-oxa-5-azabicyclo[2.2.1]hept-5-yl]methanone,
{(7S)-4-[(6-methoxy-1H-pyrazolo[3,4-b]pyridin-5-yl)amino]-5,6,7,8-tetrahydro[1]benzothieno[2,3-d]pyrimidin-7-y}[(1R,4R)-2-oxa-5-azabicyclo[2.2.1]hept-5-yl]methanone,
{(7S)-4-[(6-methoxy-1H-pyrazolo[3,4-b]pyridin-5-yl)amino]-5,6,7,8-tetrahydro[1]benzothieno[2,3-d]pyrimidin-7-y}(4-methylpiperazin-1-yl)methanone,
[4-(dimethylamino)piperidin-1-yl]{(7S)-4-[(6-methoxy-1H-pyrazolo[3,4-b]pyridin-5-yl)amino]-5,6,7,8-tetrahydro[1]benzothieno[2,3-d]pyrimidin-7-yl}methanone,
{(7S)-4-[(6-methoxy-1H-pyrazolo[3,4-b]pyridin-5-yl)amino]-5,6,7,8-tetrahydro[1]benzothieno[2,3-d]pyrimidin-7-yl}[(3R)-3-methylmorpholin-4-yl]methanone,
{(7S)-4-[(6-methoxy-1H-pyrazolo[3,4-b]pyridin-5-yl)amino]-5,6,7,8-tetrahydro[1]benzothieno[2,3-d]pyrimidin-7-yl}[(3S)-3-methylmorpholin-4-yl]methanone,
(7S)—N-ethyl-N-(propan-2-yl)-4-(1H-pyrazolo[3,4-c]pyridin-5-ylamino)-5,6,7,8-tetrahydro[1]benzothieno[2,3-d]pyrimidine-7-carboxamide,
(7S)—N-(2-hydroxy-2-methylpropyl)-N-methyl-4-(1H-pyrazolo[3,4-c]pyridin-5-ylamino)-5,6,7,8-tetrahydro[1]benzothieno[2,3-d]pyrimidine-7-carboxamide,
(7S)—N-methyl-4-(1H-pyrazolo[3,4-c]pyridin-5-ylamino)-N-(3,3,3-trifluoropropyl)-5,6,7,8-tetrahydro[1]benzothieno[2,3-d]pyrimidine-7-carboxamide,
(7S)—N-(2-methoxyethyl)-4-[(6-methoxy-1H-pyrazolo[3,4-b]pyridin-5-yl)amino]-N-propyl-5,6,7,8-tetrahydro[1]benzothieno[2,3-d]pyrimidine-7-carboxamide,
(7S)-4-[(6-hydroxy-1H-pyrazolo[3,4-b]pyridin-5-yl)amino]-N-(2-methoxyethyl)-N-propyl-5,6,7,8-tetrahydro[1]benzothieno[2,3-d]pyrimidine-7-carboxamide,
(7S)—N-butyl-4-[(6-methoxy-1H-pyrazolo[3,4-b]pyridin-5-yl)amino]-N-methyl-5,6,7,8-tetrahydro[1]benzothieno[2,3-d]pyrimidine-7-carboxamide,
(7S)—N-butyl-4-[(6-hydroxy-1H-pyrazolo[3,4-b]pyridin-5-yl)amino]-N-methyl-5,6,7,8-tetrahydro[1]benzothieno[2,3-d]pyrimidine-7-carboxamide,
(7S)-4-[(6-methoxy-1H-pyrazolo[3,4-b]pyridin-5-yl)amino]-N,N-dimethyl-5,6,7,8-tetrahydro[1]benzothieno[2,3-d]pyrimidine-7-carboxamide,
(7S)-4-[(6-hydroxy-1H-pyrazolo[3,4-b]pyridin-5-yl)amino]-N,N-dimethyl-5,6,7,8-tetrahydro[1]benzothieno[2,3-d]pyrimidine-7-carboxamide,
azetidin-1-yl[(7S)-4-(1H-pyrazolo[3,4-c]pyridin-5-ylamino)-5,6,7,8-tetrahydro[1]benzothieno[2,3-d]pyrimidin-7-yl]methanone,
(7S)—N,N-bis(2-methoxyethyl)-4-(1H-pyrazolo[3,4-c]pyridin-5-ylamino)-5,6,7,8-tetrahydro[1]benzothieno[2,3-d]pyrimidine-7-carboxamide,
(7S)—N-(2-methoxyethyl)-N-methyl-4-(1H-pyrazolo[3,4-c]pyridin-5-ylamino)-5,6,7,8-tetrahydro[1]benzothieno[2,3-d]pyrimidine-7-carboxamide,
(7S)—N-ethyl-N-methyl-4-(1H-pyrazolo[3,4-c]pyridin-5-ylamino)-5,6,7,8-tetrahydro[1]benzothieno[2,3-d]pyrimidine-7-carboxamide,
(7S)—N,N-dimethyl-4-(1H-pyrazolo[3,4-b]pyridin-5-ylamino)-5,6,7,8-tetrahydro[1]benzothieno[2,3-d]pyrimidine-7-carboxamide,
(7S)—N,N-dimethyl-4-(1H-pyrazolo[3,4-c]pyridin-5-ylamino)-5,6,7,8-tetrahydro[1]benzothieno[2,3-d]pyrimidine-7-carboxamide,
[5-bromo-4-(1H-pyrazolo[3,4-b]pyridin-5-ylamino)-7H-pyrrolo[2,3-d]pyrimidin-6-y](piperidin-1-yl)methanone,
5-bromo-N-[2-(dimethylamino)ethyl]-4-[(6-methoxy-1H-pyrazolo[3,4-b]pyridin-5-yl)amino]-N-methyl-7H-pyrrolo[2,3-d]pyrimidine-6-carboxamide,
5-bromo-N-[2-(dimethylamino)ethyl]-4-[(6-hydroxy-1H-pyrazolo[3,4-b]pyridin-5-yl)amino]-N-methyl-7H-pyrrolo[2,3-d]pyrimidine-6-carboxamide,
{5-bromo-4-[(6-methoxy-1H-pyrazolo[3,4-b]pyridin-5-yl)amino]-7H-pyrrolo[2,3-d]pyrimidin-6-yl}[4-(dimethylamino)piperidin-1-yl]methanone,
{5-Bromo-4-[(6-hydroxy-1H-pyrazolo[3,4-b]pyridin-5-yl)amino]-7H-pyrrolo[2,3-d]pyrimidin-6-yl}[4-(dimethylamino)piperidin-1-yl]methanone,
{5-bromo-4-[(6-methoxy-1H-pyrazolo[3,4-b]pyridin-5-yl)amino]-7H-pyrrolo[2,3-d]pyrimidin-6-yl}[(3R)-3-methylmorpholin-4-yl]methanone,
{5-bromo-4-[(6-methoxy-1H-pyrazolo[3,4-b]pyridin-5-yl)amino]-7H-pyrrolo[2,3-d]pyrimidin-6-yl}(morpholin-4-y)methanone,
{5-bromo-4-[(6-hydroxy-1H-pyrazolo[3,4-b]pyridin-5-yl)amino]-7H-pyrrolo[2,3-d]pyrimidin-6-yl}(morpholin-4-y)methanone,
[5-bromo-4-(1H-pyrazolo[3,4-c]pyridin-5-ylamino)-7H-pyrrolo[2,3-d]pyrimidin-6-yl][4-(dimethylamino)piperidin-1-yl]methanone,
[5-bromo-4-(1H-pyrazolo[3,4-c]pyridin-5-ylamino)-7H-pyrrolo[2,3-d]pyrimidin-6-yl][(3R)-3-methylmorpholin-4-yl]methanone,
[5-bromo-4-(1H-pyrazolo[3,4-c]pyridin-5-ylamino)-7H-pyrrolo[2,3-d]pyrimidin-6-yl](morpholin-4-yl)methanone,
[5-bromo-4-(1H-pyrazolo[3,4-c]pyridin-5-ylamino)-7H-pyrrolo[2,3-d]pyrimidin-6-yl](piperidin-1-yl)methanone,
[5-bromo-4-(1H-pyrazolo[3,4-b]pyridin-5-ylamino)-7H-pyrrolo[2,3-d]pyrimidin-6-y][(3R)-3-methylmorpholin-4-yl]methanone,
[5-bromo-4-(1H-pyrazolo[3,4-b]pyridin-5-ylamino)-7H-pyrrolo[2,3-d]pyrimidin-6-y](morpholin-4-yl)methanone,
[5-bromo-4-(1H-pyrazolo[3,4-b]pyridin-5-ylamino)-7H-pyrrolo[2,3-d]pyrimidin-6-y][(3S)-3-methylmorpholin-4-yl]methanone,
N-[2-(dimethylamino)-2-oxoethyl]-4-(1H-pyrazolo[3,4-c]pyridin-5-ylamino)-7H-pyrrolo[2,3-d]pyrimidine-6-carboxamide,
N-[2-(dimethylamino)ethyl]-N-methyl-4-(1H-pyrazolo[3,4-c]pyridin-5-ylamino)-7H-pyrrolo[2,3-d]pyrimidine-6-carboxamide,
N-{2-[benzyl(methyl)amino]ethyl}-N-methyl-4-(1H-pyrazolo[3,4-c]pyridin-5-ylamino)-7H-pyrrolo[2,3-d]pyrimidine-6-carboxamide,
2-(2-phenylethyl)-N-(1H-pyrazolo[3,4-c]pyridin-5-y)[1,3]thiazolo[5,4-d]pyrimidin-7-amine,
(7S)—N-(propan-2-yl)-4-(1H-pyrazolo[3,4-c]pyridin-5-ylamino)-5,6,7,8-tetrahydro[1]benzothieno[2,3-d]pyrimidine-7-carboxamide,
(7S)—N-methyl-N-propyl-4-(1H-pyrazolo[3,4-c]pyridin-5-ylamino)-5,6,7,8-tetrahydro[1]benzothieno[2,3-d]pyrimidine-7-carboxamide,
1-{[(7S)-4-(1H-pyrazolo[3,4-c]pyridin-5-ylamino)-5,6,7,8-tetrahydro[1]benzothieno[2,3-d]pyrimidin-7-yl]carbonyl}azetidine-3-carbonitrile,
2-oxa-6-azaspiro[3.3]hept-6-y[(7S)-4-(1H-pyrazolo[3,4-c]pyridin-5-ylamino)-5,6,7,8-tetrahydro[1]benzothieno[2,3-d]pyrimidin-7-yl]methanone,
[(3R)-3-(dimethylamino)pyrrolidin-1-yl][(7S)-4-(1H-pyrazolo[3,4-c]pyridin-5-ylamino)-5,6,7,8-tetrahydro[1]benzothieno[2,3-d]pyrimidin-7-yl]methanone,
[(3S)-3-(dimethylamino)pyrrolidin-1-yl][(7S)-4-(1H-pyrazolo[3,4-c]pyridin-5-ylamino)-5,6,7,8-tetrahydro[1]benzothieno[2,3-d]pyrimidin-7-yl]methanone,
morpholin-4-yl[(7S)-4-(1H-pyrazolo[3,4-c]pyridin-5-ylamino)-5,6,7,8-tetrahydro[1]benzothieno[2,3-d]pyrimidin-7-yl]methanone,
[(3S)-3-methylmorpholin-4-y][(7S)-4-(1H-pyrazolo[3,4-c]pyridin-5-ylamino)-5,6,7,8-tetrahydro[1]benzothieno[2,3-d]pyrimidin-7-yl]methanone,
[(3R)-3-methylmorpholin-4-y][(7S)-4-(1H-pyrazolo[3,4-c]pyridin-5-ylamino)-5,6,7,8-tetrahydro[1]benzothieno[2,3-d]pyrimidin-7-yl]methanone,
[(2R,6S)-2,6-dimethylmorpholin-4-y][(7S)-4-(1H-pyrazolo[3,4-c]pyridin-5-ylamino)-5,6,7,8-tetrahydro[1]benzothieno[2,3-d]pyrimidin-7-yl]methanone,
(4-methylpiperazin-1-yl)[(7S)-4-(1H-pyrazolo[3,4-c]pyridin-5-ylamino)-5,6,7,8-tetrahydro[1]benzothieno[2,3-d]pyrimidin-7-yl]methanone,
[4-(dimethylamino)piperidin-1-yl][(7S)-4-(1H-pyrazolo[3,4-c]pyridin-5-ylamino)-5,6,7,8-tetrahydro[1]benzothieno[2,3-d]pyrimidin-7-yl]methanone,
(7S)—N-ethyl-N-methyl-4-(1H-pyrazolo[3,4-b]pyridin-5-ylamino)-5,6,7,8-tetrahydro[1]benzothieno[2,3-d]pyrimidine-7-carboxamide,
(7S)—N-methyl-N-propyl-4-(1H-pyrazolo[3,4-b]pyridin-5-ylamino)-5,6,7,8-tetrahydro[1]benzothieno[2,3-d]pyrimidine-7-carboxamide,
(7S)—N-methyl-4-(1H-pyrazolo[3,4-b]pyridin-5-ylamino)-N-(3,3,3-trifluoropropyl)-5,6,7,8-tetrahydro[1]benzothieno[2,3-d]pyrimidine-7-carboxamide,
(7S)—N-methyl-N-(propan-2-yl)-4-(1H-pyrazolo[3,4-b]pyridin-5-ylamino)-5,6,7,8-tetrahydro[1]benzothieno[2,3-d]pyrimidine-7-carboxamide,
(7S)—N-(2-methoxyethyl)-N-methyl-4-(1H-pyrazolo[3,4-b]pyridin-5-ylamino)-5,6,7,8-tetrahydro[1]benzothieno[2,3-d]pyrimidine-7-carboxamide,
azetidin-1-yl[(7S)-4-(1H-pyrazolo[3,4-b]pyridin-5-ylamino)-5,6,7,8-tetrahydro[1]benzothieno[2,3-d]pyrimidin-7-yl]methanone,
2-oxa-6-azaspiro[3.3]hept-6-y[(7S)-4-(1H-pyrazolo[3,4-b]pyridin-5-ylamino)-5,6,7,8-tetrahydro[1]benzothieno[2,3-d]pyrimidin-7-yl]methanone,
1-{[(7S)-4-(1H-pyrazolo[3,4-b]pyridin-5-ylamino)-5,6,7,8-tetrahydro[1]benzothieno[2,3-d]pyrimidin-7-yl]carbonyl}azetidine-3-carbonitrile,
[(3S)-3-(dimethylamino)pyrrolidin-1-yl][(7S)-4-(1H-pyrazolo[3,4-b]pyridin-5-ylamino)-5,6,7,8-tetrahydro[1]benzothieno[2,3-d]pyrimidin-7-yl]methanone,
[(3R)-3-(dimethylamino)pyrrolidin-1-yl][(7S)-4-(1H-pyrazolo[3,4-b]pyridin-5-ylamino)-5,6,7,8-tetrahydro[1]benzothieno[2,3-d]pyrimidin-7-yl]methanone,
morpholin-4-yl[(7S)-4-(1H-pyrazolo[3,4-b]pyridin-5-ylamino)-5,6,7,8-tetrahydro[1]benzothieno[2,3-d]pyrimidin-7-yl]methanone,
[(3R)-3-methylmorpholin-4-y][(7S)-4-(1H-pyrazolo[3,4-b]pyridin-5-ylamino)-5,6,7,8-tetrahydro[1]benzothieno[2,3-d]pyrimidin-7-yl]methanone,
[(3S)-3-methylmorpholin-4-y][(7S)-4-(1H-pyrazolo[3,4-b]pyridin-5-ylamino)-5,6,7,8-tetrahydro[1]benzothieno[2,3-d]pyrimidin-7-yl]methanone,
[(2R,6S)-2,6-dimethylmorpholin-4-y][(7S)-4-(1H-pyrazolo[3,4-b]pyridin-5-ylamino)-5,6,7,8-tetrahydro[1]benzothieno[2,3-d]pyrimidin-7-yl]methanone,
(4-methylpiperazin-1-yl) [(7S)-4-(1H-pyrazolo[3,4-b]pyridin-5-ylamino)-5,6,7,8-tetrahydro[1]benzothieno[2,3-d]pyrimidin-7-yl]methanone,
[4-(dimethylamino)piperidin-1-yl][(7S)-4-(1H-pyrazolo[3,4-b]pyridin-5-ylamino)-5,6,7,8-tetrahydro[1]benzothieno[2,3-d]pyrimidin-7-yl]methanone,
1-({(7S)-4-[(6-methoxy-1H-pyrazolo[3,4-b]pyridin-5-yl)amino]-5,6,7,8-tetrahydro[1]benzothieno[2,3-d]pyrimidin-7-yl}carbonyl)azetidine-3-carbonitrile,
{(7S)-4-[(6-methoxy-1H-pyrazolo[3,4-b]pyridin-5-yl)amino]-5,6,7,8-tetrahydro[1]benzothieno[2,3-d]pyrimidin-7-y}(2-oxa-6-azaspiro[3.3]hept-6-yl)methanone,
[(3R)-3-(dimethylamino)pyrrolidin-1-yl]{(7S)-4-[(6-methoxy-1H-pyrazolo[3,4-b]pyridin-5-yl)amino]-5,6,7,8-tetrahydro[1]benzothieno[2,3-d]pyrimidin-7-yl}methanone,
[(3S)-3-(dimethylamino)pyrrolidin-1-yl]{(7S)-4-[(6-methoxy-1H-pyrazolo[3,4-b]pyridin-5-yl)amino]-5,6,7,8-tetrahydro[1]benzothieno[2,3-d]pyrimidin-7-yl}methanone,
(7S)—N-ethyl-4-[(6-hydroxy-1H-pyrazolo[3,4-b]pyridin-5-yl)amino]-N-methyl-5,6,7,8-tetrahydro[1]benzothieno[2,3-d]pyrimidine-7-carboxamide,
(7S)-4-[(6-hydroxy-1H-pyrazolo[3,4-b]pyridin-5-yl)amino]-N-methyl-N-propyl-5,6,7,8-tetrahydro[1]benzothieno[2,3-d]pyrimidine-7-carboxamide,
(7S)-4-[(6-hydroxy-1H-pyrazolo[3,4-b]pyridin-5-yl)amino]-N-methyl-N-(propan-2-yl)-5,6,7,8-tetrahydro[1]benzothieno[2,3-d]pyrimidine-7-carboxamide,
{(7S)-4-[(6-hydroxy-1H-pyrazolo[3,4-b]pyridin-5-yl)amino]-5,6,7,8-tetrahydro[1]benzothieno[2,3-d]pyrimidin-7-y}[(1S,4S)-2-oxa-5-azabicyclo[2.2.1]hept-5-yl]methanone,
{(7S)-4-[(6-hydroxy-1H-pyrazolo[3,4-b]pyridin-5-yl)amino]-5,6,7,8-tetrahydro[1]benzothieno[2,3-d]pyrimidin-7-y}(4-methylpiperazin-1-yl)methanone,
[4-(dimethylamino)piperidin-1-yl]{(7S)-4-[(6-hydroxy-1H-pyrazolo[3,4-b]pyridin-5-y)amino]-5,6,7,8-tetrahydro[1]benzothieno[2,3-d]pyrimidin-7-yl}methanone,
5-bromo-N-[3-(dimethylamino)propyl]-4-(1H-pyrazolo[3,4-c]pyridin-5-ylamino)-7H-pyrrolo[2,3-d]pyrimidine-6-carboxamide,
5-bromo-N-[3-(dimethylamino)propyl]-N-methyl-4-(1H-pyrazolo[3,4-c]pyridin-5-ylamino)-7H-pyrrolo[2,3-d]pyrimidine-6-carboxamide,
5-bromo-N-[2-(dimethylamino)ethyl]-4-(1H-pyrazolo[3,4-c]pyridin-5-ylamino)-7H-pyrrolo[2,3-d]pyrimidine-6-carboxamide,
[5-bromo-4-(1H-pyrazolo[3,4-c]pyridin-5-ylamino)-7H-pyrrolo[2,3-d]pyrimidin-6-yl][(3S)-3-methylmorpholin-4-yl]methanone,
N-[2-(dimethylamino)-2-oxoethyl]-N-methyl-7-(1H-pyrazolo[3,4-c]pyridin-5-ylamino)[1,3]thiazolo[5,4-d]pyrimidine-2-carboxamide,
N,N-dimethyl-7-(1H-pyrazolo[3,4-b]pyridin-5-ylamino)[1,3]thiazolo[5,4-d]pyrimidine-2-carboxamide,
5-bromo-N-[2-(dimethylamino)ethyl]-4-(1H-pyrazolo[3,4-b]pyridin-5-ylamino)-7H-pyrrolo[2,3-d]pyrimidine-6-carboxamide,
5-bromo-N-[3-(dimethylamino)propyl]-4-(1H-pyrazolo[3,4-b]pyridin-5-ylamino)-7H-pyrrolo[2,3-d]pyrimidine-6-carboxamide,
piperidin-1-yl[7-(1H-pyrazolo[3,4-b]pyridin-5-ylamino)[1,3]thiazolo[5,4-d]pyrimidin-2-yl]methanone,
[5-bromo-4-(1H-pyrazolo[3,4-b]pyridin-5-ylamino)-7H-pyrrolo[2,3-d]pyrimidin-6-yl][4-(dimethylamino)piperidin-1-yl]methanone,
N-[2-(dimethylamino)-2-oxoethyl]-N-methyl-7-(1H-pyrazolo[3,4-b]pyridin-5-ylamino)[1,3]thiazolo[5,4-d]pyrimidine-2-carboxamide,
N-[3-(dimethylamino)-3-oxopropyl]-N-methyl-7-(1H-pyrazolo[3,4-b]pyridin-5-ylamino)[1,3]thiazolo[5,4-d]pyrimidine-2-carboxamide,
N-[2-(dimethylamino)-2-oxoethyl]-7-[(6-methoxy-1H-pyrazolo[3,4-b]pyridin-5-yl)amino]-N-methyl[1,3]thiazolo[5,4-d]pyrimidine-2-carboxamide,
5-bromo-N-[3-(dimethylamino)propyl]-4-[(6-methoxy-1H-pyrazolo[3,4-b]pyridin-5-yl)amino]-N-methyl-7H-pyrrolo[2,3-d]pyrimidine-6-carboxamide,
5-bromo-N-[3-(dimethylamino)propyl]-4-[(6-hydroxy-1H-pyrazolo[3,4-b]pyridin-5-yl)amino]-N-methyl-7H-pyrrolo[2,3-d]pyrimidine-6-carboxamide,
{7-[(6-methoxy-1H-pyrazolo[3,4-b]pyridin-5-yl)amino][1,3]thiazolo[5,4-d]pyrimidin-2-yl}(piperidin-1-yl)methanone,
{5-bromo-4-[(6-hydroxy-1H-pyrazolo[3,4-b]pyridin-5-yl)amino]-7H-pyrrolo[2,3-d]pyrimidin-6-yl}(piperidin-1-yl)methanone,
{5-bromo-4-[(6-methoxy-1H-pyrazolo[3,4-b]pyridin-5-yl)amino]-7H-pyrrolo[2,3-d]pyrimidin-6-yl}[(3S)-3-methylmorpholin-4-yl]methanone;
or a tautomer, an N-oxide, a hydrate, a solvate, or a salt thereof, or a mixture of same.

11. A method of preparing a compound of general formula I according to any one of claims 1 to 10, in which method an intermediate compound of general formula II: 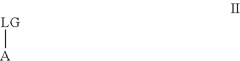
in which A is as defined in any one of claims 1 to 10 and LG represents a leaving group;
is allowed to react with an intermediate compound of general formula IV: 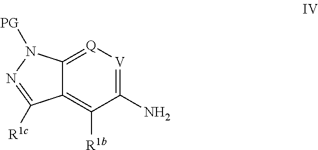
in which R^{1b}, R^{1c}, and Q-V are as defined in any one of claims 1 to 10 and PG represents a protective group or a hydrogen atom;
thus providing a compound of general formula I′ or I: 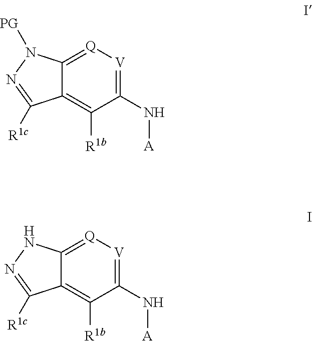
in which R^{1b}, R^{1c}, Q-V, and A are as defined in any one of claims 1 to 10, and PG represents a protective group or a hydrogen atom.

12. A compound of general formula I, or a stereoisomer, a tautomer, an N-oxide, a hydrate, a solvate, or a salt thereof, particularly a pharmaceutically acceptable salt thereof, or a mixture of same, according to any one of claims 1 to 10, for use in the treatment or prophylaxis of a disease.

13. A pharmaceutical composition comprising a compound of general formula I, or a stereoisomer, a tautomer, an N-oxide, a hydrate, a solvate, or a salt thereof, particularly a pharmaceutically acceptable salt thereof, or a mixture of same, according to any one of claims 1 to 10, and a pharmaceutically acceptable diluent or carrier.

14. A pharmaceutical combination comprising:
one or more first active ingredients selected from a compound of general formula I according to any of claims 1 to 10, and
one or more second active ingredients selected from chemotherapeutic anti-cancer agents.

15. Use of a compound of general formula I, or a stereoisomer, a tautomer, an N-oxide, a hydrate, a solvate, or a salt thereof, particularly a pharmaceutically acceptable salt thereof, or a mixture of same, according to any one of claims 1 to 10, for the prophylaxis or treatment of a disease.

16. Use of a compound of general formula I, or a stereoisomer, a tautomer, an N-oxide, a hydrate, a solvate, or a salt thereof, particularly a pharmaceutically acceptable salt thereof, or a mixture of same, according to any one of claims 1 to 10, for the preparation of a medicament for the prophylaxis or treatment of a disease.

17. Use according to claims 12, 15 or 16, wherein said disease is a disease of uncontrolled cell growth, proliferation and/or survival, an inappropriate cellular immune response, or an inappropriate cellular inflammatory response, particularly in which the uncontrolled cell growth, proliferation and/or survival, inappropriate cellular immune response, or inappropriate cellular inflammatory response is mediated by the MKNK1 pathway, more particularly in which the disease of uncontrolled cell growth, proliferation and/or survival, inappropriate cellular immune response, or inappropriate cellular inflammatory response is a haematological tumour, a solid tumour and/or metastases thereof, e.g. leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof.

18. Intermediate compound of formula IV: 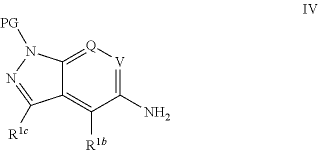
in which R^{1b}, R^{1c}, and Q-V are as defined in any one of claims 1 to 10 and PG represents a protective group or a hydrogen atom.

19. Use of a compound of formula II or IV as defined in claim 11 for the preparation of a compound of formula I as defined in any one of claims 1 to 10.
